(19) United States
(12) Patent Application Publication
(10) Pub. No.: US 2026/0048138 A1
(43) Pub. Date: Feb. 19, 2026

(54) NOVEL PI3K AND/OR PIKK INHIBITORS, ANTIBODY-DRUG CONJUGATES COMPRISING THE SAME AND USES THEREOF

(71) Applicant: HUTCHMED LIMITED, Shanghai (CN)

(72) Inventors: Weihan ZHANG, Shanghai (CN); Haibin YANG, Shanghai (CN); Wei-Guo SU, Shanghai (CN)

(21) Appl. No.: 19/374,186

(22) Filed: Oct. 30, 2025

Related U.S. Application Data
(63) Continuation of application No. PCT/CN2025/098520, filed on May 30, 2025.

(30) Foreign Application Priority Data
May 31, 2024 (CN) 202410705411.4
Oct. 30, 2024 (CN) 202411535539.7

(51) Int. Cl. A61K 47/68; A61K 31/437; A61P 35/00; C07K 16/32

(52) U.S. Cl. CPC A61K 47/6855; A61K 31/437; A61P 35/00; C07K 16/32; C07K 2317/92; C07K 2317/94

(57) ABSTRACT

The present invention relates to the field of biopharmaceuticals, in particular to novel PI3K and/or PIKK inhibitors, antibody-drug conjugates (ADCs) comprising the same, methods of preparing the same, and uses thereof.

### CROSS-REFERENCE TO RELATED APPLICATIONS

This application is a continuation of International Application No. PCT/CN2025/098520 filed May 30, 2025, which claims priority to Chinese Patent Application No. 202411535539.7 filed Oct. 30, 2024, and Chinese Patent Application No. 202410705411.4 filed May 31, 2024, the contents of each of which are hereby incorporated by reference in their entireties.

### REFERENCE TO A SEQUENCE LISTING

The contents of the electronic sequence listing (2025-10-30-HML-16-PCTC-SequenceListing.xml; Size: 58,182 bytes; and Date of Creation: Oct. 30, 2025) is herein incorporated by reference in its entirety.

### TECHNICAL FIELD

The present invention relates to the field of biopharmaceuticals, in particular to novel PI3K and/or PIKK inhibitors, antibody-drug conjugates (ADCs) comprising the same, methods of preparing the same, and uses thereof.

### BACKGROUND ART

Phosphatidylinositol kinases are a large class of kinases with phosphoinositol lipids as substrates, including several family subgroups such as phosphatidylinositol-3 kinases (PI3Ks) and phosphatidylinositol-3 kinase-related kinases (PIKKs) (Burke J E et al. Mol Cell. 2018; 71 (5): 653-673). PI3Ks can be divided into class I, class II, and class III PI3Ks in mammals. Among them, the class I PI3Ks are the most common and are more closely related to the occurrence and development of tumors, which can be further divided into class IA and class IB PI3Ks. The class IA PI3K consists of three genes encoding catalytic subunits (PIK3CA, PIK3CB, and PIK3CD) and three genes encoding regulatory subunits (PIK3R1, PIK3R2, and PIK3R3). The three catalytic subunit genes encode three highly homologous class IA catalytic isomers, respectively: p110α, p110β, and p110δ, while the three regulatory subunit genes produce 5 regulatory subunits collectively referred to as p85-type (p85α, p55α, p50α, p85β, and p55γ). The class IB PI3K is a heterodimer consisting of a p110γ catalytic subunit encoded by PIK3CG and a regulatory isomer p101 encoded by PIK3R5 or a p87 encoded by PIK3R6. The p110α and p110β are widely expressed, while the expressions of the p110δ and p110γ are generally restricted to leukocytes (Thorpe L M et al. 2015; 15 (1): 7-24). The activation of PI3Ks is initiated by the binding of extracellular growth factors, cytokines, and chemokines to a tyrosine kinase receptor (RTK) or a G protein coupled receptor (GPCR), which phosphorylates the regulatory subunits of PI3K and activates the catalytic subunit p110 of PI3K. PI3Ks activate the 3′ hydroxyl group of phosphorylated phosphatidylinositol lipids (PtdIns) to produce phosphatidylinositol 3,4,5-triphosphate (PIP3), and further activates the downstream effectors such as protein kinase B (AKT) and mammalian target protein of rapamycin (mTOR), which in turn control the physiological and cellular functions such as cell growth, proliferation, survival, movement, and metabolism. Phosphatase and tensin homolog (PTEN) inactivate the PI3K signaling pathway by dephosphorylating PIP3 (Millis S Z et al. JAMA Oncol. 2016; 2 (12): 1565-1573). PIKKs are a subgroup of phosphatidylinositol kinase family closely related to the kinase domain of PI3K protein family, which has family members including dysregulated telangiectasia mutant kinase (ATM), ATM- and Rad3-related kinase (ATR), DNA-dependent protein kinase catalytic subunit (DNA-PK), mammalian target of rapamycin (mTOR), etc. The family members of PIKKs are involved in sensing and repairing cellular DNA damages as well as regulating cell growth, proliferation, survival, and metabolism (Shaik A et al. 2020; 25 (8): 1510-1537).

The PI3K signaling pathway is one of the most common activation signals in many types of cancers. This pathway controls tumor cell growth, proliferation, survival, differentiation, protein synthesis, glucose metabolism, migration, and apoptosis. In cancers, the PI3K signaling pathway often leads to constitutive activation due to protein abnormalities in the signaling pathway. Common signal protein abnormalities include: activating mutation and/or amplification of the catalytic subunit α (PIK3CA); no expression, decreased expression or inactivating mutation of regulatory subunits, such as PIK3R1; no expression, decreased expression or inactivating mutation of PTENs; mutation and/or amplification of AKT; mutation and/or amplification of mTOR; activating mutation and/or amplification of the upstream RTKs (e.g., HER2, HER3, c-Met, FGFR1, FGFR2, FGFR3, IGF1R, RET, ROS1, ALK, etc.); activating mutation and/or amplification of Ras family proteins, and so forth. In pre-clinical animal models, these alterations are sufficient to induce tumorigenesis. Highly frequent abnormalities of the PI3K signaling pathway-related proteins and genes have been observed in various solid tumors, including but not limited to breast cancer, endometrial cancer, prostate cancer, anal cancer, hepatocellular cancer, colorectal cancer, renal cancer, bladder cancer, head and neck squamous cell carcinoma, gastric cancer, esophageal cancer, ovarian cancer, pancreatic cancer, non-small cell lung cancer, small cell lung cancer, sarcoma, thyroid cancer, and various squamous cell carcinomas, melanoma, and glioblastoma (Millis S Z et al. JAMA Oncol. 2016; 2 (12): 1565-1573).

The critical roles of PI3Ks in cancers make them become very attractive therapeutic targets, and various small molecule inhibitors of different types targeting the key components in this signaling pathway have entered clinical development, involving a range of patients with different cancers. However, currently only selective PI3Kα, AKT and mTOR inhibitors need to be in combination with other drugs for approval in the treatment of HR-positive, HER2-negative metastatic breast cancer. Moreover, pan-PI3K and PIKK targeting inhibitors should produce stronger anti-tumor activities because they can simultaneously inhibit various different signal proteins in the PI3K signaling pathway and can overcome the feedback activation produced by the selective PI3K inhibitors; it is possible to better inhibit DNA damage repair induced by radiotherapy or chemotherapy, induce tumor cell apoptosis, and is capable of further enhancing anti-tumor activity if PIKKs (such as ATM, ATR, DNA-PK, and mTOR) are simultaneously targeted. Nevertheless, pan-PI3K inhibitors, pan-PI3K and mTOR dual-target inhibitors, and pan-PI3K and PIKK targeting inhibitors progress slowly due to the relatively high clinical toxicity. If the clinical toxicity can be effectively controlled and the safety window can be improved in cancer treatment, it can produce better clinical benefits in the treatment of more types of cancers with abnormal activation of the PI3K signaling pathway. Currently, antibody-drug conjugates (ADCs) are considered to be the most effective technical means to achieve this goal. An antibody-conjugated drug is composed of an antibody, a payload, and a linker. A biologically active molecular payload (e.g., a pan-PI3K and PIKK targeting inhibitor) is covalently conjugated to an antibody via a linker; the antibody (such as monoclonal antibody) can specifically recognize a specific target on the surface of tumor cells, thereby targeting the ADC to the surface of cancer cells and enabling it to enter into cancer cells through endocytosis. The linker is then cleaved under the action of low pH values, lysosomal enzymatic reactions, etc. in the cells, and the payload is released, thereby achieving the effect of specifically killing cancer cells without damaging normal tissue cells, accordingly reducing the non-tumor tissue toxicity or systemic toxicity of the biologically active molecular payload. An ideal linker should ensure that the ADC drug maintains sufficient stability and integrity in the blood circulation, and upon reaching therapeutic targets (tumor cells), can efficiently release the payload (Chau C H et al. 2019; 394(10200): 793-804; Drago J Z et al. Nat Rev Clin Oncol. 2021; 18(6): 327-344).

Human epidermal growth factor receptor-2 (HER2) as an ADC produced by a tumor surface antigen has achieved positive clinical benefits in the clinic. High expression, amplification or mutation of HER2 is found in a variety of solid tumors, including but not limited to breast cancer, bladder cancer, esophageal cancer, gastric cancer, gallbladder cancer, extrahepatic bile duct cancer, cervical cancer, endometrial cancer, ovarian cancer, testicular cancer, colorectal cancer, pancreatic cancer, head and neck squamous cell carcinoma, and non-small cell lung cancer (Yan M et al. Cancer Metastasis Rev. 2015; 34 (1): 157-164). Aberrant expression of HER2 leads to the constitutive activation of the PI3K signaling pathway, promoting the progression of a variety of cancers. Studies have found that HER2 amplification or over-expression occurs in about 20% of breast cancers. HER2-targeting therapy, including the antibody drug trastuzumab, the ADC drug trastuzumab deruxtecan (DS-8201), the small molecule kinase inhibitor lapatinib, etc., has significantly improved the survival of the patients with HER2-positive (HER2+) breast cancer. However, the primary and acquired resistance to anti-HER2 therapies are common. PIK3CA gene activating mutation occurs in about 30% of HER2-positive breast cancers, and the PIK3CA activating mutations confer resistance to anti-HER2 therapies in vivo. But the PI3K inhibitors alone or in combination with the anti-HER2 therapies can well overcome such resistance (Cataldo M L et al. Front Oncol. 2023; 13: 1108242, published on Jul. 4, 2023).

Compared with the small molecule inhibitors, the ADCs loaded with pan-PI3K and PIKK targeting inhibitors conjugate the antibodies targeting tumor surface antigens with potent payloads through cleavable linkers, thereby achieving specifically killing cancer cells expressing such tumor surface antigens without damaging normal tissue cells, reducing the normal tissue toxicity and systematic toxicity of the payloads, and thus improving the safety window and enhancing anti-tumor activity. At present, no ADC loaded with pan-PI3K and PIKK targeting inhibitors has entered clinical research. The clinical medical needs have not been met obviously. The present invention is intended to develop novel antibody-conjugated drugs loaded with pan-PI3K and PIKK targeting inhibitors.

### SUMMARY OF THE INVENTION

In a first aspect, the present invention provides an antibody-drug conjugate having a structure represented by formula (I) or a pharmaceutically acceptable salt thereof, or a solvate, a racemic mixture, an enantiomer, a diastereomer or a tautomer thereof:

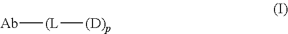

wherein
Ab is an antibody or an antigen-binding fragment thereof;
D is a PI3K and/or PIKK inhibitor;
L is a linker, which links Ab to D; and
p is an integer from 1 to 20.

In some embodiments, the PI3K and/or PIKK inhibitor has a structure represented

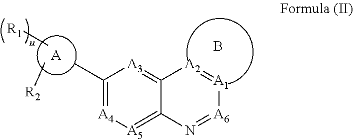

wherein
A₃, A₄, A₅, and A₆ are independently N or CR₅; R₅ is independently selected from: H, C₁₋₆ alkyl, C₃₋₉ cycloalkyl, —CN, —NH₂, —OH, —SH, —O(C₁₋₆ alkyl), halogen, or C₁₋₆ haloalkyl;

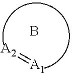

represents a 5- to 6-membered heteroaryl ring or a 3- to 8-membered heterocyclyl ring, and the 5- to 6-membered heteroaryl ring or 3- to 8-membered heterocyclyl ring is optionally substituted with one or more groups selected from oxo, ═NCN, or R₃;
ring A is 5- to 14-membered heteroaryl;
R₁, R₂, and R₃ are independently selected from: H, C₁₋₆ alkyl, C₂₋₈ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)R^{e}, —NR^{c}S(O)ₙNR^{f}R^{g}, —NR^{c}C(O)OR^{b}, —NR^{c}C(O)NR^{d}R^{e}, —NO₂, —OR^{b}, —SR^{b}, —S(O)ₙR^{e}, —S(O)ₙR^{c}R^{d}, halogen, C₁₋₆ haloalkyl, 5- to 6-membered heteroaryl, and 3- to 12-membered heterocyclyl;
R^{a}, R^{b}, R^{c}, R^{d}, R^{e}, R^{f}, and R^{g} are each independently selected from hydrogen, C₁₋₆ alkyl, C₂₋₈ alkenyl, C₂₋₈ alkynyl, C₆-C₁₀ aryl, C₃₋₉ cycloalkyl, C₁₋₆ haloalkyl, 5- to 6-membered heteroaryl, and 3- to 12-membered heterocyclyl;
or R^{a} and R^{c}, and/or R^{c} and R^{d}, and/or R^{c} and R^{e}, and/or R^{c} and R^{f}, and/or R^{d} and R^{e}, and/or R^{g} and R^{f} together with the atoms which they are attached to form a 3- to 12-membered heterocyclyl;
wherein the C₁₋₆ alkyl, C₂₋₈ alkenyl, C₂₋₈ alkynyl, C₁₋₆ haloalkyl, C₆-C₁₀ aryl, C₃₋₉ cycloalkyl, 5- to 6-membered heteroaryl, and 3- to 12-membered heterocyclyl, at each occurrence, can independently and optionally be substituted with one or more groups selected from: deuterium, C₁₋₆ alkyl, C₂₋₈ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)ₙR^{e}, —NR^{c}S(O)ₙNR^{f}R^{g}, —NR^{c}C(O)OR^{b}, —NR^{c}C(O)NR^{d}R^{e}, —NO₂, —C₁₋₆ alkylene-OR^{b}, —OR^{b}, —S(O)ₙR^{e}, —S(O)ₙR^{c}R^{d}, halogen, C₁₋₆ haloalkyl, 5- to 6-membered heteroaryl, and 3- to 8-membered heterocyclyl;
n, at each occurrence, is independently 0, 1, or 2;
u, at each occurrence, is independently 0, 1, 2, or 3; and
[symbol] represents a single bond or a double bond; and
wherein one of the R₁, R₂, or R₃, when present, is covalently linked to the L, and the valence of the atom at the linking site does not exceed the normal valence of the atom due to the one or more substituents to which it is linked.

In a second aspect, the present invention provides a linker-payload compound of formula (IV) or a pharmaceutically acceptable salt thereof, or a solvate, a racemic mixture, an enantiomer, a diastereomer or a tautomer thereof:

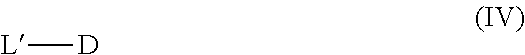

wherein L′ is a linker, which is used to link with Ab; and
D is a PI3K and/or PIKK inhibitor, the structure of the PI3K and/or PIKK inhibitor being as defined in the first aspect.

In a third aspect, the present invention provides a PI3K and/or PIKK inhibitor of formula (II-1) or a pharmaceutically acceptable salt thereof, or a solvate, a racemic mixture, an enantiomer, a diastereomer or a tautomer thereof,

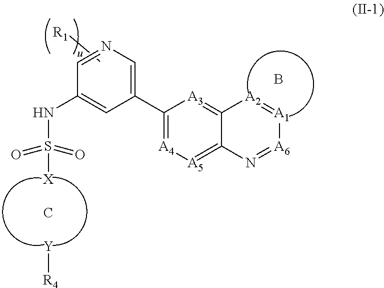

wherein the definitions of groups and symbols are as described in the detailed description section. The PI3K and/or PIKK inhibitor of formula (II-1) in the present invention can be used as a payload, which can be stably linked to a linker while maintaining drug properties, and exhibit good efficacy.

The present invention further provides a pharmaceutical composition comprising the antibody-drug conjugate, the linker-payload compound, or the PI3K and/or PIKK inhibitor and/or at least one pharmaceutically acceptable salt thereof, as described in the first, second, or third aspect of the invention, and at least one pharmaceutically acceptable carrier.

The present invention further provides a medicament for inhibiting the activity of PI3Ks and/or PIKKs comprising an effective amount of at least one linker-payload compound or PI3K and/or PIKK inhibitor and/or pharmaceutically acceptable salt thereof described in the invention. The present invention further provides a medicament for inhibiting the activity of PI3Ks and/or PIKKs comprising an effective amount of at least one antibody-drug conjugate and/or at least one pharmaceutically acceptable salt thereof described in the invention.

The present invention further provides the use of the PI3K and/or PIKK inhibitor and antibody-drug conjugate and/or at least one pharmaceutically acceptable salt thereof in the manufacture of a drug for the treatment of a disease or condition mediated at least in part by PI3K and/or PIKK. The disease or condition mediated at least in part by PI3K and/or PIKK is cancer or autoimmune disease. Preferably, the cancer is a cancer associated with the activity of the PI3K and/or PIKK kinase.

The present invention further provides a method of inhibiting the PI3K and/or PIKK activity comprising contacting an effective amount of the at least one antibody-drug conjugate, linker-payload compound or PI3K and/or PIKK inhibitor and/or at least one pharmaceutically acceptable salt thereof with the kinase. In another preferred embodiment, the method of inhibition is in vitro and non-therapeutic.

The present invention further provides a method of treating a disease or condition responsive to the inhibition of PI3K and/or PIKK, comprising administering an effective amount of the at least one antibody-drug conjugate, linker-payload compound, or PI3K and/or PIKK inhibitor and/or at least one pharmaceutically acceptable salt thereof to a subject in need of treatment of the disease or condition.

The present invention further provides a method of delivering a PI3K and/or PIKK inhibitor to a cell or tissue expressing a corresponding target (e.g., HER2, CD20, or CD38, CEACAM5/6, FRα, or Nectin-4), the method comprising conjugating the PI3K and/or PIKK inhibitor to an antibody that immunospecifically binds to the corresponding target (e.g., HER2, CD20, or CD38, CEACAM5/6, FRα, or Nectin-4) to obtain an antibody-conjugated drug (abbreviated as “ADC”), and exposing the cell or tissue to the ADC.

### DETAILED DESCRIPTION OF THE INVENTION

### Definitions

As used in the specification, the following words, phrases and symbols are generally intended to have the meanings as set forth below, except to the extent that the context in which they are used indicates otherwise.

A dash (“—”) that is not between two letters or symbols is used to indicate a point of attachment for a substituent. For example, —O(C₁₋₆ alkyl) refers to the attachment of C₁₋₆ alkyl to the rest of the molecule through an oxygen atom.

The term “alkyl” as used herein refers to a straight or branched saturated hydrocarbon radical containing 1-18 carbon atoms (C₁₋₁₈), preferably 1-10 carbon atoms (C₁₋₁₀), more preferably 1-6 carbon atoms (C₁₋₆), and even more preferably 1-4 carbon atoms (C₁₋₄) or 1-3 carbon atoms (C₁₋₃). When the term “alkyl” is prefixed with “C_{a-b}”, it means the number of carbon atoms in the alkyl, wherein a is the minimum number of carbon atoms in the alkyl and b is the maximum number of carbon atoms in the alkyl. For example, “C₁₋₆ alkyl” means an alkyl containing 1-6 carbon atoms. “C₁₋₃ alkyl” means an alkyl containing 1-3 carbon atoms. Examples of C₁₋₆ alkyl include, but are not limited to, methyl, ethyl, propyl (e.g., n-propyl, and i-propyl), butyl (e.g., n-butyl, iso-butyl, sec-butyl, and tert-butyl), pentyl (e.g., n-pentyl, iso-pentyl, and neo-pentyl), hexyl, and the like. When located between two dashes (“—”) (e.g., —(C₁₋₆ alkyl)-OH), the alkyl represents an alkylene.

The term “alkylene” as used herein refers to a divalent radical of an alkyl as defined above that is attached to the rest of the molecule by two single bonds on the same carbon atom or different carbon atoms, respectively. In one embodiment, the alkylene is an alkylene of 1-6 carbon atoms (C₁₋₆ alkylene). In another embodiment, the alkylene is C₁₋₃ alkylene, C₁₋₂ alkylene or C₁ alkylene. Examples of C₁₋₆ alkylene include, but are not limited to, methylene (—CH₂—), 1,2-ethylene (—CH₂CH₂—), 1,1-ethylene (—CH(CH₃)—), 1,3-propylene (—CH₂—CH₂—CH₂—), 1,1-propylene (—CH(CH₂CH₃)—), 2,2-propylene (—C(CH₃)₂—), 1,2-propylene (—CH(CH₃)CH₂—), 1,4-butylene (—CH₂—CH₂—CH₂—CH₂—), and the like.

The term “alkenyl” as used herein refers to a straight or branched unsaturated hydrocarbon radical containing one or more, for example 1, 2, or 3 carbon-carbon double bonds (C═C) and 2-18 carbon atoms (C₂₋₁₈), preferably 2-10 carbon atoms (C₂₋₁₀), more preferably 2-6 carbon atoms (C₂₋₆), and even more preferably 2-4 carbon atoms (C₂₋₄). When the term “alkenyl” is prefixed with “C_{a-b}”, it means the number of carbon atoms in the alkenyl, wherein a is the minimum number of carbon atoms in the alkenyl and b is the maximum number of carbon atoms in the alkenyl. For example, “C₂₋₆ alkenyl” means an alkenyl containing 2-6 carbon atoms. “C₂₋₄ alkenyl” means an alkenyl containing 2-4 carbon atoms. Examples of C₂₋₆ alkenyl include, but are not limited to, ethenyl, propenyl (e.g., 2-propenyl), and butenyl (e.g., 2-butenyl), and the like. The point of attachment for the alkenyl can be on or not on the double bonds.

The term “alkynyl” as used herein refers to a straight or branched unsaturated hydrocarbon radical containing one or more, for example 1, 2, or 3 carbon-carbon triple bonds (C≡C) and 2-18 carbon atoms (C₂₋₁₈), preferably 2-10 carbon atoms (C₂₋₁₀), more preferably 2-6 carbon atoms (C₂₋₆), and even more preferably 2-4 carbon atoms (C₂₋₄). When the term “alkynyl” is prefixed with “Cₐ-b”, it means the number of carbon atoms in the alkynyl, wherein a is the minimum number of carbon atoms in the alkynyl and b is the maximum number of carbon atoms in the alkynyl. For example, “C₂₋₆ alkynyl” means an alkynyl containing 2-6 carbon atoms. “C₂₋₄ alkynyl” means an alkynyl containing 2-4 carbon atoms. Examples of C₂₋₆ alkynyl include, but are not limited to, ethynyl, propynyl (e.g., 2-propynyl), and butynyl (e.g., 2-butynyl), and the like. The point of attachment for the alkynyl can be on or not on the triple bonds.

The term “halogen” or “halo” as used herein refers to fluoro, chloro, bromo, and iodo, preferably fluoro, chloro, and bromo, more preferably fluoro and chloro.

The term “haloalkyl” as used herein refers to an alkyl as defined herein, in which one or more, for example 1, 2, 3, 4, or 5, or all hydrogen atoms are replaced with halogen atoms, and when more than one hydrogen atoms are replaced by halogen atoms, the halogen atoms can be the same or different from each other. In one embodiment, the term “haloalkyl” as used herein refers to an alkyl as defined herein, in which two or more, for example 2, 3, 4, or 5, or all hydrogen atoms are replaced by halogen atoms, wherein the halogen atoms are identical to each other. In another embodiment, the term “haloalkyl” as used herein refers to an alkyl as defined herein, in which two or more hydrogen atoms, for example 2, 3, 4, or 5, or all hydrogen atoms are replaced by halogen atoms, wherein the halogen atoms are different from each other. When the term “haloalkyl” is prefixed with “C_{a-b}”, it means the number of carbon atoms in the haloalkyl, wherein a is the minimum number of carbon atoms in the haloalkyl and b is the maximum number of carbon atoms in the haloalkyl. For example, “C₁₋₆ haloalkyl” means a haloalkyl as defined herein containing 1-6 carbon atoms. “C₁₋₄ haloalkyl” means a haloalkyl as defined herein containing 1-4 carbon atoms. Examples of C₁₋₆ haloalkyl include, but are not limited to, —CF₃, —CHF₂, —CH₂F, —CH₂CH₂F, —CH₂CHF₂, —CH₂CF₃, —CH(CF₃)₂, and the like.

As used herein, the term “imino” or written as “═N—R” refers to an amino group as a divalent substituent, in which two valences of the same nitrogen atom are attached to an atom optionally selected from the rest of the compound to form a double bond, and the third valence of the nitrogen atom is attached to the R group as defined by the context. The nitrogen atom, when attached to a carbon atom, forms an imine, amidine, guanidine, etc., or when attached to a heteroatom, forms a sulfoxide imine, etc.

The term “cycloalkyl” as used herein refers to a saturated or partially unsaturated cyclic hydrocarbyl containing 3-12 ring carbon atoms (C₃₋₁₂) (e.g., 3-10 ring carbon atoms (C₃₋₁₀), 3-9 ring carbon atoms (C₃₋₉), 3-8 ring carbon atoms (C₃₋₈), 5-7 ring carbon atoms (C₅₋₇), 4-7 ring carbon atoms (C₄₋₇), or 3-6 ring carbon atoms (C₃₋₆)); it may have one or more rings, for example 1, 2, or 3 rings, preferably 1 or 2 rings. When the term “cycloalkyl” is prefixed with “C_{a-b}”, it means the number of carbon atoms in the cycloalkyl, wherein a is the minimum number of carbon atoms in the cycloalkyl and b is the maximum number of carbon atoms in the cycloalkyl. For example, “C₃₋₈ cycloalkyl” or “3- to 8-membered cycloalkyl” means a cycloalkyl having 3-8 ring carbon atoms; “C₃₋₆ cycloalkyl” or “3- to 6-membered cycloalkyl” means a cycloalkyl having 3-6 ring carbon atoms. The cycloalkyl can include a fused or bridged ring, or a spirocyclic ring. The rings of the cycloalkyl can be saturated, or can have one or more, for example, one or two double bonds (i.e., partially unsaturated), but not fully conjugated, and not an “aryl” as defined in the invention. Examples of cycloalkyl include, but are not limited to, cyclopropyl, cyclobutyl, cyclopentyl, cyclohexyl, spiro[2.2]pentyl, cyclopropenyl, cyclobutenyl, cyclopentenyl, cyclopentadienyl, cyclohexenyl, and the like.

The term “heterocyclyl” or “heterocycle” as used herein can be used interchangeably and each refers to a saturated or partially unsaturated ring having 3-14 ring atoms (e.g., 4-14 ring atoms (4- to 14-membered heterocyclyl), 4-12 ring atoms (4- to 12-membered heterocyclyl), 3-12 ring atoms (3- to 12-membered heterocyclyl), 4-10 ring atoms (4- to 10-membered heterocyclyl), 3-8 ring atoms (3- to 8-membered heterocyclyl), 4-8 ring atoms (4- to 8-membered heterocyclyl), 3-6 ring atoms (3- to 6-membered heterocyclyl), or 4-5 ring atoms (4- to 5-membered heterocyclyl)), wherein the ring atoms include one or more (e.g., 1, 2, or 3, preferably 1 or 2) heteroatoms independently selected from N, O, and S, and the remaining ring atoms are carbon atoms; it may have one or more rings, for example 1, 2, or 3 rings, preferably 1 or 2 rings. N and S can be optionally oxidized to various oxidation states. The point of attachment of heterocyclyl can be on the N heteroatom or carbon atoms. For example, “4- to 10-membered heterocyclyl” means a heterocyclyl having 4-10 (e.g., 4, 5, 6, 7, 8, 9, or 10) ring atoms comprising at least one, such as 1, 2, 3, or 4, preferably 1, 2, or 3 heteroatoms independently selected from N, O, and S; “3- to 8-membered heterocyclyl” means a heterocyclyl having 3-8 (e.g., 3, 4, 5, 6, 7, or 8) ring atoms comprising at least one, such as 1, 2, or 3, preferably 1 or 2 heteroatoms independently selected from N, O, and S; “3- to 6-membered heterocyclyl” means a heterocyclyl having 3-6 (e.g., 3, 4, 5 or 6) ring atoms comprising at least one, preferably 1 or 2 heteroatoms independently selected from N, O, and S (preferably N and O), which is preferably a monocyclic ring. The heterocyclyl can include a fused or bridged ring, or a spirocyclic ring. The rings of the heterocyclyl may be saturated or can have one or more, for example, one or two double bonds (i.e., partially unsaturated), but not fully conjugated and not a “heteroaryl” as defined in the invention. Examples of heterocyclyl include, but are not limited to: 3-12 membered heterocyclyl, 4- to 10-membered heterocyclyl, 3- to 8-membered heterocyclyl, 3- to 6-membered heterocyclyl, and 4- to 5-membered heterocyclyl, for example, oxetanyl, azetidinyl, pyrrolidinyl, tetrahydrofuryl, dioxolanyl, tetrahydropyranyl, morpholinyl, thiomorpholinyl, piperidinyl, piperazinyl, 1-methylpiperazinyl, 4-(2-hydroxyethyl)-piperazin-1-yl, tetrahydropyridinyl, dihydropyrimidinyl, dihydropyrazinyl, pyrazolidinyl, hexahydro-1H-pyrrolizinyl, hexahydrospiro[cyclopropane-pyrrolizinyl](e.g., tetrahydro-1′H,3′H-spiro[cyclopropane-1,2′-pyrrolizinyl], hexahydrospiro[cyclopropane-1,3′-pyrrolizinyl] and hexahydrospiro[cyclopropane-1,1′-pyrrolizinyl]), octahydrocyclopropeno[a]pyrrolidinyl and oxaspiro[3.3]heptanyl, 2,7-diazaspiro[3.5]nonanyl, octahydropyrrolo[3,4-c]pyrrolyl, 3,7-diazabicyclo[4.2.0]octanyl, 4,7-diazaspiro[2.5]octanyl, 2,6-diazaspiro[3.5]nonanyl, 2,7-diazaspiro[4.4]nonanyl, and 2,6-diazaspiro[3.4]octanyl, preferably oxetanyl, azetidinyl, pyrrolidinyl, tetrahydropyranyl, morpholinyl, piperidinyl, 2-(piperazin-1-yl)ethanolyl, piperazinyl, hexahydro-1H-pyrrolizinyl, tetrahydro-1′H,3′H-spiro[cyclopropane-1,2′-pyrrolizinyl], octahydrocyclopropeno[a]pyrrolizinyl, 2,7-diazaspiro[3.5]nonanyl, octahydropyrrolo[3,4-c]pyrrolyl, 3,7-diazabicyclo[4.2.0]octanyl, 4,7-diazaspiro[2.5]octanyl, 2,6-diazaspiro[3.5]nonanyl, 2,7-diazaspiro[4.4]nonanyl, and 2,6-diazaspiro[3.4]octanyl.

The terms “aryl” or “aromatic hydrocarbon” as used herein are used interchangeably to refer to a carbocyclic hydrocarbyl consisting of one ring or a plurality of, such as two, fused rings having 6-14 carbon atoms (e.g., 6-14 carbon atoms (6- to 14-membered aryl or C₆-C₁₄ aryl), 6-12 carbon atoms (6- to 12-membered aryl or C₆-C₁₂ aryl), 6-10 carbon atoms (6- to 10-membered aryl or C₆-C₁₀ aryl)), wherein at least one ring is an aromatic ring and the point of attachment to the rest of the molecule is on the aromatic ring. Examples of aryl include, but are not limited to, phenyl, naphthyl, 1,2,3,4-tetrahydronaphthyl, phenanthryl, indenyl, indanyl, azulenyl, and benzocyclobutenyl, preferably phenyl and naphthyl.

The terms “heteroaryl” and “heteroaryl ring” as used herein are used interchangeably to refer to a monocyclic, dicyclic or tricyclic ring system having 5-15 ring atoms (e.g., 5-14 ring atoms (5- to 14-membered heteroaryl), 5-13 ring atoms (5- to 13-membered heteroaryl), 5-12 ring atoms (5- to 12-membered heteroaryl), 5-6 ring atoms (5- to 6-membered heteroaryl), 8-13 ring atoms (8- to 13-membered heteroaryl), 9-12 ring atoms (9- to 12-membered heteroaryl)), wherein the ring atoms include one or more, such as 1, 2, 3, or 4, preferably 1, 2, or 3 heteroatoms independently selected from N, O, and S, and the remaining ring atoms are carbon atoms, wherein at least one ring is an aromatic ring and the point of attachment to the rest of the molecule is on the aromatic ring, and wherein the S and N heteroatoms can be optionally oxidized to various oxidation states. When the total number of S and O atoms in the heteroaryl exceeds 1, the S and O heteroatoms are not adjacent to one another. Preferably, the heteroaryl is 5- to 13-membered heteroaryl. For example, the heteroaryl includes:
5- to 6-membered monocyclic heteroaryl, i.e., a monocyclic aromatic heterocyclic group having 5 or 6 ring atoms, wherein the ring atoms include one or more, such as 1, 2, or 3 heteroatoms independently selected from N, O, and S (preferably N), and the remaining ring atoms are carbon atoms; and
8- to 13-membered bicyclic or tricyclic heteroaryl, i.e., a bicyclic or tricyclic aromatic heterocyclic group having 8, 9, 10, 11, 12, or 13 ring atoms, wherein the ring atoms include one or more, such as 1, 2, 3, or 4, preferably 1, 2, or 3 heteroatoms independently selected from N, O, and S (preferably N), and the remaining ring atoms are carbon atoms, wherein at least one ring is an aromatic ring.

Examples of heteroaryl include, but are not limited to: 5- to 6-membered monocyclic heteroaryl, for example, pyridinyl, N-oxide pyridinyl, pyrazinyl, pyrimidinyl, triazinyl (e.g., 1,2,4-triazinyl, 1,3,5-triazinyl), pyrazolyl, imidazolyl, oxazolyl, isoxazolyl, oxadiazolyl (e.g., 1,2,4-oxadiazolyl, 1,2,5-oxadiazolyl, and 1,3,4-oxadiazolyl), thiazolyl, isothiazolyl, thiadiazolyl, tetrazolyl, triazolyl, thienyl, furyl, pyranyl, pyrrolyl, and pyridazinyl, preferably triazolyl, pyridinyl, N-oxide pyridinyl, pyrazinyl, pyridazinyl, pyrimidinyl, pyrazolyl, imidazolyl, isoxazolyl, triazinyl (e.g., 1,2,4-triazinyl), oxazolyl, thiadiazolyl, and more preferably pyridinyl (e.g., pyridin-4-yl, pyridin-3-yl), pyrazinyl, pyridazinyl, and pyrimidinyl; and 8- to 13-membered bicyclic or tricyclic heteroaryl, for example benzoxazolyl, benzisoxazolyl, benzothienyl, benzothiazolyl, benzisothiazolyl, imidazopyrimidinyl (e.g., imidazo[1,2-c]pyrimidinyl), imidazopyrazinyl (e.g., imidazo[1,2-a]pyrazinyl and imidazo[1,5-a]pyrazinyl), imidazopyridinyl (e.g., imidazo[1,2-a]pyridinyl), imidazopyridazinyl (e.g., imidazo[1,2-b]pyridazinyl), pyrrolopyrazinyl (e.g., pyrrolo[1,2-a]pyrazinyl), pyrrolopyridinyl (e.g., 1H-pyrrolo[2,3-b]pyridinyl), pyrrolopyrimidinyl (e.g., pyrrolo[3,4-d]pyrimidinyl), pyrazolopyrazinyl (e.g., pyrazolo[1,5-a]pyrazinyl), pyrazolopyridinyl (e.g., 1H-pyrazolo[3,4-b]pyridinyl), pyrazolopyrimidinyl (e.g., pyrazolo[1,5-a]pyrimidinyl), triazolopyrimidinyl (e.g., [1,2,4]triazolo[4,3-c]pyrimidinyl and [1,2,4]triazolo[1,5-c]pyrimidinyl), triazolopyrazinyl (e.g., [1,2,4]triazolo[1,5-a]pyrazinyl), triazolopyridinyl (e.g., [1,2,4]triazolo[4,3-a]pyridinyl and [1,2,4]triazolo[1,5-a]pyridinyl), tetrazolopyridinyl (e.g., tetrazolo[1,5-a]pyridinyl), benzofuryl, benzimidazolyl, indolyl, indazolyl, purinyl, quinolinyl, tetrahydroquinolinyl (e.g., 1,2,3,4-tetrahydroquinolinyl), isoquinolinyl, tetrahydroisoquinolinyl (e.g., 5,6,7,8-tetrahydroisoquinolinyl), tetrahydronaphthyridinyl (e.g., 1,2,3,4-tetrahydro-2,7-naphthyridinyl), dihydro-cyclopentadienopyridinyl (e.g., 6,7-dihydro-5H-cyclopentadieno[c]pyridinyl), dihydro-pyrrolopyridinyl (e.g., 2,3-dihydro-1H-pyrrolo[3,4-c]pyridinyl), 6,7-dihydro-4H-pyrazolo[5,1-c][1,4]oxazinyl, 1,2,3,4-tetrahydro-1,5-naphthyridinyl, benzoindazolyl (e.g., 1H-benzo[f]indazolyl), tetrahydrocyclopentadienoindazolyl (e.g., 1,5,6,7-tetrahydrocyclopentadieno[f]indazolyl), dihydroindenothiazolyl (e.g., 6,7-dihydro-5H-indeno[5,6-d]thiazolyl), hexahydroindenoxazinyl (e.g., 2,3,4,6,7,8-hexahydroindeno[5,6-b][1,4]oxazinyl), tetrahydrocyclopentadienoindolyl (e.g., 1,5,6,7-tetrahydrocyclopentadieno[f]indolyl), tetrahydroindenoimidazolyl (e.g., 1,5,6,7-tetrahydroindeno[5,6-d]imidazolyl), preferably indazolyl, indolyl, benzimidazolyl, benzothiazolyl, quinolinyl, 1,2,3,4-tetrahydroquinolinyl, isoquinolinyl, 5,6,7,8-tetrahydroisoquinolinyl, 1,2,3,4-tetrahydro-2,7-naphthyridinyl, 6,7-dihydro-5H-cyclopentadieno[c]pyridinyl, 2,3-dihydro-1H-pyrrolo[3,4-c]pyridinyl, imidazolo[1,2-c]pyrimidinyl, imidazolo[1,2-a]pyridinyl, 1H-pyrrolo[2,3-b]pyridinyl, [1,2,4]triazolo[1,5-a]pyridinyl, 1,2,3,4-tetrahydro-1,5-naphthyridinyl, 1H-benzo[f]indazolyl, 1,5,6,7-tetrahydrocyclopentadieno[f]indazolyl, 6,7-dihydro-5H-indeno[5,6-d]thiazolyl, 2,3,4,6,7,8-hexahydroindeno[5,6-b][1,4]oxazinyl, 1,5,6,7-tetrahydrocyclopentadieno[f]indolyl), and 1,5,6,7-tetrahydroindeno[5,6-d]imidazolyl.

The term “—OH” as described herein refers to hydroxyl radical.

The term “—CN” as used herein refers to cyano radical.

The term “oxo” as used herein refers to ═O.

The term “optional” or “optionally” as used herein means that the subsequently described event or circumstance may or may not occur, and the description includes instances in which the event or circumstance occur and instances in which it does not occur. For example, “optionally substituted with one or more” includes unsubstituted and substituted with 1, 2, or more substituents as described. It should be appreciated by those skilled in the art that, with respect to any radical containing one or more substituents, the radical is not intended to introduce any substitution pattern or patterns that are sterically impractical, chemically incorrect, synthetically non-feasible and/or inherently unstable.

The term “substituted” or “substituted with . . . ”, as used herein, means that one or more (such as, 1, 2, 3, or 4) hydrogens on the designated atom or group are replaced by one or more (such as 1, 2, 3, or 4) substituents, preferably the substituents selected from the indicated group of substituents or radicals, provided that the designated atom's normal valence is not exceeded. The substituents can be the same or different from each other. The term “substituted with one or more radicals selected from” or “substituted with one or more” as used herein means that one or more hydrogens on the designated atom or radical are independently replaced by one or more radicals selected from the indicated group of substituents or radicals, wherein the radicals can be the same or different from each other. Preferably, “substituted with one or more radicals selected from” or “substituted with one or more” means that the designated atom or radical is substituted with 1, 2, 3, or 4 radicals independently selected from the indicated group of substituents or radicals, wherein the radicals can be the same or different from each other. In some embodiments, when a substituent is oxo (i.e., ═O) or ═NR₃, then two hydrogens on a single atom are replaced. An optional substituent can be any radicals, provided that combinations of substituents and/or variables result in a chemically correct and stable compound. A chemically correct and stable compound is meant to imply a compound that is sufficiently stable so as to be able to survive the isolation from a reaction mixture. Preferably, substituents are those exemplified in the compounds of the examples of the present application.

Unless otherwise specified, substituents are named into the core structure. For example, it should be understood that when (cycloalkyl)alkyl is listed as a possible substituent, the point of attachment of this substituent to the core structure is in the alkyl portion.

A “pharmaceutically acceptable salt” refers to a free acid or base salt of a drug conjugate represented by formula (I), a linker-payload compound represented by formula (IV), or a compound represented by formula (II-1), that is non-toxic, biologically tolerable, or otherwise biologically suitable for administration to a subject for treatment or prophylaxis. For example, acid addition salts include, for example, addition salts derived from inorganic acids and organic acids. For a general description of pharmaceutically acceptable salts, see, for example: S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

Furthermore, if a compound, a drug conjugate, or a linker-payload described herein is obtained in the form of an acid addition salt, its free base form can be obtained by basifying a solution of the acid addition salt. Conversely, if the product is in the form of a free base, an acid addition salt, particularly a pharmaceutically acceptable acid addition salt, can be produced by dissolving the free base in a suitable solvent and treating the solution with an acid, in accordance with conventional procedures for preparing acid addition salts from base compounds. A person skilled in the art will recognize various synthetic methodologies that can be used without under experimentation to prepare non-toxic pharmaceutically acceptable acid addition salts or base addition salts.

The term “solvates” means solvent addition forms that contain either stoichiometric or non-stoichiometric amounts of solvent. Some compounds or drug conjugates have a tendency to trap a fixed molar ratio of solvent molecules in the solid state, thereby forming solvates. If the solvent is water, the solvate formed is a hydrate. When the solvent is alcohol, the solvate formed is an alcoholate. Hydrates are formed by the combination of one or more molecules of water, or less than one molecule of water, with one molecule of the substances in which the water retains its molecular state as H₂O, such combination being able to form one or more hydrates, for example, hemihydrate, monohydrate, and dihydrate.

The racemic mixture can be used as such or can be resolved into their individual isomers. The resolution can afford stereo-chemically pure compounds or mixtures enriched in one or more isomers. Methods for separation of isomers are well known (see, Allinger N. L. and Eliel E. L. in “Topics in Stereochemistry”, Vol. 6, Wiley Interscience, 1971) and include physical methods such as chromatography using chiral adsorbents. Individual isomers can be prepared in chiral forms from chiral precursors. Alternatively, individual isomers can be separated chemically from a mixture by: forming diastereomeric salts with a chiral acid (e.g., the individual enantiomers of 10-camphorsulfonic acid, camphoric acid, alpha-bromocamphoric acid, tartaric acid, diacetyltartaric acid, malic acid, pyrrolidone-5-carboxylic acid, and the like), fractionally crystallizing the salts, and then freeing one or both of the resolved bases, optionally repeating this process, so as to obtain either isomer or both isomers substantially free of the other; i.e., an isomer having an optical purity of >95%. Alternatively, the racemates can be covalently attached to a chiral compound (auxiliary) to produce diastereomers which can be separated by chromatography or by fractional crystallization after which time the chiral auxiliary is chemically removed to afford the pure enantiomers.

The term “stereoisomers” as used herein refers to compounds that have the same chemical constitution but differ in the arrangement of atoms or groups in space. Stereoisomers include enantiomers, diastereomers, and the like.

The terms “enantiomers” and “enantiomeric forms” as used herein can be used interchangeably and refer to two stereoisomers of a compound that are non-superimposable mirror images of each other.

The terms “diastereomers” and “diastereomeric forms” as used herein can be used interchangeably and refer to stereoisomers that have two or more chiral centers and are not mirror images of each other. Diastereomers have different physical properties, such as melting points, boiling points, spectral properties, or biological activities. A mixture of diastereomers can be separated by high-resolution analytical methods such as electrophoresis and chromatography such as HPLC.

The term “tautomers” as used herein refer to constitutional isomers of compounds generated by rapid movement of a certain atom in two positions in a molecule. Tautomers readily interconvert into each other, e.g., the enol form and the ketone form are typical tautomers.

The compounds of the present invention also encompass isotopically labeled compounds in which one or more atoms are replaced by atoms having an atomic mass or mass number different from those commonly found in nature. All isotopes of any particular atom or element specified, and uses thereof, are contemplated herein. Exemplary isotopes that can be incorporated into the compounds of the present invention include isotopes of hydrogen, carbon, nitrogen, oxygen, phosphorus, sulfur, fluorine, chlorine, and iodine, for example, ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I. Certain isotopically labeled compounds of the invention (e.g., those labeled with ³H and ¹⁴C) can be used for the studies on the compound and/or tissue distribution of an organism. Tritium (i.e., ³H) and carbon-14 (i.e., ¹⁴C) isotopes are particularly useful for this purpose because they are easy to prepare and detectable. Furthermore, substitution with heavier isotopes such as deuterium (i.e., ²H) may provide some therapeutic advantages due to higher metabolic stability (e.g., increased half-life in vivo or reduced dosage required) and is therefore preferred in some cases. It should be appreciated that the hydrogen (¹H) atom present in the compounds described herein can be replaced by deuterium (²H) atom to afford the deuterates. In any given compound, any number of hydrogen atoms can be replaced by the same number of deuterium atoms. Isotopically labeled compounds can typically be prepared by conventional techniques known to those skilled in the art or by procedures analogous to those described in the examples described below using appropriate isotopically labeled reagents instead of non-labeled reagents.

As used herein, the terms “group(s)” and “radical(s)” are synonymous and are intended to indicate the functional groups or molecular fragments attachable to other molecular fragments.

The term “active ingredient” is intended to indicate a chemical substance which has a biological activity. In some embodiments, an “active ingredient” is a chemical substance having a pharmaceutical utility.

The term “pharmaceutical combination” as used herein means a product obtained by mixing or combining two or more active ingredients, including fixed and non-fixed combinations of active ingredients, such as a kit and a pharmaceutical composition. The term “fixed combination” means that two or more active ingredients (e.g., compounds, drug conjugates or linker-payloads of the present invention and additional therapeutic agents) are simultaneously administered to a patient in the form of a single entity or dose. The term “non-fixed combination” means that two or more active ingredients (e.g., compounds, drug conjugates, or linker-payloads of the present invention and additional therapeutic agents) are administered simultaneously, concurrently or successively to a patient in separate entities, wherein the administration provides the patient with a therapeutically effective level of the compounds, drug conjugates, or linker-payloads.

The terms “treating” or “treatment” or “prevention” of a disease or disorder refer to administering one or more pharmaceutical substances, especially a compound, a drug conjugate or a linker-payload of the invention to a subject that suffers from or has a symptom of or has a predisposition toward the disease or disorder, with the purpose to cure, heal, alleviate, relieve, alter, remedy, ameliorate, improve, or affect the disease or disorder, the symptoms of the disease or disorder, or the predisposition toward the disease or disorder. In some embodiments, the disease or disorder is a cancer, such as a solid tumor or hematologic malignancy.

The terms “treating”, “contacting” and “reacting,” in the context of a chemical reaction, mean adding or mixing two or more reagents under appropriate conditions to produce the indicated and/or desired product. It should be appreciated that the reaction which produces the indicated and/or desired product may not necessarily result directly from the combination of two reagents which were initially added, i.e., there can be one or more intermediates which are produced in the mixture and ultimately lead to the formation of the indicated and/or desired product.

The term “effective amount” refers to an amount or dose of an PI3K and/or PIKK inhibitor sufficient to generally bring about therapeutic benefits in patients in need of treatment for a disease or disorder involving PI3Ks and/or PIKKs. An effective amount or dose of an active ingredient in the present invention can be determined by conventional methods, such as modeling, dose escalation studies, or clinical trials, in conjunction with conventional influencing factors, such as the mode or route of dosing or administration, the pharmacokinetics of the pharmaceutical ingredients, the severity and course of the disease or disorder, the prior or ongoing therapies for the subject, the subject's health status and response to the drug, and the judgment of the attending physician. In the case of cancer, the effective amount can cause any one of the visible or detectable changes of the subject as described above with regard to the definition of “treating” or “treatment” or “preventing”. For example, an effective amount is capable of reducing the number of cancer or tumor cells; reducing the size of tumors; inhibiting or preventing the invasion of tumor cells into peripheral organs, for example, the spread of tumors into soft tissues or bone; inhibiting or preventing the metastasis of tumors; inhibiting or preventing the growth of tumors; alleviating one or more symptoms associated with the cancer to some extent; reducing morbidity and mortality; improving quality of life; or a combination of the above effects. An effective amount can be a dosage that reduces the symptoms of a disease by inhibiting the PI3K and/or PIKK activity. For the treatment of cancers, the effects of in vivo experiments can be measured by assessments such as survival, time to progression (TTP), response rate (RR), response duration, and/or quality of life. Those skilled in the art have realized that the effective amount can be varied depending on the route of administration, the dosage of excipients, and the combination with other drugs.

The term “inhibit”, “inhibiting” or “inhibition” refers to a decrease in the baseline activity of a biological activity or process. The term “inhibiting the PI3K and/or PIKK activity” is an actual pharmaceutical activity for the purposes of the present invention, which means that direct or indirect responses to the presence of a compound, a drug conjugate, or a linker-payload of the present invention result in a decreased PI3K and/or PIKK activity relative to the PI3K and/or PIKK activity in the absence of the compound, the drug conjugate, or the linker-payload of the present invention. The decrease in activity may be caused by the direct interaction of a compound, a drug conjugate, or a linker-payload of the present invention with the PI3Ks and/or PIKKs, or by the interaction of a compound, a drug conjugate, or a linker-payload of the present invention with one or more other factors which ultimately affects the PI3K and/or PIKK activity. For example, a compound, a drug conjugate, or a linker-payload of the present invention may reduce the PI3K and/or PIKK activity by directly binding to the PI3Ks and/or PIKKs, may directly or indirectly affect another factor to reduce the PI3K and/or PIKK activity, or reduce the PI3K and/or PIKK activity by directly or indirectly reducing the amount of the PI3Ks and/or PIKKs present in cells or an organism.

In general, the term “about” is used herein to modify a numerical value above or below the stated value by a variance of 20%.

Technical and scientific terms as used and not specifically defined herein have the meaning commonly understood by those skilled in the art to which the present invention belongs.

All numerical ranges herein shall be interpreted as disclosing each numerical value and subset of numerical values within the range, regardless of whether they are specifically otherwise disclosed. For example, when referring to any numerical range, it should be regarded as referring to every numerical value within the numerical range, for example, every integer within the numerical range. For example, C₁₋₆ as used herein represents the inclusion of 1, 2, 3, 4, 5, or 6 C. The present invention relates to all values falling within the ranges, all smaller ranges and the upper or lower limits of the numerical range.

In the present invention, unless otherwise specified, the laboratory operation steps for cell culture, molecular genetics, nucleic acid chemistry, and immunology used herein are all conventional steps widely used in the corresponding fields.

The term “amino acid residue” refers to an amino acid unit in a polypeptide, that is, the part remained after dehydration of an amino acid linked by a peptide bond. The abbreviation for an amino acid residue is a standard three-letter and/or one-letter code used in the art to refer to one of the commonly used amino acids. For example, the amino acids forming amino acid residues can be selected from phenylalanine (Phe), tyrosine (Tyr), leucine (Leu), glycine (Gly), alanine (Ala), valine (Val), lysine (Lys), citrulline (Cit), serine (Ser), glutamic acid (Glu), aspartic acid (Asp), asparagine (Asn), isoleucine (Ile), arginine (Arg), proline (Pro), glutamine (Gln), etc. The amino acids can be L- or D-isomers, preferably L-isomers.

Unless explicitly stated otherwise, singular forms used in the present application (including the claims) include the corresponding plural forms thereof.

When used to connect two or more optional items, the term “and/or” should be understood to mean any one of the optional items or any two or more of the optional items.

The term “comprising” or “including” as used in the present invention means to include the mentioned elements, integers, or steps, but does not exclude any other elements, integers, or steps. As used herein, when the term “comprising” or “including” is used, unless otherwise stated, examples consisting of the mentioned elements, integers, or steps are also encompassed. For example, when referring to an antibody variable region “comprising” a specific sequence, it is also intended to encompass an antibody variable region composed of the specific sequence.

The term “subject” or “patient” as used herein means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and so on. The term “subject” or “patient” does not denote a particular age or sex. In some embodiments, the subject or patient is a human.

In the present invention, the term “conjugate” refers to a substance obtained by linking a payload or active molecule to a targeting moiety. In some embodiments of the present invention, the payload or active molecule is linked to the targeting moiety via a linker. Preferably, the linker can be cleaved under specific conditions (e.g., a low pH environment in a cell) or under specific actions (e.g., the action of a lysosomal protease), thereby release the active molecule from the targeting moiety. In some embodiments of the present invention, the linker comprises a cleavable unit, such as a peptide or a disulfide bond.

The term “stretcher group” is a part of the linker, which is used to link the target moiety with the remaining part of the linker.

The “bridged spacer group” described herein refers to a moiety that helps separate a part of the linker from other moieties described in the present invention (such as separate a stretcher group from Lp group). In some embodiments, the bridged spacer can lead a change in the properties of payload compound described in the present invention. In some embodiments, the bridged spacer group can modulate the hydrophilicity of the conjugate-payload compound, and provide improved overall antibody-drug conjugate properties.

In some embodiments, the payload or active molecule is an active drug molecule. In the present invention, the term “active drug molecule” refers to a substance that can regulate biological processes, especially change or prevent pathological processes, particularly refers to a substance that inhibits or prevents cell functions and/or causes cell death or destruction. In some embodiments of the present invention, the bioactive substance or active drug molecule in a conjugate is a molecule with anti-tumor biological activity, in particular a PI3K and/or PIKK inhibitor, more preferably a PI3K and/or PIKK inhibitor as defined in the present invention.

In the present invention, the term “linker” refers to a fragment that links an active drug molecule to a targeting moiety. It should be understood that a linker, prior to linking to a targeting moiety, has a functional group that can form a bond with a functional group of the targeting moiety. Non-limiting examples of linkers include those involved in the embodiments and examples of the present invention.

In the present invention, when a targeting moiety in a conjugate is an antibody or an antigen-binding fragment thereof, the conjugate is referred to as an “immunoconjugate”.

In the present invention, when an active molecule in an immunoconjugate is an active drug molecule, the immunoconjugate can be referred to as an “antibody-drug conjugate (ADC)”. In the present invention, the “drug conjugate” and “antibody-drug conjugate” can be used interchangeably.

The term “linker-payload” refers to a compound formed by attaching a payload, such as an active drug molecule (e.g., a small molecule drug), to a linker.

The term “targeting moiety” refers to a moiety of a conjugate that can specifically bind to a target (or a portion of the target) on the surface of a cell. Through the interaction of the targeting moiety with the target, the conjugate can be delivered to a specific cell population. Non-limiting examples of cell surface targets include ACTA2, ADGRE1, AGS-16, AIF1, AKR1C1, AKR1C2, ANGPTL4, ApoE, ASLG659, BAFF-R, BCMA, BMPR1B, BNIP3, Brevican, C1QA, C1QB, CA6, CADM1, CCL5, CCR5, CD11b, CD11c, CD123, CD138, CD19, CD19, CD20, CD21, CD22, CD223, CD30, CD33, CD37, CD38, CD40, CD45 (PTPRC), CD49D (ITGA4), CD56, CD66e, CD70, CD72, CD74, CD79a, CD79b, CD79b, CD80, CDCP1, CDH11, COL6A3, COL7A1, CRIPTO, CSF1R, CTGF, CTSD, CTSS, CXCL10, CXCL11, CXCR5, DDIT4, DLL3, DLL4, DR5, E16, EFNA4, EGLN, EGLN3, EMR2, endothelin receptor, ENPP3, EpCAM, EphA5, EphB2R, ETBR, FcRH1, FcRH2, FGF2, FGFR2, fibronectin extra domain B, folate receptor1, FR alpha, GEDA, GPNMB, guanylyl cyclase C, GZMB, HLA-DOB, HMOX1, IF16, IFNG, IGF-1R, IGFBP3, IL10RA1, IL20Rα, IL-6, IRTA2, KISS1R, KRT33A, LIV1, LOX, LRRC15, LUM, LY64, LY6E, Ly86, LYPD3, MCPT8, MDP, mesothelin, MFI2, MMP10, MMP14, MMP16, MPF, MS4A7, MSG783, Mucin-1, Mucin16, NaPi2b, Napi3b, NCA, Nectin-4, NOG, P2X5, pCAD, P-cadherin, PDGFRA, PDK1, PFKFB3, PGF, PGK1, PIK3AP1, PIK3CD, PLOD2, PSCA, PSCA hlg, PSMA, RNF43, ROR1, Sema5b, SERPINE1, SLC39A6, SLITRK6, SLTRK6, sodium phosphate cotransporter 2B, STAT1, STC2, STEAP1, STEAP2, TCF4, TENB2, TGF, TGFB1, TGFB2, TGFBR1, TNFRSF21, TNFSF9, Trf1, trophoblast glycoprotein, TrpM4, Tyro7, UPK1B, VEGFA, WNT5A, integrins α5β6 and α4β7, 0772P, and 5T4.

The term “DAR” refers to the ratio of a drug moiety (D) to a targeting moiety (e.g., an Ab moiety, preferably an antibody) described herein, wherein the drug moiety is conjugated to the targeting moiety (e.g., the Ab moiety, preferably an antibody). In some embodiments described herein, the DAR can be determined by p in formula (I), for example, the DAR can be 1 to 16, for example, 2-16, 4-16, 5-12, 6-10, 2-8, 3-8, 2-6, 4-6, or 6-10, such as about 2, about 3, about 4, about 5, about 6, about 7, about 8, about 9, about 10, about 11, about 12, about 13, about 14, or about 15. The DAR can also be calculated as an average DAR of a molecule population in a product, i.e., the overall ratio of a drug moiety (D) to an Ab moiety described herein in a product as measured by a detection method (e.g., by conventional methods such as mass spectrometry and/or HIC-HPLC), wherein the drug moiety is conjugated to the Ab moiety; and the DAR is referred to herein as an average DAR. In some embodiments, the conjugate of the present invention has an average DAR value of 1-16, such as 2-16, 4-16, 5-12, 6-10, 2-8, 3-8, 2-6, 4-6, or 6-10, such as 1.0-8.0 or 2.0-6.0, such as about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9 or about 10.0, and any range with any two of these values as endpoints. It should be understood that when referring to the average DAR value, the ADC of the present invention refers to an ADC molecule population or an ADC molecule mixture, containing ADC molecules with the same and/or different DARs.

As used herein, the “pharmaceutically acceptable” and “pharmaceutical” can be used interchangeably unless there is any contradiction in the context.

As used herein, the term “cancer” refers to a cellular disorder characterized by uncontrolled or dysregulated cell proliferation, decreased cellular differentiation, inappropriate invasion of surrounding tissues, and/or ability to establish new growth foci at other sites. The term “cancer” includes, but is not limited to, solid tumors and hematologic malignancies, such as leukemia, lymphoma, or myeloma. The term “cancer” includes cancers of skin, tissues, organs, bones, cartilage, blood, and vessels. The term “cancer” further includes primary cancer, and metastatic cancer, recurrent cancer, and refractory cancer.

The term “pharmaceutical composition” refers to a composition that exists in a form that allows the biological activity of the active ingredient(s) contained therein to be effective, and does not contain additional components that have unacceptable toxicity to the subject to whom the composition is administered. In addition to the active ingredient, e.g., ADCs, it generally contains components such as pharmaceutically acceptable carriers, stabilizers, diluents, dispersants, suspending agents, thickeners, and/or excipients. The pharmaceutical compositions provided herein are in a form that permits administration and subsequently provides the desired biological activity of active ingredient(s) and/or achieves therapeutic effects.

The terms “pharmaceutically acceptable carrier” and “physiologically acceptable carrier” refer to carriers or diluents that do not cause significant irritation to a subject and does not abrogate the biological activity and properties of the administered active ingredients such as ADC compounds or compositions and/or any additional therapeutic agents in the compositions. The pharmaceutically acceptable carrier can enhance or stabilize the composition, or can be used to facilitate the preparation of the composition.

The term “antibody” refers to an antibody in any form having a desired bioactivity. Therefore, it is used in the broadest sense, specifically including but not limited to a monoclonal antibody (including a full-length monoclonal antibody), a polyclonal antibody, a multispecific antibody (such as a bispecific antibody), a humanized antibody, a human antibody, a chimeric antibody, a CrossMab antibody, or a camelized single-domain antibody.

The term “monoclonal antibody” refers to an antibody obtained from a population of substantially homogeneous antibodies, i.e., the various antibodies constituting the population are identical except for possible naturally occurring mutations that may be present in minor amounts. The modifier “monoclonal” indicates the feature of the antibody as being obtained from a substantially homogeneous population of antibodies, and is not to be constructed as requiring any particular method to produce the antibody.

The term “isolated antibody or antigen-binding fragment” refers to the purified state of the antibody or antigen-binding fragment. For example, “isolated” may mean that the molecule is substantially free of other biomolecules, such as nucleic acids, proteins, lipids, sugars or other substances, such as cell debris and growth medium. However, as is known to a person skilled in the art, the term “isolated” does not mean the complete absence of such substances unless they are present in an amount that significantly interferes with the experimental or therapeutic application of the antibodies described herein. In some embodiments, the isolated antibody or antigen-binding fragment can have a purity of greater than 95%, greater than 96%, greater than 97%, greater than 98%, or greater than 99%, which purity is determined by, for example, electrophoresis (e.g., SDS-PAGE, isoelectric focusing (IEF), or capillary electrophoresis) or chromatography (e.g., ion exchange or reverse phase HPLC). For a review of methods of evaluating antibody purity, see, for example, Flatman, S. et al., J. Chrom. B 848 (2007) 79-87.

The terms “full-length antibody” and “intact antibody” can be used interchangeably herein and refer to an antibody that has a structure substantially similar to a native antibody structure. “Native antibodies” refer to naturally occurring immunoglobulin molecules with varied structures. For example, native IgG class antibodies are heterotetrameric glycoproteins of approximately 150,000 Dalton's, composed of two light chains and two heavy chains interconnected by disulfide bonds. Each of the heavy chains is comprised of a heavy chain variable region (abbreviated herein as VH) and a heavy chain constant region. The heavy chain constant region consists of 3 domains (CH1, CH2, and CH3). Each of the light chains is comprised of a light chain variable region (abbreviated herein as VL) and a light chain constant region. The light chain constant region consists of the domain CL. The VH and VL regions can be further divided into hypervariable regions (complementarity determining regions or CDRs) interspersed with regions that are more conserved (framework regions or FRs). Each VH or VL consists of three CDRs and four FRs, arranged in the following order from N-terminus to C-terminus: FR1, CDR1, FR2, CDR2, FR3, CDR3, and FR4. The constant region is not directly involved in the binding of an antibody to an antigen, but shows multiple effector functions. The heavy chain of an antibody can be classified into one of five types, i.e., α (IgA), δ (IgD), ε (IgE), γ (IgG), or μ (IgM), some of which can be further divided into subtypes, such as γ1 (IgG1), γ2 (IgG2), γ3 (IgG3), γ4 (IgG4), α1 (IgA1), and α2 (IgA2). The light chains of the antibody can be classified into one of two types, called κ (kappa) and λ (lambda), based on the amino acid sequences of their constant regions.

A “complementary determining region” or “CDR region” or “CDR” is a region in an antibody variable region, which is hypervariable in sequence and forms a structurally established loop (“hypervariable loop”) and/or contains an antigen contact residue (“antigen contact point”). CDR is mainly responsible for binding to epitopes. CDRs of heavy chain and light chain are generally called CDR1, CDR2, and CDR3, which are numbered sequentially from the N-terminus. The CDRs located in the antibody heavy chain variable region are called HCDR1, HCDR2, and HCDR3, respectively, while the CDRs located in the antibody light chain variable region are called LCDR1, LCDR2, and LCDR3, respectively. Each VH or VL consists of three CDRs and 4 FRs, which are arranged in the following order from the amino terminus to the carboxyl terminus: FR1, CDR1, FR2, CDR2, FR3, CDR3, and FR4. In a designated VH or VL amino acid sequence, the exact amino acid sequence boundary of each CDR can be determined by using any one of or a combination of various well-known schemes, including, for example: Chothia scheme (Chothia et al., Canonical structures for the hypervariable regions of immunoglobulins, Journal of Molecular Biology, 196, 901-917 (1987)); Kabat scheme (Kabat et al., Sequences of Proteins of Immunological Interest, 4th edition, U.S. Department of Health and Human Services, National Institutes of Health (1987)), AbM (University of Bath) and Contact (University College London); North scheme (North et al., A New Clustering of Antibody CDR Loop Conformations, Journal of Molecular Biology, 406, 228-256 (2011)). The boundary of the CDRs of the antibody in the present invention can be determined according to any schemes or a combination thereof in the art and personal evaluation. In some embodiments, the CDRs of the antibody of the present invention can be determined according to the Kabat scheme.

The term “antibody fragment” refers to a molecule other than a full antibody, which contains a portion of the full antibody that binds to the antigen that antigen bound by the full antibody. Examples of an antibody fragment include, but are not limited to, Fv, such as a disulfide-stabilized Fv fragment (dsFv), (dsFv)₂, bispecific dsFv (dsFv-dsFv′), Fab, Fab′, Fab′-SH, and F(ab′)₂; a diabody such as a disulfide-stabilized diabody (ds diabody), a triabody, a tetrabody, and a crossFab fragment; a linear antibody; a single-chain antibody molecule (e.g., scFv) and an scFv dimer (bivalent diabody); and a single-domain antibody. With regard to a review of certain antibody fragments, reference is made to Hudson et al., Nat Med 9, 129-134 (2003). Antibody fragments can be prepared by a variety of techniques, including but not limited to proteolytic digestion of full antibodies and production by recombinant host cells (e.g., Escherichia coli or phage).

The term “antigen-binding fragment” includes a fragment of an antibody that binds the antigen. Generally, the antigen-binding fragment includes at least one fragment (such as one or more CDRs) of the antigen-binding region or variable region of the antibody, and maintains at least some of the binding properties of the antibody. Examples of an antigen-binding fragment include, but are not limited to, Fv, such as a disulfide-stabilized Fv fragment (dsFv), (dsFv)₂, bispecific dsFv (dsFv-dsFv′), Fab, Fab′, Fab′-SH, and F(ab′)₂; a diabody such as a disulfide-stabilized diabody (ds diabody), a triabody, a tetrabody, and a crossFab fragment; a linear antibody; a single-chain antibody molecule (e.g., scFv) and an scFv dimer (bivalent diabody); a single-domain antibody; a nanobody and a multispecific antibody. When the antigen binding activity is expressed on a molar concentration basis, the antigen binding fragments generally maintain at least 10% of the antigen binding activity of the antibody from which they are derived. Preferably, the antigen-binding fragments maintain at least 20%, 50%, 70%, 80%, 90%, 95%, or 100% or more of the antigen binding activity of the antibody from which they are derived.

The term “biosimilar” refers to a biological product that is highly similar to an approved reference biological product (Reference Biologic) in terms of quality, safety, and efficacy, and has no clinically meaningful differences. For example, a “biosimilar of rituximab” is a monoclonal antibody that is highly similar to rituximab in terms of quality, safety, and efficacy, and has no clinically meaningful differences. In some embodiments, the biosimilar has the same heavy and light chain amino acid sequences as the antibody, but differs in post-translational modifications such as glycosylation.

### BRIEF DESCRIPTION OF THE DRAWINGS

FIG. 1: Effects of ADC-1 and ADC-6 on the growth of HCC1954 subcutaneous xenograft model.

### SPECIFIC EMBODIMENTS

### Embodiment I

Embodiment I-1. A drug conjugate having a structure represented by formula (I):

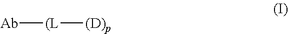

or a pharmaceutically acceptable salt thereof, or a solvate, a racemic mixture, an enantiomer, a diastereomer or a tautomer thereof, wherein
Ab is an antibody or an antigen-binding fragment thereof;
D is a PI3K and/or PIKK inhibitor;
L is a linker attaching Ab to D; and
p is an integer from 1 to 20.

Embodiment I-2. The drug conjugate or the pharmaceutically acceptable salt thereof, or the solvate, the racemic mixture, the enantiomer, the diastereomer or the tautomer thereof according to embodiment I-1, wherein the structure of the D is as represented by Formula (II):

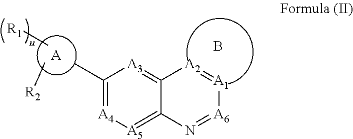

wherein
A₃, A₄, A₅, and A₆ are independently N or CR₅; R₅ is independently selected from: H, C₁₋₆ alkyl, C₃₋₉ cycloalkyl, —CN, —NH₂, —OH, —SH, —O(C₁₋₆ alkyl), halogen, or C₁₋₆ haloalkyl;

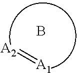

represents a 5- to 6-membered heteroaryl ring or a 3- to 8-membered heterocyclyl ring, and the 5- to 6-membered heteroaryl or 3- to 8-membered heterocyclyl is optionally substituted with one or more groups selected from oxo, ═NCN, or R₃;
ring A is 5- to 14-membered heteroaryl;
R₁, R₂, and R₃ are independently selected from: H, C₁₋₆ alkyl, C₂₋ₛ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)R^{e}, —NR^{c}S(O)ₙNR^{f}R^{g}, —NR^{c}C(O)OR^{b}, —NR^{c}C(O)NR^{d}R^{e}, —NO₂, —OR^{b}, —SR^{b}, —S(O)ₙR^{e}, —S(O)ₙR^{c}R^{d}, halogen, C₁₋₆ haloalkyl, 5- to 6-membered heteroaryl, and 3- to 12-membered heterocyclyl;
R^{a}, R^{b}, R^{c}, R^{d}, R^{e}, R^{f}, and R^{g} are each independently selected from hydrogen, C₁₋₆ alkyl, C₂₋₈ alkenyl, C₂₋₈ alkynyl, C₆-C₁₀ aryl, C₃₋₉ cycloalkyl, C₁₋₆ haloalkyl, 5- to 6-membered heteroaryl, and 3- to 12-membered heterocyclyl;
or R^{a} and R^{c}, and/or R^{c} and R^{d}, and/or R^{c} and R^{e}, and/or R^{c} and R^{f}, and/or R^{d} and R^{e}, and/or R^{g} and R^{f} together with the atoms to which they are attached form a 3- to 12-membered heterocyclyl;
wherein the C₁₋₆ alkyl, C₂₋₈ alkenyl, C₂₋₈ alkynyl, C₁₋₆ haloalkyl, C₆-C₁₀ aryl, C₃₋₉ cycloalkyl, 5- to 6-membered heteroaryl, and 3- to 12-membered heterocyclyl, at each occurrence, can each independently and optionally be substituted with one or more groups selected from: deuterium, C₁₋₆ alkyl, C₂₋ₛ alkenyl, C₂₋₈ alkynyl, C₆-C₁₀ aryl, C₃₋₉ cycloalkyl, —C(O)R^{a}, —C(O)OR^{b}, —CN, —C(O)NR^{c}R^{d}, —NR^{c}R^{d}, —NR^{c}C(O)R^{a}, —NR^{c}S(O)ₙR^{e}, —NR^{c}S(O)ₙNR^{f}R^{g}, —NR^{c}C(O)OR^{b}, —NR^{c}C(O)NR^{d}R^{e}, —NO₂, —C₁₋₆ alkylene-OR^{b}, —OR^{b}, —S(O)ₙR^{e}, —S(O)ₙR^{c}R^{d}, halogen, C₁₋₆ haloalkyl, 5- to 6-membered heteroaryl, and 3- to 8-membered heterocyclyl;
n, at each occurrence, is independently 0, 1, or 2;
u, at each occurrence, is independently 0, 1, 2, or 3; and
[symbol] represents a single bond or a double bond; and
wherein one of the R₁, R₂ or R₃, when present, is covalently linked to the L, and the valence of the atom at the linking site does not exceed the normal valence of the atom due to the one or more substituents to which it is linked.

Embodiment I-3. The drug conjugate or the pharmaceutically acceptable salt thereof, or the solvate, the racemic mixture, the enantiomer, the diastereomer or the tautomer thereof according to embodiment I-2, wherein

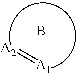

is selected from the following structures:

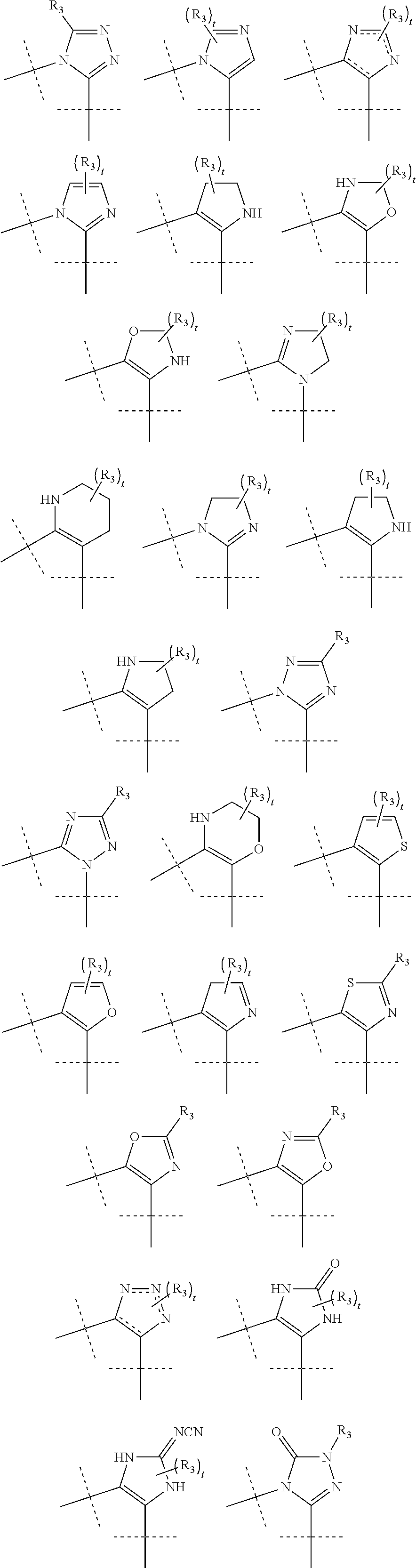

wherein t, at each occurrence, is independently 0, 1, 2, or 3; R₃ is as defined in embodiment I-2
[symbol] represents a single bond or a double bond; and
the dashed lines represent the positions to which the rings are fused.

Embodiment I-4. The drug conjugate or the pharmaceutically acceptable salt thereof, or the solvate, the racemic mixture, the enantiomer, the diastereomer or the tautomer thereof according to embodiment I-3, wherein

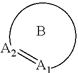

is selected from the following structures:

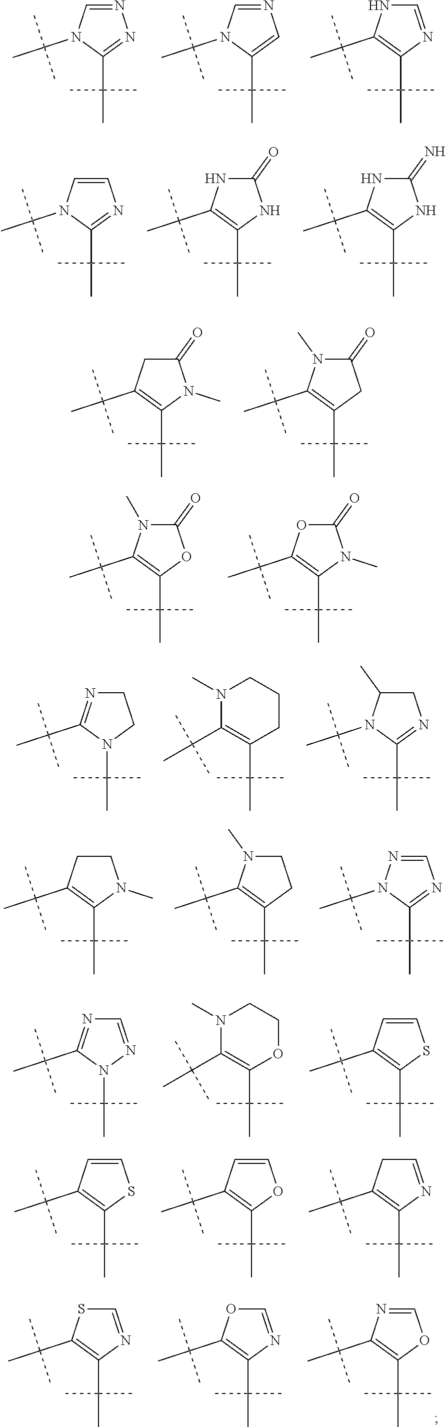

each of which is optionally substituted with one or more R₃, for example substituted with 1, 2, or 3 R₃, and R₃ is as defined in embodiment I-2.

Embodiment I-5. The drug conjugate or the pharmaceutically acceptable salt thereof, or the solvate, the racemic mixture, the enantiomer, the diastereomer or the tautomer thereof according to embodiment I-3 or I-4, wherein

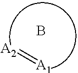

is selected from the following structures:

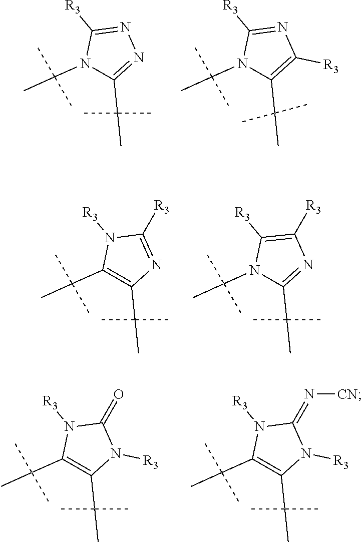

wherein R₃ is as defined in embodiment I-2, and can be the same or different from each other.

Embodiment I-6. The drug conjugate or the pharmaceutically acceptable salt thereof, or the solvate, the racemic mixture, the enantiomer, the diastereomer or the tautomer thereof according to any one of embodiments I-2 to I-5, wherein ring A is derived from the following structures:

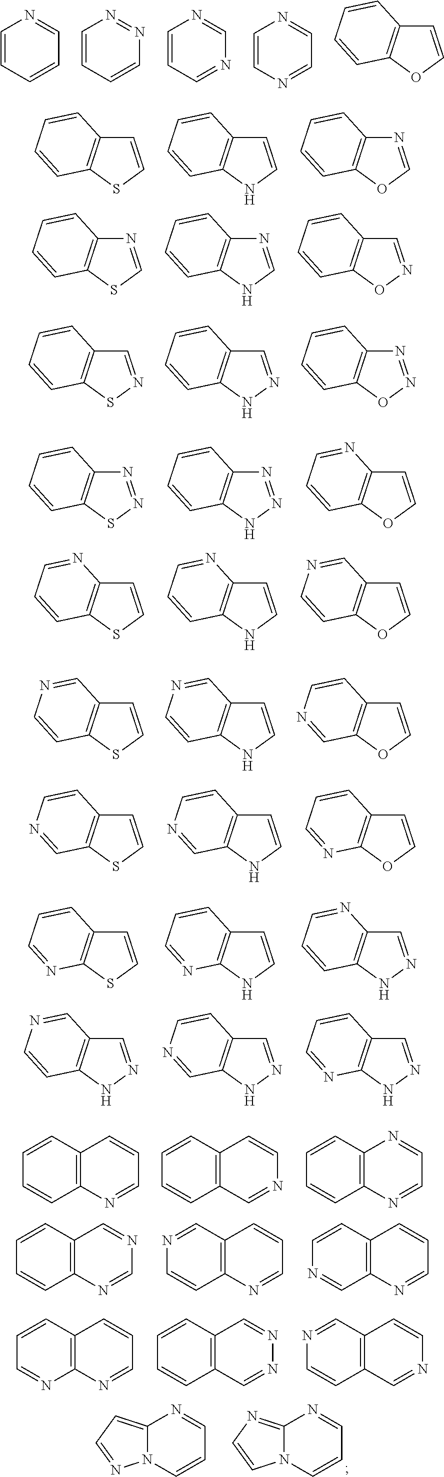

each of which is optionally substituted with u R₁ groups and R₂ group;
wherein u, R₁, and R₂ are as defined in embodiment I-2.

Embodiment I-7. The drug conjugate or the pharmaceutically acceptable salt thereof, or the solvate, the racemic mixture, the enantiomer, the diastereomer or the tautomer thereof according to any one of embodiments I-2 to I-6, wherein ring A is derived from the following structures:

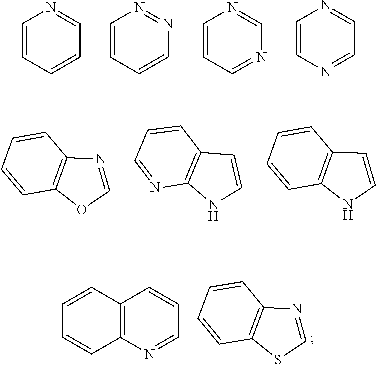

each of which is optionally substituted with u R₁ groups and R₂ group;
wherein u, R₁, and R₂ are as defined in embodiment I-2.

Embodiment I-8. The drug conjugate or the pharmaceutically acceptable salt thereof, or the solvate, the racemic mixture, the enantiomer, the diastereomer or the tautomer thereof according to any one of embodiments I-2 to I-7, wherein the structure of the ring A is:

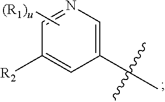

wherein R₁, R₂, and u are as defined in embodiment I-2;
the wavy line represents the site where the ring A is linked to the rest of the molecule.

In some embodiments, the structure of the ring A is:

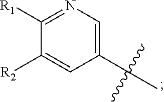

wherein R₁ and R₂ are as defined in embodiment I-2; and
the wavy line represents the site where the ring A is linked to the rest of the molecule.

Embodiment I-9. The drug conjugate or the pharmaceutically acceptable salt thereof, or the solvate, the racemic mixture, the enantiomer, the diastereomer or the tautomer thereof according to any one of embodiments I-2 to I-8, wherein A₃ is N or CH.

Embodiment I-10. The drug conjugate or the pharmaceutically acceptable salt thereof, or the solvate, the racemic mixture, the enantiomer, the diastereomer or the tautomer thereof according to any one of embodiments I-2 to I-9, wherein A₄ is N or CH.

Embodiment I-11. The drug conjugate or the pharmaceutically acceptable salt thereof, or the solvate, the racemic mixture, the enantiomer, the diastereomer or the tautomer thereof according to any one of embodiments I-2 to I-10, wherein A₅ is N or CH.

Embodiment I-12. The drug conjugate or the pharmaceutically acceptable salt thereof, or the solvate, the racemic mixture, the enantiomer, the diastereomer or the tautomer thereof according to any one of embodiments I-2 to I-11, wherein A₆ is N or CH.

Embodiment I-13. The drug conjugate or the pharmaceutically acceptable salt thereof, or the solvate, the racemic mixture, the enantiomer, the diastereomer or the tautomer thereof according to any one of embodiments I-2 to I-12, wherein A₃ is N, and A₄, A₅, and A₆ are all CH.

Embodiment I-14. The drug conjugate or the pharmaceutically acceptable salt thereof, or the solvate, the racemic mixture, the enantiomer, the diastereomer or the tautomer thereof according to any one of embodiments I-2 to I-13, wherein A₃, A₄, As, and A₆ are all CH.

Embodiment I-15. The drug conjugate or the pharmaceutically acceptable salt thereof, or the solvate, the racemic mixture, the enantiomer, the diastereomer or the tautomer thereof according to any one of embodiments I-2 to I-14, wherein R₁ is selected from: —C₁₋₆ alkyl, —O(C₁₋₆ alkyl), —OC₁₋₆ haloalkyl, or C₃₋₉ cycloalkyl, wherein the C₁₋₆ alkyl is optionally substituted with one or more deuterium; in some specific embodiments, R₁ is selected from: —C₁₋₆ alkyl, —OC₁₋₆ haloalkyl, or —O(C₁₋₆ alkyl), in particular is —O(C₁₋₆ alkyl) and —OC₁₋₆ haloalkyl, such as —OCH₃ or —OCH₂CF₃.

Embodiment I-16. The drug conjugate or the pharmaceutically acceptable salt thereof, or the solvate, the racemic mixture, the enantiomer, the diastereomer or the tautomer thereof according to any one of embodiments I-2 to I-15, wherein the linker L is linked to an active drug molecule via R₂, and R₂ is

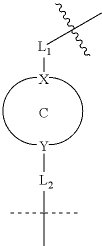

wherein [symbol] represents the site to which the ring A is linked;
[symbol] represents the site to which the linker L is linked;
L₁ is absent or is selected from: —C₁₋₆ alkylene-, —C(O)—, —C(O)O—, —NH—, —NHC(O)—, —NHS(O)ₙ₂—, —NHC(O)O—, —C(O)NH—, —C₁₋₆ alkylene-O—, —O—, —S—, or —S(O)ₙ₂—;
X and Y are each independently C, CH, or N;
L₂ is absent or is selected from: —C₁₋₆ alkylene-NH—, —NH—, —C₁₋₆ alkylene-O—, or —O—; ring C is selected from C₆-C₁₀ aryl, a 5- to 6-membered heteroaryl, or a 3- to 12-membered heterocyclic ring, each of which is independently and optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH; and
n₂, at each occurrence, is independently 1 or 2.

Embodiment I-17. The drug conjugate or the pharmaceutically acceptable salt thereof, or the solvate, the racemic mixture, the enantiomer, the diastereomer or the tautomer thereof according to embodiment I-16, wherein L₁ is —NHS(O)₂—, X and Y are both N, and L₂ is absent.

Embodiment I-18. The drug conjugate or the pharmaceutically acceptable salt thereof, or the solvate, the racemic mixture, the enantiomer, the diastereomer or the tautomer thereof according to embodiment I-16, wherein L₁ is —NHS(O)₂—, X is —CH— or N, Y is N, and L₂ is —C₁₋₆ alkylene-NH—, —NH—, —C₁₋₆ alkylene-O—, or —O—.

Embodiment I-19. The drug conjugate or the pharmaceutically acceptable salt thereof, or the solvate, the racemic mixture, the enantiomer, the diastereomer or the tautomer thereof according to any one of embodiments I-16 to I-18, wherein the ring C is a 3- to 12-membered heterocyclic ring which is optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH.

Embodiment I-20. The drug conjugate or the pharmaceutically acceptable salt thereof, or the solvate, the racemic mixture, the enantiomer, the diastereomer or the tautomer thereof according to embodiment I-19, wherein
the structure of

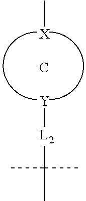

is selected from:

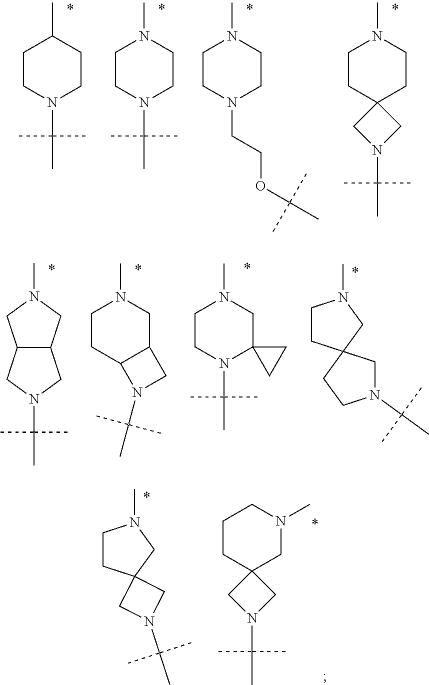

in some specific embodiments, the structure of

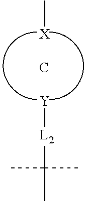

is selected from:

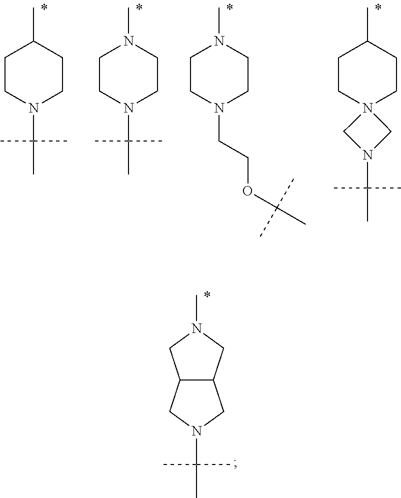

wherein * represents the site to which L₁ is linked; and
[symbol] represents the site to which the linker L is linked.

Embodiment I-21. The drug conjugate or the pharmaceutically acceptable salt thereof, or the solvate, the racemic mixture, the enantiomer, the diastereomer or the tautomer thereof according to any one of embodiments I-2 to I-15, wherein the linker L is linked to the active drug molecule via R₁ or R₃, and R₂ is selected from:

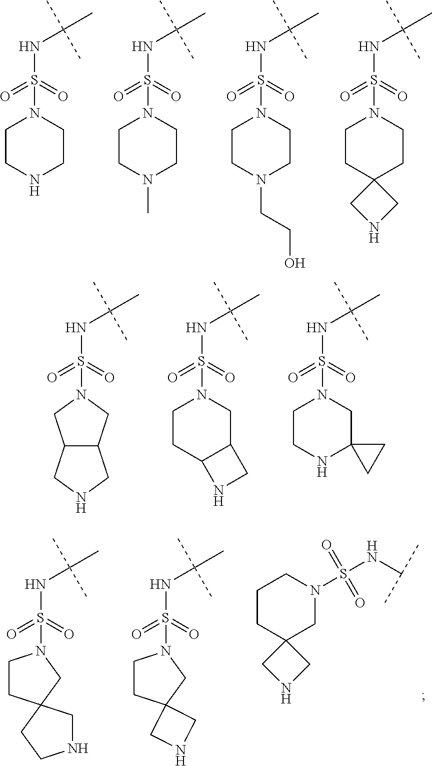

preferably, the structure of R₂ is:

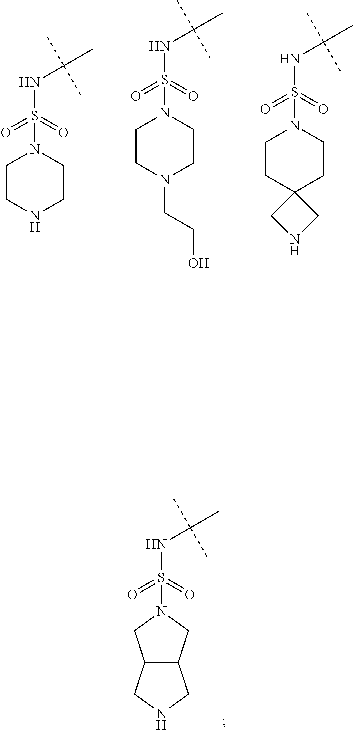

and

the dashed lines represent the position where R₂ is linked to the ring A.

Embodiment I-22. The drug conjugate or the pharmaceutically acceptable salt thereof, or the solvate, the racemic mixture, the enantiomer, the diastereomer or the tautomer thereof according to any one of embodiments I-2 to I-21, wherein each R₃ is independently selected from: H, CN, NO₂, halogen, C₁₋₆ alkyl, C₆-C₁₀ aryl, C₃₋₉ cycloalkyl, 5- to 6-membered heteroaryl, and 3- to 12-membered heterocyclyl, wherein the C₁₋₆ alkyl, C₆-C₁₀ aryl, C₃₋₉ cycloalkyl, 5- to 6-membered heteroaryl, and 3- to 12-membered heterocyclyl can each optionally be substituted with one or more groups independently selected from: deuterium, C₁₋₆ alkyl, C₂₋₈ alkenyl, C₂₋₈ alkynyl, C₃₋₉ cycloalkyl, —CN, —C(O)NH₂, —NH₂, —NHC(O)C₁₋₆ alkyl, —NO₂, —OH, —C₁₋₆ alkylene-OH, halogen, C₁₋₆ haloalkyl, or —C₁₋₆ alkylene-OC₁₋₆ alkyl.

Embodiment I-23. The drug conjugate or the pharmaceutically acceptable salt thereof, or the solvate, the racemic mixture, the enantiomer, the diastereomer or the tautomer thereof according to embodiment I-22, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, C₁₋₆ alkyl, —CN, —NH₂, —NO₂, —OH, —C₁₋₆ alkylene-OH, halogen, C₁₋₆ haloalkyl, or —C₁₋₆ alkylene-OC₁₋₆ alkyl;

In some embodiments, each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, —OH, F, or —C₁₋₆ alkylene-OH; and

In some specific embodiments, each R₃ is independently selected from: hydrogen, —CH₃, —CD₃, —CH(CH₃)₂, —CH₂CF₃, or the following groups:

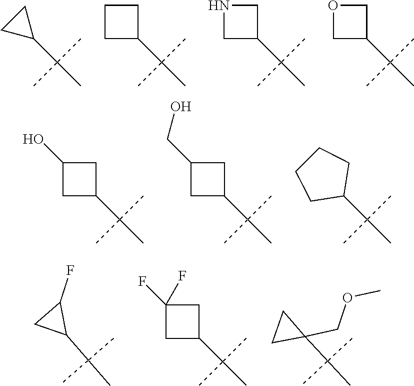

Embodiment I-24. The drug conjugate or the pharmaceutically acceptable salt thereof, or the solvate, the racemic mixture, the enantiomer, the diastereomer or the tautomer thereof according to any one of embodiments I-2 to I-15, or I-21, wherein the linker L is linked to the active drug molecule via R₃, and the structure of R₃ is

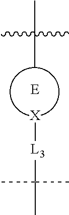

wherein [symbol] represents the site to which ring B is linked;
[symbol] represents the site to which the linker L is linked;
X is —CH— or N;
L₃ is absent or is selected from —C₁₋₆ alkylene-NH—, —NH—, —C₁₋₆ alkylene-O—, or —O—; and
ring E is selected from C₃₋₉ cycloalkyl and a 3- to 12-membered heterocyclic ring, which are optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH.

In some embodiments, X is N, and L₃ is absent; and

In some embodiments, X is —CH—, and L₃ is —C₁₋₆ alkylene-O— or —O—.

Embodiment I-25. The drug conjugate or the pharmaceutically acceptable salt thereof, or the solvate, the racemic mixture, the enantiomer, the diastereomer or the tautomer thereof according to embodiment I-24, wherein the ring E is selected from:

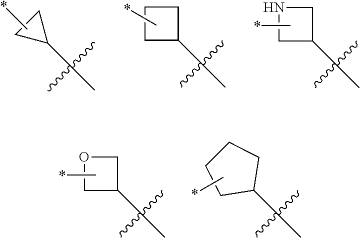

wherein [symbol] represents the site to which ring B is linked; and
* represents the site to which L₃ is linked.

Embodiment I-26. The drug conjugate or the pharmaceutically acceptable salt thereof, or the solvate, the racemic mixture, the enantiomer, the diastereomer or the tautomer thereof according to any one of embodiments I-2 to I-15, wherein the structure of the D is:

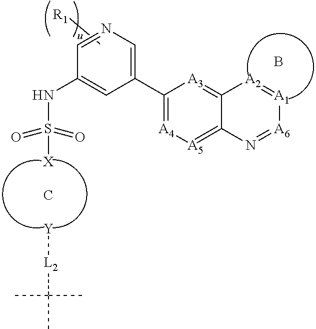

[symbol] represents the site to which the linker L is linked;
X and Y are each independently C, —CH—, or N;
L₂ is absent or is selected from —C₁₋₆ alkylene-NH—, —NH—, —C₁₋₆ alkylene-O—, or —O—;
ring C is selected from C₆-C₁₀ aryl, a 5- to 6-membered heteroaryl, or a 3- to 12-membered heterocyclic ring, each of which is independently and optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH;
u, at each occurrence, is independently 0, 1, 2, or 3; and
R₁, A₃, A₄, A₅, A₆, and

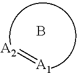

are as defined in embodiment I-2.

Embodiment I-27. The drug conjugate or the pharmaceutically acceptable salt thereof, or the solvate, the racemic mixture, the enantiomer, the diastereomer or the tautomer thereof according to embodiments I-26, wherein A₃, A₄, A₅, and A₆ are all CH, or A₃ is N and A₄, A₅, and A₆ are all CH.

Embodiment I-28. The drug conjugate or the pharmaceutically acceptable salt thereof, or the solvate, the racemic mixture, the enantiomer, the diastereomer or the tautomer thereof according to embodiment I-26 or I-27, wherein

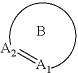

is selected from the following structures:

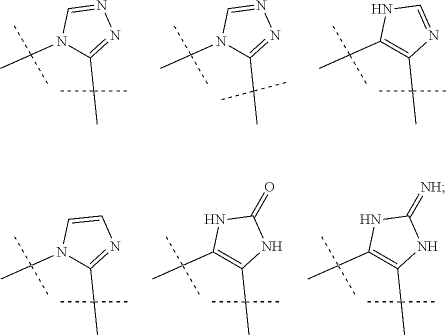

each of which is optionally substituted with one or more R₃, wherein R₃ is as defined in embodiment I-2.

Embodiment I-29. The drug conjugate or the pharmaceutically acceptable salt thereof, or the solvate, the racemic mixture, the enantiomer, the diastereomer or the tautomer thereof according to embodiment I-28, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, C₁₋₆ alkyl, —CN, —NH₂, —NO₂, —OH, —C₁₋₆ alkylene-OH, halogen, C₁₋₆ haloalkyl, or —C₁₋₆ alkylene-OC₁₋₆ alkyl.

Embodiment I-30. The drug conjugate or the pharmaceutically acceptable salt thereof, or the solvate, the racemic mixture, the enantiomer, the diastereomer or the tautomer thereof according to embodiment I-29, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, —OH, F, or —C₁₋₆ alkylene-OH; and

In some specific embodiments, each R₃ is independently selected from: hydrogen, —CH₃, —CD₃, —CH(CH₃)₂, —CH₂CF₃, or the following groups:

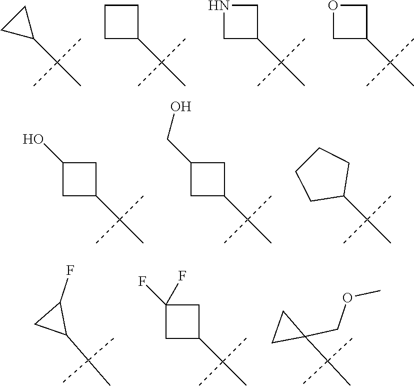

Embodiment I-31. The drug conjugate or the pharmaceutically acceptable salt thereof, or the solvate, the racemic mixture, the enantiomer, the diastereomer or the tautomer thereof according to any one of embodiments I-26 to I-30, wherein

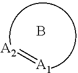

is

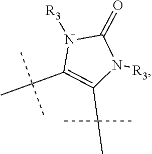

each R₃ is independently as defined in embodiments I-26 to I-30.

Embodiment I-32. The drug conjugate or the pharmaceutically acceptable salt thereof, or the solvate, the racemic mixture, the enantiomer, the diastereomer or the tautomer thereof according to any one of embodiments I-26 to I-30, wherein

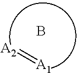

is

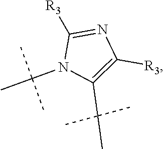

each R₃ is independently as defined in embodiments I-26 to I-30;

In some specific embodiments, each R₃ is independently H, —CH₃, or C₃₋₉ cycloalkyl.

Embodiment I-33. The drug conjugate or the pharmaceutically acceptable salt thereof, or the solvate, the racemic mixture, the enantiomer, the diastereomer or the tautomer thereof according to any one of embodiments I-26 to I-32, wherein X and Y are both N, and L₂ is absent.

Embodiment I-34. The drug conjugate or the pharmaceutically acceptable salt thereof, or the solvate, the racemic mixture, the enantiomer, the diastereomer or the tautomer thereof according to any one of embodiments I-26 to I-32, wherein X is —CH— or N, Y is N, and L₂ is —C₁₋₆ alkylene-NH—, —NH—, —C₁₋₆ alkylene-O—, or —O—.

Embodiment I-35. The drug conjugate or the pharmaceutically acceptable salt thereof, or the solvate, the racemic mixture, the enantiomer, the diastereomer or the tautomer thereof according to any one of embodiments I-26 to I-34, wherein the ring C is a 3- to 12-membered heterocyclic ring which is optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH.

Embodiment I-36. The drug conjugate or the pharmaceutically acceptable salt thereof, or the solvate, the racemic mixture, the enantiomer, the diastereomer or the tautomer thereof according to embodiment I-35, wherein the structure of

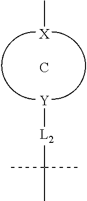

is selected from:

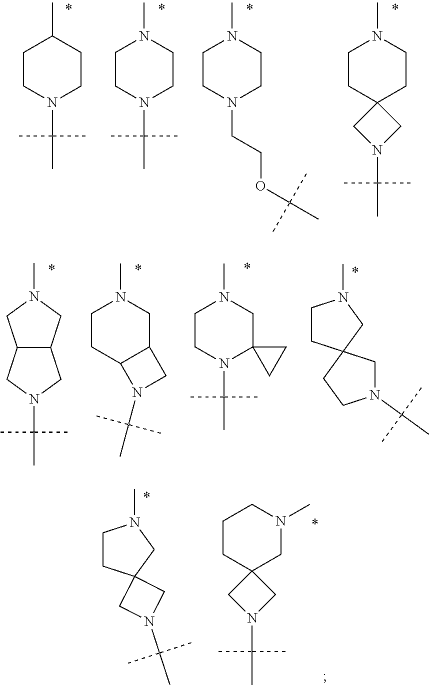

In some specific embodiments, the structure of

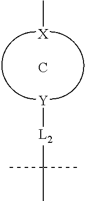

is selected from:

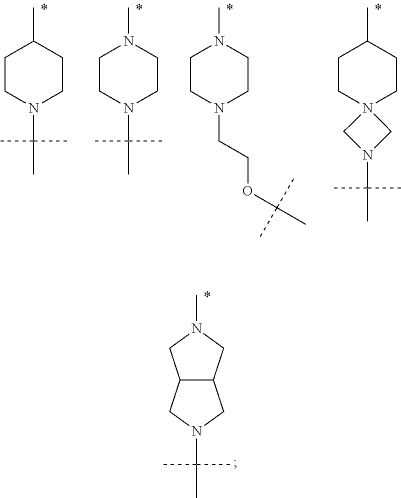

wherein * represents the site to which the parent core structure is linked; and
[symbol] represents the site to which the linker L is linked.

Embodiment I-37. The drug conjugate or the pharmaceutically acceptable salt thereof, or the solvate, the racemic mixture, the enantiomer, the diastereomer or the tautomer thereof according to any one of embodiments I-26 to I-36, wherein R₁ is selected from: —O(C₁₋₆ alkyl), —OC₁₋₆ haloalkyl, or C₃₋₉ cycloalkyl, wherein the C₁₋₆ alkyl can be optionally substituted with one or more deuterium; in some specific embodiments, R₁ is —O(C₁₋₆ alkyl); in some specific embodiments, R₁ is —O—CH₂—CF₃.

Embodiment I-38. The drug conjugate or the pharmaceutically acceptable salt thereof, or the solvate, the racemic mixture, the enantiomer, the diastereomer or the tautomer thereof according to any one of embodiments I-1 to I-37, wherein D is selected from:

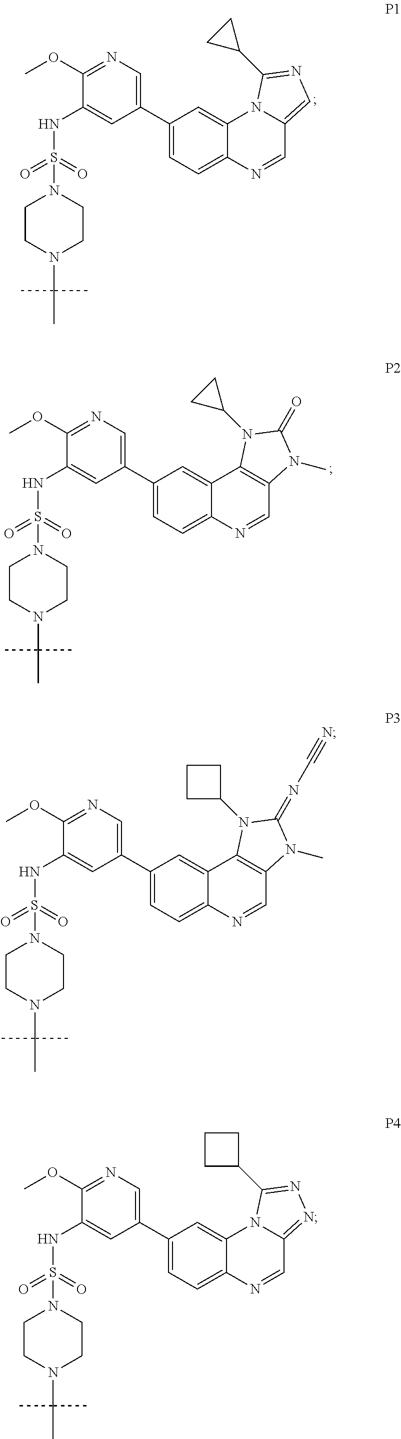

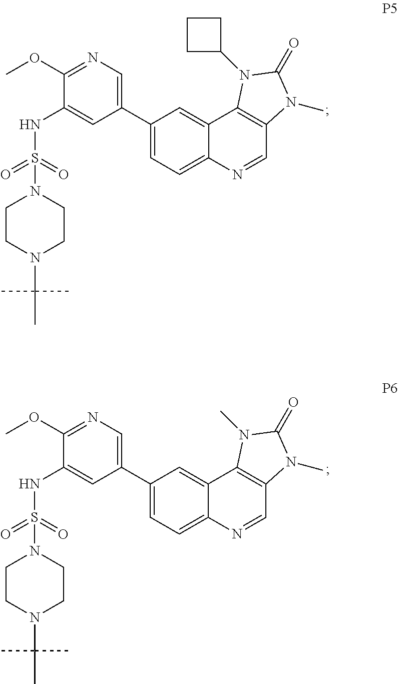

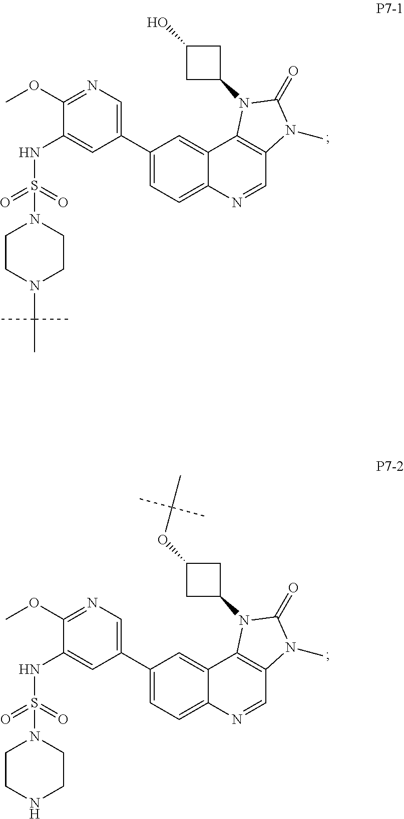

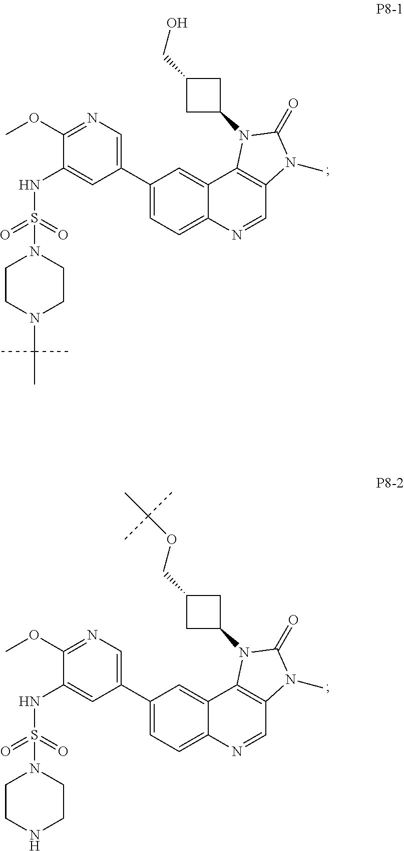

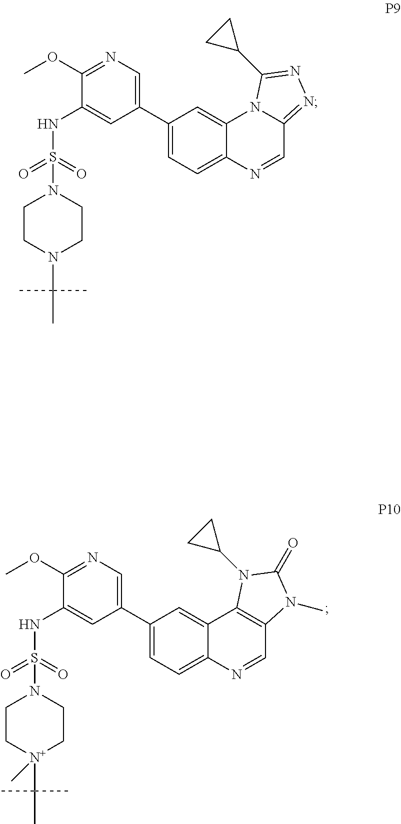

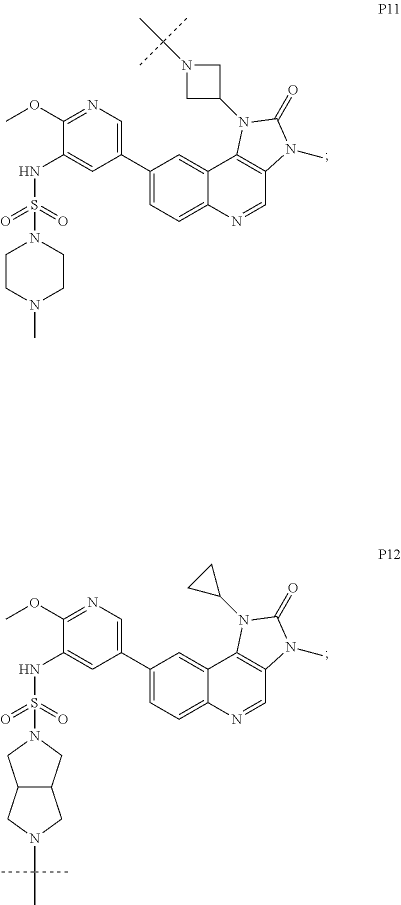

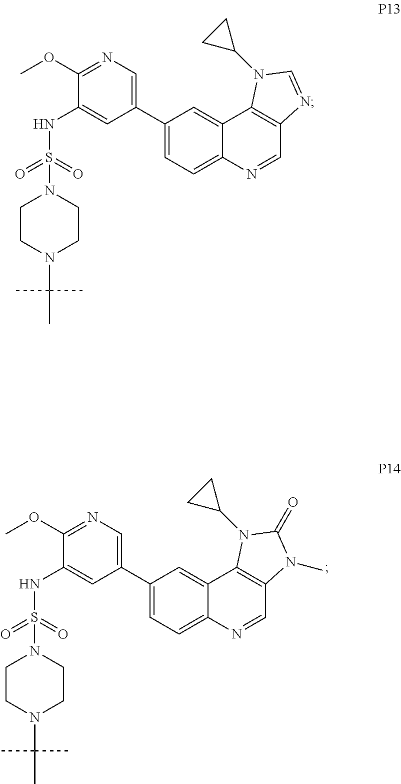

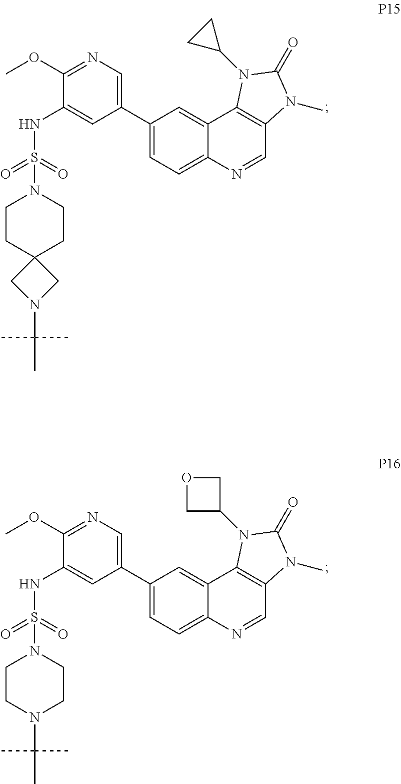

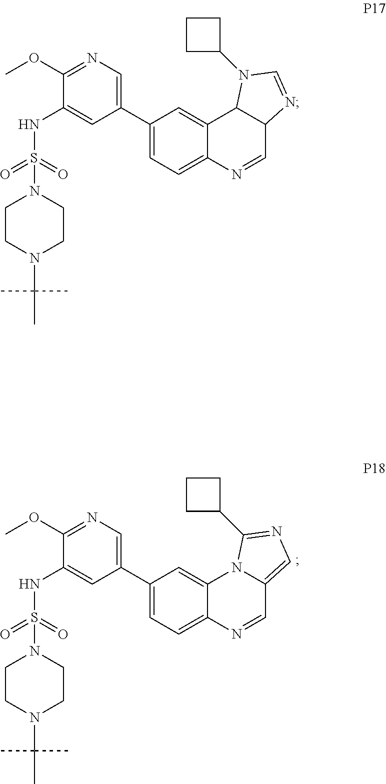

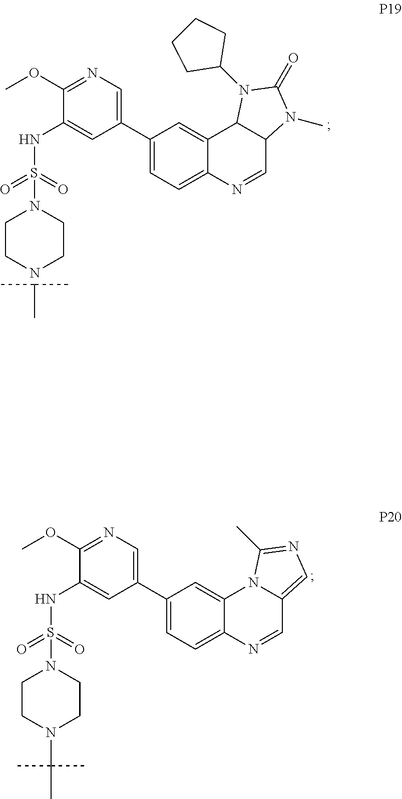

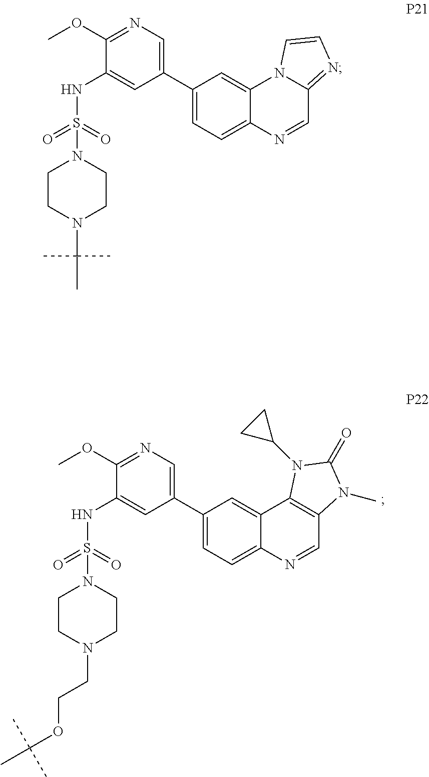

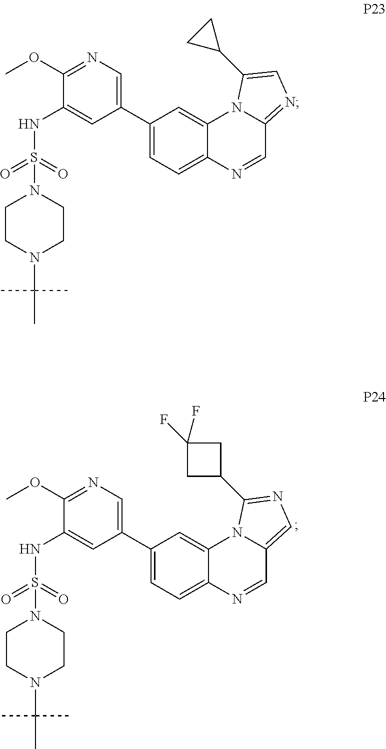

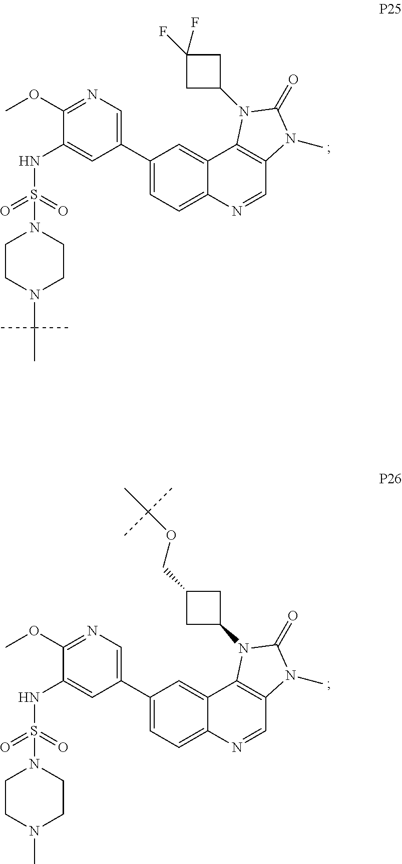

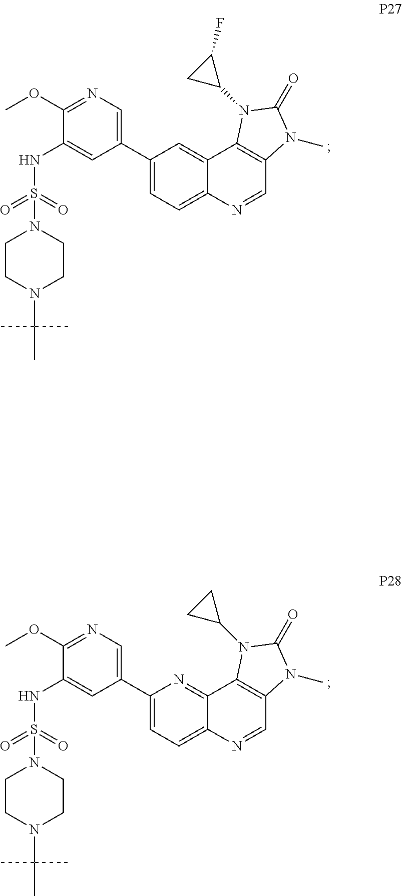

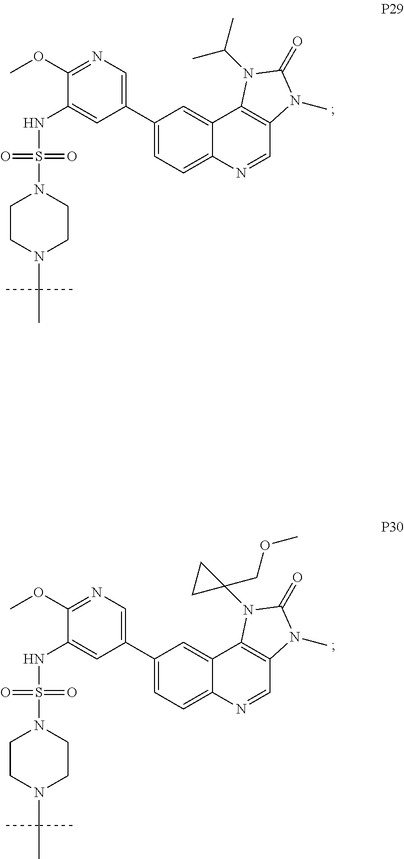

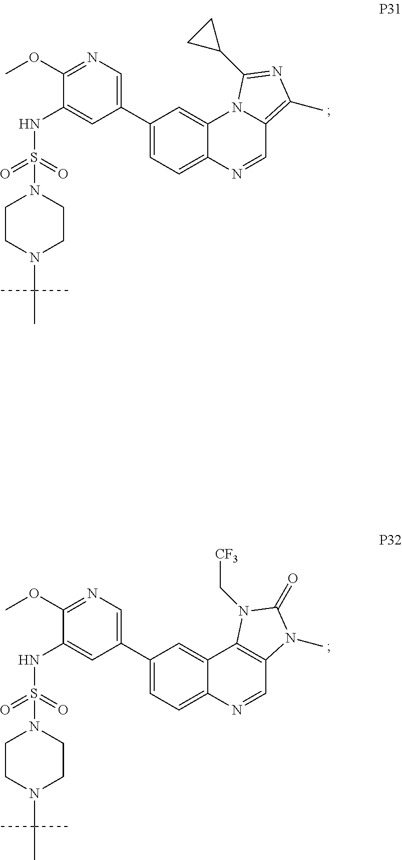

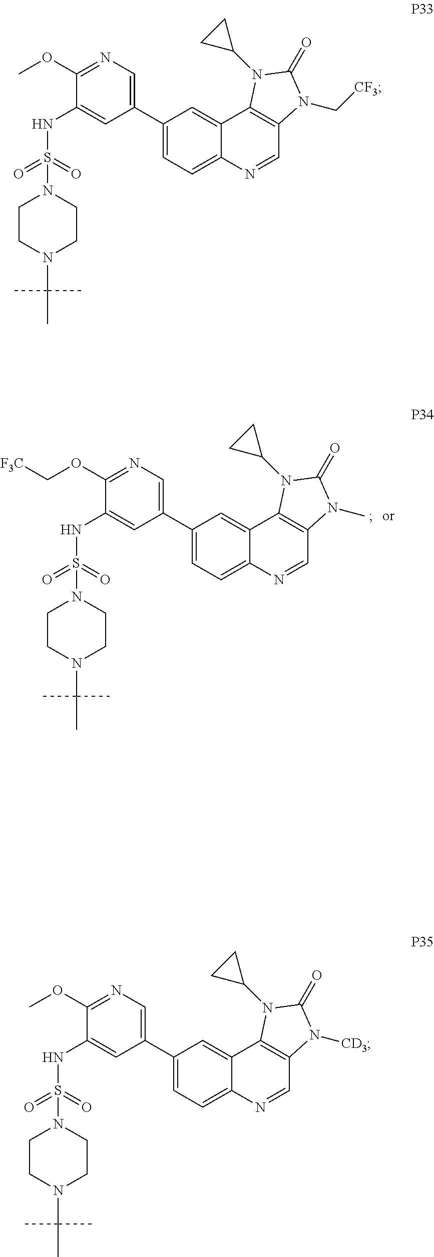

wherein the dashed lines represent the site where D is linked to the linker L.

Embodiment I-39. The drug conjugate or the pharmaceutically acceptable salt thereof, or the solvate, the racemic mixture, the enantiomer, the diastereomer or the tautomer thereof according to any one of embodiments I-1 to I-38, wherein -L- has a structure represented by formula (III):

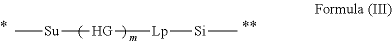

wherein,
Su is a stretcher group;
HG is a bridged spacer group;
Lp is a short chain polypeptide consisting of 2-10 amino acids, wherein the amino acids are natural amino acids or non-natural amino acids;
Si is a self-immolative group;
m is 0 or 1;
the symbol * represents the site to which Ab is linked; and the symbol ** represents the site to which D is linked.

Embodiment I-40. The drug conjugate or the pharmaceutically acceptable salt thereof, or the solvate, the racemic mixture, the enantiomer, the diastereomer or the tautomer thereof according to embodiment I-39, wherein the Su is selected from the following structures:

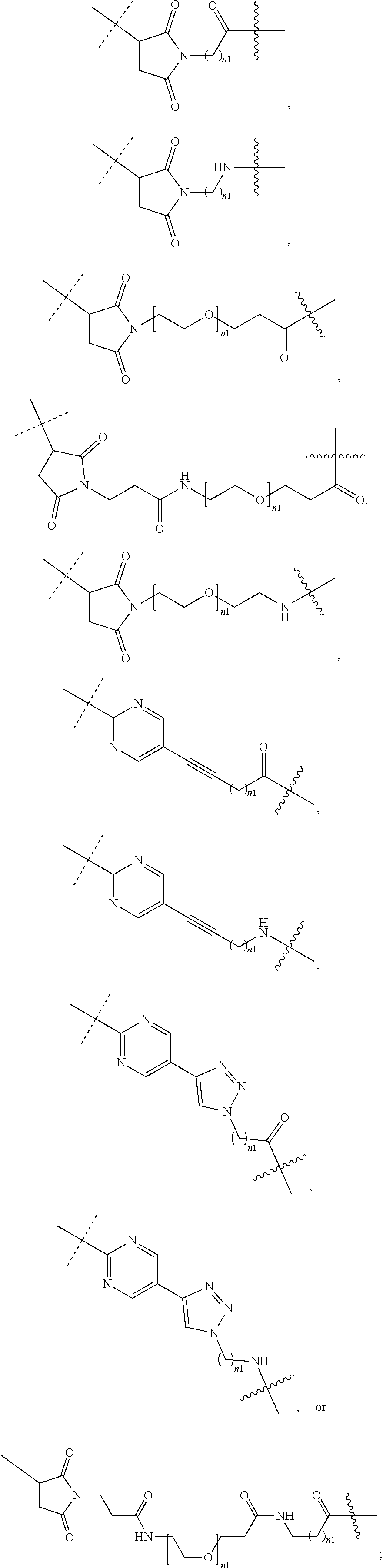

wherein each n₁ is independently selected from an integer from 1 to 10, such as 1, 2, 3, 4, 5, 6, 7, 8, 9, or 10;
[symbol] represents the site to which the Ab is linked; and
[symbol] represents the site to which a structure (e.g., HG or Lp) on the right side of Su is linked;

In some specific embodiments, Su is selected from:

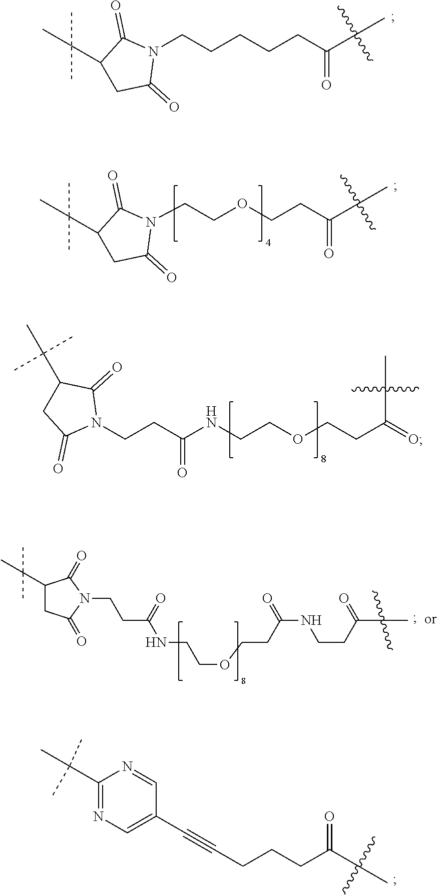

and the remaining symbols are as defined in the embodiments hereinbefore.

Embodiment I-41. The drug conjugate or the pharmaceutically acceptable salt thereof, or the solvate, the racemic mixture, the enantiomer, the diastereomer or the tautomer thereof according to embodiment I-39 or I-40, wherein the amino acids contained in the Lp are independently selected from the L- or D-isomers of the following amino acids: alanine (including β-alanine), arginine, aspartic acid, asparagine, cysteine, histidine, glycine, glutamic acid, valine, glutamine, phenylalanine, lysine, leucine, isoleucine, methionine, serine, tyrosine, threonine, tryptophan, proline, ornithine, penicillamine, aminoalkynoic acid, aminoalkanedioic acid, heterocyclyl-carboxylic acid, citrulline, selenocysteine, pyrrolysine, diaminoalkanoic acid, and a derivative thereof; preferably, Lₚ is selected from: -Phe-Lys-, -Val-Lys-, -Phe-Phe-Lys-, -D-Phe-Phe-Lys-, -Gly-Phe-Lys-, -Ala-Lys-, -Val-Cit-, -Phe-Cit-, -Leu-Cit-, -Ile-Cit-, -Trp-Cit-, -Phe-Ala-, -Gly-Phe-Leu-Gly-, -Ala-Leu-Ala-Leu-, -Val-Ala-, -Gly-Gly-Phe-Gly, -Gly-Cit-, -Cit-Val-, -Cit-Ala-, -Lys-Val-, -Val-Lys(Ac)-, -Phe-Lys(Ac)-, -Ala-Ala-, -Ala-Ala-Ala-, -Ala-Ala-Asn-, -Ala-Ala-Asp-, -Lys-Ala-Ala-Asn-, -Lys-Ala-Ala-Asp-, -D-Val-Leu-Lys-, -Gly-Gly-Arg-, -Gly-Gly-Phe-, -Val-Lys-Gly-, -Val-Lys-Gly-Gly-, and -Lys-Ala-Asn-, or a derivative thereof; and more preferably, Lp is -Val-Cit-, -Val-Ala-, -Gly-Gly-Phe-Gly, or a derivative thereof.

Embodiment I-42. The drug conjugate or the pharmaceutically acceptable salt thereof, or the solvate, the racemic mixture, the enantiomer, the diastereomer or the tautomer thereof according to embodiment I-39 or I-40, wherein the structure of Si is:

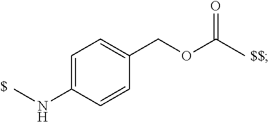

wherein the symbol $ represents the site to which Lp is linked, and the symbol $$ represents the site to which D is linked.

Embodiment I-43. The drug conjugate or the pharmaceutically acceptable salt thereof, or the solvate, the racemic mixture, the enantiomer, the diastereomer or the tautomer thereof according to any one of embodiments I-39 to I-42, wherein the structure of HG is:

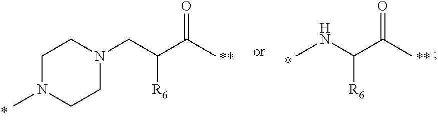

wherein each R₆ is independently selected from: —OR^{x1}, —NR^{x1}R^{x2}, —C₁₋₆ alkylene-OR^{x1}, —C₁₋₆ alkylene-NR^{x1}R^{x2}, C₁₋₆ alkyl, or —C₁₋₆ alkylene-COOH, wherein R^{x1} and R^{x2} are each independently selected from H or C₁₋₆ alkyl;
the symbol * represents the site to which the Su group is linked; and the symbol ** represents the site to which the Lp group is linked; and

In some specific embodiments, each R₆ is independently —OH or —C₁₋₆ alkylene-COOH.

Embodiment I-44. The drug conjugate or the pharmaceutically acceptable salt thereof, or the solvate, the racemic mixture, the enantiomer, the diastereomer or the tautomer thereof according to embodiment I-43, wherein HG is selected from:

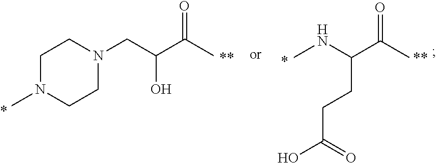

such as selected from:

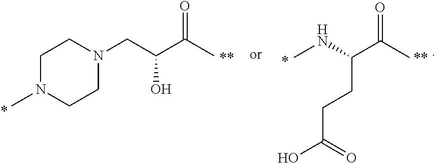

Embodiment I-45. The drug conjugate or the pharmaceutically acceptable salt thereof, or the solvate, the racemic mixture, the enantiomer, the diastereomer or the tautomer thereof according to any one of embodiments I-1 to I-44, wherein the structure of -L-D is selected from:

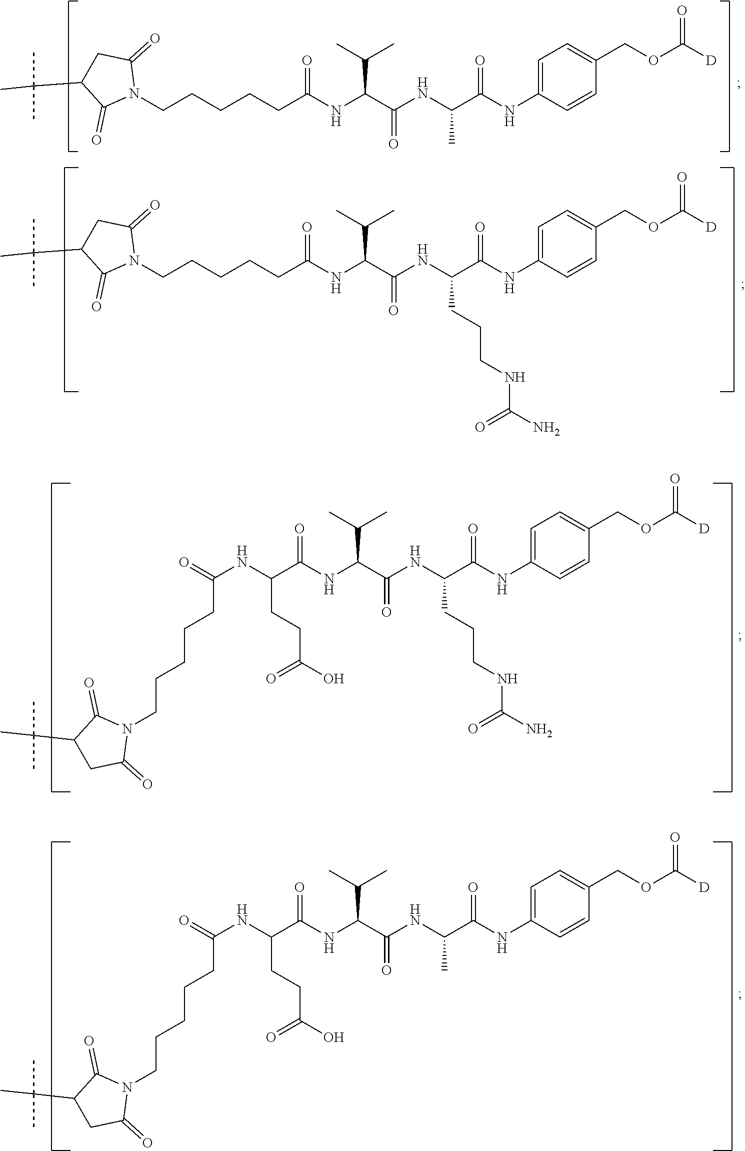

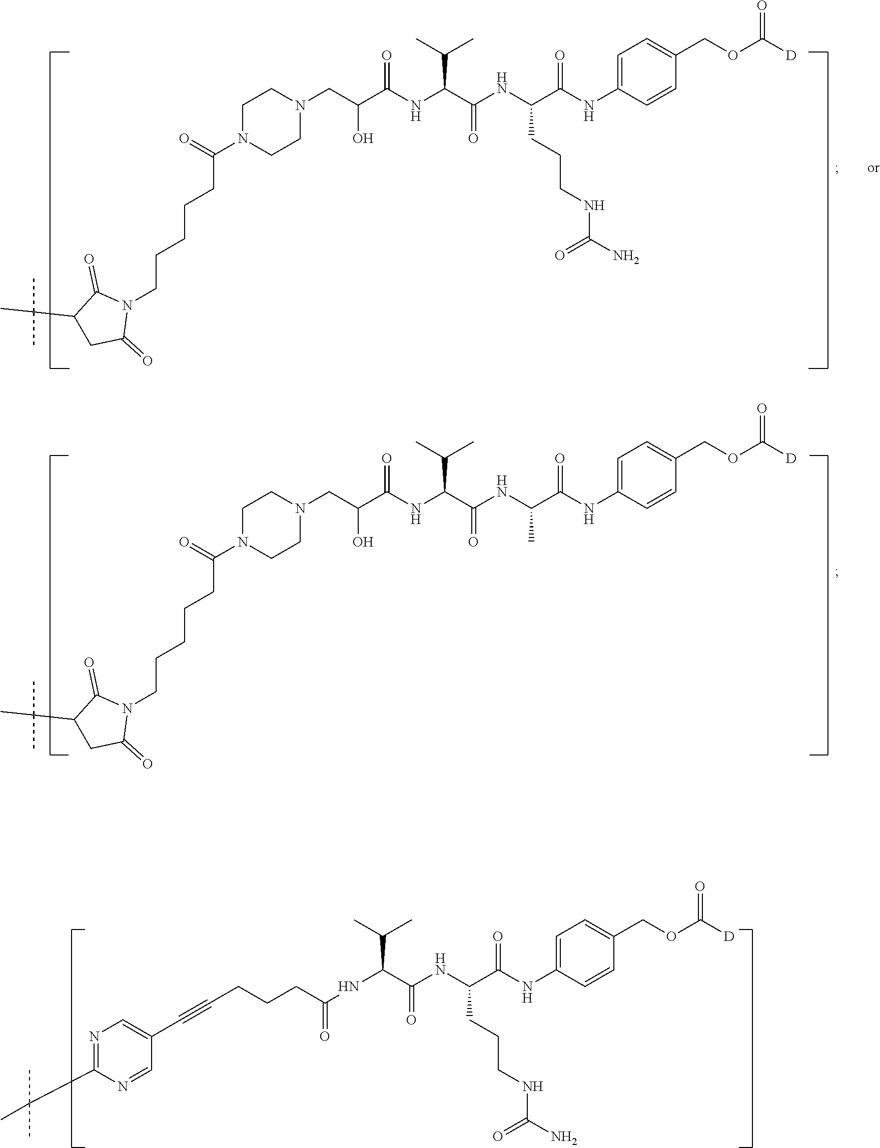

wherein the dashed lines represent the site to which Ab is linked; and
D is as defined in embodiments I-1 to I-38.

Embodiment I-46. The drug conjugate or the pharmaceutically acceptable salt thereof, or the solvate, the racemic mixture, the enantiomer, the diastereomer or the tautomer thereof according to any one of embodiments I-1 to I-45, wherein -L-D is selected from:

Structure

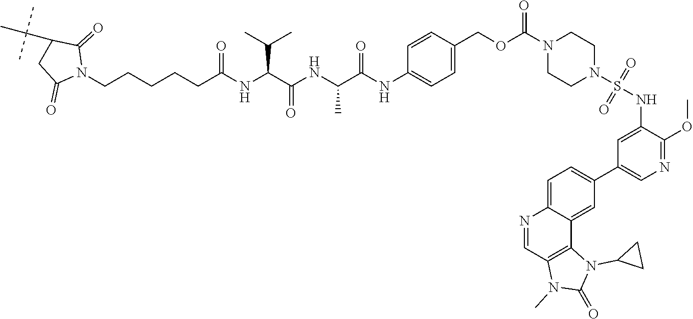

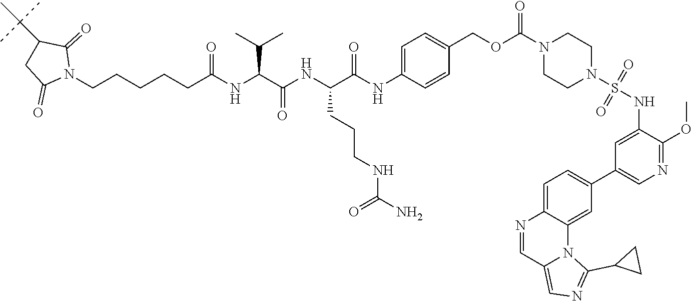

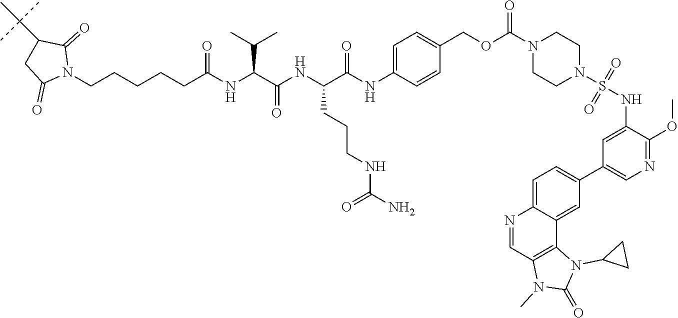

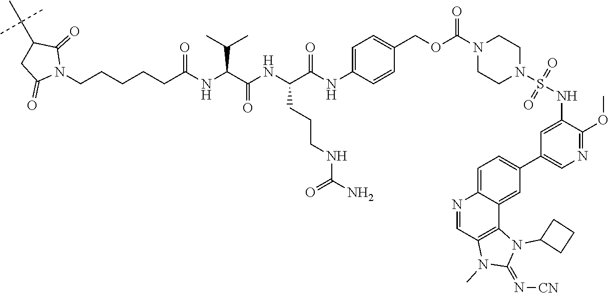

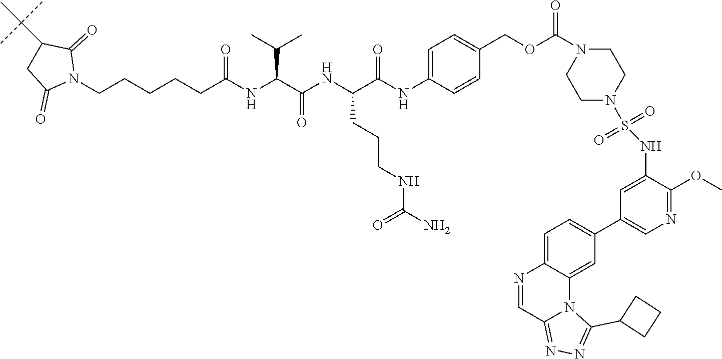

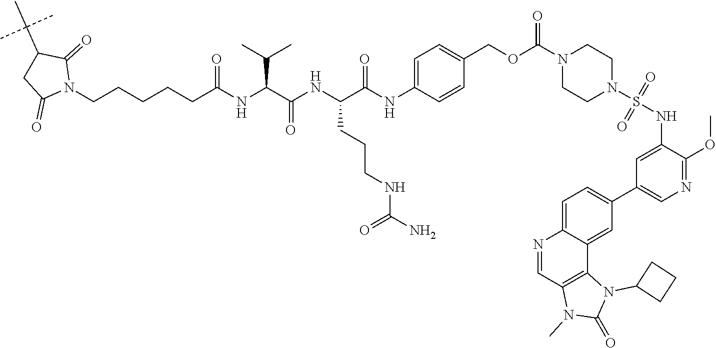

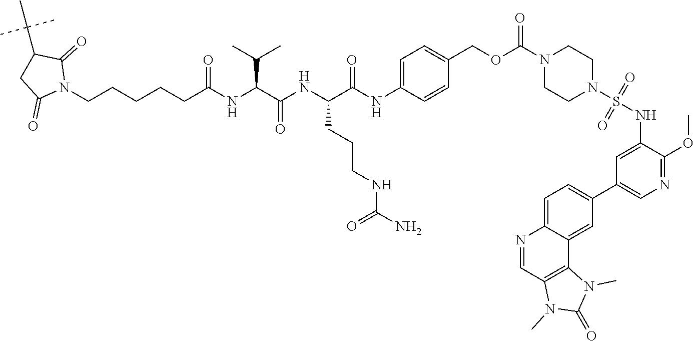

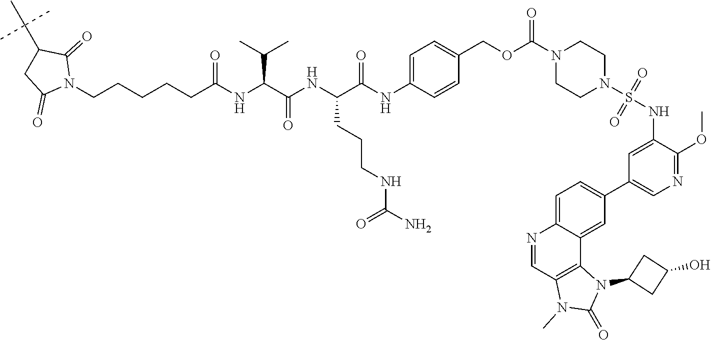

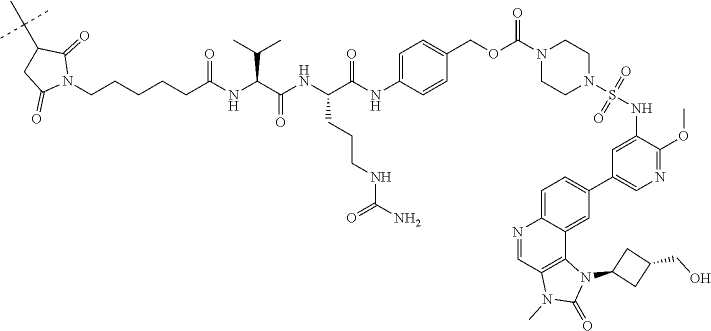

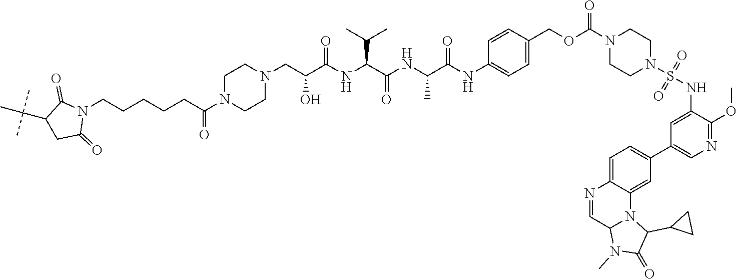

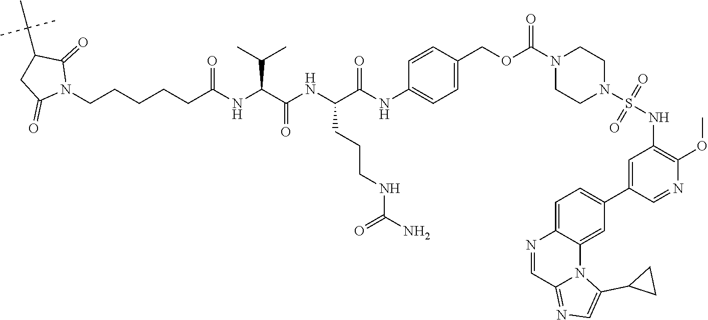

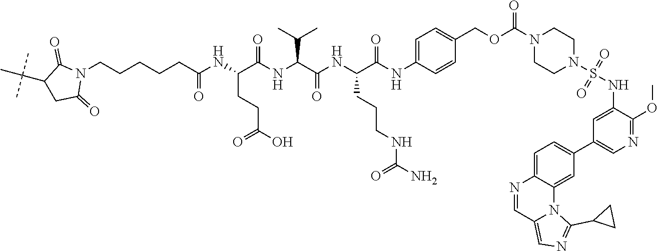

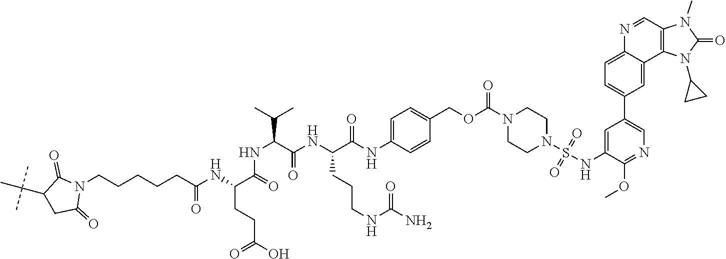

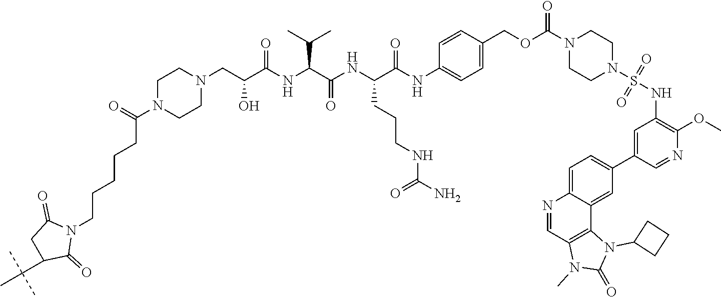

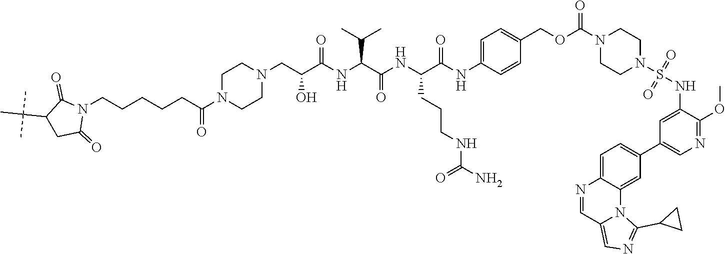

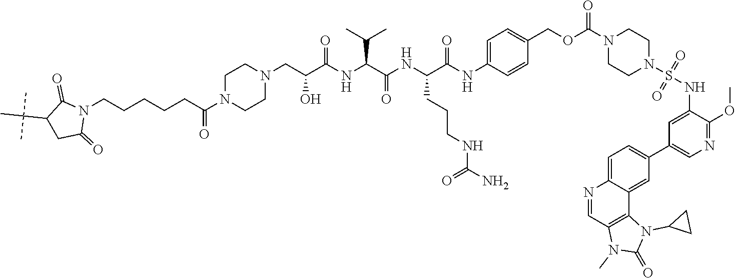

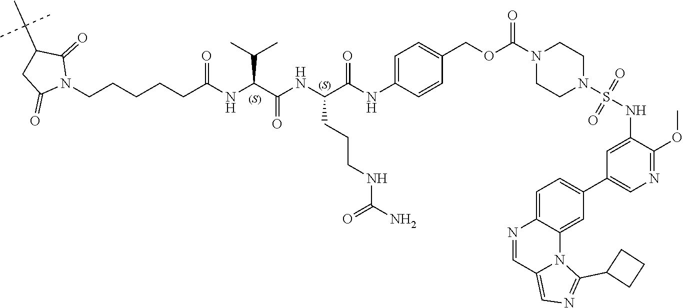

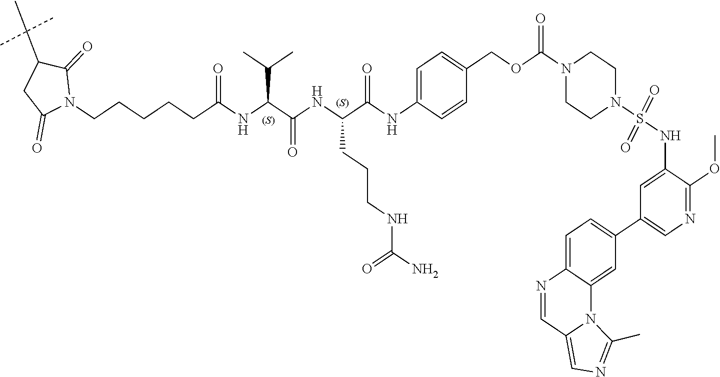

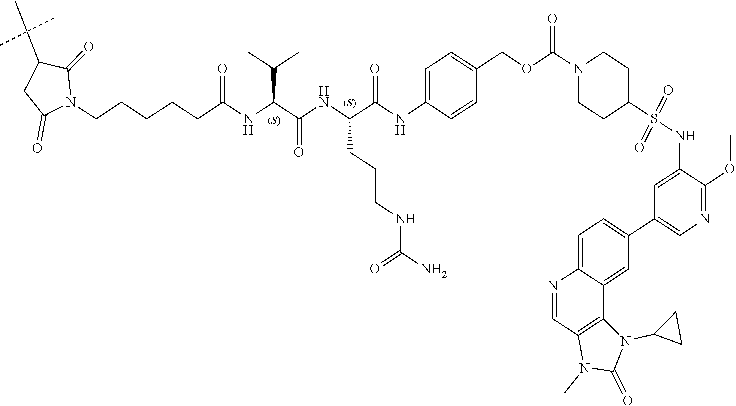

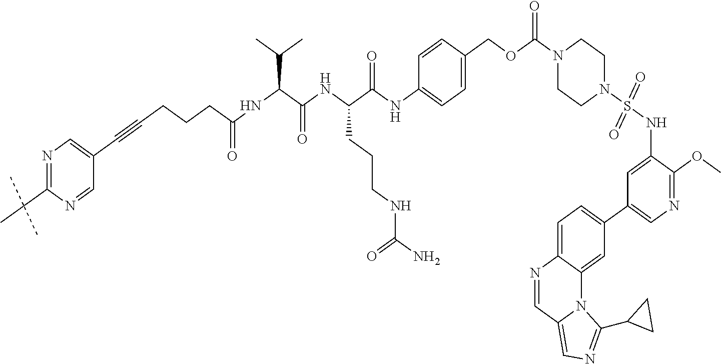

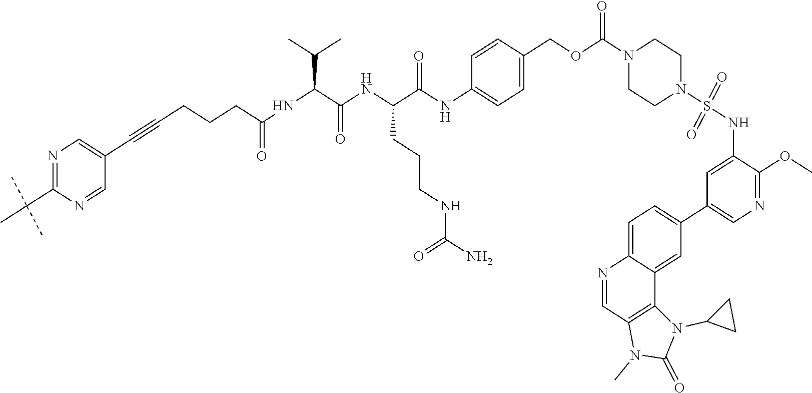

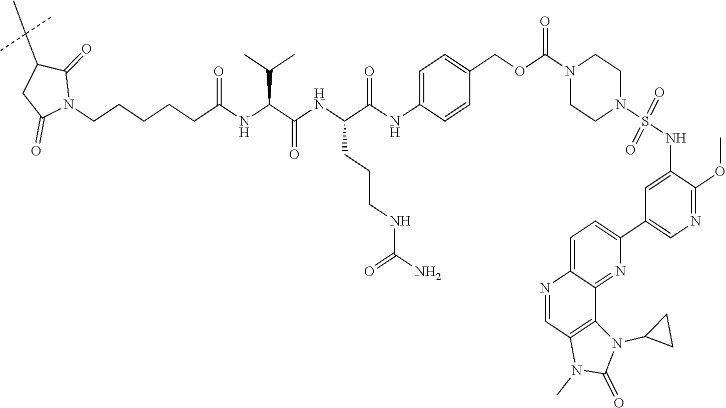

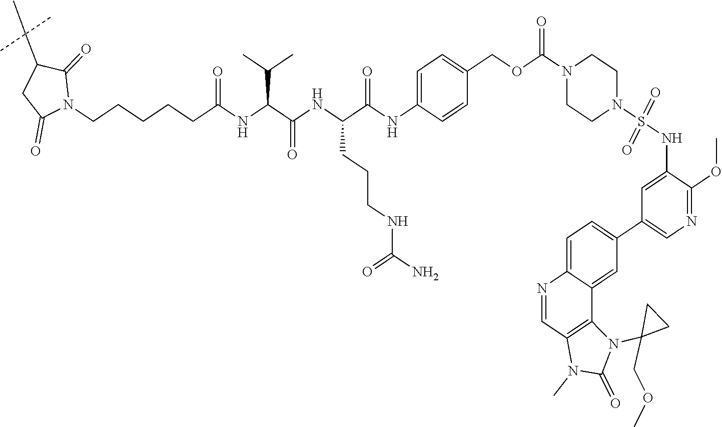

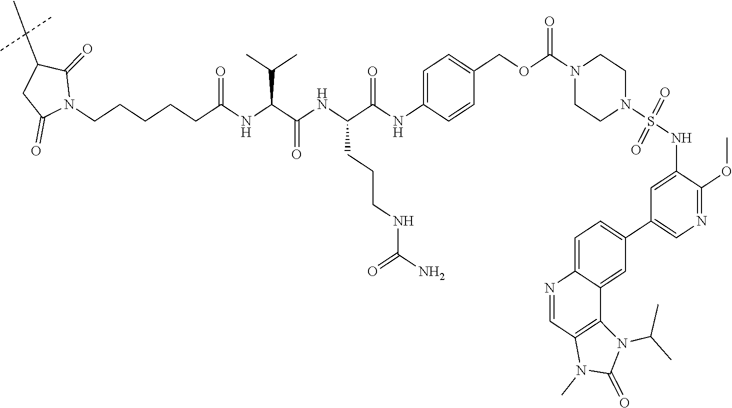

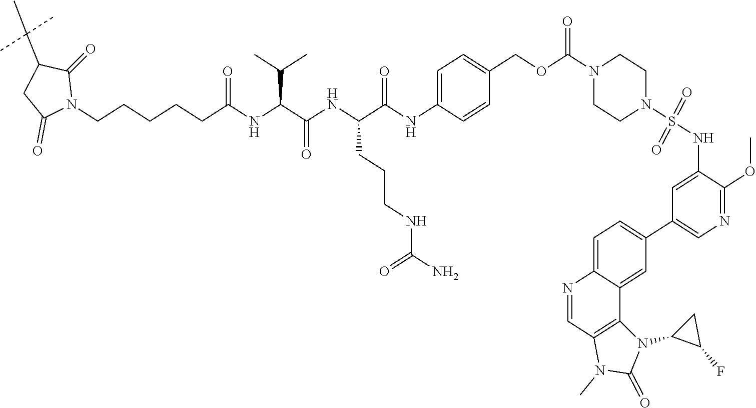

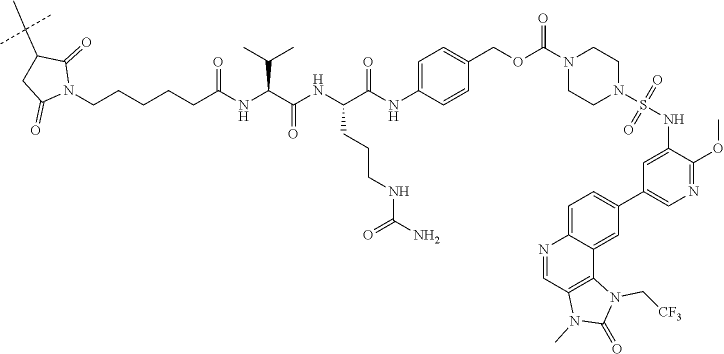

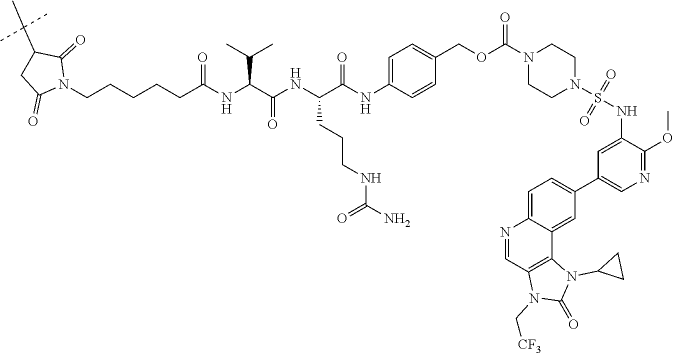

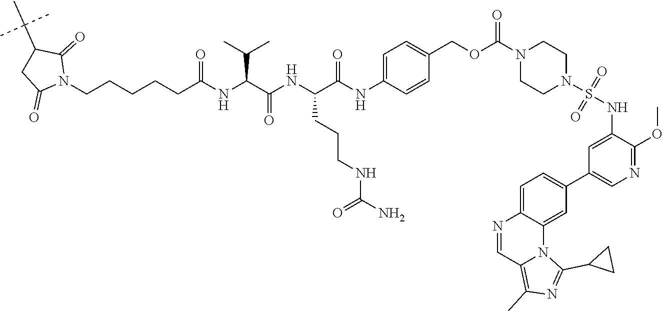

and wherein the dashed lines represent the site to which Ab is linked.

Embodiment I-47. The drug conjugate or the pharmaceutically acceptable salt thereof, or the solvate, the racemic mixture, the enantiomer, the diastereomer or the tautomer thereof according to embodiment I-46, wherein the drug conjugate is selected from:

Structure

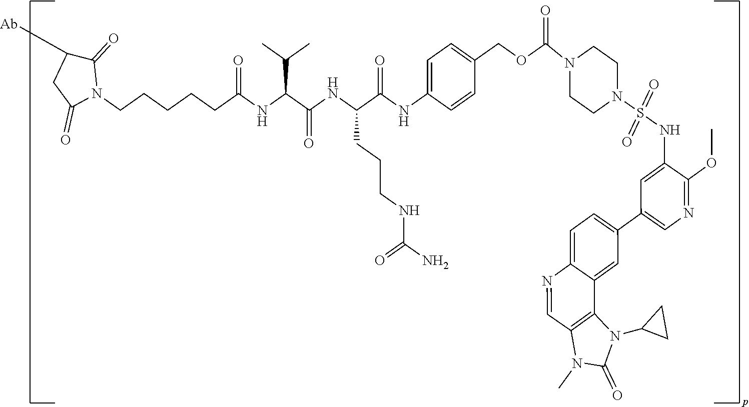

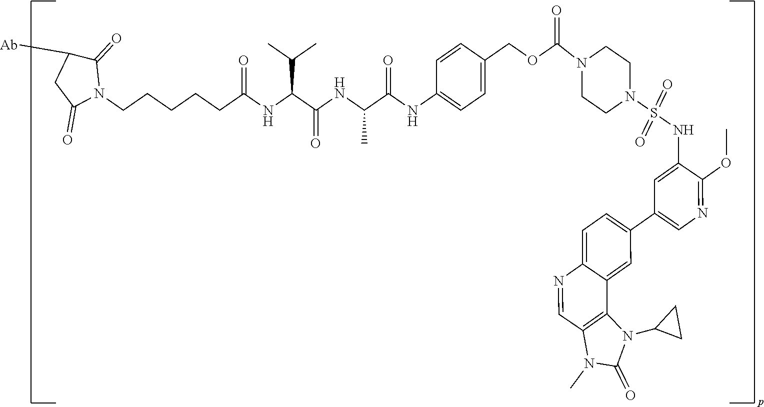

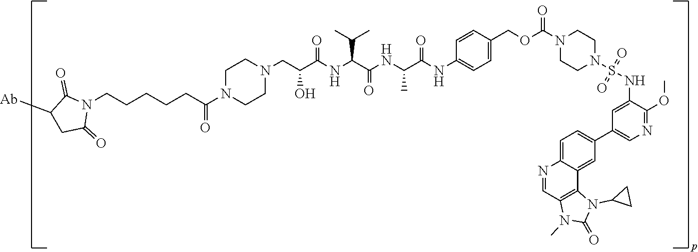

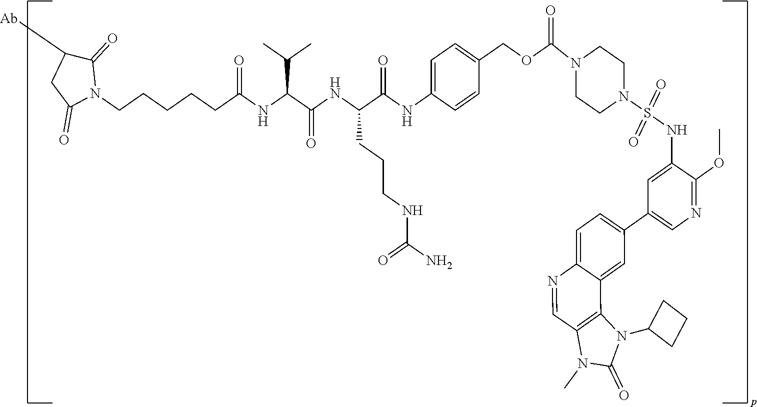

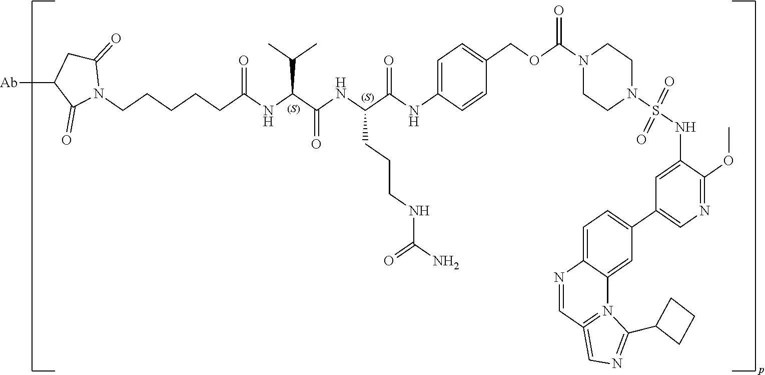

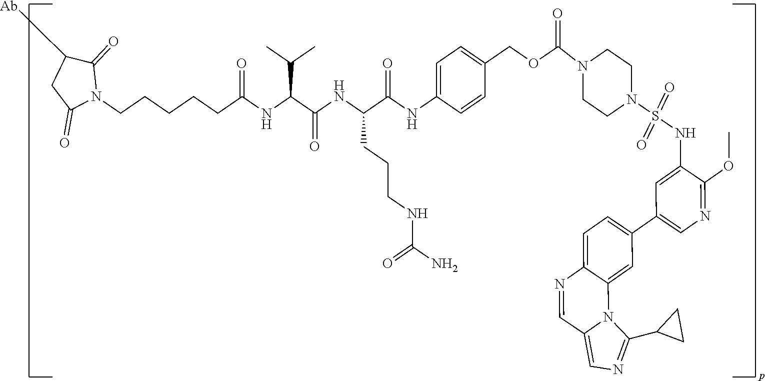

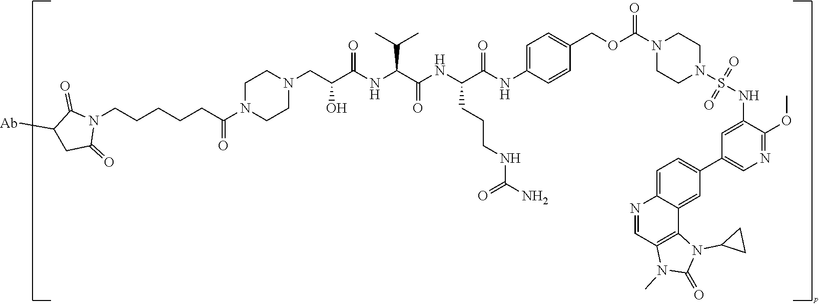

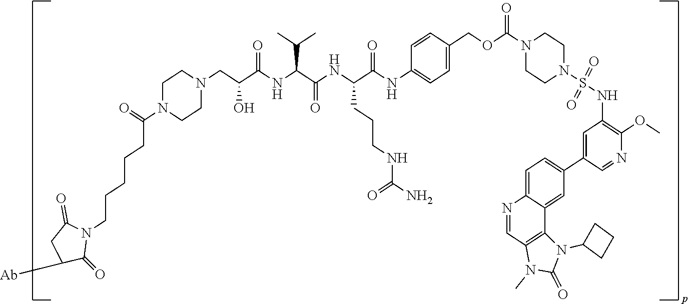

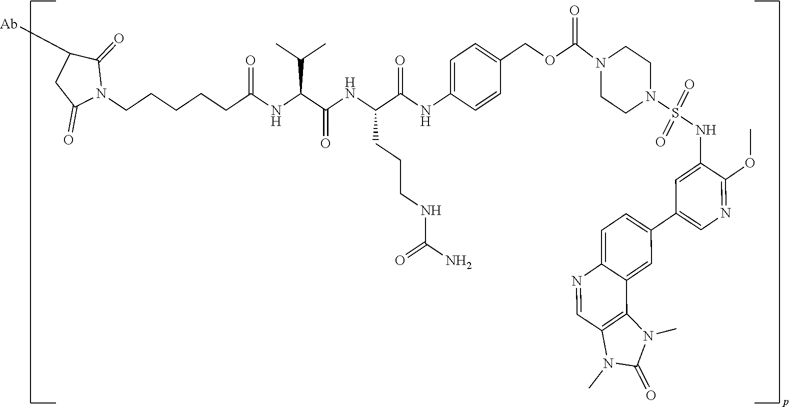

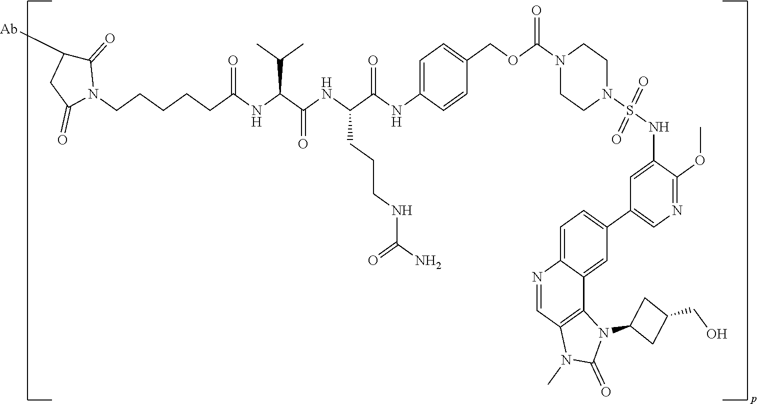

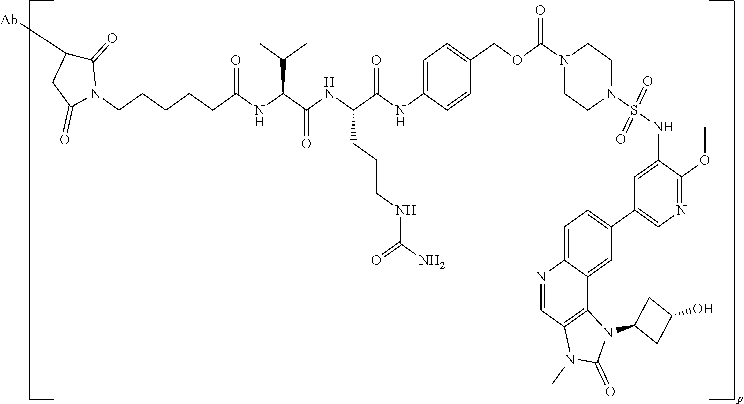

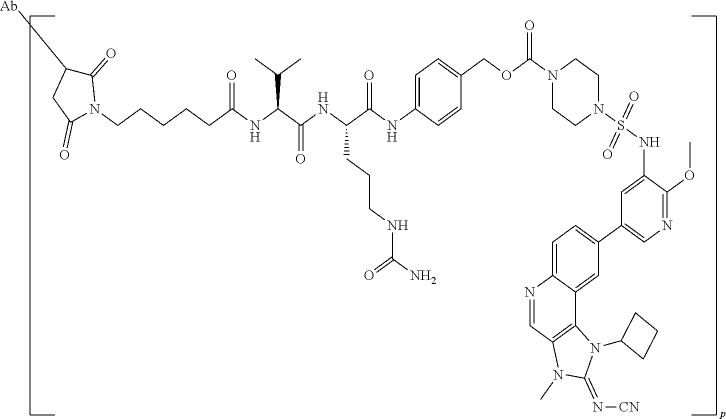

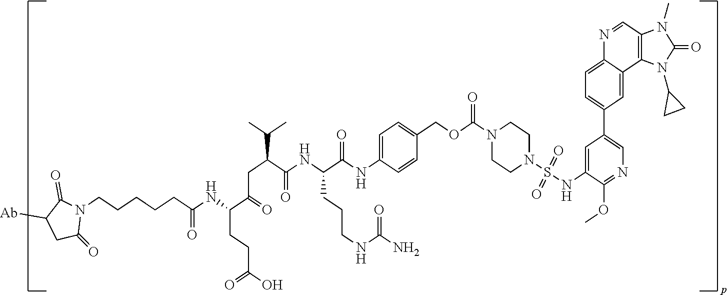

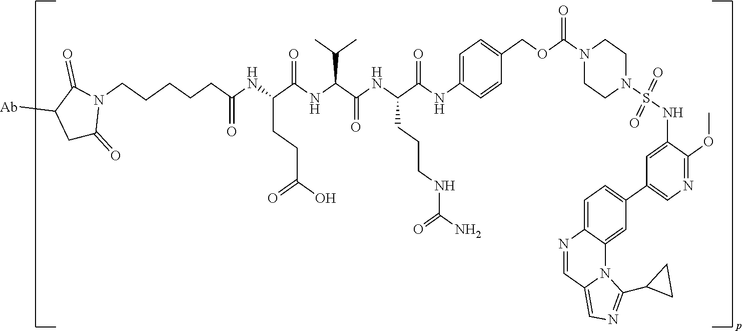

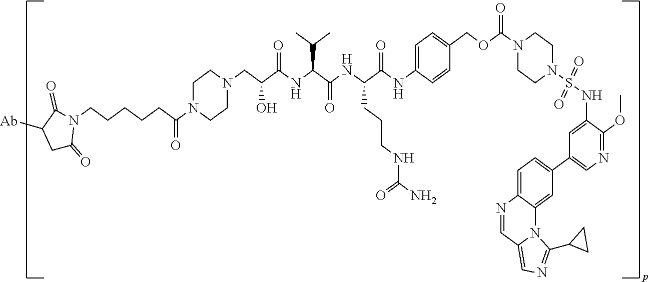

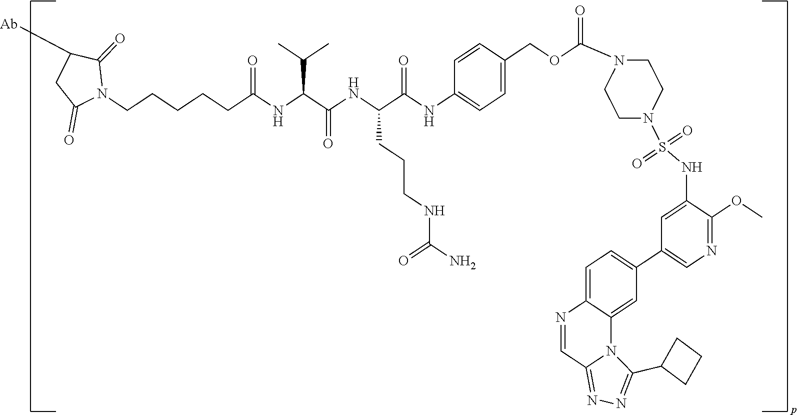

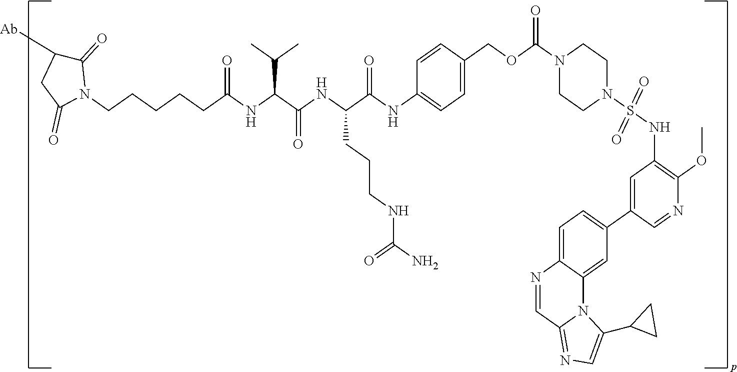

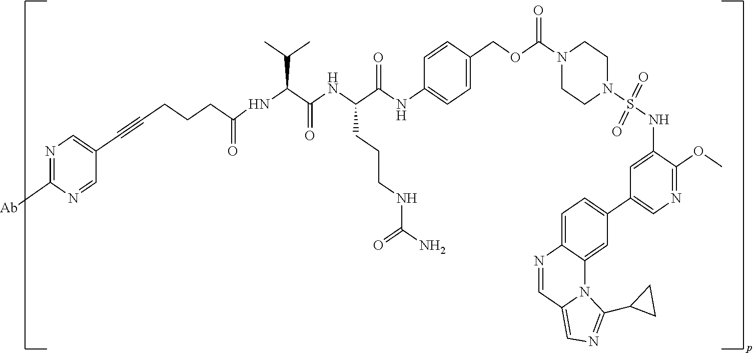

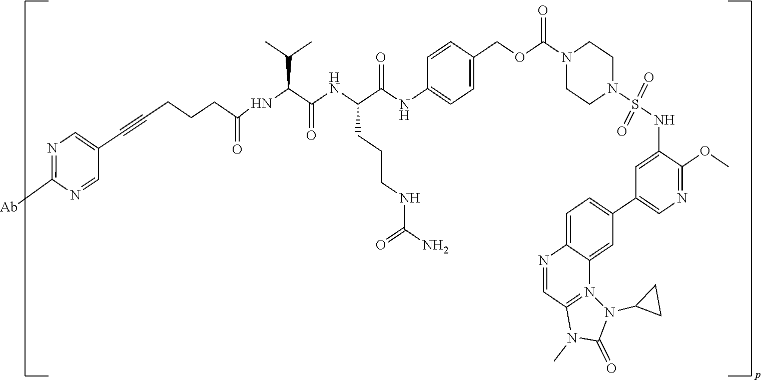

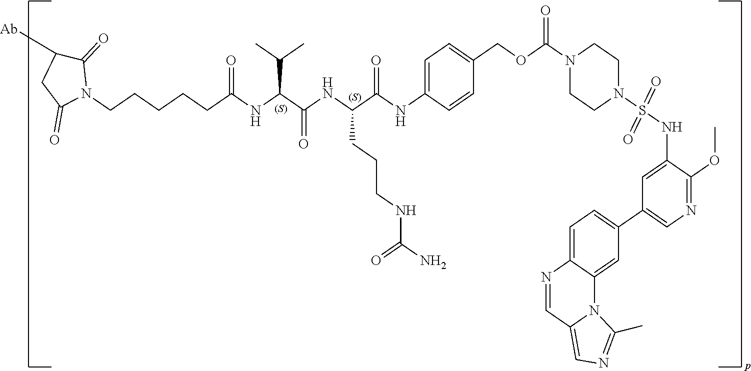

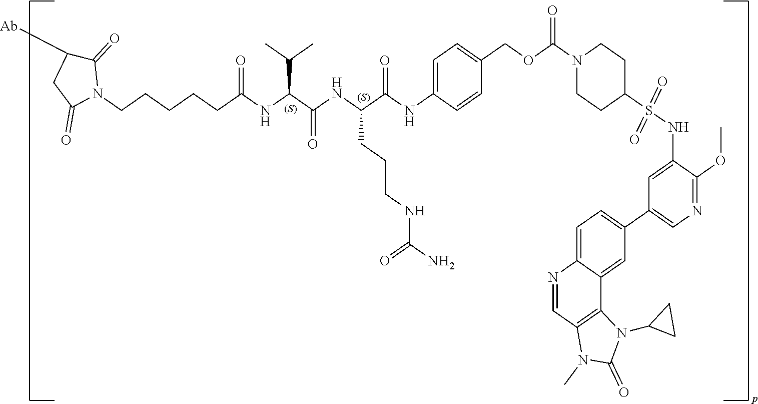

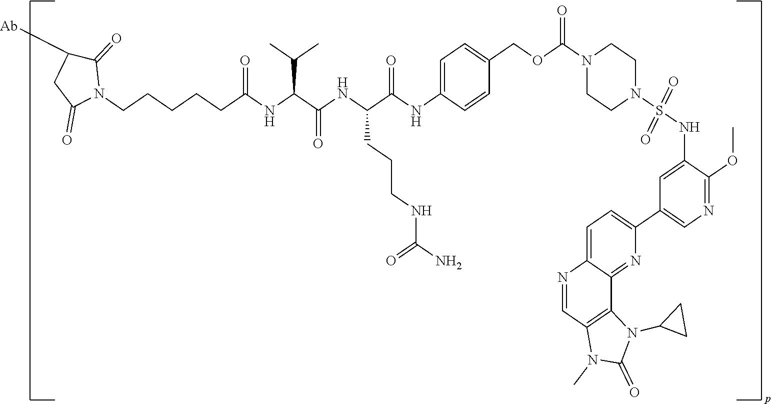

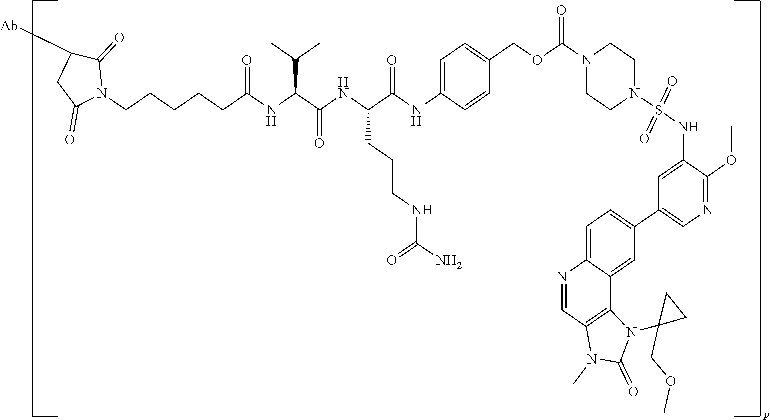

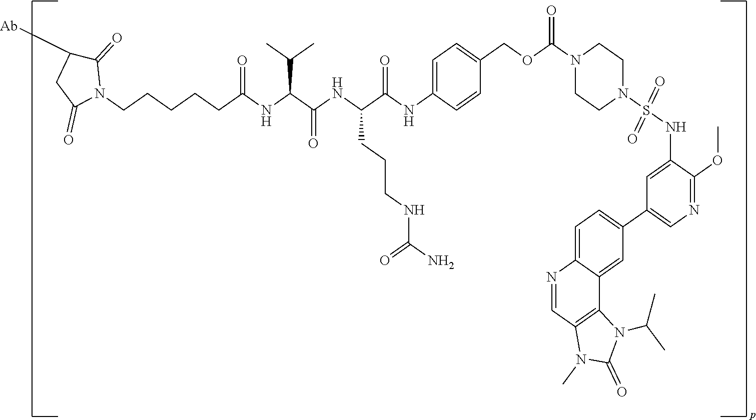

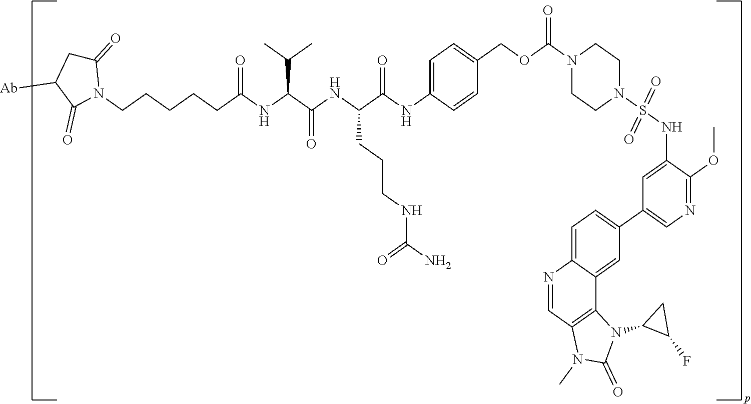

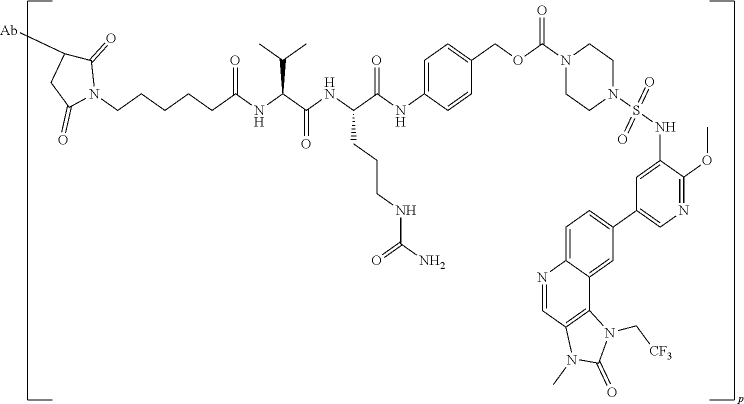

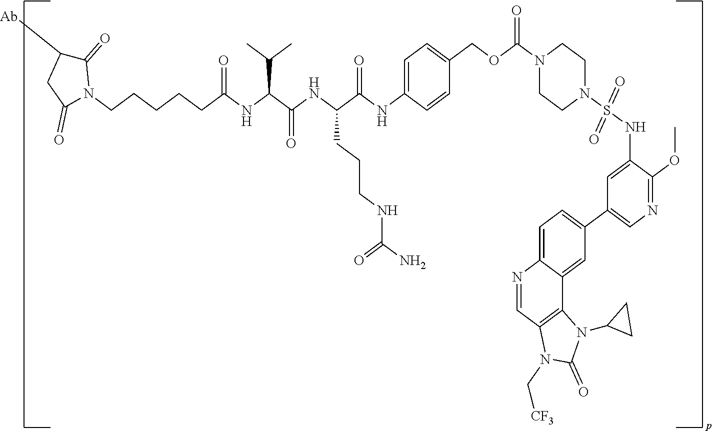

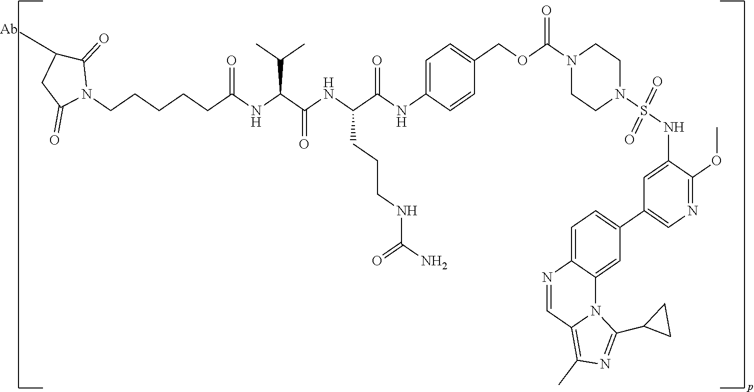

and wherein Ab and p are as defined in embodiment I-1.

Embodiment I-48. The drug conjugate or the pharmaceutically acceptable salt thereof, or the solvate, the racemic mixture, the enantiomer, the diastereomer or the tautomer thereof according to embodiment I-47, wherein p is selected from 2, 3, 4, 5, 6, 7, and 8.

Embodiment I-49. The drug conjugate or the pharmaceutically acceptable salt thereof, or the solvate, the racemic mixture, the enantiomer, the diastereomer or the tautomer thereof according to any one of embodiments I-1 to I-48, wherein Ab is an antibody or an antigen-binding fragment thereof that specifically binds to a tumor-associated antigen, such as an anti-HER2 antibody or an antigen-binding fragment thereof, an anti-CD38 antibody or an antigen-binding fragment thereof, an anti-CD20 antibody or an antigen-binding fragment thereof, an anti-CEACAM5/6 antibody or an antigen-binding fragment thereof, an anti-FRα antibody or an antigen-binding fragment thereof, an anti-Nectin-4 antibody or an antigen-binding fragment thereof.

Embodiment I-50. The drug conjugate or the pharmaceutically acceptable salt thereof, or the solvate, the racemic mixture, the enantiomer, the diastereomer or the tautomer thereof according to any one of embodiments I-1 to I-49, wherein

In some embodiments, the anti-HER2 antibody or the antigen-binding fragment thereof is any anti-HER2 antibody or antigen-binding fragment thereof known in the art; for example, the anti-HER2 antibody or the antigen-binding fragment thereof comprises 1, 2, 3, 4, 5, or 6 CDRs of an antibody (e.g., trastuzumab) that is known to specifically bind to HER2. In some embodiments, the anti-HER2 antibody or the antigen-binding fragment thereof comprises 1, 2, and 3 CDRs of the heavy chain variable region, i.e., HCDR1, HCDR2, and HCDR3, of the antibody (e.g., trastuzumab) that is known to specifically bind to HER2. In some embodiments, the anti-HER2 antibody or the antigen-binding fragment thereof comprises 1, 2, and 3 CDRs of the light chain variable region, i.e., LCDR1, LCDR2, and LCDR3, of the antibody (e.g., trastuzumab) that is known to specifically bind to HER2. In some embodiments, the anti-HER2 antibody or the antigen-binding fragment thereof comprises 3 CDRs of the heavy chain variable region and 3 CDRs of the light chain variable region of the antibody (e.g., trastuzumab) that is known to specifically bind to HER2. In some embodiments, the anti-HER2 antibody or the antigen-binding fragment thereof comprises a heavy chain variable region of the antibody (e.g., trastuzumab) that is known to specifically bind to HER2. In some embodiments, the anti-HER2 antibody or the antigen-binding fragment thereof comprises a light chain variable region of the antibody (e.g., trastuzumab) that is known to specifically bind to HER2. In some embodiments, the anti-HER2 antibody or the antigen-binding fragment thereof comprises a heavy chain variable region and a light chain variable region of the antibody (e.g., trastuzumab) that is known to specifically bind to HER2. In some embodiments, the anti-HER2 antibody or the antigen-binding fragment thereof comprises a heavy chain of the antibody (e.g., trastuzumab) that is known to specifically bind to HER2. In some embodiments, the anti-HER2 antibody or the antigen-binding fragment thereof comprises a light chain of the antibody (e.g., trastuzumab) that is known to specifically bind to HER2. In some embodiments, the anti-HER2 antibody or the antigen-binding fragment thereof comprises a heavy chain and a light chain of the antibody (e.g., trastuzumab) that is known to specifically bind to HER2. In some embodiments, the anti-HER2 antibody or antigen-binding fragment thereof comprises or consists of two heavy chains and two light chains of the antibody (e.g., trastuzumab) that is known to specifically bind to HER2.

In some embodiments, the anti-HER2 antibody or the antigen-binding fragment thereof comprises complementary determining regions LCDR1, LCDR2, LCDR3, HCDR1, HCDR2 and HCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of trastuzumab, respectively; or the HCDR1, HCDR2, HCDR3, LCDR1, LCDR2 and LCDR3 are the HCDR1, HCDR2, HCDR3, LCDR1, LCDR2 and LCDR3 of trastuzumab, respectively. In some embodiments, the anti-HER2 antibody or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of trastuzumab, respectively; or the heavy chain variable region and the light chain variable region are the heavy chain variable region and the light chain variable region of trastuzumab, respectively. In some embodiments, the anti-HER2 antibody or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of trastuzumab, respectively; or the heavy chain and the light chain are the heavy chain and the light chain of trastuzumab, respectively. In some embodiments, the anti-HER2 antibody has the same heavy chain and the same light chain as trastuzumab.

In some embodiments, the anti-HER2 antibody or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein
the HCDR1 comprises an amino acid sequence as set forth in SEQ ID NO: 1, HCDR2 comprises an amino acid sequence as set forth in SEQ ID NO: 2, and HCDR3 comprises an amino acid sequence as set forth in SEQ ID NO: 3; and the LCDR1 comprises an amino acid sequence as set forth in SEQ ID NO: 4, LCDR2 comprises an amino acid sequence as set forth in SEQ ID NO: 5, and LCDR3 comprises an amino acid sequence as set forth in SEQ ID NO: 6; or
the HCDR1 consists of an amino acid sequence as set forth in SEQ ID NO: 1, HCDR2 consists of an amino acid sequence as set forth in SEQ ID NO: 2, and HCDR3 consists of an amino acid sequence as set forth in SEQ ID NO: 3; and the LCDR1 consists of an amino acid sequence as set forth in SEQ ID NO: 4, LCDR2 consists of an amino acid sequence as set forth in SEQ ID NO: 5, and LCDR3 consists of an amino acid sequence as set forth in SEQ ID NO: 6.

In some embodiments, the anti-HER2 antibody or the antigen-binding fragment thereof comprises a heavy chain variable region and a light chain variable region, wherein the heavy chain variable region comprises an amino acid sequence as set forth in SEQ ID NO: 7, and the light chain variable region comprises an amino acid sequence as set forth in SEQ ID NO: 8; or the heavy chain variable region consists of an amino acid sequence as set forth in SEQ ID NO: 7, and the light chain variable region consists of an amino acid sequence as set forth in SEQ ID NO: 8.

In some embodiments, the anti-HER2 antibody or the antigen-binding fragment thereof comprises a heavy chain and a light chain, wherein the heavy chain comprises an amino acid sequence as set forth in SEQ ID NO: 9, and the light chain comprises an amino acid sequence as set forth in SEQ ID NO: 10; or the heavy chain consists of an amino acid sequence as set forth in SEQ ID NO: 9, and the light chain consists of an amino acid sequence as set forth in SEQ ID NO: 10. In some embodiments, the anti-HER2 antibody or the antigen-binding fragment thereof comprises the two said heavy chains and the two said light chains. In some embodiments, the anti-HER2 antibody is a full-length antibody. In some embodiments, the anti-HER2 antibody is selected from trastuzumab or the biosimilar of trastuzumab, or the anti-HER2 antibodies or antigen-binding fragments thereof as defined herein.

In some embodiments, the anti-CD38 antibody or antigen-binding fragment thereof is any anti-CD38 antibody or antigen-binding fragment thereof known in the art; for example, the anti-CD38 antibody or antigen-binding fragment thereof comprises 1, 2, 3, 4, 5, or 6 CDRs of an antibody (e.g., daratumumab) that is known to specifically bind to CD38. In some embodiments, the anti-CD38 antibody or antigen-binding fragment thereof comprises 1, 2, and 3 CDRs of the heavy chain variable region, i.e., HCDR1, HCDR2, and HCDR3, of the antibody (e.g., daratumumab) that is known to specifically bind to CD38. In some embodiments, the anti-CD38 antibody or antigen-binding fragment thereof comprises 1, 2, and 3 CDRs of the light chain variable region, i.e., LCDR1, LCDR2, and LCDR3, of the antibody (e.g., daratumumab) that is known to specifically bind to CD38. In some embodiments, the anti-CD38 antibody or antigen-binding fragment thereof comprises 3 CDRs of the heavy chain variable region and 3 CDRs of the light chain variable region of the antibody (e.g., daratumumab) that is known to specifically bind to CD38. In some embodiments, the anti-CD38 antibody or antigen-binding fragment thereof comprises a heavy chain variable region of the antibody (e.g., daratumumab) that is known to specifically bind to CD38. In some embodiments, the anti-CD38 antibody or antigen-binding fragment thereof comprises a light chain variable region of the antibody (e.g., daratumumab) that is known to specifically bind to CD38. In some embodiments, the anti-CD38 antibody or antigen-binding fragment thereof comprises a heavy chain variable region and a light chain variable region of the antibody (e.g., daratumumab) that is known to specifically bind to CD38. In some embodiments, the anti-CD38 antibody or antigen-binding fragment thereof comprises a heavy chain of the antibody (e.g., daratumumab) that is known to specifically bind to CD38. In some embodiments, the anti-CD38 antibody or antigen-binding fragment thereof comprises a light chain of the antibody (e.g., daratumumab) that is known to specifically bind to CD38. In some embodiments, the anti-CD38 antibody or antigen-binding fragment thereof comprises a heavy chain and a light chain of the antibody (e.g., daratumumab) that is known to specifically bind to CD38. In some embodiments, the anti-CD38 antibody or antigen-binding fragment thereof comprises or consists of two heavy chains and two light chains of the antibody (e.g., daratumumab) that is known to specifically bind to CD38.

In some embodiments, the anti-CD38 antibody or the antigen-binding fragment thereof comprises complementary determining regions LCDR1, LCDR2, LCDR3, HCDR1, HCDR2 and HCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of daratumumab, respectively; or the HCDR1, HCDR2, HCDR3, LCDR1, LCDR2 and LCDR3 are the HCDR1, HCDR2, HCDR3, LCDR1, LCDR2 and LCDR3 of daratumumab, respectively. In some embodiments, the anti-CD38 antibody or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of daratumumab, respectively; or the heavy chain variable region and the light chain variable region are the heavy chain variable region and the light chain variable region of daratumumab, respectively. In some embodiments, the anti-CD38 antibody or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of daratumumab, respectively; or the heavy chain and the light chain are the heavy chain and the light chain of daratumumab, respectively. In some embodiments, the anti-CD38 antibody has the same heavy chain and the same light chain as daratumumab.

In some embodiments, the anti-CD38 antibody or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein
the HCDR1 comprises an amino acid sequence as set forth in SEQ ID NO: 11, HCDR2 comprises an amino acid sequence as set forth in SEQ ID NO: 12, and HCDR3 comprises an amino acid sequence as set forth in SEQ ID NO: 13; and the LCDR1 comprises an amino acid sequence as set forth in SEQ ID NO: 14, LCDR2 comprises an amino acid sequence as set forth in SEQ ID NO: 15, and LCDR3 comprises an amino acid sequence as set forth in SEQ ID NO: 16; or
the HCDR1 consists of an amino acid sequence as set forth in SEQ ID NO: 11, HCDR2 consists of an amino acid sequence as set forth in SEQ ID NO: 12, and HCDR3 consists of an amino acid sequence as set forth in SEQ ID NO: 13; and the LCDR1 consists of an amino acid sequence as set forth in SEQ ID NO: 14, LCDR2 consists of an amino acid sequence as set forth in SEQ ID NO: 15, and LCDR3 consists of an amino acid sequence as set forth in SEQ ID NO: 16.

In some embodiments, the anti-CD38 antibody or the antigen-binding fragment thereof comprises a heavy chain variable region and a light chain variable region, wherein the heavy chain variable region comprises an amino acid sequence as set forth in SEQ ID NO: 17, and the light chain variable region comprises an amino acid sequence as set forth in SEQ ID NO: 18; or the heavy chain variable region consists of an amino acid sequence as set forth in SEQ ID NO: 17, and the light chain variable region consists of an amino acid sequence as set forth in SEQ ID NO: 18.

In some embodiments, the anti-CD38 antibody or the antigen-binding fragment thereof comprises a heavy chain and a light chain, wherein the heavy chain comprises an amino acid sequence as set forth in SEQ ID NO: 19, and the light chain comprises an amino acid sequence as set forth in SEQ ID NO: 20; or the heavy chain consists of an amino acid sequence as set forth in SEQ ID NO: 19, and the light chain consists of an amino acid sequence as set forth in SEQ ID NO: 20. In some embodiments, the anti-CD38 antibody or the antigen-binding fragment thereof comprises the two said heavy chains and the two said light chains.

In some embodiments, the anti-CD38 antibody is a full-length antibody. In some embodiments, the anti-CD38 antibody is selected from daratumumab or the biosimilar of daratumumab, or the anti-CD38 antibodies or antigen-binding fragments thereof as defined herein.

In some embodiments, the anti-CD20 antibody or antigen-binding fragment thereof is any anti-CD20 antibody or antigen-binding fragment thereof known in the art; for example, the anti-CD20 antibody or antigen-binding fragment thereof comprises 1, 2, 3, 4, 5, or 6 CDRs of an antibody (e.g., rituximab) that is known to specifically bind to CD20. In some embodiments, the anti-CD20 antibody or antigen-binding fragment thereof comprises 1, 2, and 3 CDRs of the heavy chain variable region, i.e., HCDR1, HCDR2, and HCDR3, of the antibody (e.g., rituximab) that is known to specifically bind to CD20. In some embodiments, the anti-CD20 antibody or antigen-binding fragment thereof comprises 1, 2, and 3 CDRs of the light chain variable region, i.e., LCDR1, LCDR2, and LCDR3, of the antibody (e.g., rituximab) that is known to specifically bind to CD20. In some embodiments, the anti-CD20 antibody or antigen-binding fragment thereof comprises 3 CDRs of the heavy chain variable region and 3 CDRs of the light chain variable region of the antibody (e.g., rituximab) that is known to specifically bind to CD20. In some embodiments, the anti-CD20 antibody or antigen-binding fragment thereof comprises a heavy chain variable region of the antibody (e.g., rituximab) that is known to specifically bind to CD20. In some embodiments, the anti-CD20 antibody or antigen-binding fragment thereof comprises a light chain variable region of the antibody (e.g., rituximab) that is known to specifically bind to CD20. In some embodiments, the anti-CD20 antibody or antigen-binding fragment thereof comprises a heavy chain variable region and a light chain variable region of the antibody (e.g., rituximab) that is known to specifically bind to CD20. In some embodiments, the anti-CD20 antibody or antigen-binding fragment thereof comprises a heavy chain of the antibody (e.g., rituximab) that is known to specifically bind to CD20. In some embodiments, the anti-CD20 antibody or antigen-binding fragment thereof comprises a light chain of the antibody (e.g., rituximab) that is known to specifically bind to CD20. In some embodiments, the anti-CD20 antibody or antigen-binding fragment thereof comprises a heavy chain and a light chain of the antibody (e.g., rituximab) that is known to specifically bind to CD20. In some embodiments, the anti-CD20 antibody or antigen-binding fragment thereof comprises or consists of two heavy chains and two light chains of the antibody (e.g., rituximab) that is known to specifically bind to CD20.

In some embodiments, the anti-CD20 antibody or the antigen-binding fragment thereof comprises complementary determining regions LCDR1, LCDR2, LCDR3, HCDR1, HCDR2 and HCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of rituximab, respectively; or the HCDR1, HCDR2, HCDR3, LCDR1, LCDR2 and LCDR3 are the HCDR1, HCDR2, HCDR3, LCDR1, LCDR2 and LCDR3 of rituximab, respectively. In some embodiments, the anti-CD20 antibody or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of rituximab, respectively; or the heavy chain variable region and the light chain variable region are the heavy chain variable region and the light chain variable region of rituximab, respectively. In some embodiments, the anti-CD20 antibody or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of rituximab, respectively; or the heavy chain and the light chain are the heavy chain and the light chain of rituximab, respectively. In some embodiments, the anti-CD20 antibody has the same heavy chain and the same light chain as rituximab.

In some embodiments, the anti-CD20 antibody or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein the HCDR1 comprises an amino acid sequence as set forth in SEQ ID NO: 21, HCDR2 comprises an amino acid sequence as set forth in SEQ ID NO: 22, and HCDR3 comprises an amino acid sequence as set forth in SEQ ID NO: 23; and the LCDR1 comprises an amino acid sequence as set forth in SEQ ID NO: 24, LCDR2 comprises an amino acid sequence as set forth in SEQ ID NO: 25, and LCDR3 comprises an amino acid sequence as set forth in SEQ ID NO: 26; or
the HCDR1 consists of an amino acid sequence as set forth in SEQ ID NO: 21, HCDR2 consists of an amino acid sequence as set forth in SEQ ID NO: 22, and HCDR3 consists of an amino acid sequence as set forth in SEQ ID NO: 23; and the LCDR1 consists of an amino acid sequence as set forth in SEQ ID NO: 24, LCDR2 consists of an amino acid sequence as set forth in SEQ ID NO: 25, and LCDR3 consists of an amino acid sequence as set forth in SEQ ID NO: 26.

In some embodiments, the anti-CD20 antibody or the antigen-binding fragment thereof comprises a heavy chain variable region and a light chain variable region, wherein the heavy chain variable region comprises an amino acid sequence as set forth in SEQ ID NO: 27, and the light chain variable region comprises an amino acid sequence as set forth in SEQ ID NO: 28; or the heavy chain variable region consists of an amino acid sequence as set forth in SEQ ID NO: 27, and the light chain variable region consists of an amino acid sequence as set forth in SEQ ID NO: 28.

In some embodiments, the anti-CD20 antibody or the antigen-binding fragment thereof comprises a heavy chain and a light chain, wherein the heavy chain comprises an amino acid sequence as set forth in SEQ ID NO: 29, and the light chain comprises an amino acid sequence as set forth in SEQ ID NO: 30; or the heavy chain consists of an amino acid sequence as set forth in SEQ ID NO: 29, and the light chain consists of an amino acid sequence as set forth in SEQ ID NO: 30. In some embodiments, the anti-CD20 antibody or the antigen-binding fragment thereof comprises the two said heavy chains and the two said light chains. In some embodiments, the anti-CD20 antibody is a full-length antibody. In some embodiments, the anti-CD20 antibody is selected from rituximab or the biosimilar of rituximab, or the anti-CD20 antibodies or antigen-binding fragments thereof as defined herein.

In some embodiments, the anti-CEACAM5/6 antibody or antigen-binding fragment thereof is any anti-CEACAM5/6 antibody or antigen-binding fragment thereof known in the art; for example, the anti-CEACAM5/6 antibody or antigen-binding fragment thereof comprises 1, 2, 3, 4, 5, or 6 CDRs of an antibody (e.g., NEO-201) that is known to specifically bind to CEACAM5/6. In some embodiments, the anti-CEACAM5/6 antibody or antigen-binding fragment thereof comprises 1, 2, and 3 CDRs of the heavy chain variable region, i.e., HCDR1, HCDR2, and HCDR3, of the antibody (e.g., NEO-201) that is known to specifically bind to CEACAM5/6. In some embodiments, the anti-CEACAM5/6 antibody or antigen-binding fragment thereof comprises 1, 2, and 3 CDRs of the light chain variable region, i.e., LCDR1, LCDR2, and LCDR3, of the antibody (e.g., NEO-201) that is known to specifically bind to CEACAM5/6. In some embodiments, the anti-CEACAM5/6 antibody or antigen-binding fragment thereof comprises 3 CDRs of the heavy chain variable region and 3 CDRs of the light chain variable region of the antibody (e.g., NEO-201) that is known to specifically bind to CEACAM5/6. In some embodiments, the anti-CEACAM5/6 antibody or antigen-binding fragment thereof comprises a heavy chain variable region of the antibody (e.g., NEO-201) that is known to specifically bind to CEACAM5/6. In some embodiments, the anti-CEACAM5/6 antibody or antigen-binding fragment thereof comprises a light chain variable region of the antibody (e.g., NEO-201) that is known to specifically bind to CEACAM5/6. In some embodiments, the anti-CEACAM5/6 antibody or antigen-binding fragment thereof comprises a heavy chain variable region and a light chain variable region of the antibody (e.g., NEO-201) that is known to specifically bind to CEACAM5/6. In some embodiments, the anti-CEACAM5/6 antibody or antigen-binding fragment thereof comprises a heavy chain of the antibody (e.g., NEO-201) that is known to specifically bind to CEACAM5/6. In some embodiments, the anti-CEACAM5/6 antibody or antigen-binding fragment thereof comprises a light chain of the antibody (e.g., NEO-201) that is known to specifically bind to CEACAM5/6. In some embodiments, the anti-CEACAM5/6 antibody or antigen-binding fragment thereof comprises a heavy chain and a light chain of the antibody (e.g., NEO-201) that is known to specifically bind to CEACAM5/6. In some embodiments, the anti-CEACAM5/6 antibody or antigen-binding fragment thereof comprises or consists of two heavy chains and two light chains of the antibody (e.g., NEO-201) that is known to specifically bind to CEACAM5/6.

In some embodiments, the anti-CEACAM5/6 antibody or the antigen-binding fragment thereof comprises complementary determining regions LCDR1, LCDR2, LCDR3, HCDR1, HCDR2 and HCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of NEO-201, respectively; or the HCDR1, HCDR2, HCDR3, LCDR1, LCDR2 and LCDR3 are the HCDR1, HCDR2, HCDR3, LCDR1, LCDR2 and LCDR3 of NEO-201, respectively. In some embodiments, the anti-CEACAM5/6 antibody or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of NEO-201, respectively; or the heavy chain variable region and the light chain variable region are the heavy chain variable region and the light chain variable region of NEO-201, respectively. In some embodiments, the anti-CEACAM5/6 antibody or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of NEO-201, respectively; or the heavy chain and the light chain are the heavy chain and the light chain of NEO-201, respectively. In some embodiments, the anti-CEACAM5/6 antibody has the same heavy chain and the same light chain as NEO-201.

In some embodiments, the anti-CEACAM5/6 antibody or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein the HCDR1 comprises an amino acid sequence as set forth in SEQ ID NO: 31, HCDR2 comprises an amino acid sequence as set forth in SEQ ID NO: 32, and HCDR3 comprises an amino acid sequence as set forth in SEQ ID NO: 33; and the LCDR1 comprises an amino acid sequence as set forth in SEQ ID NO: 34, LCDR2 comprises an amino acid sequence as set forth in SEQ ID NO: 35, and LCDR3 comprises an amino acid sequence as set forth in SEQ ID NO: 36; or the HCDR1 consists of an amino acid sequence as set forth in SEQ ID NO: 31, HCDR2 consists of an amino acid sequence as set forth in SEQ ID NO: 32, and HCDR3 consists of an amino acid sequence as set forth in SEQ ID NO: 33; and the LCDR1 consists of an amino acid sequence as set forth in SEQ ID NO: 34, LCDR2 consists of an amino acid sequence as set forth in SEQ ID NO: 35, and LCDR3 consists of an amino acid sequence as set forth in SEQ ID NO: 36.

In some embodiments, the anti-CEACAM5/6 antibody or the antigen-binding fragment thereof comprises a heavy chain variable region and a light chain variable region, wherein the heavy chain variable region comprises an amino acid sequence as set forth in SEQ ID NO: 37, and the light chain variable region comprises an amino acid sequence as set forth in SEQ ID NO: 38; or the heavy chain variable region consists of an amino acid sequence as set forth in SEQ ID NO: 37, and the light chain variable region consists of an amino acid sequence as set forth in SEQ ID NO: 38.

In some embodiments, the anti-CEACAM5/6 antibody or the antigen-binding fragment thereof comprises a heavy chain and a light chain, wherein the heavy chain comprises an amino acid sequence as set forth in SEQ ID NO: 39, and the light chain comprises an amino acid sequence as set forth in SEQ ID NO: 40; or the heavy chain consists of an amino acid sequence as set forth in SEQ ID NO: 39, and the light chain consists of an amino acid sequence as set forth in SEQ ID NO: 40. In some embodiments, the anti-CEACAM5/6 antibody or the antigen-binding fragment thereof comprises the two said heavy chains and the two said light chains. In some embodiments, the anti-CEACAM5/6 antibody is a full-length antibody. In some embodiments, the anti-CEACAM5/6 antibody is selected from NEO-201 or a biosimilar of NEO-201, or the anti-CEACAM5/6 antibodies or antigen-binding fragments thereof as defined herein.

In some embodiments, the anti-FRα antibody or antigen-binding fragment thereof is any anti-FRα antibody or antigen-binding fragment thereof known in the art; for example, the anti-FRα antibody or antigen-binding fragment thereof comprises 1, 2, 3, 4, 5, or 6 CDRs of an antibody (e.g., Mirvetuximab) that is known to specifically bind to FRα. In some embodiments, the anti-FRα antibody or antigen-binding fragment thereof comprises 1, 2, and 3 CDRs of the heavy chain variable region, i.e., HCDR1, HCDR2, and HCDR3, of the antibody (e.g., Mirvetuximab) that is known to specifically bind to FRα. In some embodiments, the anti-FRα antibody or antigen-binding fragment thereof comprises 1, 2, and 3 CDRs of the light chain variable region, i.e., LCDR1, LCDR2, and LCDR3, of the antibody (e.g., Mirvetuximab) that is known to specifically bind to FRα. In some embodiments, the anti-FRα antibody or antigen-binding fragment thereof comprises 3 CDRs of the heavy chain variable region and 3 CDRs of the light chain variable region of the antibody (e.g., Mirvetuximab) that is known to specifically bind to FRα. In some embodiments, the anti-FRα antibody or antigen-binding fragment thereof comprises a heavy chain variable region of the antibody (e.g., Mirvetuximab) that is known to specifically bind to FRα. In some embodiments, the anti-FRα antibody or antigen-binding fragment thereof comprises a light chain variable region of the antibody (e.g., Mirvetuximab) that is known to specifically bind to FRα. In some embodiments, the anti-FRα antibody or antigen-binding fragment thereof comprises a heavy chain variable region and a light chain variable region of the antibody (e.g., Mirvetuximab) that is known to specifically bind to FRα. In some embodiments, the anti-FRα antibody or antigen-binding fragment thereof comprises a heavy chain of the antibody (e.g., Mirvetuximab) that is known to specifically bind to FRα. In some embodiments, the anti-FRα antibody or antigen-binding fragment thereof comprises a light chain of the antibody (e.g., Mirvetuximab) that is known to specifically bind to FRα. In some embodiments, the anti-FRα antibody or antigen-binding fragment thereof comprises a heavy chain and a light chain of the antibody (e.g., Mirvetuximab) that is known to specifically bind to FRα. In some embodiments, the anti-FRα antibody or antigen-binding fragment thereof comprises or consists of two heavy chains and two light chains of the antibody (e.g., Mirvetuximab) that is known to specifically bind to FRα.

In some embodiments, the anti-FRα antibody or the antigen-binding fragment thereof comprises complementary determining regions LCDR1, LCDR2, LCDR3, HCDR1, HCDR2 and HCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of Mirvetuximab, respectively; or the HCDR1, HCDR2, HCDR3, LCDR1, LCDR2 and LCDR3 are the HCDR1, HCDR2, HCDR3, LCDR1, LCDR2 and LCDR3 of Mirvetuximab, respectively. In some embodiments, the anti-FRα antibody or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of Mirvetuximab, respectively; or the heavy chain variable region and the light chain variable region are the heavy chain variable region and the light chain variable region of Mirvetuximab, respectively. In some embodiments, the anti-FRα antibody or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of Mirvetuximab, respectively; or the heavy chain and the light chain are the heavy chain and the light chain of Mirvetuximab, respectively. In some embodiments, the anti-FRα antibody has the same heavy chain and the same light chain as Mirvetuximab.

In some embodiments, the anti-FRα antibody or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein
the HCDR1 comprises an amino acid sequence as set forth in SEQ ID NO: 51, HCDR2 comprises an amino acid sequence as set forth in SEQ ID NO: 52, and HCDR3 comprises an amino acid sequence as set forth in SEQ ID NO: 53; and the LCDR1 comprises an amino acid sequence as set forth in SEQ ID NO: 54, LCDR2 comprises an amino acid sequence as set forth in SEQ ID NO: 55, and LCDR3 comprises an amino acid sequence as set forth in SEQ ID NO: 56; or
the HCDR1 consists of an amino acid sequence as set forth in SEQ ID NO: 51, HCDR2 consists of an amino acid sequence as set forth in SEQ ID NO: 52, and HCDR3 consists of an amino acid sequence as set forth in SEQ ID NO: 53; and the LCDR1 consists of an amino acid sequence as set forth in SEQ ID NO: 54, LCDR2 consists of an amino acid sequence as set forth in SEQ ID NO: 55, and LCDR3 consists of an amino acid sequence as set forth in SEQ ID NO: 56.

In some embodiments, the anti-FRα antibody or the antigen-binding fragment thereof comprises a heavy chain variable region and a light chain variable region, wherein the heavy chain variable region comprises an amino acid sequence as set forth in SEQ ID NO: 57, and the light chain variable region comprises an amino acid sequence as set forth in SEQ ID NO: 58; or the heavy chain variable region consists of an amino acid sequence as set forth in SEQ ID NO: 57, and the light chain variable region consists of an amino acid sequence as set forth in SEQ ID NO: 58.

In some embodiments, the anti-FRα antibody or the antigen-binding fragment thereof comprises a heavy chain and a light chain, wherein the heavy chain comprises an amino acid sequence as set forth in SEQ ID NO: 59, and the light chain comprises an amino acid sequence as set forth in SEQ ID NO: 60; or the heavy chain consists of an amino acid sequence as set forth in SEQ ID NO: 59, and the light chain consists of an amino acid sequence as set forth in SEQ ID NO: 60. In some embodiments, the anti-FRα antibody or the antigen-binding fragment thereof comprises the two said heavy chains and the two said light chains. In some embodiments, the anti-FRα antibody is a full-length antibody. In some embodiments, the anti-FRα antibody is selected from Mirvetuximab or a biosimilar of Mirvetuximab, or the anti-FRα antibodies or antigen-binding fragments thereof as defined herein.

In some embodiments, the anti-Nectin-4 antibody or antigen-binding fragment thereof is any anti-Nectin-4 antibody or antigen-binding fragment thereof known in the art; for example, the anti-Nectin-4 antibody or antigen-binding fragment thereof comprises 1, 2, 3, 4, 5, or 6 CDRs of an antibody (e.g., Enfortumab) that is known to specifically bind to Nectin-4. In some embodiments, the anti-Nectin-4 antibody or antigen-binding fragment thereof comprises 1, 2, and 3 CDRs of the heavy chain variable region, i.e., HCDR1, HCDR2, and HCDR3, of the antibody (e.g., Enfortumab) that is known to specifically bind to Nectin-4. In some embodiments, the anti-Nectin-4 antibody or antigen-binding fragment thereof comprises 1, 2, and 3 CDRs of the light chain variable region, i.e., LCDR1, LCDR2, and LCDR3, of the antibody (e.g., Enfortumab) that is known to specifically bind to Nectin-4. In some embodiments, the anti-Nectin-4 antibody or antigen-binding fragment thereof comprises 3 CDRs of the heavy chain variable region and 3 CDRs of the light chain variable region of the antibody (e.g., Enfortumab) that is known to specifically bind to Nectin-4. In some embodiments, the anti-Nectin-4 antibody or antigen-binding fragment thereof comprises a heavy chain variable region of the antibody (e.g., Enfortumab) that is known to specifically bind to Nectin-4. In some embodiments, the anti-Nectin-4 antibody or antigen-binding fragment thereof comprises a light chain variable region of the antibody (e.g., Enfortumab) that is known to specifically bind to Nectin-4. In some embodiments, the anti-Nectin-4 antibody or antigen-binding fragment thereof comprises a heavy chain variable region and a light chain variable region of the antibody (e.g., Enfortumab) that is known to specifically bind to Nectin-4. In some embodiments, the anti-Nectin-4 antibody or antigen-binding fragment thereof comprises a heavy chain of the antibody (e.g., Enfortumab) that is known to specifically bind to Nectin-4. In some embodiments, the anti-Nectin-4 antibody or antigen-binding fragment thereof comprises a light chain of the antibody (e.g., Enfortumab) that is known to specifically bind to Nectin-4. In some embodiments, the anti-Nectin-4 antibody or antigen-binding fragment thereof comprises a heavy chain and a light chain of the antibody (e.g., Enfortumab) that is known to specifically bind to Nectin-4. In some embodiments, the anti-Nectin-4 antibody or antigen-binding fragment thereof comprises or consists of two heavy chains and two light chains of the antibody (e.g., Enfortumab) that is known to specifically bind to Nectin-4.

In some embodiments, the anti-Nectin-4 antibody or the antigen-binding fragment thereof comprises complementary determining regions LCDR1, LCDR2, LCDR3, HCDR1, HCDR2 and HCDR3; wherein the amino acid sequences of the HCDR1, HCDR2, HCDR3, LCDR1, LCDR2 and LCDR3 are the same as those of HCDR1, HCDR2, HCDR3, LCDR1, LCDR2 and LCDR3 of Enfortumab, respectively; or the HCDR1, HCDR2, HCDR3, LCDR1, LCDR2 and LCDR3 are the HCDR1, HCDR2, HCDR3, LCDR1, LCDR2 and LCDR3 of Enfortumab, respectively. In some embodiments, the anti-Nectin-4 antibody or the antigen-binding fragment thereof comprises a heavy chain variable region and a light chain variable region, and the amino acid sequence of the heavy chain variable region and the amino acid sequence of the light chain variable region are the same as the amino acid sequence of the heavy chain variable region and the amino acid sequence of the light chain variable region of Enfortumab, respectively; or the heavy chain variable region and the light chain variable region are the heavy chain variable region and the light chain variable region of Enfortumab, respectively. In some embodiments, the anti-Nectin-4 antibody or the antigen-binding fragment thereof comprises a heavy chain and a light chain, and the amino acid sequence of the heavy chain and the amino acid sequence of the light chain are the same as the amino acid sequence of the heavy chain and the amino acid sequence of the light chain of Enfortumab, respectively; or the heavy chain and the light chain are the heavy chain and the light chain of Enfortumab, respectively. In some embodiments, the anti-Nectin-4 antibody has the same heavy chain and the same light chain as Enfortumab.

In some embodiments, the anti-Nectin-4 antibody or the antigen-binding fragment thereof comprises LCDR1, LCDR2, LCDR3, HCDR1, HCDR2 and HCDR3, or comprises a light chain variable region comprising LCDR1, LCDR2 and LCDR3 and a heavy chain variable region comprising HCDR1, HCDR2 and HCDR3; wherein
the HCDR1 comprises an amino acid sequence as set forth in SEQ ID NO: 41, HCDR2 comprises an amino acid sequence as set forth in SEQ ID NO: 42, and HCDR3 comprises an amino acid sequence as set forth in SEQ ID NO: 43; and the LCDR1 comprises an amino acid sequence as set forth in SEQ ID NO: 44, LCDR2 comprises an amino acid sequence as set forth in SEQ ID NO: 45, and LCDR3 comprises an amino acid sequence as set forth in SEQ ID NO: 46; or
the HCDR1 consists of an amino acid sequence as set forth in SEQ ID NO: 41, HCDR2 consists of an amino acid sequence as set forth in SEQ ID NO: 42, and HCDR3 consists of an amino acid sequence as set forth in SEQ ID NO: 43; and the LCDR1 consists of an amino acid sequence as set forth in SEQ ID NO: 44, LCDR2 consists of an amino acid sequence as set forth in SEQ ID NO: 45, and LCDR3 consists of an amino acid sequence as set forth in SEQ ID NO: 46.

In some embodiments, the anti-Nectin-4 antibody or the antigen-binding fragment thereof comprises a heavy chain variable region and a light chain variable region, wherein the heavy chain variable region comprises an amino acid sequence as set forth in SEQ ID NO: 47, and the light chain variable region comprises an amino acid sequence as set forth in SEQ ID NO: 48; or the heavy chain variable region consists of an amino acid sequence as set forth in SEQ ID NO: 47, and the light chain variable region consists of an amino acid sequence as set forth in SEQ ID NO: 48.

In some embodiments, the anti-Nectin-4 antibody or the antigen-binding fragment thereof comprises a heavy chain and a light chain, wherein the heavy chain comprises an amino acid sequence as set forth in SEQ ID NO: 49, and the light chain comprises an amino acid sequence as set forth in SEQ ID NO: 50; or the heavy chain consists of an amino acid sequence as set forth in SEQ ID NO: 49, and the light chain consists of an amino acid sequence as set forth in SEQ ID NO: 50. In some embodiments, the anti-Nectin-4 antibody or the antigen-binding fragment thereof comprises the two said heavy chains and the two said light chains. In some embodiments, the anti-Nectin-4 antibody is a full-length antibody. In some embodiments, the anti-Nectin-4 antibody is selected from Enfortumab or a biosimilar of Enfortumab, or the anti-Nectin-4 antibodies or antigen-binding fragments thereof as defined herein.

Embodiment I-51. The drug conjugate or the pharmaceutically acceptable salt thereof, or the solvate, the racemic mixture, the enantiomer, the diastereomer or the tautomer thereof according to any one of embodiments I-1 to I-50, having an average DAR of 2-8, such as an average DAR of 3-4, 3.8-4.2, 3-5, 4-5.5, 5-7, 6.5-8, or 6-8, preferably, the drug conjugates are selected from ADC-1 to ADC-34 disclosed in the embodiments herein.

Embodiment I-52. A pharmaceutical composition, comprising the drug conjugate or the pharmaceutically acceptable salt thereof or the solvate, the racemic mixture, the enantiomer, the diastereomer or the tautomer thereof according to any one of embodiments I-1 to I-51, and optionally comprising a pharmaceutically acceptable carrier.

Embodiment I-53. Use of the drug conjugate or the pharmaceutically acceptable salt thereof, or the solvate, the racemic mixture, the enantiomer, the diastereomer or the tautomer thereof according to any one of embodiments I-1 to I-51 in the manufacture of a drug for the treatment of a disease or condition mediated at least in part by PI3K and/or PIKK.

Embodiment I-54. The use according to embodiment I-53, wherein the disease or condition mediated at least in part by PI3K and/or PIKK is selected from: a cancer or an autoimmune disease.

Embodiment I-55. The use according to embodiment I-54, wherein the autoimmune disease is selected from: rheumatoid arthritis, chronic obstructive pulmonary disease, allergic rhinitis, asthma, acquired hemophilia A (AHA), idiopathic thrombocytopenia (ITP), and activated phosphoinositide 3-kinase-delta syndrome (APDS).

Embodiment I-56. The use according to embodiment I-54, wherein the cancer is a solid tumor or a hematological malignancy, preferably breast cancer, multiple myeloma, Burkitt lymphoma, diffuse large B-cell lymphoma, or non-small cell lung cancer.

### Embodiment II

Embodiment II-1. A compound of formula (IV), or a pharmaceutically acceptable salt thereof, or a solvate, a racemic mixture, an enantiomer, a diastereomer or a tautomer thereof

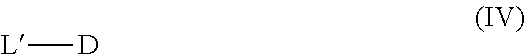

wherein L′ is a linker, which is used to link with Ab;
D is a PI3K and/or PIKK inhibitor; and preferably, the structure of D is as defined in any one of embodiments I-2 to I-38.

Embodiment II-2. The compound or the pharmaceutically acceptable salt thereof, or the solvate, the racemic mixture, the enantiomer, the diastereomer or the tautomer thereof according to embodiment II-1, wherein
L′ has a structure represented by formula (V):

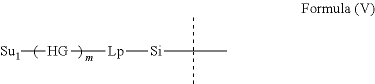

wherein
Su₁ is a stretcher group, having a structure selected from:

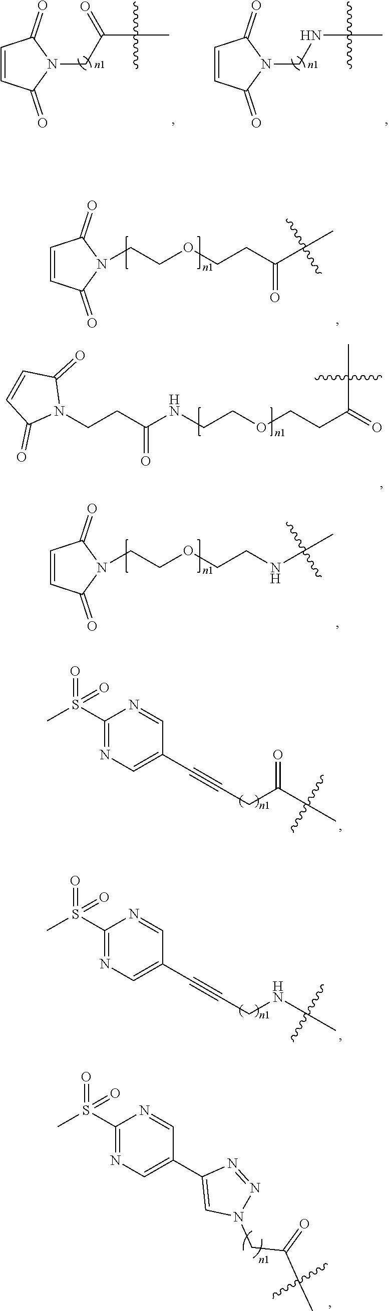

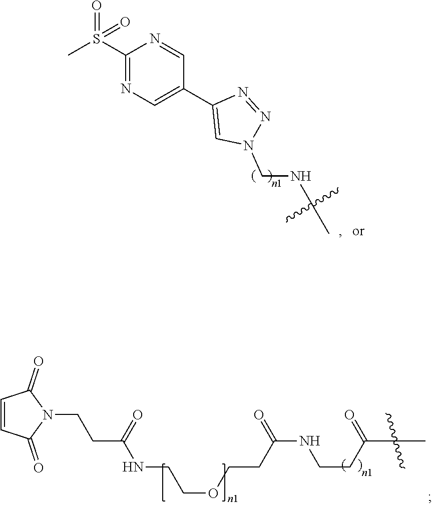

wherein [symbol] represents the linking site to which the structure (e.g., HG or Lp) on the right side of Su₁ is linked, and each n₁ is independently selected from an integer from 1 to 10, such as 1, 2, 3, 4, 5, 6, 7, 8, 9, and 10;
the remaining symbols HG, Lp, Si, and m are as defined in embodiment I-39 to I-44;
the dashed lines represent the site to which D is linked; and preferably, the structure of Su₁ is selected from:

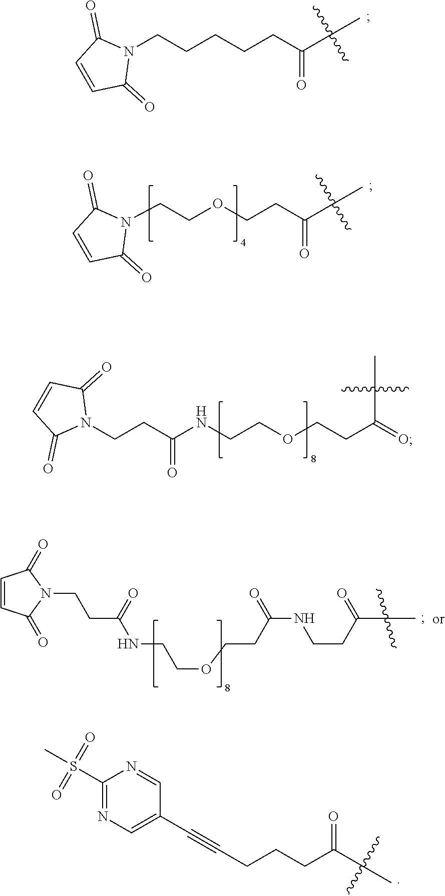

Embodiment II-3. The compound or the pharmaceutically acceptable salt thereof, or the solvate, the racemic mixture, the enantiomer, the diastereomer or the tautomer thereof according to any one of embodiments II-1 to II-2, wherein the compound is selected from:

| Structure | No. |
|---|---|
| 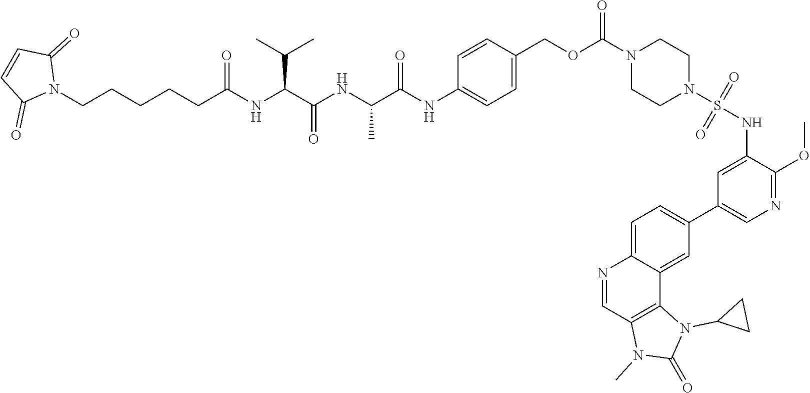 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](C)NC(=O)[C@@H](NC(=O)CCCCCN3C(=O)C=CC3=O)C(C)C)cc2)CC1] | LP-1 |
| 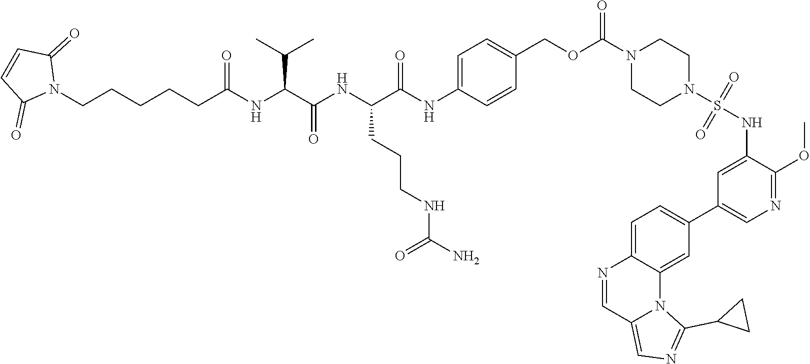 [SMILES: COc1ncc(-c2ccc3ncc4cnc(C5CC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-2 |
| 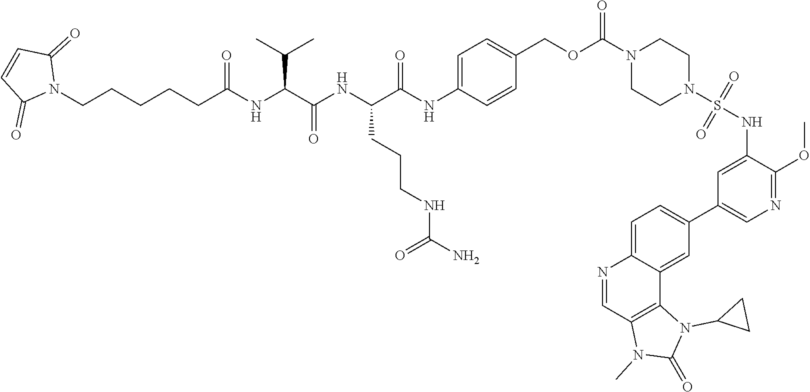 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-3 |
| 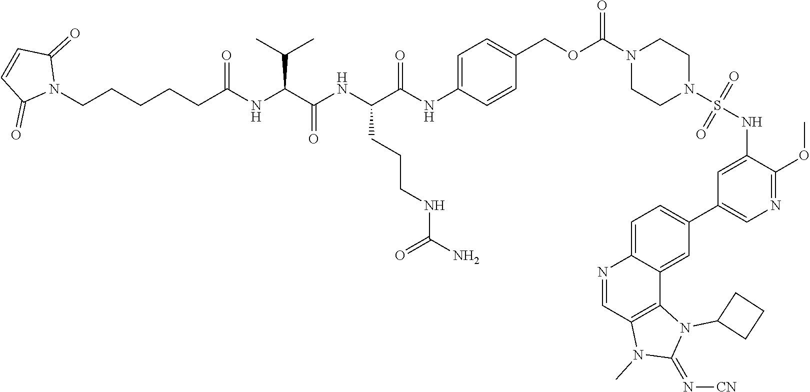 [SMILES: COc1ncc(-c2ccc3ncc4c(c3c2)n(C2CCC2)/c(=N\C#N)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-4 |
| 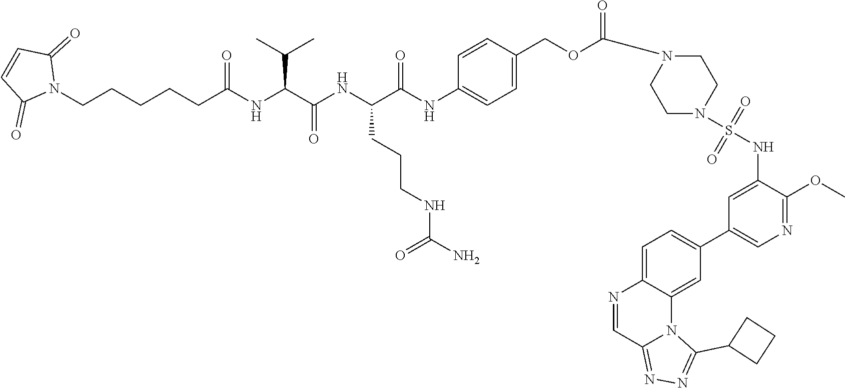 [SMILES: COc1ncc(-c2ccc3ncc4nnc(C5CCC5)n4c3c2)cc1NS(=O)(=O)N1CCN(CC(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-5 |
| 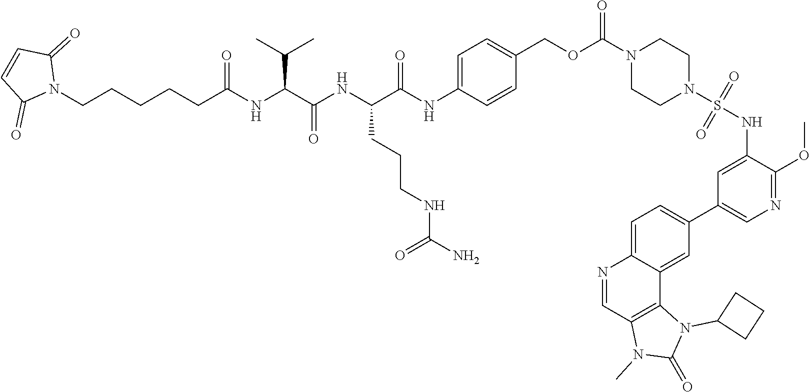 [SMILES: COc1ncc(-c2ccc3ncc4c(c3c2)n(C2CCC2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-6 |
| 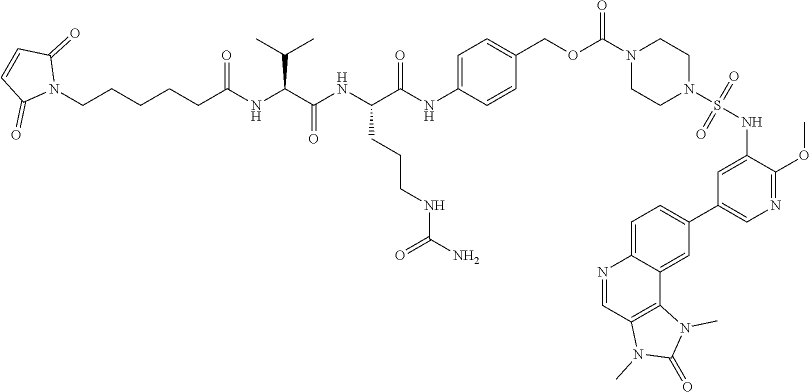 [SMILES: COc1ncc(-c2ccc3ncc4c(c3c2)n(C)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-7 |
| 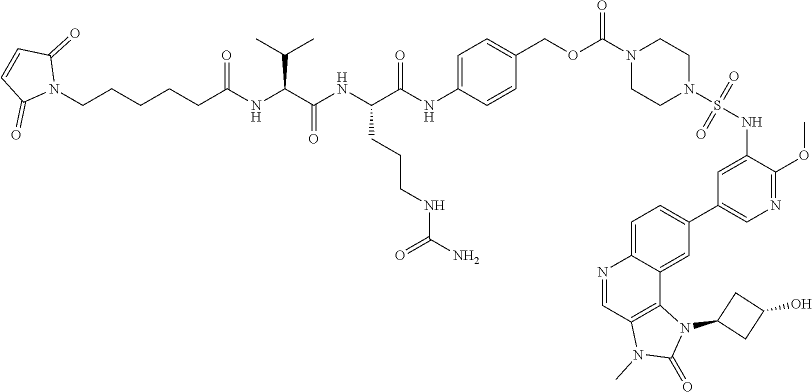 [SMILES: COc1ncc(-c2ccc3ncc4c(c3c2)n([C@H]2C[C@H](O)C2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-8 |
| 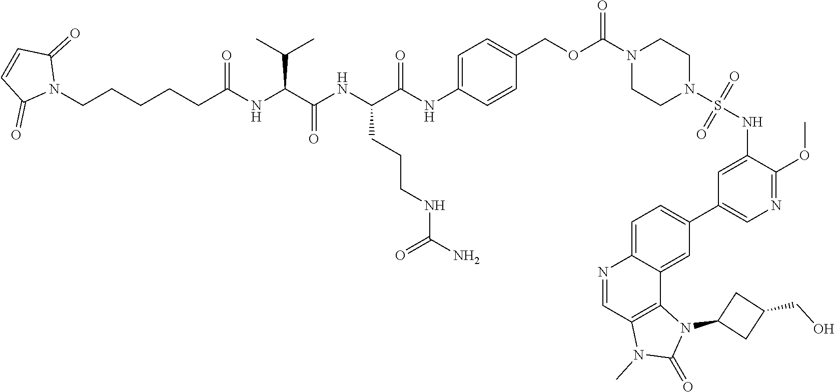 [SMILES: COc1ncc(-c2ccc3ncc4c(c3c2)n([C@H]2C[C@H](CO)C2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-9 |
| 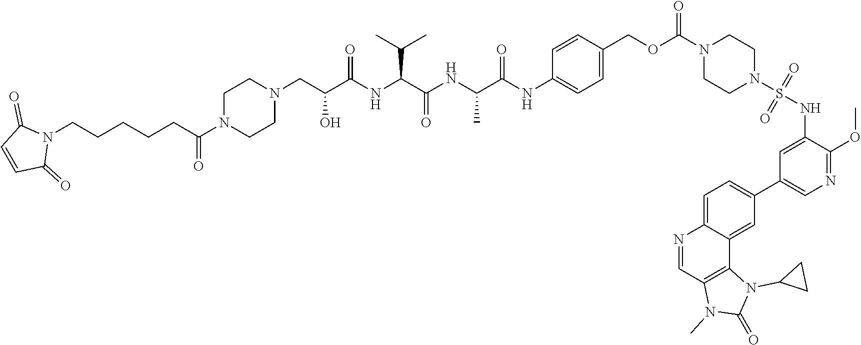 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](C)NC(=O)[C@@H](NC(=O)[C@H](O)CN3CCN(C(=O)CCCCCN4C(=O)C=CC4=O)CC3)C(C)C)cc2)CC1] | LP-10 |
| 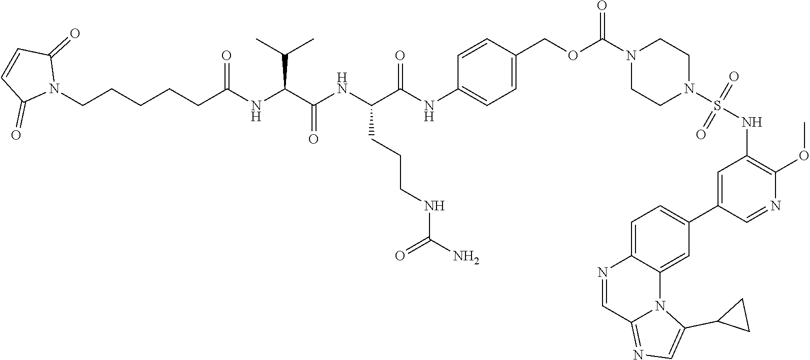 [SMILES: COc1ncc(-c2ccc3ncc4ncc(C5CC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-11 |
| 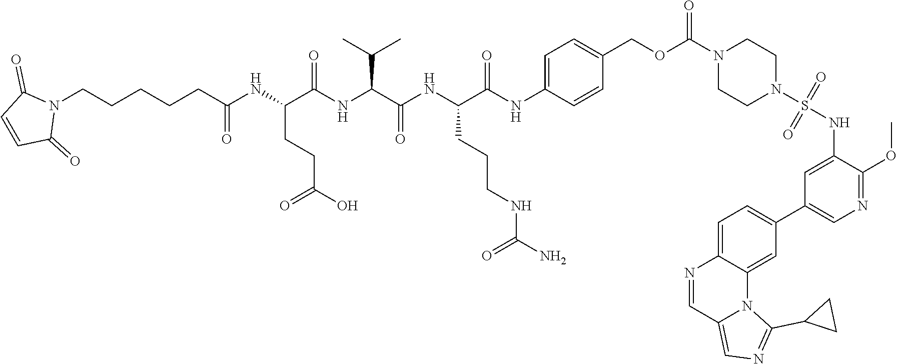 [SMILES: COc1ncc(-c2ccc3ncc4cnc(C5CC5)n4c3c2)cc1NS(=O)(=O)N1CCN(C(=O)OCc2ccc(NC(=O)[C@H](CCCNC(N)=O)NC(=O)[C@@H](NC(=O)[C@H](CCC(=O)O)NC(=O)CCCCCN3C(=O)C=CC3=O)C(C)C)cc2)CC1] | LP-12 |
| 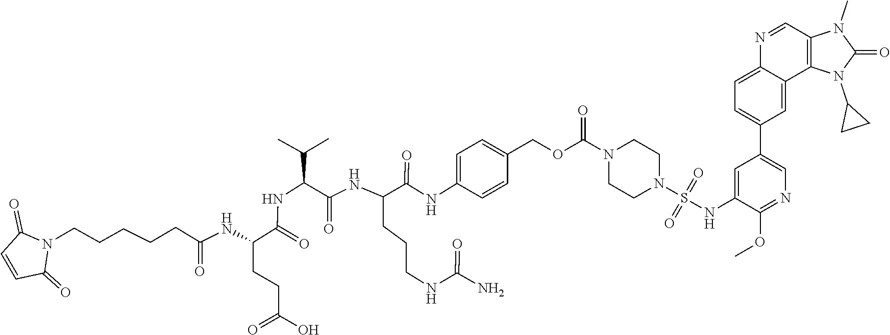 [SMILES: COc1ncc(-c2ccc3ncc4c(c3c2)n(C2CC2)c(=O)n4C)cc1NS(=O)(=O)N1CCN(C(=O)OCc2ccc(NC(=O)C(CCCNC(N)=O)NC(=O)[C@@H](NC(=O)[C@H](CCC(=O)O)NC(=O)CCCCCN3C(=O)C=CC3=O)C(C)C)cc2)CC1] | LP-13 |
| 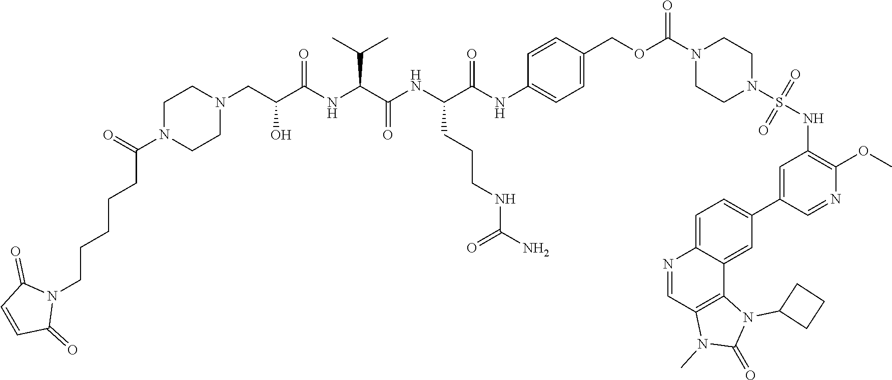 | LP-14 |
| 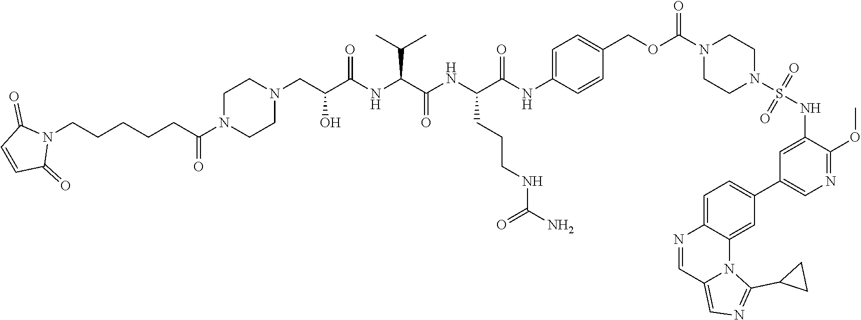 [SMILES: COc1ncc(-c2ccc3ncc4cnc(C5CC5)n4c3c2)cc1NS(=O)(=O)N1CCN(C(=O)OCc2ccc(NC(=O)[C@H](CCCNC(N)=O)NC(=O)[C@@H](NC(=O)[C@H](O)CN3CCN(C(=O)CCCCCN4C(=O)C=CC4=O)CC3)C(C)C)cc2)CC1] | LP-15 |
| 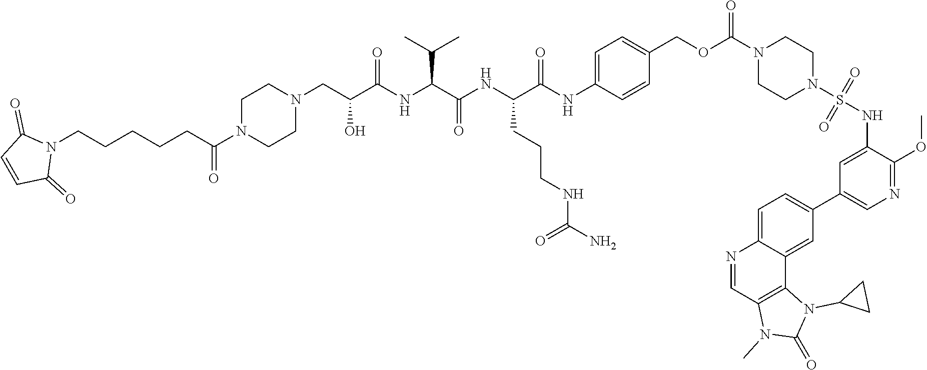 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)[C@H](O)CN3CCN(C(=O)CCCCCN4C(=O)C=CC4=O)CC3)C(C)C)cc2)CC1] | LP-16 |
| 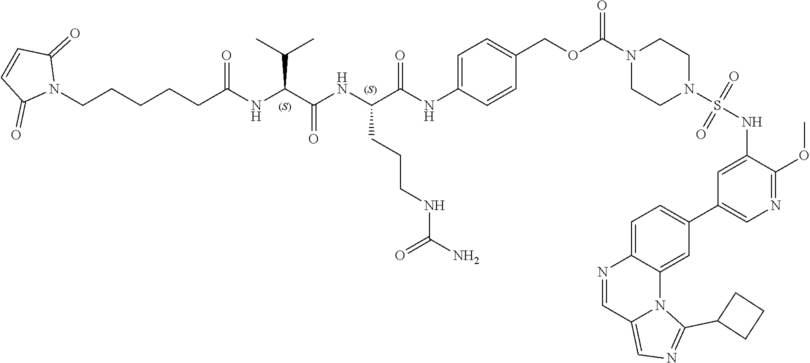 | LP-17 |
| 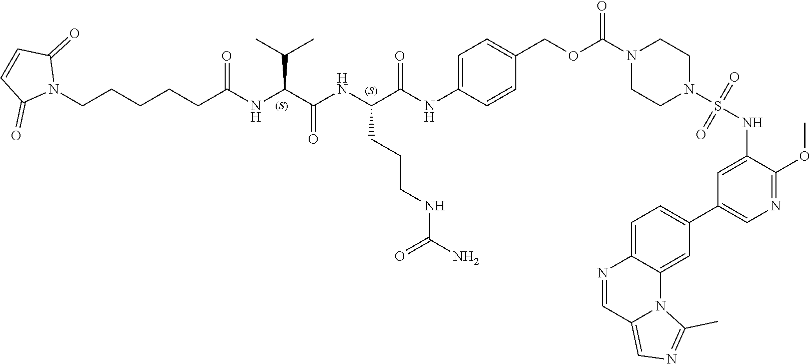 | LP-18 |
| 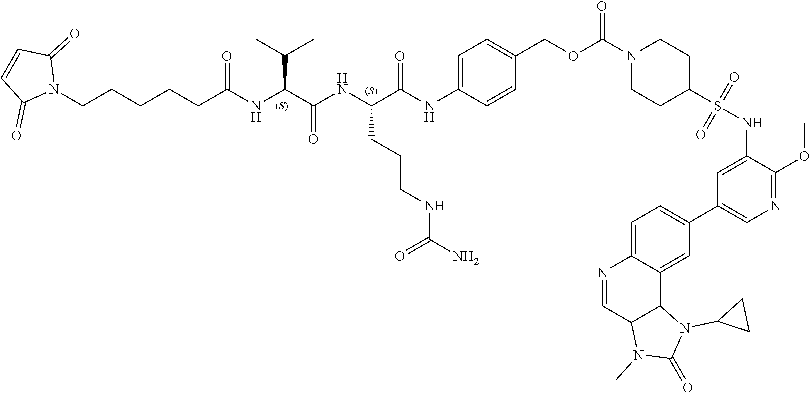 | LP-19 |
| 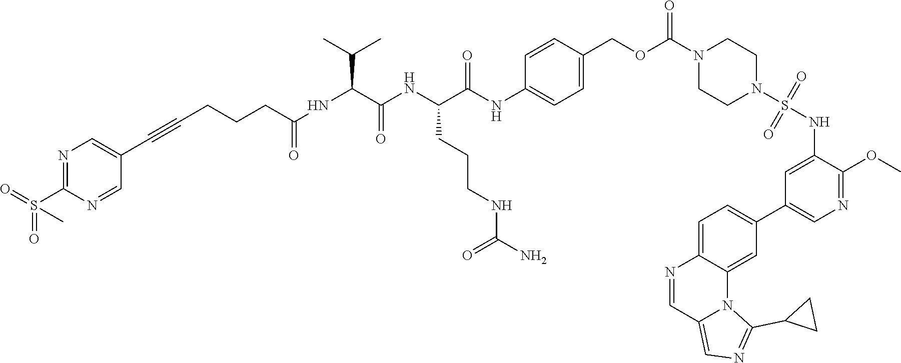 [SMILES: COc1ncc(-c2ccc3ncc4cnc(C5CC5)n4c3c2)cc1NS(=O)(=O)N1CCN(C(=O)OCc2ccc(NC(=O)[C@H](CCCNC(N)=O)NC(=O)[C@@H](NC(=O)CCCC#Cc3cnc(S(C)(=O)=O)nc3)C(C)C)cc2)CC1] | LP-20 |
| 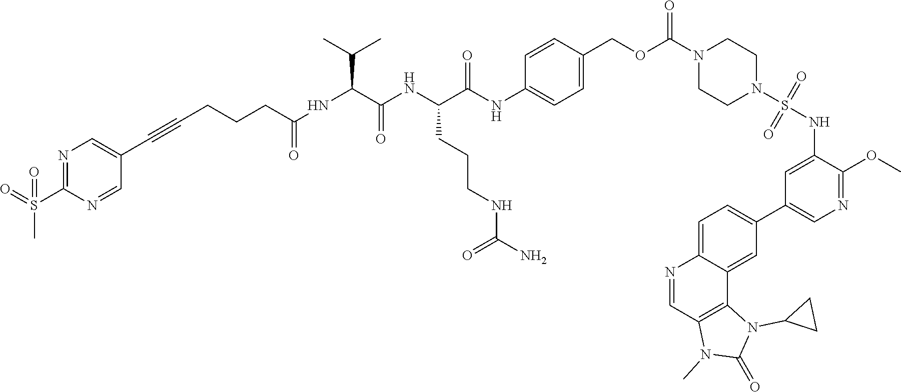 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)CCCC#Cc3cnc(S(C)(=O)=O)nc3)C(C)C)cc2)CC1] | LP-21 |
| 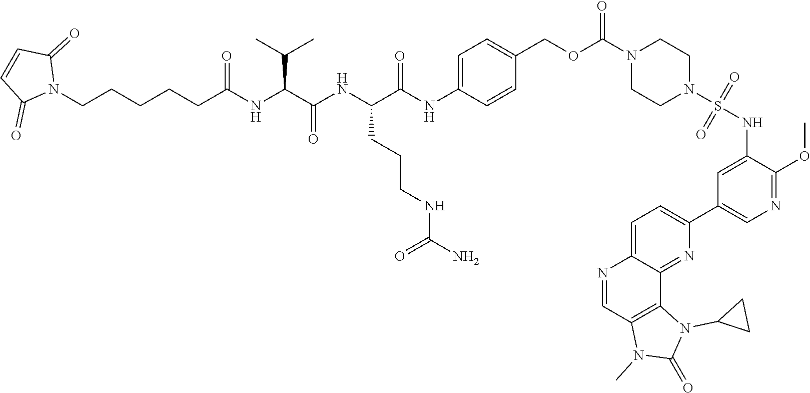 [SMILES: COc1ncc(-c2ccc3ncc4c(c3n2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-22 |
| 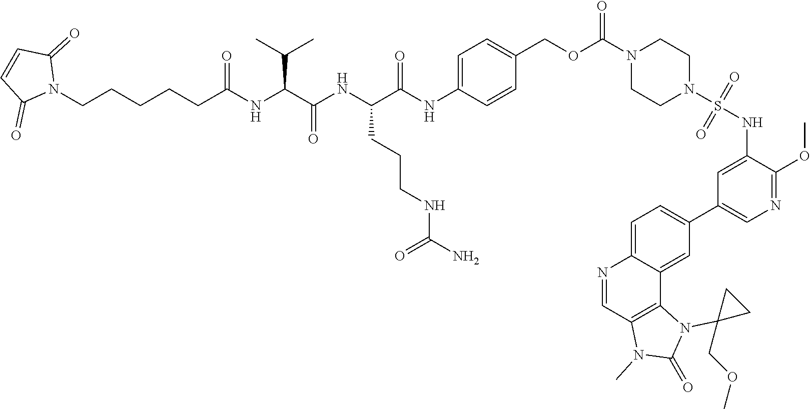 [SMILES: COCC1(n2c(=O)n(C)c3cnc4ccc(-c5cnc(OC)c(NS(=O)(=O)N6CCN(C(=O)OCc7ccc(NC(=O)[C@H](CCCNC(N)=O)NC(=O)[C@@H](NC(=O)CCCCCN8C(=O)C=CC8=O)C(C)C)cc7)CC6)c5)cc4c32)CC1] | LP-23 |
| 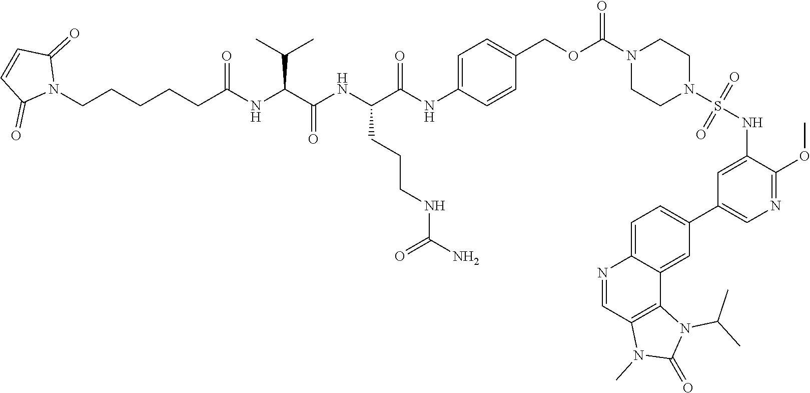 [SMILES: COc1ncc(-c2ccc3ncc4c(c3c2)n(C(C)C)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-24 |
| 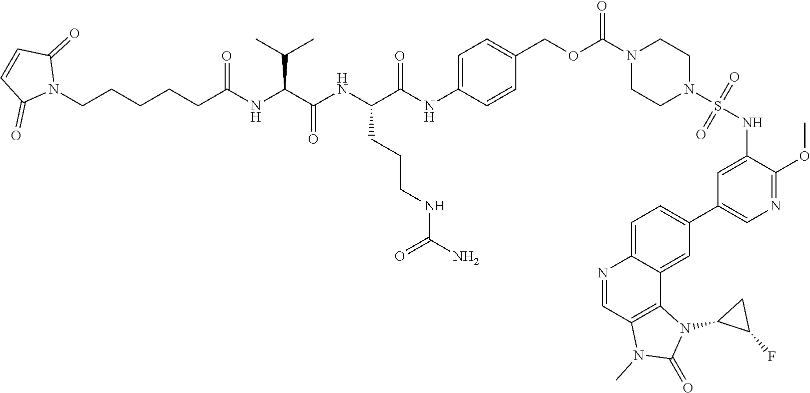 [SMILES: COc1ncc(-c2ccc3ncc4c(c3c2)n([C@@H]2C[C@@H]2F)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-25 |
| 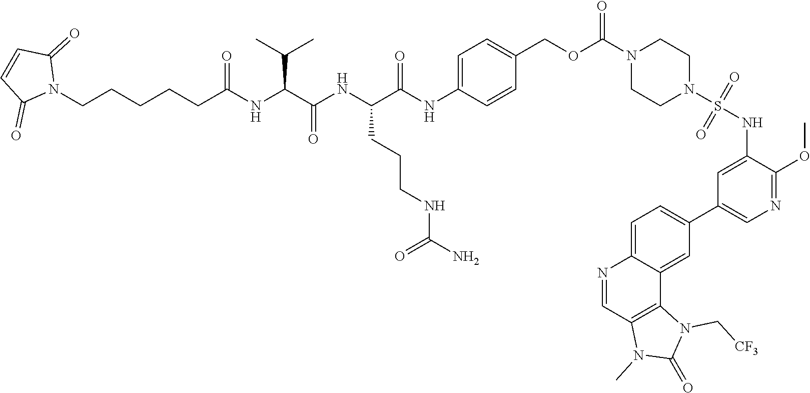 [SMILES: COc1ncc(-c2ccc3ncc4c(c3c2)n(CC(F)(F)F)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-26 |
| 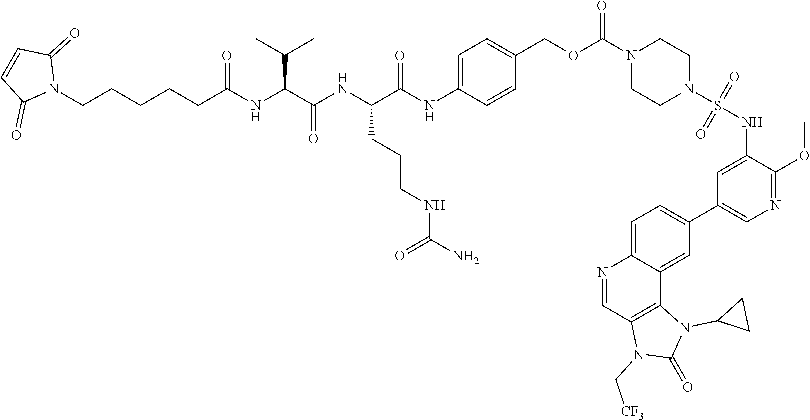 [SMILES: COc1ncc(-c2ccc3ncc4c(c3c2)n(C2CC2)c(=O)n4CC(F)(F)F)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-27 |
| 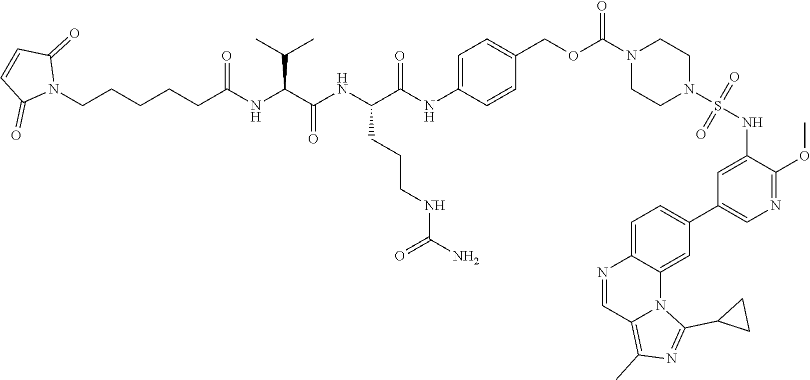 [SMILES: COc1ncc(-c2ccc3ncc4c(C)nc(C5CC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | LP-28 |

Embodiment II-4. Use of the compound according to embodiment II-3 in the manufacture of a drug for the treatment of a disease or condition mediated at least in part by PI3K and/or PIKK.

Embodiment II-5. The use according to embodiment II-4, wherein the disease or condition mediated at least in part by PI3Ks and/or PIKKs is selected from: a cancer or an autoimmune disease.

Embodiment II-6. The use according to embodiment II-5, wherein the autoimmune disease is selected from: rheumatoid arthritis, chronic obstructive pulmonary disease, allergic rhinitis, asthma, acquired hemophilia A (AHA), idiopathic thrombocytopenia (ITP), and activated phosphoinositide 3-kinase-delta syndrome (APDS).

Embodiment II-7. The use according to embodiment II-5, wherein the cancer is a solid tumor or a hematological malignancy, preferably selected from: breast cancer, lymphoma, multiple myeloma, Burkitt lymphoma, diffuse large B-cell lymphoma, or non-small cell lung cancer.

### Embodiment III

Embodiment III-1. A compound of formula (II-1), or a pharmaceutically acceptable salt thereof, or a solvate, a racemic mixture, an enantiomer, a diastereomer or a tautomer thereof,

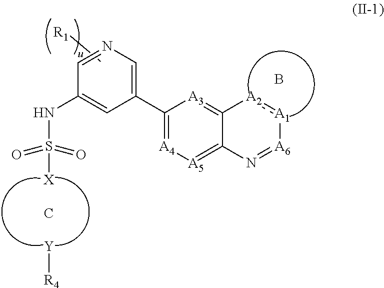

wherein
A₃, A₄, A₅, and A₆ are independently N or CR₅; R₅ is independently selected from: H, C₁₋₆ alkyl, C₃₋₉ cycloalkyl, —CN, —NH₂, —SH, —OH, —O(C₁₋₆ alkyl), halogen, or C₁₋₆ haloalkyl;

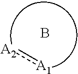

represents a 5- to 6-membered heteroaryl ring or a 3- to 8-membered heterocyclyl ring, and the 5- to 6-membered heteroaryl or 3- to 8-membered heterocyclyl is optionally substituted with one or more groups selected from oxo, ═NCN, or R₃;
ring A is 5- to 14-membered heteroaryl;
R₁ is selected from: H, —C₁₋₆ alkyl, —O(C₁₋₆ alkyl), —OC₁₋₆ haloalkyl, —CN, —C(O)NHC₁₋₆ alkyl, —NH₂, —NHC(O)C₁₋₆ alkyl, —NHC(O)O(C₁₋₆ alkyl), —NO₂, —OH, —SH, halogen, C₁₋₆ haloalkyl, 5- to 6-membered heteroaryl, and 3- to 12-membered heterocyclyl;
u, at each occurrence, is independently 0, 1, 2, or 3;
each R₃ is independently selected from: H, CN, NO₂, halogen, C₁₋₆ alkyl, C₆-C₁₀ aryl, C₃₋₉ cycloalkyl, 5- to 6-membered heteroaryl, and 3- to 12-membered heterocyclyl, wherein the C₁₋₆ alkyl, C₆-C₁₀ aryl, C₃₋₉ cycloalkyl, 5- to 6-membered heteroaryl, and 3- to 12-membered heterocyclyl can each independently and optionally be substituted with one or more groups selected from: C₁₋₆ alkyl, C₂₋₈ alkenyl, C₂₋₈ alkynyl, C₃₋₉ cycloalkyl, —CN, —C(O)NH₂, —NH₂, —NHC(O)C₁₋₆ alkyl, —NO₂, —OH, —C₁₋₆ alkylene-OH, halogen, or C₁₋₆ haloalkyl;
X and Y are each independently C, CH, or N;
R₄ is absent or is selected from H, —C₁₋₆ alkylene-NH₂, —NH₂, —C₁₋₆ alkylene-OH, or —OH;
ring C is selected from C₆-C₁₀ aryl, a 5- to 6-membered heteroaryl, or a 3- to 12-membered heterocyclic ring, each of which is independently and optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH; and
[symbol] represents a single bond or a double bond.

Embodiment III-2. The compound or the pharmaceutically acceptable salt thereof, or the solvate, the racemic mixture, the enantiomer, the diastereomer or the tautomer thereof according to embodiment III-1, wherein A₃, A₄, A₅, and A₆ are all CH, or A₃ is N, A₄, A₅, and A₆ are all CH.

Embodiment III-3. The compound or the pharmaceutically acceptable salt thereof, or the solvate, the racemic mixture, the enantiomer, the diastereomer or the tautomer thereof according to embodiment III-1 or III-2, wherein

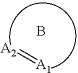

is selected from the following structures:

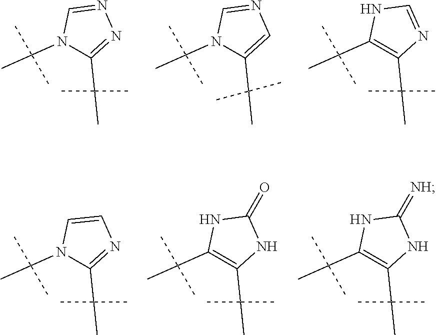

and the above cyclic structures are optionally substituted with one or more R₃, for example substituted with 1, 2, or 3 R₃.

Embodiment III-4. The compound or the pharmaceutically acceptable salt thereof, or the solvate, the racemic mixture, the enantiomer, the diastereomer or the tautomer thereof according to embodiment III-3, wherein each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, C₁₋₆ alkyl, —CN, —NH₂, —NO₂, —OH, —C₁₋₆ alkylene-OH, halogen, C₁₋₆ haloalkyl, or —C₁₋₆ alkylene-OC₁₋₆ alkyl.

Embodiment III-5. The compound or the pharmaceutically acceptable salt thereof, or the solvate, the racemic mixture, the enantiomer, the diastereomer or the tautomer thereof according to embodiment III-3 or III-4, wherein the R₃ is selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, —OH, F, or —C₁₋₆ alkylene-OH; and

In some specific embodiments, R₃ is selected from: hydrogen, —CH₃, —CD₃, —CH(CH₃)₂, —CH₂CF₃, or the following groups:

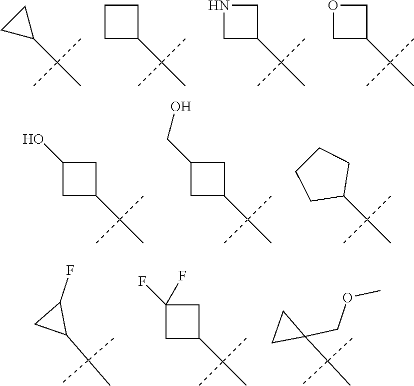

Embodiment III-6. The compound or the pharmaceutically acceptable salt thereof, or the solvate, the racemic mixture, the enantiomer, the diastereomer or the tautomer thereof according to any one of embodiments III-1 to III-5, wherein

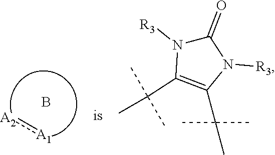

each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: deuterium, —OH or —C₁₋₆ alkylene-OH.

Embodiment III-7. The compound or the pharmaceutically acceptable salt thereof, or the solvate, the racemic mixture, the enantiomer, the diastereomer or the tautomer thereof according to any one of embodiments III-1 to III-5, wherein

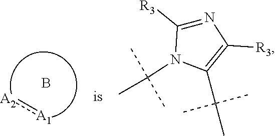

each R₃ is independently selected from: H, C₁₋₆ alkyl, C₃₋₉ cycloalkyl, and 3- to 12-membered heterocyclyl, wherein the C₁₋₆ alkyl, C₃₋₉ cycloalkyl, and 3- to 12-membered heterocyclyl can each optionally be substituted with one or more groups independently selected from: —OH or —C₁₋₆ alkylene-OH;

In some specific embodiments, each R₃ is independently H or C₃₋₉ cycloalkyl.

Embodiment III-8. The compound or the pharmaceutically acceptable salt thereof, or the solvate, the racemic mixture, the enantiomer, the diastereomer or the tautomer thereof according to any one of embodiments III-1 to III-7, wherein X and Y are both N, and R₄ is absent or is H.

Embodiment III-9. The compound or the pharmaceutically acceptable salt thereof, or the solvate, the racemic mixture, the enantiomer, the diastereomer or the tautomer thereof according to any one of embodiments III-1 to III-7, wherein X is —CH— or N, Y is N, and R₄ is selected from H, —C₁₋₆ alkylene-NH₂, —NH₂, —C₁₋₆ alkylene-OH, or —OH.

Embodiment III-10. The compound or the pharmaceutically acceptable salt thereof, or the solvate, the racemic mixture, the enantiomer, the diastereomer or the tautomer thereof according to any one of embodiments III-1 to III-9, wherein the ring C is a 3- to 12-membered heterocyclic ring which is optionally substituted with one or more groups selected from: —CN, —NO₂, halogen, —C₁₋₆ alkyl, —C₁₋₆ haloalkyl, C₃₋₉ cycloalkyl, —C₁₋₆ alkylene-NH₂, —NH₂, —C₁₋₆ alkylene-OH, or —OH.

Embodiment III-11. The compound or the pharmaceutically acceptable salt thereof, or the solvate, the racemic mixture, the enantiomer, the diastereomer or the tautomer thereof according to embodiment III-10, wherein the structure of the ring C is selected from:

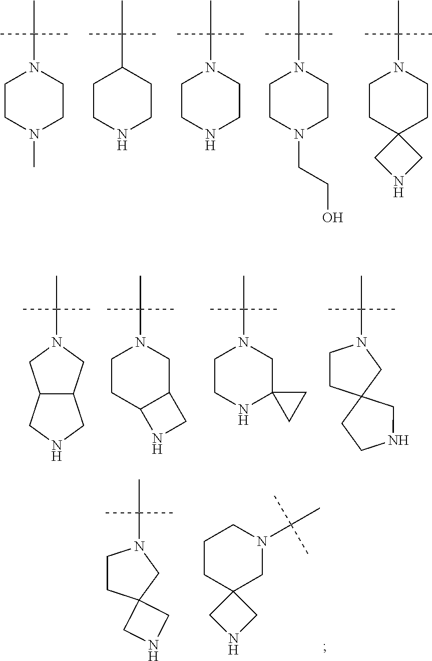

In some specific embodiments, the structure of the ring C is:

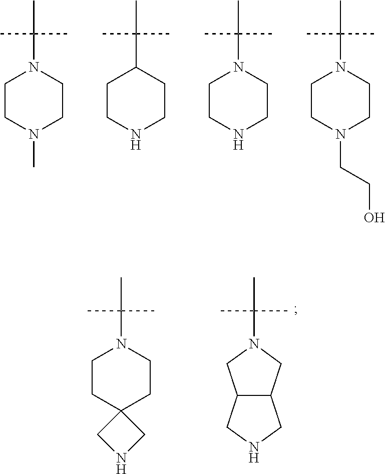

and wherein the dashed lines represent the position where the ring C is linked to S(O)₂.

Embodiment III-12. The compound or the pharmaceutically acceptable salt thereof, or the solvate, the racemic mixture, the enantiomer, the diastereomer or the tautomer thereof according to any one of embodiments III-1 to III-11, wherein R₁ is: —O(C₁₋₆ alkyl), —OC₁₋₆ haloalkyl, and C₃₋₉ cycloalkyl, wherein the C₁₋₆ alkyl is optionally substituted with one or more deuterium; in some specific embodiments, R₁ is —O(C₁₋₆ alkyl); in some specific embodiments, R₁ is —OCH₂CF₃.

Embodiment III-13. The compound or the pharmaceutically acceptable salt thereof, or the solvate, the racemic mixture, the enantiomer, the diastereomer or the tautomer thereof according to any one of embodiments III-1 to III-12, wherein the compound is selected from:

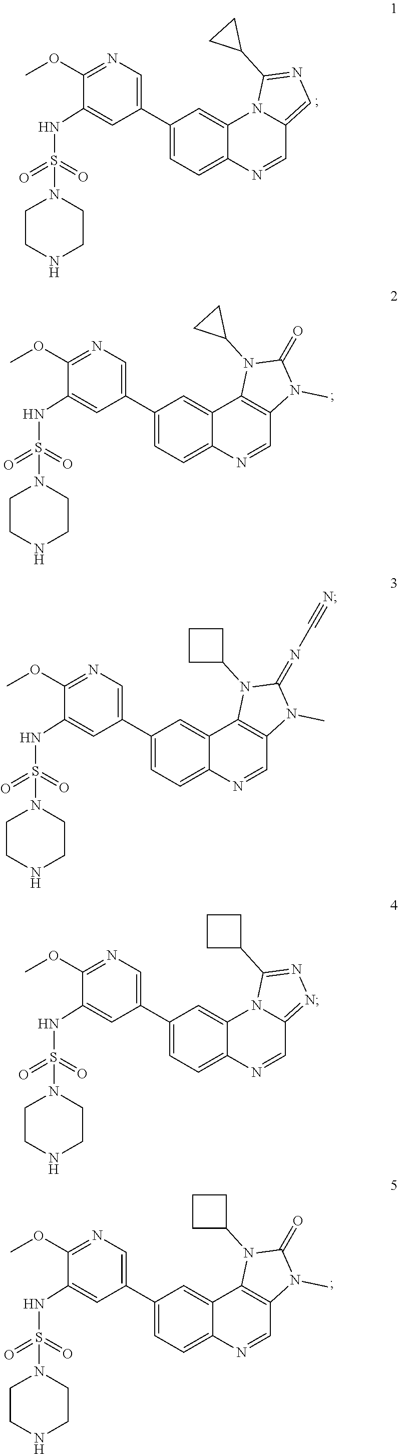 [SMILES: COc1ncc(-c2ccc3ncc4cnc(C5CC5)n4c3c2)cc1NS(=O)(=O)N1CCNCC1 ; COc1ncc(-c2ccc3ncc4c(c3c2)n(C2CC2)c(=O)n4C)cc1NS(=O)(=O)N1CCNCC1 ; COc1ncc(-c2ccc3ncc4c(c3c2)n(C2CCC2)c(=NC#N)n4C)cc1NS(=O)(=O)N1CCNCC1 ; COc1ncc(-c2ccc3ncc4nnc(C5CCC5)n4c3c2)cc1NS(=O)(=O)N1CCNCC1 ; COc1ncc(-c2ccc3ncc4c(c3c2)n(C2CCC2)c(=O)n4C)cc1NS(=O)(=O)N1CCNCC1]

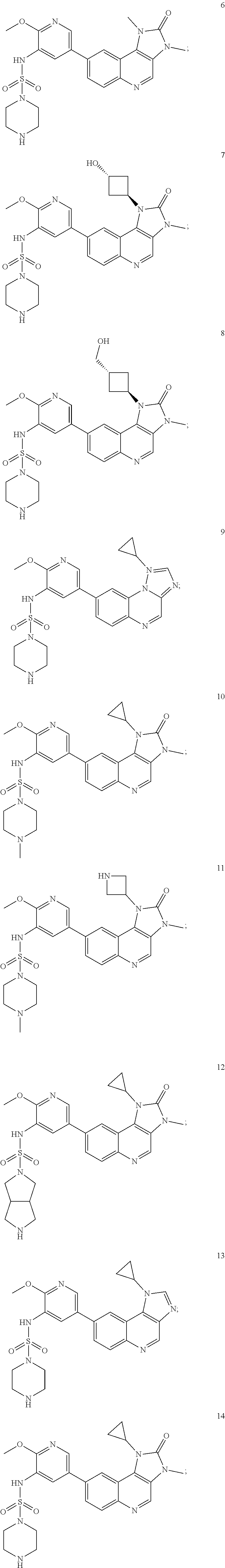

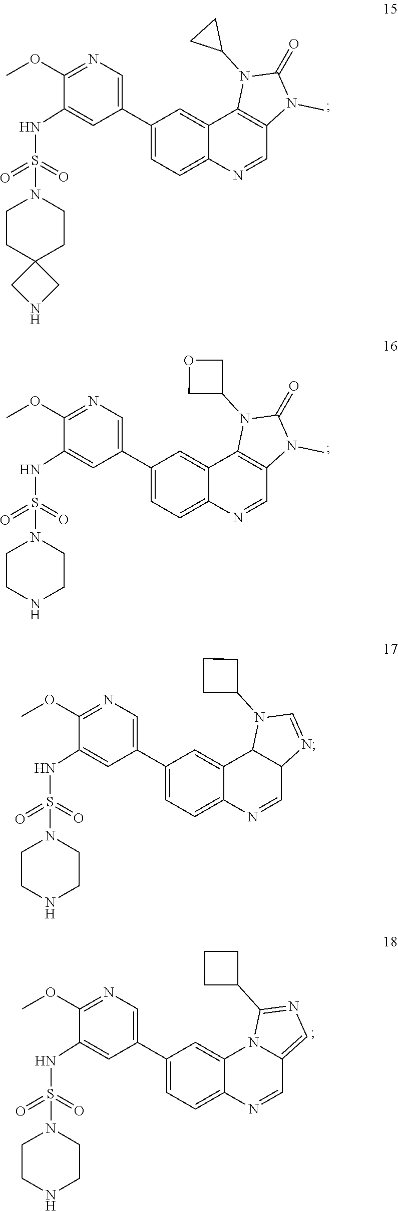 [SMILES: COc1ncc(-c2ccc3ncc4c(c3c2)n(C2CC2)c(=O)n4C)cc1NS(=O)(=O)N1CCC2(CC1)CNC2 ; COc1ncc(-c2ccc3ncc4c(c3c2)n(C2COC2)c(=O)n4C)cc1NS(=O)(=O)N1CCNCC1 ; COc1ncc(-c2ccc3c(c2)C2C(C=N3)N=CN2C2CCC2)cc1NS(=O)(=O)N1CCNCC1 ; COc1ncc(-c2ccc3ncc4cnc(C5CCC5)n4c3c2)cc1NS(=O)(=O)N1CCNCC1]

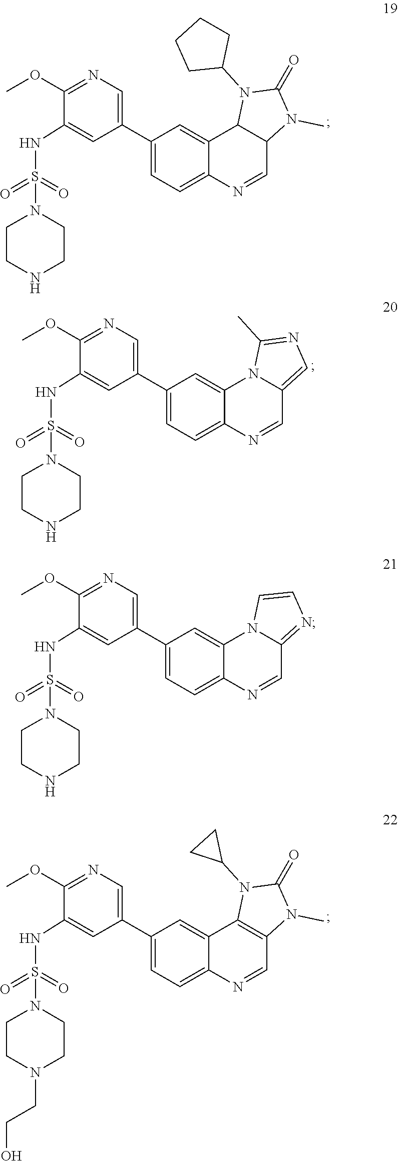 [SMILES: COc1ncc(-c2ccc3c(c2)C2C(C=N3)N(C)C(=O)N2C2CCCC2)cc1NS(=O)(=O)N1CCNCC1 ; COc1ncc(-c2ccc3ncc4cnc(C)n4c3c2)cc1NS(=O)(=O)N1CCNCC1 ; COc1ncc(-c2ccc3ncc4nccn4c3c2)cc1NS(=O)(=O)N1CCNCC1 ; COc1ncc(-c2ccc3ncc4c(c3c2)n(C2CC2)c(=O)n4C)cc1NS(=O)(=O)N1CCN(CCO)CC1]

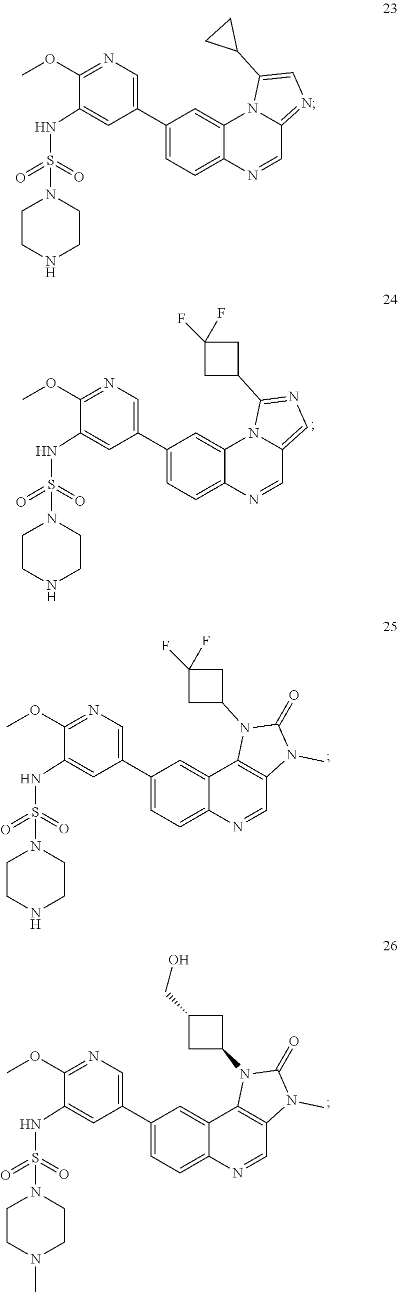 [SMILES: COc1ncc(-c2ccc3ncc4ncc(C5CC5)n4c3c2)cc1NS(=O)(=O)N1CCNCC1 ; COc1ncc(-c2ccc3ncc4cnc(C5CC(F)(F)C5)n4c3c2)cc1NS(=O)(=O)N1CCNCC1 ; COc1ncc(-c2ccc3ncc4c(c3c2)n(C2CC(F)(F)C2)c(=O)n4C)cc1NS(=O)(=O)N1CCNCC1 ; COc1ncc(-c2ccc3ncc4c(c3c2)n([C@H]2C[C@H](CO)C2)c(=O)n4C)cc1NS(=O)(=O)N1CCN(C)CC1]

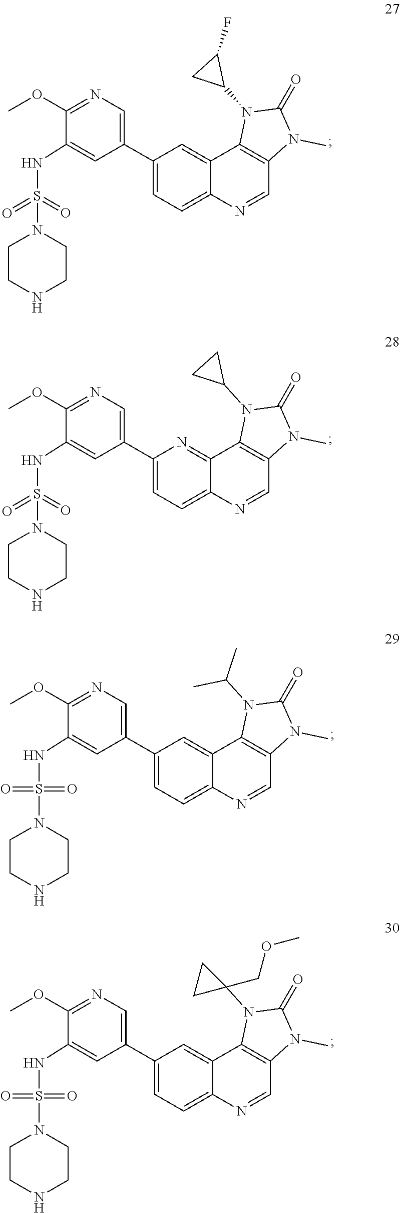

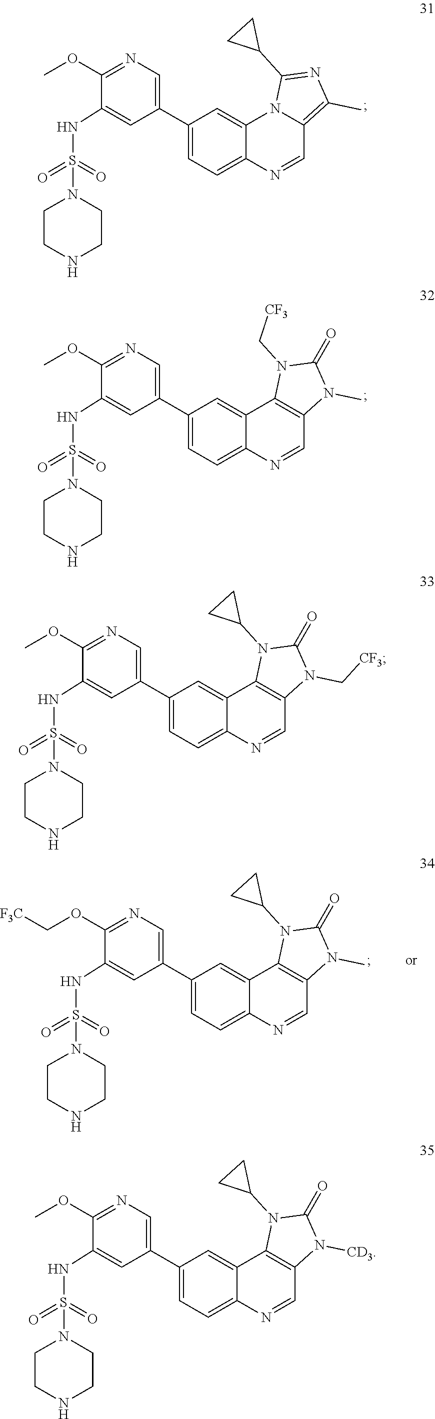 [SMILES: COc1ncc(-c2ccc3ncc4c(C)nc(C5CC5)n4c3c2)cc1NS(=O)(=O)N1CCNCC1 ; COc1ncc(-c2ccc3ncc4c(c3c2)n(CC(F)(F)F)c(=O)n4C)cc1NS(=O)(=O)N1CCNCC1 ; COc1ncc(-c2ccc3ncc4c(c3c2)n(C2CC2)c(=O)n4CC(F)(F)F)cc1NS(=O)(=O)N1CCNCC1 ; Cn1c(=O)n(C2CC2)c2c3cc(-c4cnc(OCC(F)(F)F)c(NS(=O)(=O)N5CCNCC5)c4)ccc3ncc21 ; [2H]C([2H])([2H])n1c(=O)n(C2CC2)c2c3cc(-c4cnc(OC)c(NS(=O)(=O)N5CCNCC5)c4)ccc3ncc21]

Embodiment III-14. Use of the compound according to any one of embodiments III-1 to III-13 in the manufacture of a drug for the treatment of a disease or condition mediated at least in part by PI3K and/or PIKK.

Embodiment III-15. The use according to embodiment III-14, wherein the disease or condition mediated at least in part by PI3K and/or PIKK is selected from: a cancer or an autoimmune disease.

Embodiment III-16. The use according to embodiment III-15, wherein the autoimmune disease is selected from: rheumatoid arthritis, chronic obstructive pulmonary disease, allergic rhinitis, asthma, acquired hemophilia A (AHA), idiopathic thrombocytopenia (ITP), and activated phosphoinositide 3-kinase-delta syndrome (APDS).

Embodiment III-17. The use according to embodiment III-15, wherein the cancer is a solid tumor or a hematological malignancy, preferably selected from: breast cancer, multiple myeloma, Burkitt lymphoma, diffuse large B-cell lymphoma, or non-small cell lung cancer.

Embodiment III-18. A pharmaceutical composition comprising the compound and/or the pharmaceutically acceptable salt, the solvate thereof according to any one of embodiments III-1 to III-13, and at least one pharmaceutically acceptable carrier.

The compound obtained in the present invention can be further modified at their peripheral positions to provide other target compounds of the present invention. Synthetic chemistry transformations are techniques well known in the art, as published in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette (ed.), Encyclopedia of Reagents for Organic Synthesis, John Wiley and Sons (1995), and subsequent versions thereof.

Before use, the at least one compound and/or at least one pharmaceutically acceptable salt described herein, can be purified by column chromatography, high performance liquid chromatography, crystallization, or other suitable methods.

The antibody-drug conjugate (ADC) of the present invention can be prepared by methods well known in the art, for example, a commonly used ADC conjugation method which comprises the following steps:
(a) Ab (a targeting moiety, preferably an antibody or an antigen-binding fragment thereof) was added to a buffer solution, and a reducing agent was added, and then incubation was performed;
(b) A compound of formula (IV) or a pharmaceutically acceptable salt thereof, or a solvate thereof described in the present invention was added to the reaction solution in step (a) for conjugation, thus to obtain a crude product;
(c) The crude product was optionally purified to afford the drug conjugate represented by formula (I).

In some embodiments, the drug conjugate represented by formula (I) can be obtained by conjugating the linker moiety of the compound of formula (IV) with the active group of the antibody moiety (such as daratumumab) in water and/or an organic solvent. Suitable organic solvents can be selected from N,N-dimethylformamide, dimethyl sulfoxide, N-methylpyrrolidone, nitriles (such as acetonitrile), alcohols (such as methanol or ethanol), and any combination thereof. The conjugation method may involve opening a disulfide bond of the antibody by using a reducing agent (such as TCEP) to yield sulfhydryl, then reacting with the linker moiety of the compound of formula (IV) to form a C—S bond, and then performing conjugation. The resulting conjugation product can be purified by a chromatography method (such as one or more of ion exchange chromatography, hydrophobic chromatography, reverse phase chromatography, or affinity chromatography).

In some embodiments, the method comprises the following two steps:
Step I: A buffer, a TCEP solution, and a DTPA solution were successively added to a monoclonal antibody solution of an appropriate concentration formulated with a reaction buffer, and the resulting solution was reacted at 15-37° C. for 60-180 minutes. The reaction buffer was one of PBS-D buffer (10 mmol/L, pH 7.0), histidine-acetic acid buffer (20 mmol/L, pH 5.5), histidine-hydrochloric acid buffer (10 mmol/L, pH 6.5), histidine buffer (10 mmol/L, pH 7.0), or histidine buffer (10 mmol/L, pH 9.0). TCEP was dissolved in water or a corresponding reaction buffer;
Step II: Adding a solution of the compound of formula (IV) in DMSO:H₂O, the reaction was further performed at 15-37° C. for 1-18 h. The reaction solution was transferred to an ultrafiltration centrifuge tube (Millipore Amicon® Ultra), and the corresponding buffer solution was added for solution exchange, thus a target product was obtained. The reaction buffer was one of PBS-D buffer (10 mmol/L, pH 7.0), histidine-acetic acid buffer (20 mmol/L, pH 5.5), histidine-hydrochloric acid buffer (10 mmol/L, pH 6.5), histidine buffer (10 mmol/L, pH 7.0), or histidine buffer (10 mmol/L, pH 9.0).

Suitable in vitro assays can be used to preliminarily evaluate the effects of at least one of the antibody-drug conjugates of formula (I), the linker-payload compound of formula (IV), the compound of formula (II-1) and/or at least one pharmaceutically acceptable salt thereof described herein, in inhibiting the activity of PI3K and/or PIKK kinases, and can be further examined for efficacy in treating cancers or inflammatory diseases by in vivo experiments. For example, the antibody-drug conjugate of formula (I), the linker-payload compound of formula (IV), the compound of formula (II-1), and/or the pharmaceutically acceptable salt thereof can be administered to an animal suffering from a cancer or inflammatory disease, such as a mouse model, and then their therapeutic effects can be accessed. Based on the above results, an appropriate dosage range and administration route for animals (such as humans) can also be determined.

The present invention further provides a method of inhibiting the activity of PI3K and/or PIKK kinases, wherein the method comprises contacting an effective amount of at least one antibody-drug conjugate of formula (I), linker-payload compound of formula (IV), compound of formula (II-1), and/or at least one pharmaceutically acceptable salt thereof described herein with at least one kinase. The antibody-drug conjugate of formula (I), the linker-payload compound of formula (IV), the compound of formula (II-1) and/or the at least one pharmaceutically acceptable salt thereof described herein can be used to achieve a beneficial therapeutic or prophylactic effect.

The present invention further provides a method of treating a disease state or a condition associated with the PI3K and/or PIKK activity or mediated in part by PI3Ks and/or PIKKs (mediated at least in part by PI3Ks and/or PIKKs), wherein the method comprises administering to a subject in need thereof a therapeutically effective amount of the antibody-conjugated drug of formula (I), the linker-payload compound of formula (IV), or the compound of formula (II-1) described herein. Any disease or condition that is ameliorated by inhibition of phosphoinositide 3-kinases (referred to herein as a “disease mediated at least in part by PI3Ks and/or PIKKs”) can be treated using the antibody-conjugated drug of formula (I), the linker-payload compound of formula (IV), or the compound of formula (II-1), and compositions thereof described herein.

The term “diseases mediated at least in part by PI3Ks and/or PIKKs” refers to diseases with a pathogenesis comprising at least a portion of factors associated with the PI3Ks and/or PIKKs, these diseases including but not limited to: cancers, neurodegenerative diseases, viral infections, bacterial infections, ocular diseases, kidney diseases, autoimmune diseases, cardiovascular and cerebrovascular diseases, and psychological diseases.

The term “cancer (or tumor)” as used refers to a cellular disorder characterized by uncontrolled or dysregulated cell proliferation, decreased cellular differentiation, inappropriate ability to invade surrounding tissues, and/or ability to establish new growth at ectopic sites. Cancers include, but are not limited to, solid tumors and hematological tumors.

Non-limiting examples of solid tumors include biliary tract cancer, pancreatic cancer, bladder cancer, colorectal cancer, breast cancer (including metastatic breast cancer), prostate cancer (including androgen-dependent and non-androgen-dependent prostate cancer), kidney cancer (including metastatic renal cell carcinoma), hepatocellular carcinoma, lung cancer (including non-small cell lung cancer (NSCLC), bronchioloalveolar carcinoma (BAC)), esophageal cancer, urothelial carcinoma, ovarian cancer (including progressive ovarian carcinoma or progressive primary peritoneal carcinoma), cervical cancer, endometrial cancer, gastric cancer, esophageal cancer, head and neck tumors (including head and neck squamous cell carcinoma), skin cancer (including malignant melanoma), neuroendocrine cancer (including metastatic neuroendocrine neoplasma), brain tumor (including for example glioma, anaplastic oligodendroglioma, and adult glioblastoma multiforme), bone cancer, soft-tissue sarcoma, and thyroid cancer.

Non-limiting examples of hematological tumors include acute myeloid leukemia (AML), chronic myelogenous leukemia (CML), including accelerated phase of chronic myelogenous leukemia and blast phase of chronic myelogenous leukemia (CML-BP), lymphoma, acute lymphoblastic leukemia (ALL), chronic lymphocytic leukemia (CLL), Hodgkin's disease (HD), non-Hodgkin's lymphoma (NHL) (including follicular lymphoma and mantle cell lymphoma), B-cell lymphoma, T-cell lymphoma, multiple myeloma (MM), diffuse large B-cell lymphoma (DLBL or DLBCL), Waldenstrom's macroglobulinemia, Burkitt's lymphoma, myelodysplastic syndromes (MDS) (including refractory anemia (RA), refractory anemia with ringed siderblasts (RARS), refractory anemia with excess blasts (RAEB), and RAEB in transformation (RAEB-T), and myeloproliferative syndrome.

In some embodiments, examples of cancers that can be treated include, but are not limited to, esophageal cancer, lung cancer, head and neck tumor, pancreatic cancer, colorectal cancer, cholangiocarcinoma, breast cancer, ovarian cancer, uterine cancer, cervical cancer, prostate cancer, bladder cancer, urothelial carcinoma, gastric cancer, kidney cancer, liver cancer, brain cancer, bone cancer, lymphoma, and leukemia.

The term “autoimmune disease” refers to a disease or condition caused by the immune response of body to its own antigens leading to damage to its own tissues or organs.

Non-limiting examples of autoimmune diseases include, but are not limited to, chronic obstructive pulmonary disease (COPD), allergic rhinitis, myasthenia gravis, systemic lupus erythematosus, autoimmune hematological diseases (e.g., hemolytic anemia, acquired hemophilia A (AHA), aplastic anemia, pure erythrocytic anemia and idiopathic thrombocytopenia (ITP), autoimmune inflammatory bowel disease (e.g., ulcerative colitis and Crohn's disease)), asthma and idiopathic thrombocytopenic purpura, Grave's disease, multiple sclerosis (MS), psoriasis, psoriatic arthritis, rheumatoid arthritis (RA), activated phosphoinositide 3-kinase-delta syndrome (APDS), and myeloproliferative disease (e.g., myelofibrosis, and post-polycythemia vera/essential thrombocytosis myelofibrosis (post-PV/ET myelofibrosis));

the neurodegenerative diseases include, but are not limited to, Alzheimer's disease, Huntington's disease, cerebellar atrophy, and Pick's disease; the viral infections include, but are not limited to, acquired immunodeficiency syndrome (AIDS); the bacterial infections include, but are not limited to, streptococcal infections; the ocular diseases include, but are not limited to, uveitis (anterior and posterior), retinopathy (e.g., diabetic retinopathy or hyperbaric oxygen-induced retinopathy), and conditions characterized by aqueous humor secretion or raised intraocular pressure (e.g., glaucoma); the kidney diseases include, but are not limited to, glomerulonephritis; the cardiovascular and cerebrovascular diseases include, but are not limited to, atherosclerosis, hypertension, deep vein thrombosis, stroke, myocardial infarction, unstable angina pectoris, thromboembolism, pulmonary embolism, thrombolytic disease, acute arterial ischemia, peripheral thrombotic occlusion, coronary artery disease, and reperfusion injury; the psychological diseases include, but are not limited to, depression, anxiety, and psychological disorders.

In some embodiments, the present invention provides a method of delivering a PI3K and/or PIKK inhibitor to a cell or tissue expressing a corresponding target (e.g., HER2, CD20, CD38, CEACAM5/6, FRα, or Nectin-4), the method comprising conjugating the PI3K and/or PIKK inhibitor to an antibody that immunospecifically binds to the corresponding target (e.g., HER2, CD20, CD38, CEACAM5/6, FRα, or Nectin-4) to obtain an antibody-conjugated drug (abbreviated as “ADC”), and exposing the cell or tissue to the ADC.

### DETAILED DESCRIPTION OF EMBODIMENTS

The examples below are intended to be purely exemplary and should not be considered to be limiting the invention in any way. Efforts have been made to ensure the accuracy with respect to numbers used (such as amounts, temperature, etc.), but a person skilled in the art should understand that some experimental errors and deviations should be accounted for. Unless indicated otherwise, parts are parts by weight, temperature is in degrees Centigrade, and pressure is at or near atmospheric. All MS data were determined by Agilent 6120 and Agilent 1100. All NMR data were generated using a Varian 400 MR machine. All reagents and starting materials, except synthesized intermediates, used in the present invention were commercially available. All compound names except the reagents were generated by Chemdraw 20.1.

If there is any atom with empty valence(s) in any one of the structures disclosed herein, the empty balance(s) is (are) the hydrogen atom(s) which is (are) omitted for convenience purpose.

In the present application, in the case of inconsistency of the name and structure of a compound, when the two of which are both given for the compound, it is subject to the structure of the compound, unless the context shows that the structure of the compound is incorrect, and the name is correct.

List of abbreviations used in the following examples:

| | |
|---|---|
| ADC | Antibody-drug conjugate |
| B₂pin₂ | Bis(pinacolato)diboron |
| CDI | N,N′-carbonyldiimidazole |
| CH₃CN | Acetonitrile |
| DBU | 1,8-diazabicyclo-undec-7-ene |
| DCM | Dichloromethane |
| DIEA | N,N-diisopropylethylamine |
| DMA | N,N-dimethylacetylamide |
| DMAP | 4-dimethylaminopyridine |
| DMF | N,N-dimethylformamide |
| DMSO | Dimethyl sulfoxide |
| Dowtherm A | Diphenyl ether-biphenyl eutectic |
| DTPA | Diethylenetriaminepentaacetic acid |
| EA | Ethyl acetate |
| EDCI | 1-ethyl-(3-dimethylaminopropyl)carbodiimide |
| Et₂N | Diethylamine |
| Et₃N | Triethylamine |
| Fmoc-OSU | 9-fluorenylmethyl-N-succinimidyl carbonate |
| g | Gram |
| h | Hour |
| HATU | 2-(7-azabenzotriazol-1-yl)-N,N,N′,N′-tetramethyluronium |
| | hexafluorophosphate |
| His | Histidine |
| HIC-HPLC | Hydrophobic interaction chromatography |
| H₂O | Water |
| HOBt | 1-hydroxybenzotriazole |
| ISCO | Fast preparative liquid chromatography |
| L | Liter |
| M | Mole/liter |
| MeOH | Methanol |
| mg | Milligram |
| min | Minute |
| mL | Milliliter |
| mmol | Millimole |
| mol | Mole |
| MS | Mass spectrometry |
| PBS-D | Phosphate buffered solution- |
| | Diethylenetriaminepentaacetic acid |
| Pd(dppf)Cl₂ | [1,1′-bis(diphenylphosphino)ferrocene]palladium |
| | dichloride |
| PdCl₂(PPh₃)₂ | Bis(triphenylphosphine)palladium (II) dichloride |
| PE | Petroleum ether |
| pH | Power of hydrogen |
| POCl₃ | Phosphorus oxychloride |
| SO₂Cl₂ | Sulfonyl chloride |
| TCEP | Tris(2-carboxyethyl)phosphine |
| TFA | Trifluoroacetic acid |
| THF | Tetrahydrofuran |

### Example 1: Preparation of Intermediates

### Preparation of Intermediate-1:

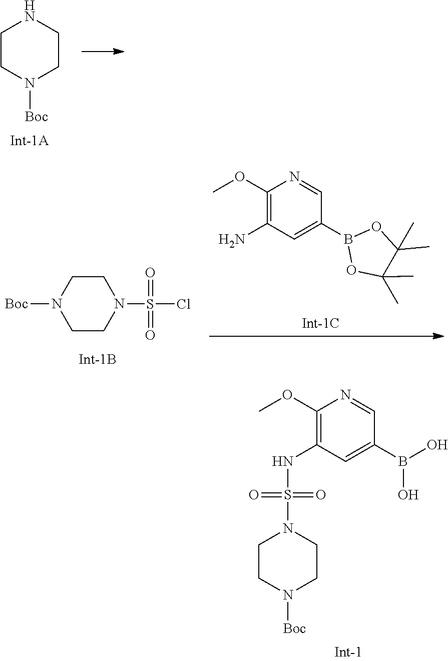

### 1) Synthesis of Compound Int-1B

In an ice-water bath, SO₂Cl₂ (19.93 g, 147.65 mmol) was added to a solution of compound Int-1A (CAS: 57260-71-6) (25 g, 134.22 mmol) and pyridine (15.91 g, 201.34 mmol) in dichloromethane. After the completion of dropwise addition, the reaction solution was stirred continuously in the ice-water bath for 2 hours. After the reaction was ended, the reaction solution was poured into water, and extracted with dichloromethane (200 mL×3). The extract was washed once with 1 N hydrochloric acid (100 mL). The organic phases were combined, dried over anhydrous sodium sulfate, and concentrated under reduced pressure to afford compound Int-1B as a yellow solid (18 g, 47% yield).

### 2) Synthesis of Intermediate-1

At room temperature, to a solution of compound Int-1B (CAS: 1000339-10-5) (17.08 g, 59.97 mmol) and compound Int-1C (10 g, 39.98 mmol) in 1,2-dichloroethane was added DMAP (9.77 g, 79.96 mmol) and Et₃N (6.06 g, 59.97 mmol). After the addition was completed, the reaction solution was stirred at 75° C. for 24 hours. After the reaction was ended, the mixture was filtered, and the resulting filtrate was concentrated. The resulting residue was dissolved in ethyl acetate (200 mL), and washed with 1 N hydrochloric acid (60 mL). The organic phase was collected. At the same time, the aqueous phase was extracted with ethyl acetate (100 mL×3). The organic phases were combined and dried over anhydrous sodium sulfate. The solvent was removed by concentration under reduced pressure. The resulting residue was purified by ISCO (mobile phase: water (0.1% formic acid):methanol=100:0-0:100) to afford intermediate-1 as a yellow solid (7 g, 42% yield). MS (m/z)=439.0 [M+Na]⁺

### Preparation of Intermediate-2:

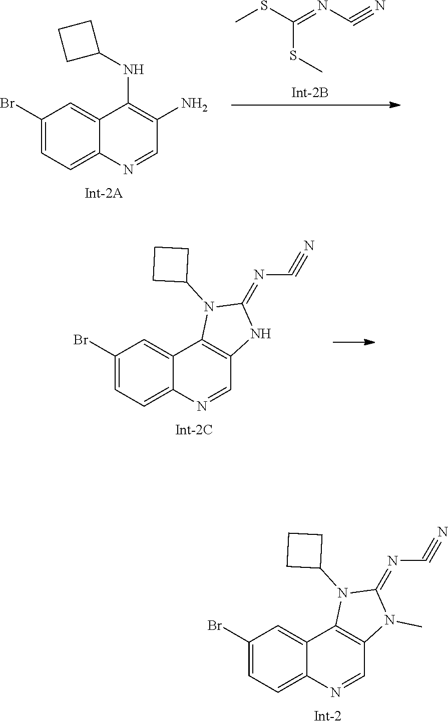

### 1) Synthesis of Compound Int-2C

Under nitrogen protection, a solution of compound Int-2A (according to the preparation procedure of intermediate-5) (190 mg, 0.65 mmol), compound Int-2B (CAS: 10191-60-3) (190 mg, 1.30 mmol), and DBU (297 mg, 1.95 mmol) in dioxane was stirred at 100° C. overnight. After the reaction was completed, the mixture was concentrated and the residue was purified by ISCO (mobile phase: MeOH:H₂O=0:100-100:0) to afford compound Int-2C as a white solid (102 mg, 46% yield). MS (m/z)=318, 320 [M−24]⁺

### 2) Synthesis of Intermediate-2

In an ice-water bath and under nitrogen protection, to a solution of compound Int-2C (102 mg, 0.30 mmol) in DMF was added NaH (14 mg, 0.60 mmol). After the addition was completed, the reaction solution was stirred under nitrogen protection in an ice-water bath for 10 minutes. Then, CH₃I (85 mg, 0.60 mmol) was added to the reaction solution in one portion. The reaction was stirred at room temperature for 1 h. After the reaction was completed, the reaction solution was poured into water, and extracted with ethyl acetate (20 mL×3). The organic phases were combined, dried over anhydrous sodium sulfate, and concentrated under reduced pressure. The residue was purified by ISCO (mobile phase: MeOH:H₂O=0:100-100:0) to afford intermediate-2 as a white solid (23 mg, 22% yield). MS (m/z)=332.0, 334.0 [M−24]⁺

### Preparation of Intermediate-3

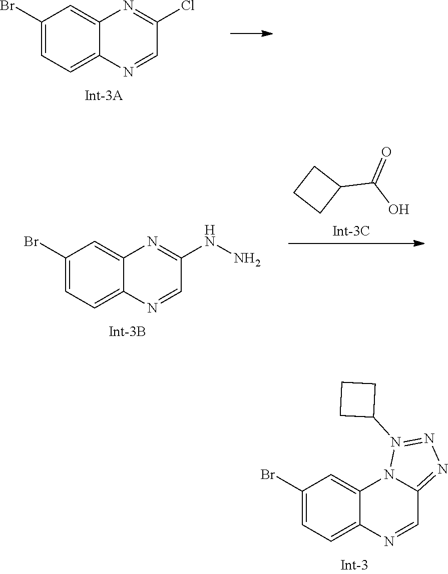

### 1) Synthesis of Compound Int-3B

Compound Int-3A (5 g, 20.53 mmol) and hydrazine hydrate (10 mL) were dissolved in ethanol (100 mL). The reaction was stirred at reflux overnight. After the reaction was completed, the mixture was concentrated under reduced pressure to make the reaction solution's volume reduce to half of the original volume. The solid was collected by filtration, and dried to afford Int-3B as a light-yellow solid (3.9 g, 79.0% yield). MS (m/z)=239.0, 241.0 [M+H]⁺

### 2) Synthesis of Intermediate-3

Compound Int-3B (500 mg, 2.09 mmol) and compound Int-3C (2 mL) were mixed, heated to 110° C. and stirred for 24 h. After the reaction was ended, purification was conducted by using ISCO (mobile phase: dichloromethane:methanol=100:0-75:25) to afford compound intermediate-3 as a light-yellow solid (460 mg, 73% yield). MS (m/z)=303.0, 305.0 [M+H]⁺

The following compounds were prepared according to the preparation procedure of intermediate-3 using corresponding intermediates and reagents under appropriate conditions recognized by a person skilled in the art.

| Intermediate | | MS |
|---|---|---|
| No. | Structural formula | (M + H)⁺ |
| Intermediate-4 | 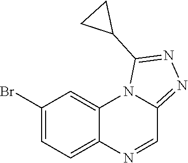 [SMILES: Brc1ccc2ncc3nnc(C4CC4)n3c2c1] | 289.0 [M + 1]⁺ |

### Preparation of Intermediate-5

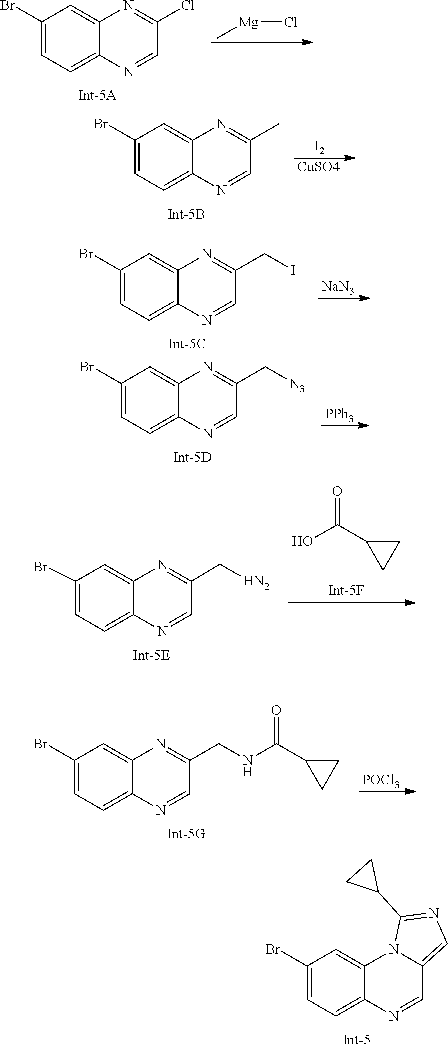

### 1) Synthesis of Compound Int-5B

In an ice-water bath and under nitrogen protection, methyl magnesium chloride (9 mL, 27.1 mmol) was slowly added to a solution of compound Int-5A (CAS: 89891-65-6) (6 g, 24.6 mmol) and iron triacetylacetonate (0.9 g, 2.5 mmol) in THF. The reaction was stirred at 0° C. for 2 h. After the reaction was completed, the reaction solution was extracted with ethyl acetate (200 mL×3). The organic phases were combined, dried over anhydrous sodium sulfate, and concentrated under reduced pressure. The residue was purified by ISCO (water (0.1% TFA):methanol=100:0-0:100 gradient elution) to afford compound Int-5B as a yellow solid (5.0 g, 90.9% yield). MS (m/z)=223.2 [M+H]⁺

### 2) Synthesis of Compound Int-5C

Under nitrogen protection, iodine (8.53 g, 33.6 mmol), compound Int-5B (5 g, 22.4 mmol) and copper sulfate (3.56 g, 22.4 mmol) was dissolved in acetonitrile, and the reaction was stirred at 70° C. for 3 h. After the reaction was completed, the reaction solution was concentrated and the residue was purified by ISCO (water (0.1% TFA):methanol=100:0-0:100 gradient elution) to afford compound Int-5C as a yellow solid (1.0 g, 12.8% yield). MS (m/z)=349.2.2 [M+H]⁺

### 3) Synthesis of Compound Int-5D

Under nitrogen protection, sodium azide (224 mg, 3.4 mmol) was added to a solution of compound Int-5C (1 g, 2.8 mmol) in DMF, and the reaction was stirred at room temperature for 2 h. After the reaction was completed, the reaction solution was extracted with ethyl acetate (100 mL×3). The organic phases were combined, dried over anhydrous sodium sulfate, and concentrated under reduced pressure. The residue was purified by ISCO (water (0.1% TFA):methanol=100:0-0:100 gradient elution) to afford compound Int-5D as a white solid (480 mg, 63.4% yield). MS (m/z)=263.8 [M+H]⁺

### 4) Synthesis of Compound Int-5E

Under nitrogen protection, triphenylphosphine (572 mg, 2.2 mmol) was added to a solution of compound Int-5D (480 mg, 1.8 mmol) in THF:H₂O (2:1), and the reaction was stirred at room temperature for 12 h. After the reaction was completed, the reaction solution was concentrated, and the residue was purified by ISCO (water (0.1% TFA):methanol=100:0-0:100 gradient elution) to afford compound Int-5E as a black solid (270 mg, 62.4% yield). MS (m/z)=237.8 [M+H]⁺

### 5) Synthesis of Compound Int-5G

Under nitrogen protection, HATU (210 mg, 0.55 mmol), DIEA (195 mg, 1.5 mmol), and compound Int-5F (43 mg, 0.5 mmol) were added to a solution of compound Int-5E (120 mg, 0.5 mmol) in DMF, and the reaction was stirred at room temperature for 1 h. After the reaction was completed, the reaction solution was concentrated and the residue was purified by ISCO (water (0.1% TFA):methanol=100:0-0:100 gradient elution) to afford compound Int-5G as a white solid (100 mg, 64.8% yield). MS (m/z)=305.8 [M+H]⁺

### 6) Synthesis of Intermediate-5

Under nitrogen protection, compound Int-5G (100 mg, 0.33 mmol) was added to POCl₃, and the reaction was stirred at 75° C. for 3 h. After the reaction was completed, the reaction solution was concentrated and the residue was purified by ISCO (water (0.1% TFA)/methanol=100:0-0:100 gradient elution) to afford intermediate-5 as a white solid (85 mg, 90.3% yield). MS (m/z)=287.8 [M+H]⁺

The following compounds were prepared according to the preparation procedure of intermediate-5 using corresponding intermediates and reagents under appropriate conditions recognized by a person skilled in the art.

| Intermediate | | |
|---|---|---|
| No. | Structural formula | MS (M + H)⁺ |
| Intermediate-6 | 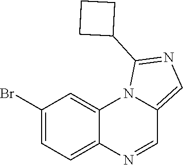 [SMILES: Brc1ccc2ncc3cnc(C4CCC4)n3c2c1] | 301.8 [M + H]⁺ |
| Intermediate-7 | 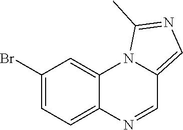 | 261.8 [M + H]⁺ |
| Intermediate-24 | 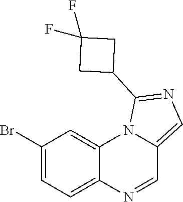 [SMILES: FC1(F)CC(c2ncc3cnc4ccc(Br)cc4n23)C1] | 338.0 [M + H]⁺ |

### Preparation of Intermediate-8:

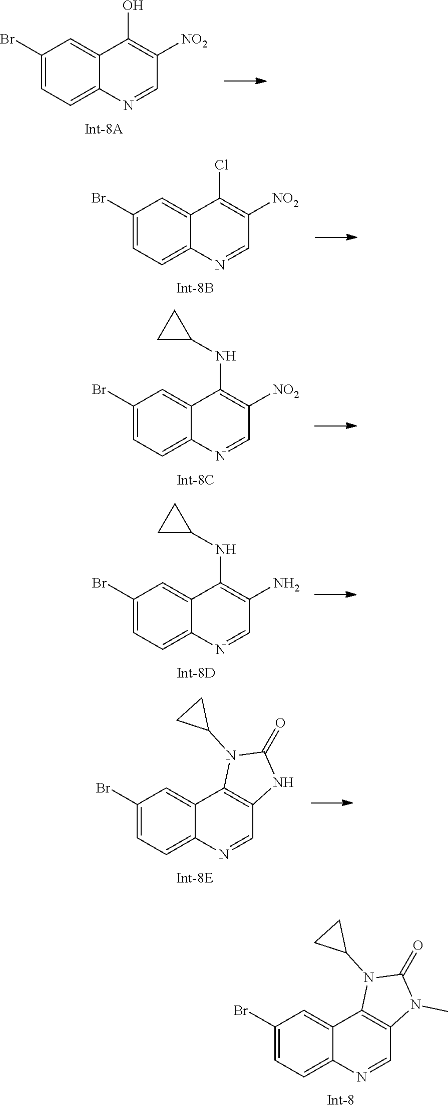 [SMILES: O=[N+]([O-])c1cnc2ccc(Br)cc2c1O ; O=[N+]([O-])c1cnc2ccc(Br)cc2c1Cl ; O=[N+]([O-])c1cnc2ccc(Br)cc2c1NC1CC1 ; Nc1cnc2ccc(Br)cc2c1NC1CC1 ; O=c1[nH]c2cnc3ccc(Br)cc3c2n1C1CC1 ; Cn1c(=O)n(C2CC2)c2c3cc(Br)ccc3ncc21]

### 1) Synthesis of Compound Int-8B

Under nitrogen protection, compound Int-8A (CAS: 853908-50-6) (2.69 g, 10 mmol) was dissolved in phosphorus oxychloride (15 mL). The reaction solution was stirred and refluxed for 5 hours. After the reaction was ended, the reaction solution was slowly poured into ice water, and extracted with dichloromethane (30 mL×3). The organic phases were combined and concentrated to afford compound Int-8B as a yellow solid (2.58 g, 90% yield). MS (m/z)=287.0 [M+H]⁺

### 2) Synthesis of Compound Int-8C

Compound Int-8B (2.58 g, 9 mmol) and cyclopropylamine (514 mg, 9 mmol) were dissolved in isopropanol (15 mL). The reaction was stirred at 80° C. for 4 h. After the reaction was completed, the reaction solution was naturally cooled to room temperature, and a solid was precipitated out obviously. The solid was collected by filtration, washed with isopropanol, and dried in vacuo to afford a yellow solid (1.94 g, 70% yield). MS (m/z)=308.0 [M+H]⁺

### 3) Synthesis of Compound Int-8D

Tin dichloride dihydrate (5.68 g, 25.2 mmol) was added to a solution of compound Int-8C (1.94 g, 6.3 mmol) in ethyl acetate (40 mL) and the reaction was stirred at reflux for 4 h. After the reaction was completed, the reaction was cooled to room temperature and the pH was adjusted to pH 9.0 with 2 N aqueous sodium hydroxide solution. The reaction solution was then filtered. The filtrate was collected, and extracted with ethyl acetate (30 mL×3). The organic phases were combined, dried over anhydrous sodium sulfate, and concentrated under reduced pressure to afford compound Int-8D as a yellow solid (1.41 g, 80% yield). MS (m/z)=278.0 [M+H]⁺

### 4) Synthesis of Compound Int-8E

In an ice-water bath, to a solution of compound Int-8D (556 mg, 2 mmol) and triethylamine (607 mg, 6 mmol) in dichloromethane (10 mL) was added triphosgene (415 mg, 1.4 mmol) in batches. After the addition was completed, the mixture was stirred at room temperature for 3 h. After the reaction was completed, the reaction solution was slowly poured into water, and extracted with dichloromethane (30 mL×3). The organic phases were combined, dried over anhydrous sodium sulfate, and concentrated under reduced pressure to afford compound Int-8E as a brown solid (610 mg, 99% yield). MS (m/z)=304.0 [M+H]⁺

### 5) Synthesis of Intermediate-8

To a solution of compound Int-8E (610 mg, 2 mmol) in DMF (10 mL) was added potassium carbonate (829 mg, 6 mmol) followed by methyl iodide (710 mg, 5 mmol). After the addition was completed, the reaction solution was stirred at room temperature for 18 hours. After the reaction was ended, the reaction solution was concentrated, and the residue was purified by ISCO (mobile phase: water (0.1% formic acid):acetonitrile=100:0-0:100) to afford compound intermediate-8 as a yellow solid (382 mg, 60% yield). MS (m/z)=318.0 [M+H]⁺

The following compounds were prepared according to the preparation procedure of intermediate-8 using corresponding intermediates and reagents under appropriate conditions recognized by a person skilled in the art.

| Intermediate | Structural | MS |
|---|---|---|
| No. | formula | (M + H)⁺ |
| Intermediate-9 | 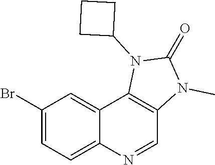 [SMILES: Cn1c(=O)n(C2CCC2)c2c3cc(Br)ccc3ncc21] | 332.2 [M + H]⁺ |
| Intermediate-10 | 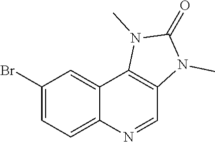 [SMILES: Cn1c(=O)n(C)c2c3cc(Br)ccc3ncc21] | 294.0 [M + H]⁺ |
| Intermediate-11 | 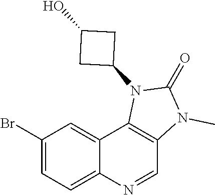 [SMILES: Cn1c(=O)n([C@H]2C[C@H](O)C2)c2c3cc(Br)ccc3ncc21] | 348.0 [M + H]⁺ |
| Intermediate-12 | 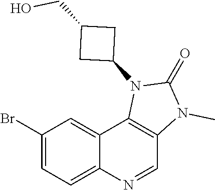 [SMILES: Cn1c(=O)n([C@H]2C[C@H](CO)C2)c2c3cc(Br)ccc3ncc21] | 362.0 [M + H]⁺ |
| Intermediate-13 | 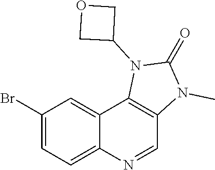 [SMILES: Cn1c(=O)n(C2COC2)c2c3cc(Br)ccc3ncc21] | 334.0 [M + H]⁺ |
| Intermediate-14 | 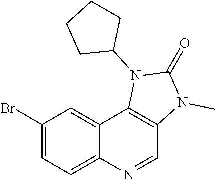 | 336.2 [M + H]⁺ |
| Intermediate-25 | 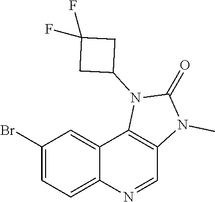 [SMILES: Cn1c(=O)n(C2CC(F)(F)C2)c2c3cc(Br)ccc3ncc21] | 368.0 [M + H]⁺ |
| Intermediate-27 | 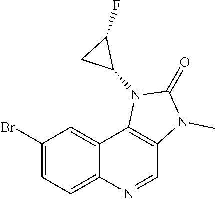 [SMILES: Cn1c(=O)n([C@@H]2C[C@@H]2F)c2c3cc(Br)ccc3ncc21] | 336.0 [M + H]⁺ |
| Intermediate-29 | 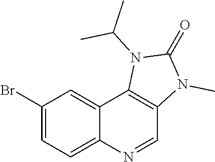 [SMILES: CC(C)n1c(=O)n(C)c2cnc3ccc(Br)cc3c21] | 320.2 [M + H]⁺ |
| Intermediate-30 | 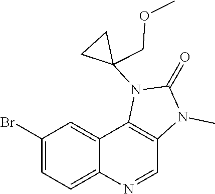 [SMILES: COCC1(n2c(=O)n(C)c3cnc4ccc(Br)cc4c32)CC1] | 364.1 [M + H]⁺ |
| Intermediate-32 | 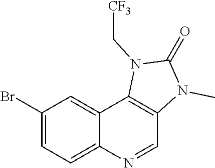 [SMILES: Cn1c(=O)n(CC(F)(F)F)c2c3cc(Br)ccc3ncc21] | 360.0 [M + H]⁺ |
| Intermediate-33 | 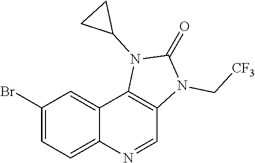 [SMILES: O=c1n(CC(F)(F)F)c2cnc3ccc(Br)cc3c2n1C1CC1] | 385.9 [M + H]⁺ |
| Intermediate-35 | 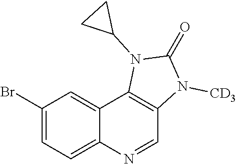 [SMILES: [2H]C([2H])([2H])n1c(=O)n(C2CC2)c2c3cc(Br)ccc3ncc21] | 321.0 [M + H]⁺ |

### Preparation of Intermediate-15:

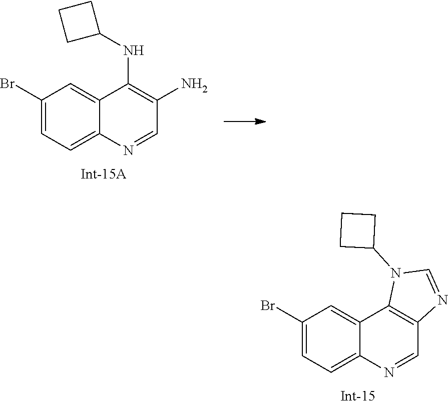 [SMILES: Nc1cnc2ccc(Br)cc2c1NC1CCC1 ; Brc1ccc2ncc3ncn(C4CCC4)c3c2c1]

Compound Int-15A (157 mg, 0.54 mmol) (prepared according to the preparation procedure of intermediate-5), triethyl orthoformate (119 mg, 0.81 mmol), and pyridine hydrochloride (1 mg, 0.005 mmol) were dissolved in toluene (20 mL). The reaction was stirred at reflux for 3 h. After the reaction was completed, the reaction solution was concentrated under reduced pressure and the residue was purified by ISCO (mobile phase: (ACN:H₂O (TFA)=0:100-100:0) to afford intermediate-15 as a white solid (100 mg, 62% yield). MS (m/z)=302.0 [M+1]+

The following compounds were prepared according to the preparation procedure of intermediate-15 above using corresponding intermediates and reagents under appropriate conditions recognized by a person skilled in the art.

| Intermediate | | MS |
|---|---|---|
| No. | Structural formula | (M + H)⁺ |
| Intermediate-16 | 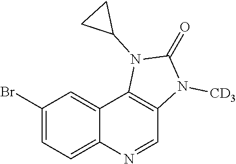 [SMILES: [2H]C([2H])([2H])n1c(=O)n(C2CC2)c2c3cc(Br)ccc3ncc21] | 288.2 [M + H]⁺ |

### Preparation of Intermediate-17:

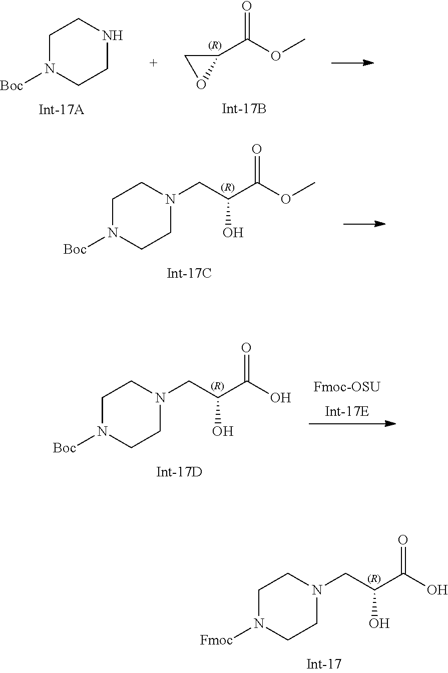

### 1) Synthesis of Compound Int-17C

Compound Int-17A (CAS: 57260-71-6) (4 g, 21.5 mmol), methyl (R)-2,3-epoxypropionate (2.63 g, 25.8 mmol), and DIEA (11.2 mL, 64.5 mmol) were dissolved in DMF (30 mL), and the reaction solution was stirred at 80° C. for 18 hours. After the reaction was cooled to room temperature, water (40 mL) was added, the mixture was extracted with ethyl acetate (25 mL×3). The organic phases were combined, washed once with water (40 mL), dried over anhydrous sodium sulfate and filtered. The resulting filtrate was concentrated to afford Int-17C as a light-yellow solid (5.26 g, 85% yield). MS (m/z): 289.2 [M+H]⁺

### 2) Synthesis of Compound Int-17D

Compound Int-17C (2.88 g, 10 mmol) and lithium hydroxide monohydrate (2 g, 50 mmol) were dissolved in a mixed solution of methanol/water (15 mL/6 mL), and the reaction solution was heated to 40° C. and stirred for 18 hours. The reaction solution was cooled to room temperature, further cooled in an ice-water bath and then adjusted to pH 7.0 with 5 N hydrochloric acid in the ice-water bath. The mixture was concentrated under reduced pressure, and separated by ISCO (mobile phase: H₂O (0.11% HCOOH):CH₃CN=100:0-0:100) to afford compound Int-17D as a light-yellow solid (2.41 g, 88.0% yield). MS (m/z): 275.2 [M+H]⁺

### 3) Synthesis of Intermediate-17

Under nitrogen protection, compound Int-17D (500 mg, 1.8 mmol) was added to a mixed solution of TFA:DCM (1:1, 5 mL) and the mixture was stirred for 20 minutes. After the reaction was completed, the reaction solution was concentrated to afford a residue. The residue was dissolved in a mixed solvent of dioxane and water (1:1, 10 mL). Sodium bicarbonate (460 mg, 5.5 mmol) and Fmoc-OSU (Int-17E, CAS: 82911-69-1, 677 mg, 2.0 mmol) were added to the above solution under ice bath. The reaction was carried out at room temperature for 3 hours. After the reaction was completed, the reaction solution was extracted with ethyl acetate (20 mL×3). The organic phases were combined, dried over anhydrous sodium sulfate, and concentrated under reduced pressure, and then purified by ISCO (water (0.1% TFA):acetonitrile=100:0-0:100 gradient elution) to afford intermediate-17 as a white solid (650 mg, 90.0% yield). MS (m/z)=397.6 [M+H]⁺

### Preparation of Intermediate-18:

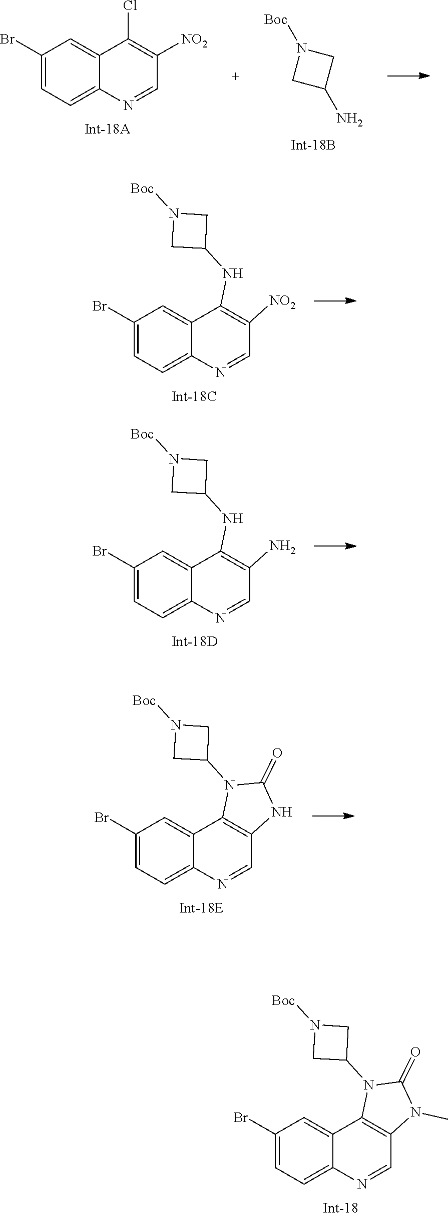 [SMILES: O=[N+]([O-])c1cnc2ccc(Br)cc2c1Cl ; CC(C)(C)OC(=O)N1CC(N)C1 ; CC(C)(C)OC(=O)N1CC(Nc2c([N+](=O)[O-])cnc3ccc(Br)cc23)C1 ; CC(C)(C)OC(=O)N1CC(Nc2c(N)cnc3ccc(Br)cc23)C1 ; CC(C)(C)OC(=O)N1CC(n2c(=O)[nH]c3cnc4ccc(Br)cc4c32)C1 ; Cn1c(=O)n(C2CN(C(=O)OC(C)(C)C)C2)c2c3cc(Br)ccc3ncc21]

### 1) Synthesis of Compound Int-18C

Compound Int-18A (prepared in Step 1 of the Int-8 preparation) (1.7 g, 6 mmol) and compound Int-18B (1.03 g, 6 mmol) were dissolved in isopropanol (15 mL). The reaction was stirred at 80° C. for 4 h. After the reaction was completed, the reaction solution was naturally cooled to room temperature, and a solid was precipitated out obviously. A filter cake was obtained by filtration, which was washed with isopropanol to afford a yellow solid (2.2 g, 86% yield). MS (m/z)=423.0 [M+H]⁺

### 2) Synthesis of Compound Int-18D

Compound Int-18C (2 g, 4.73 mmol), iron powder (1.59 g, 28.4 mmol), and ammonium chloride (1.53 g, 28.4 mmol) were added to a mixed solution of ethanol and water (30 mL/10 mL), and the reaction was stirred at 90° C. for 5 h. After the reaction was completed, the reaction solution was cooled to room temperature, and then was filtered. The filtrate was collected and concentrated under reduced pressure to afford compound Int-18D as a yellow solid (1.5 g, 80% yield). MS (m/z)=393.2 [M+H]⁺

### 3) Synthesis of Compound Int-18E

In an ice-water bath, to a solution of compound Int-18D (1.5 g, 3.81 mmol) and triethylamine (1.16 g, 3 mmol) in dichloromethane (10 mL) was added triphosgene (407 mg, 1.37 mmol) in batches. After the addition was completed, the mixture was stirred at room temperature for 3 h. After the reaction was completed, the reaction solution was slowly poured into water, and extracted with dichloromethane. The organic phase was collected, dried and concentrated to afford a brown solid (1.98 g, 99% yield). MS (m/z)=419.0 [M+H]⁺

### 4) Synthesis of Intermediate-18

To a solution of compound Int-18E (1.98 mg, 4.72 mmol) in DMF (15 mL) was added potassium carbonate (2.09 g, 15.1 mmol) followed by methyl iodide (0.44 mL, 7.09 mmol). After the addition was completed, the reaction solution was stirred at room temperature for 18 hours. After the reaction was ended, the reaction solution was concentrated, and the residue was purified by ISCO (mobile phase: water (0.1% trifluoroacetic acid):methanol=100:0-0:100) to afford intermediate-18 as a yellow solid (1.43 g, 70% yield). MS (m/z)=433.0 [M+H]+

### Preparation of Intermediate-19:

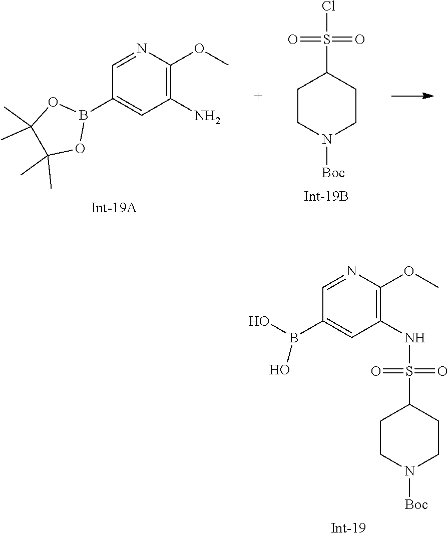

At room temperature and under nitrogen protection, a solution of DIEA (312 mg, 2.40 mmol), DMAP (20 mg, 0.16 mmol), and compound Int-19A (CAS: 893440-50-1) (400 mg, 1.60 mmol) in DCM (10 mL) was added dropwise to a solution of compound Int-19B (CAS: 782501-25-1) (544 mg, 1.92 mmol) in DCM (20 mL). After the addition was completed, the reaction solution was stirred at room temperature for 1 h. After the reaction was completed, the reaction solution was poured into water, and extracted with DCM (15 mL×3). The organic phases were combined, dried over anhydrous sodium sulfate, and concentrated under reduced pressure. The residue was purified by ISCO (mobile phase: CH₃CN:H₂O=0:100-100:0) to afford intermediate-19 as a white solid (158 mg, 24% yield). MS (m/z)=360.1 [M−56]⁺

### Preparation of Intermediate-20:

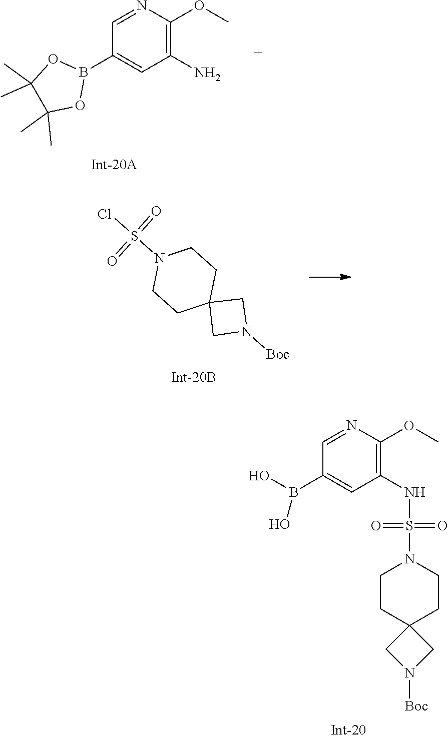

At room temperature and under nitrogen protection, pyridine (0.5 mL), DMAP (15 mg, 0.12 mmol), and compound Int-20A (CAS: 893440-50-1) (400 mg, 1.17 mmol) were added to a solution of compound Int-20B (380 mg, 1.12 mmol) in 1,2 dichloroethane. After the addition was completed, the reaction solution was stirred under nitrogen protection at 70° C. for 24 h. After the reaction was completed, the reaction solution was poured into water, and extracted with DCM (15 mL×3). The organic phases were combined, dried over anhydrous sodium sulfate, and concentrated under reduced pressure. The residue was purified by ISCO (mobile phase: CH₃CN:H₂O=0:100-100:0) to afford intermediate-20 as a white solid (190 mg, 35% yield). MS (m/z)=401.2 [M−56]⁺

### Preparation of Intermediate-21:

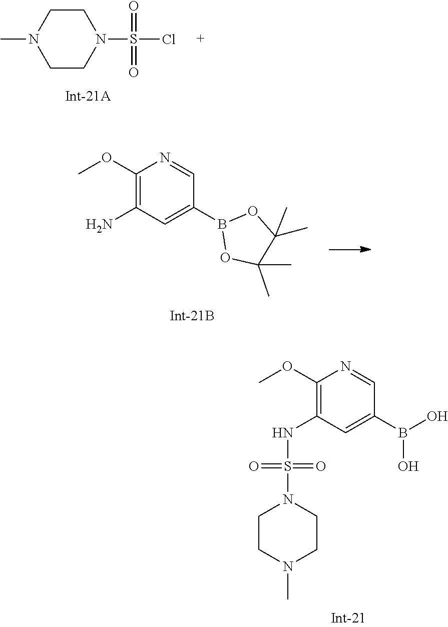

Compound Int-21B (CAS: 893440-50-1) (50 mg, 0.2 mmol) and compound Int-21A (CAS: 893440-50-1) (120 mg, 0.6 mmol) were dissolved in pyridine (4 mL) at room temperature, and placed in a sealed tube. The reaction solution was stirred at 60° C. for 18 hours. After the reaction was ended, the reaction solution was concentrated under reduced pressure, and the residue was purified by ISCO (mobile phase: water (0.1% formic acid):acetonitrile=100:0-0:100) to afford intermediate-21 as a yellow solid (66 mg, 99% yield). MS (m/z)=331.2 [M+1]⁺

### Preparation of Intermediate-22:

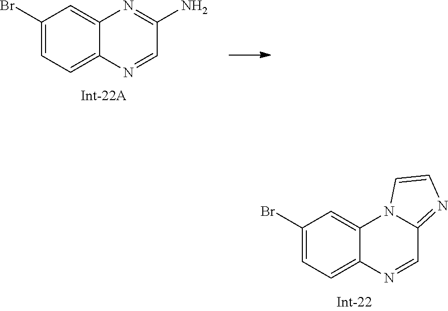 [SMILES: Nc1cnc2ccc(Br)cc2n1 ; Brc1ccc2ncc3nccn3c2c1]

Compound Int-22A (CAS: 212327-11-2) (500 mg, 2.2 mmol) and chloroacetaldehyde (1.7 g, 22 mmol) were added to an ethanol solution, and the reaction was stirred at 105° C. for 12 h. After the reaction was completed, the reaction solution was concentrated, and the residue was purified by ISCO (mobile phase: CH₃CN:H₂O (0.1% TFA)=0:100-100:0) to afford intermediate-22 as a yellow solid (400 mg, 72% yield). MS (m/z)=248.0 [M+H]⁺

### Preparation of Intermediate-23

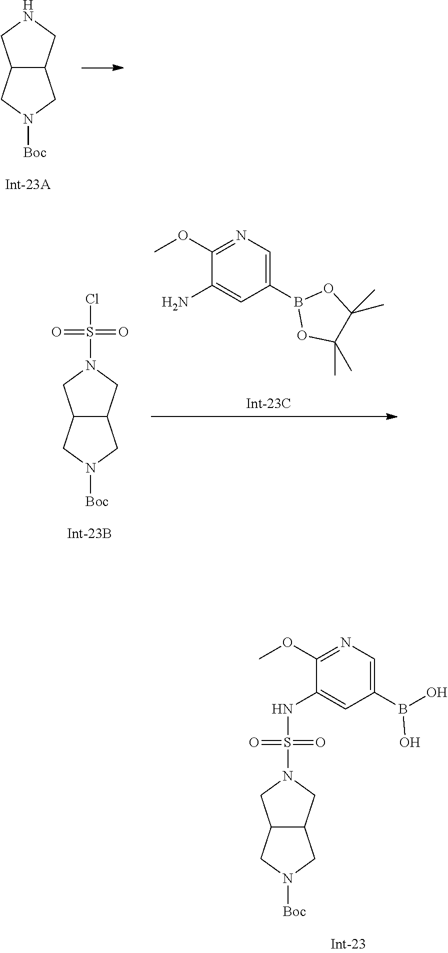

### 1) Synthesis of Compound Int-23B

In an ice-water bath, SO₂Cl₂ (3.5 g, 7.09 mmol) was added to a solution of compound Int-23A (CAS: 141449-85-6) (5 g, 23.55 mmol) and triethylamine (6.55 mL, 15.1 mmol) in dichloromethane. After the completion of dropwise addition, the reaction solution was stirred continuously at room temperature for 2 hours. After the reaction was ended, the reaction solution was poured into water, and extracted with dichloromethane (200 mL×3). The extract was washed once with 1 N hydrochloric acid (100 mL). The organic phases were combined, dried over anhydrous sodium sulfate, and concentrated under reduced pressure to afford compound Int-23B as a yellow solid (5.28 g, 72% yield). MS (m/z)=255.0 [M−55]⁺

### 2) Synthesis of Intermediate-23

Compound Int-23B (2.98 g, 9.6 mmol) and compound Int-23C (CAS: 893440-50-1) (400 mg, 1.6 mmol) were dissolved in pyridine (15 mL) at room temperature, and placed in a sealed tube. The reaction solution was stirred at 60° C. for 18 hours. After the reaction was ended, the reaction solution was concentrated under reduced pressure, and the residue was purified by ISCO (mobile phase: water (0.1% TFA):methanol=100:0-0:100) to afford intermediate-23 as a yellow solid (353 mg, 50% yield). MS (m/z)=441.2 [M−1]⁻

### Preparation of Intermediate-26

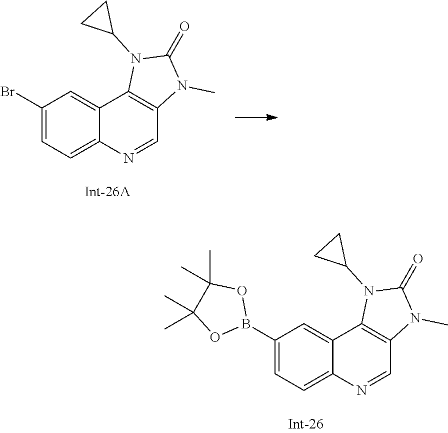

Int-26A (prepared according to the Int-8 preparation) (6.0 g, 18.8 mmol) was charged into a reaction flask, and 1,4-dioxane (900 mL), B₂Pin₂ (7.2 g, 28.3 mmol), potassium acetate (4.6 g, 46.9 mmol), and Pd(dppf)Cl₂ (0.68 g, 0.93 mmol) were added, purged with N₂, and heated to 90° C. to react for 16 hours. The reaction solution was cooled to room temperature, and filtered on celite. The filtrate was concentrated to dryness under reduced pressure. The mixed solvent of 20 mL DCM and 90 mL hexane was added and pulped at room temperature overnight, and the mixture was filtered. The filter cake was dried to afford compound intermediate-26 as a grey solid (5.6 g, 81.3% yield). MS (m/z)=366.2 [M+H]⁺

### Preparation of Intermediate-28

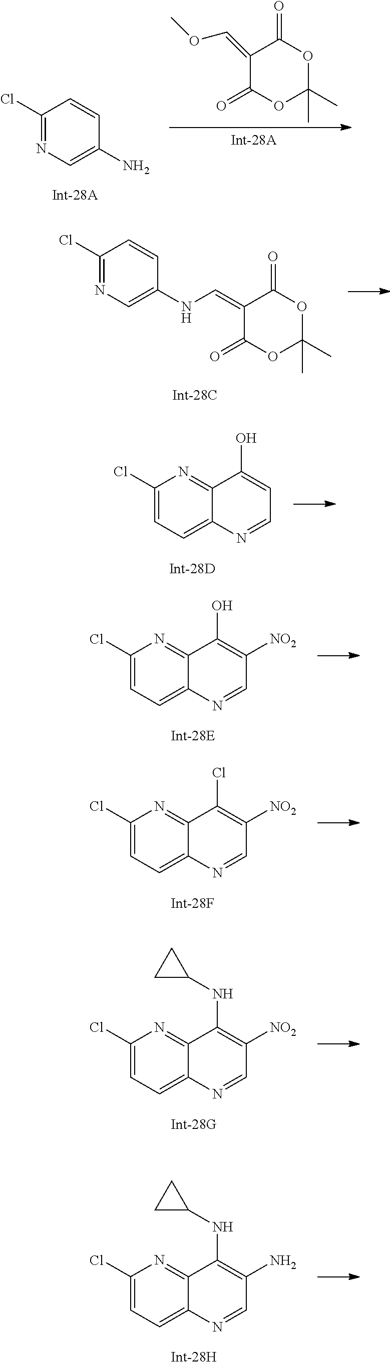

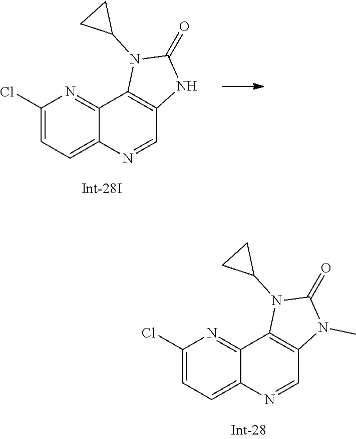 [SMILES: O=c1[nH]c2cnc3ccc(Cl)nc3c2n1C1CC1 ; Cn1c(=O)n(C2CC2)c2c3nc(Cl)ccc3ncc21]

### 1) Synthesis of Compound Int-28C

Compound Int-28A (CAS: 5350-93-6 (6.00 g, 46.67 mmol) and compound Int-28B (CAS:15568-85-1) (17.38 g, 93.34 mmol) were dissolved in isopropanol (40 mL) at room temperature, and the mixture was refluxed at 90° C. for two hours. After the reaction was completed, the reaction solution was concentrated. The residue was pulped with PE (100 mL) before filtered. The filter cake was collected to afford compound Int-28C as a brown solid (13.00 g, 98% yield). MS (m/z)=283.0 [M+H]⁺

### 2) Synthesis of Compound Int-28D

At 220° C. and under nitrogen protection, to a solution of Dowtherm A (850 mL) was added compound Int-28C (13.00 g, 45.98 mmol) in batches. After the addition was completed, the reaction solution was stirred at 220° C. and under nitrogen protection for 5 min. Then the reaction was cooled to room temperature. The reaction solution was poured into PE (1000 mL), stirred for 30 min, and filtered. And the filter cake was collected to afford compound Int-28D as a brown solid (2.90 g, 34% yield). MS (m/z)=181.2 [M+H]⁺

### 3) Synthesis of Compound Int-28E

KNO₃ (6.49 g, 64.23 mmol) was added to a solution of compound Int-28D (2.90 g, 16.02 mmol) in concentrated H₂SO₄ (9 mL). After the addition was completed, the reaction was placed at 100° C. and stirred for 4 h. After the reaction was ended, the reaction solution was poured into ice water, filtered, and the filter cake was collected and dried to afford compound Int-28E as a yellow solid (2.3 g, 61% yield). MS (m/z)=208.2 [M−17]⁺

### 4) Synthesis of Compound Int-28F

In an ice-water bath, to a solution of compound Int-28E (500 mg, 2.21 mmol) in DMF was slowly added POCl₃ (0.8 mL). After the addition was completed, the reaction solution was stirred at room temperature for 48 hours. After the reaction was ended, the reaction solution was concentrated to afford compound Int-28F as a yellow solid (540 mg, 99% yield). MS (m/z)=244.0, 246.0 [M+H]⁺

### 5) Synthesis of Compound Int-28G

In an ice-water bath, to a solution of compound Int-28F (540 mg, 2.21 mmol) in dichloromethane (20 mL) was added DIEA (863 mg, 6.63 mmol) and cyclopropylamine (252 mg, 4.42 mmol). After the addition was completed, the reaction solution was stirred at room temperature overnight. After the reaction was ended, the reaction solution was concentrated. The residue was washed with water (10 mL×2), and filtered. The solid was dried and collected to afford compound Int-28G as a yellow solid (580 mg, 99% yield). MS (m/z)=265.0 [M+H]⁺

### 6) Synthesis of Compound Int-28H

To a solution of compound Int-28G (580 mg, 2.19 mmol) in ethyl acetate (30 mL) was added stannous chloride (415 mg, 2.19 mmol). After the addition was completed, the reaction solution was stirred at room temperature for 3 h. After the reaction was ended, the reaction system was adjusted to pH=10-11 with 2 mol/L NaOH, and extracted with EA (30 mL×3). The organic phases were combined, dried over anhydrous sodium sulfate, and concentrated. The residue was purified by ISCO (mobile phase: MeOH:H₂O (TFA)=0:100-100:0) to afford compound Int-28H as a yellow solid (320 mg, 62% yield). MS (m/z)=235.2 [M+H]⁺

### 7) Synthesis of Compound Int-28I

To a solution of compound Int-28H (200 mg, 0.85 mmol) in toluene (20 mL) was added CDI (276 mg, 1.70 mmol). After the addition was completed, the reaction solution was stirred at 100° C. for 1 h. After the reaction was ended, the reaction system was concentrated, the residue was pulped with water (10 mL) and filtered. The solid was collected and dried to afford compound Int-28I as a yellow solid (190 mg, 85% yield). MS (m/z)=261.2 [M+H]⁺

### 8) Synthesis of Intermediate-28

In an ice-water bath and under nitrogen protection, to a solution of compound Int-28I (80 mg, 0.30 mmol) in anhydrous DMF (3 mL) was added NaH (25 mg, 0.61 mmol). After the addition was completed, the reaction solution was stirred at 0° C. for 15 min. CH₃I (65 mg, 0.46 mmol) was added to the reaction solution in one portion. After the addition was completed, the reaction solution was stirred at room temperature for 1 h. The reaction solution was poured into water, and extracted with EA (30 mL×3). The organic phases were combined, dried over anhydrous sodium sulfate, and concentrated. The residue was purified by ISCO (mobile phase: MeOH:H₂O (TFA)=0:100-100:0) to afford intermediate-28 as a yellow solid (70 mg, 83% yield). MS (m/z)=275.2 [M+H]⁺

### Preparation of Intermediate-31

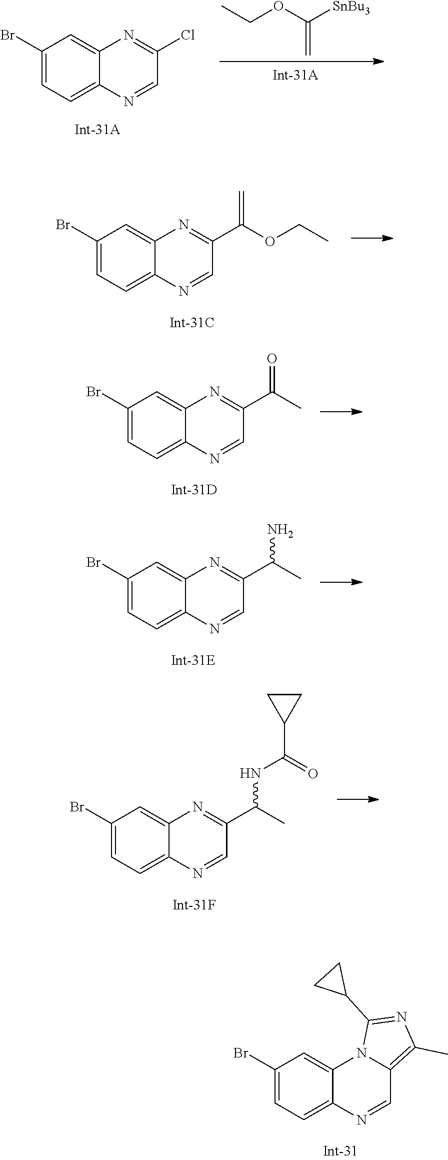

### 1) Synthesis of Compound Int-31C

Compound Int-31A (5.84 g, 24 mmol), Int-31B tributyl (1-ethoxyethylene)tin (10.83 g, 30 mmol), and PdCl₂(PPh₃)₂ (1.68 g, 2.4 mmol) were dissolved in toluene (60 mL). The reaction was stirred at 80° C. for 3 h. After the reaction was completed, the reaction solution was concentrated under reduced pressure and the residue was purified by ISCO (mobile phase: (MeOH:H₂O (TFA)=0:100-100:0) to afford compound Int-31C as a yellow solid (5.9 g, 88% yield). MS (m/z)=279.2 [M+H]⁺

### 2) Synthesis of Compound Int-31D

Compound Int-31C (5.9 g, 21.1 mmol) was dissolved in dioxane (60 mL) and 2 M (60 mL) hydrochloric acid. The reaction was stirred at room temperature for 1 h. After the reaction was completed, the reaction solution was concentrated under reduced pressure and the residue was purified by ISCO (mobile phase: (PE:EA=100:0-0:100) to afford compound Int-31D as a yellow solid (3.86 g, 72% yield). MS (m/z)=251.0 [M+H]⁺

### 3) Synthesis of Compound Int-31E

Compound Int-31D (3.86 g, 15.4 mmol), ammonium acetate (11.85 g, 153.7 mmol), and NaBH₃CN (0.97 g, 15.37 mmol) were dissolved in methanol (60 mL). The reaction was stirred at room temperature overnight. After the reaction was completed, the reaction solution was concentrated under reduced pressure and the residue was purified by ISCO (mobile phase: (MeOH:H₂O (TFA)=0:100-100:0) to afford compound Int-31E as a yellow solid (2.06 g, 53% yield). MS (m/z)=252.2 [M+H]⁺

### 4) Synthesis of Compound Int-31F

Compound Int-31E (2.06 g, 8.17 mmol), cyclopropylcarboxylic acid (0.91 g, 10.62 mmol), DIEA (3.17 g, 24.5 mmol), and HATU (3.57 g, 9.39 mmol) were dissolved in DMF (25 mL). The reaction was stirred at room temperature for 1 hour. After the reaction was completed, the reaction solution was concentrated under reduced pressure and the residue was purified by ISCO (mobile phase: (MeOH:H₂O (TFA)=0:100-100:0) to afford intermediate Int-31F as a yellow solid (2.09 g, 80% yield). MS (m/z)=320.2 [M+H]⁺

### 5) Synthesis of Intermediate-31

Compound Int-31F (2.09 g, 6.52 mmol) was dissolved in phosphorus oxychloride (12 mL). The reaction was stirred at 80° C. for 3 h. After the reaction was completed, the reaction solution was concentrated under reduced pressure and the residue was purified by ISCO (mobile phase: (MeOH:H₂O (TFA)=0:100-100:0) to afford intermediate-31 as a yellow solid (1.6 g, 81% yield). MS (m/z)=302.0 [M+H]⁺

### Preparation of Intermediate-34

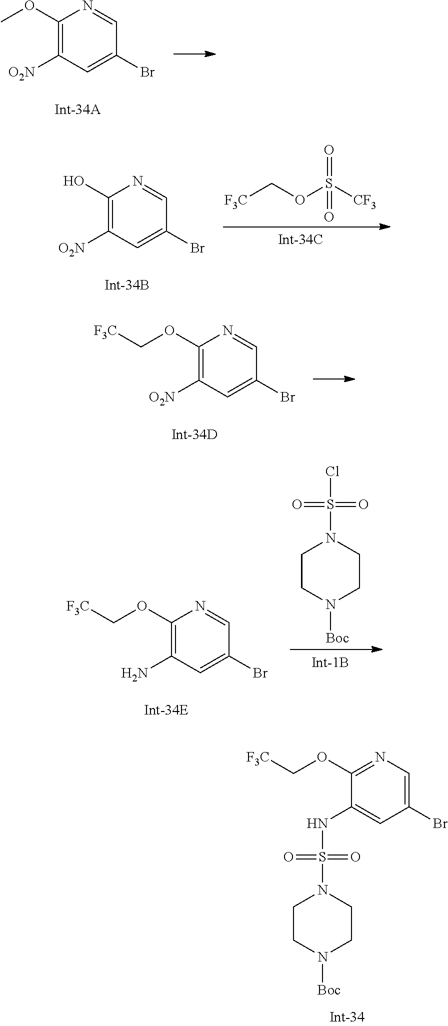

### 1) Synthesis of Compound Int-34B

Compound Int-34A (CAS: 152684-30-5) (5 g, 21.5 mmol) was dissolved in 1,4-dioxane (6 mL), and concentrated hydrochloric acid (3 mL) was slowly added thereto. The reaction was stirred at 100° C. overnight. After the reaction was completed, the reaction solution was cooled to room temperature, and filtered. The filter cake was washed with water (10 mL×3), and dried in vacuo to afford compound Int-34B as a yellow solid (4.5 g, 95.8% yield). MS (m/z)=219.0 [M+H]⁺

### 2) Synthesis of Compound Int-34D

Under nitrogen protection, compound Int-34B (2 g, 9.1 mmol) and potassium carbonate (2.52 g, 18.3 mmol) were dissolved in DMF (10 mL). Compound Int-34C (CAS: 6226-25-1) (2.54 g, 11.0 mmol) was slowly added thereto in an ice bath, and the reaction was stirred at room temperature overnight. After the reaction was completed, the reaction solution was poured into water (20 mL), and extracted with ethyl acetate (50 mL×3). The organic phases were combined and dried over anhydrous sodium sulfate, and concentrated under reduced pressure. The residue was purified by ISCO (petroleum ether:ethyl acetate=100:0-0:100 gradient elution) to afford compound Int-34D as a yellow solid (680 mg, 24.7% yield). MS (m/z)=303.0 [M+H]⁺

### 3) Synthesis of Compound Int-34E

Under nitrogen protection, stannous chloride dihydrate (1.53 g, 6.8 mmol) was added to a solution of compound Int-34D (680 mg, 2.3 mmol) in ethyl acetate (50 mL), and the reaction was stirred at 40° C. overnight. After the reaction was completed, 2 N NaOH solution was added to the reaction solution until the pH was adjusted to 9. The reaction solution was extracted with ethyl acetate (15 mL×3). The organic phases were combined, dried over anhydrous sodium sulfate, and concentrated under reduced pressure. The residue was purified by ISCO (petroleum ether:ethyl acetate=100:0-0:50 gradient elution) to afford compound Int-34E as a brown solid (220 mg, 35.9% yield). MS (m/z)=272.9 [M+H]⁺

### 4) Synthesis of Intermediate-34

Under nitrogen protection, compound Int-1B (367.7 mg, 1.3 mmol) was added to a solution of compound Int-34E (175 mg, 0.6 mmol) in pyridine (3 mL), and the reaction was stirred at 60° C. overnight. After the reaction was completed, the reaction solution was concentrated, and the residue was purified by ISCO (water (0.1% TFA):acetonitrile=100:0-0:100 gradient elution) to afford compound intermediate-34 as a gray solid (100 mg, 30.0% yield). MS (m/z)=419.0 [M−100]⁺

### Example 2: Preparation of Payloads

### Preparation of Compound-1:

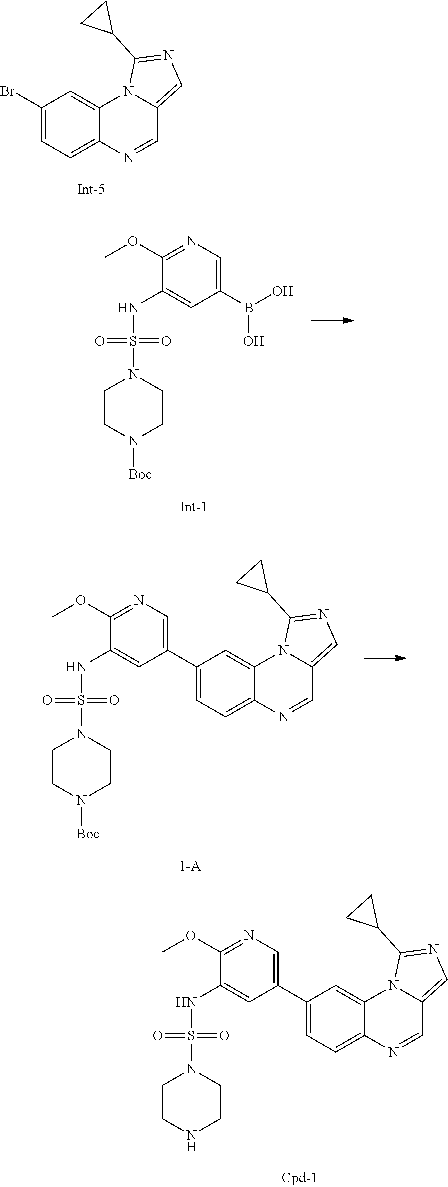 [SMILES: Brc1ccc2ncc3cnc(C4CC4)n3c2c1 ; COc1ncc(B(O)O)cc1NS(=O)(=O)N1CCN(C(=O)OC(C)(C)C)CC1 ; COc1ncc(-c2ccc3ncc4cnc(C5CC5)n4c3c2)cc1NS(=O)(=O)N1CCN(C(=O)OC(C)(C)C)CC1 ; COc1ncc(-c2ccc3ncc4cnc(C5CC5)n4c3c2)cc1NS(=O)(=O)N1CCNCC1]

### 1) Synthesis of Compound 1-A

Under nitrogen protection, to a solution of intermediate-5 (85 mg, 0.3 mmol) in DMF:H₂O (3:1, 4 mL), intermediate-1 (147 mg, 0.35 mmol) was added, Pd(dppf)Cl₂ (10.8 mg, 0.015 mmol), and potassium carbonate (163 mg, 1.2 mmol), and the reaction was stirred at 100° C. for 2 h. After the reaction was completed, the reaction solution was concentrated, and the residue was purified by ISCO (water (0.1% TFA):methanol=100:0-0:100 gradient elution) to afford compound 1-A as a white solid (100 mg, 58.5% yield). MS (m/z)=580.2 [M+H]⁺

### 2) Synthesis of Compound-1

Under nitrogen protection, compound 1-A (100 mg, 0.17 mmol) was added to a mixed solution of TFA:DCM (1:1, 5 mL), and the mixture was stirred for 20 minutes. After the reaction was completed, the reaction solution was concentrated, and the residue was purified by ISCO (water (0.1% TFA):acetonitrile=100:0-0:100 gradient elution) to afford compound-1 (abbreviated as Cpd-1) as a white solid (50 mg, 60.400 yield). MS (m/z)=479.8 [M+H]⁺

The following compounds were prepared according to the preparation procedure of compound 1 using corresponding intermediates and reagents under appropriate conditions recognized by a person skilled in the art.

| | Intermediates | | MS |
|---|---|---|---|
| Compound | used | Structural formula | (M + H)⁺ |
| Compound 2 | Intermediate-8; Intermediate-1; | 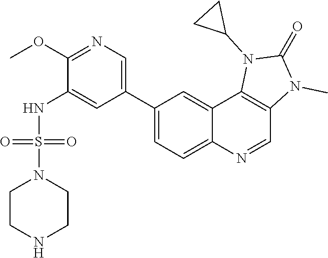 [SMILES: COc1ncc(-c2ccc3ncc4c(c3c2)n(C2CC2)c(=O)n4C)cc1NS(=O)(=O)N1CCNCC1] | 510.2 [M + H]⁺ |
| Compound 3 | Intermediate-2; Intermediate-1; | 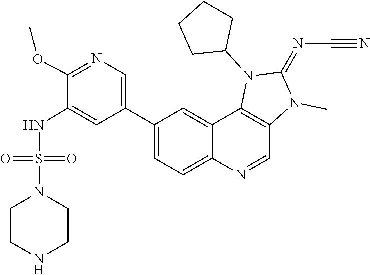 | 523.8 [M + H]⁺ |
| Compound 4 | Intermediate-3; Intermediate-1; | 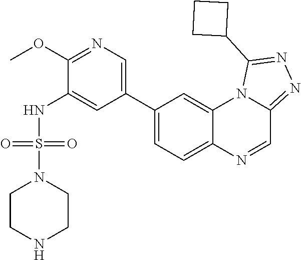 [SMILES: COc1ncc(-c2ccc3ncc4nnc(C5CCC5)n4c3c2)cc1NS(=O)(=O)N1CCNCC1] | 495.2 [M + H]⁺ |
| Compound 5 | Intermediate-9; Intermediate-1; | 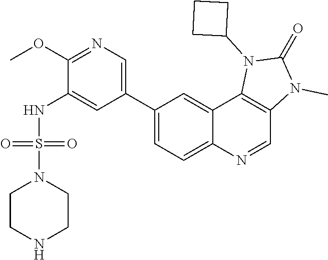 [SMILES: COc1ncc(-c2ccc3ncc4c(c3c2)n(C2CCC2)c(=O)n4C)cc1NS(=O)(=O)N1CCNCC1] | 524.2 [M + H]⁺ |
| Compound 6 | Intermediate-10; Intermediate-1; | 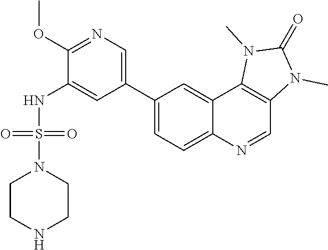 [SMILES: COc1ncc(-c2ccc3ncc4c(c3c2)n(C)c(=O)n4C)cc1NS(=O)(=O)N1CCNCC1] | 484.4 [M + H]⁺ |
| Compound 7 | Intermediate-11; Intermediate-1; | 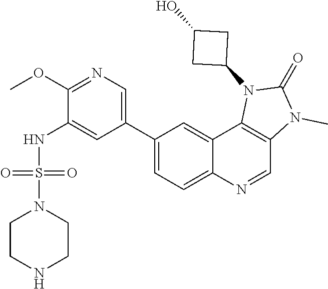 [SMILES: COc1ncc(-c2ccc3ncc4c(c3c2)n([C@H]2C[C@H](O)C2)c(=O)n4C)cc1NS(=O)(=O)N1CCNCC1] | 540.0 [M + H]⁺ |
| Compound 8 | Intermediate-12; Intermediate-1; | 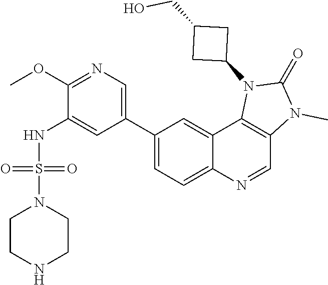 [SMILES: COc1ncc(-c2ccc3ncc4c(c3c2)n([C@H]2C[C@H](CO)C2)c(=O)n4C)cc1NS(=O)(=O)N1CCNCC1] | 554.8 [M + H]⁺ |
| Compound 9 | Intermediate-4; Intermediate-1; | 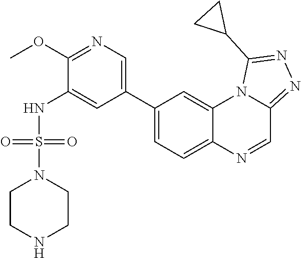 | 418.2 [M + H]⁺ |
| Compound 10 | Intermediate-8; Intermediate-21; | 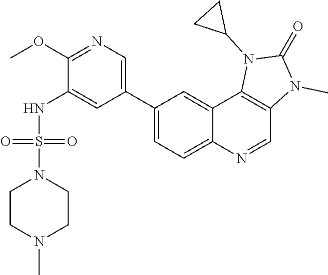 [SMILES: COc1ncc(-c2ccc3ncc4c(c3c2)n(C2CC2)c(=O)n4C)cc1NS(=O)(=O)N1CCN(C)CC1] | 524.4 [M + H]⁺ |
| Compound 11 | Intermediate-18; Intermediate-21; | 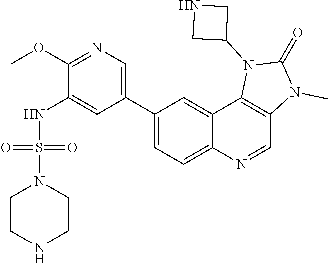 | 538.8 [M + H]⁺ |
| Compound 12 | Intermediate-8; Intermediate-23; | 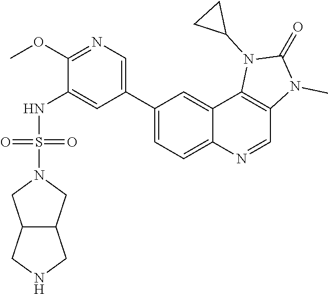 [SMILES: COc1ncc(-c2ccc3ncc4c(c3c2)n(C2CC2)c(=O)n4C)cc1NS(=O)(=O)N1CC2CNCC2C1] | 535.8 [M + H]⁺ |
| Compound 13 | Intermediate-16; Intermediate-1; | 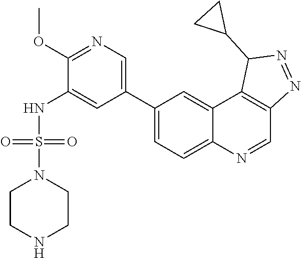 [SMILES: COc1ncc(-c2ccc3ncc4c(c3c2)C(C2CC2)N=N4)cc1NS(=O)(=O)N1CCNCC1] | 480.2 [M + H]⁺ |
| Compound 14 | Intermediate-8; Intermediate-19; | 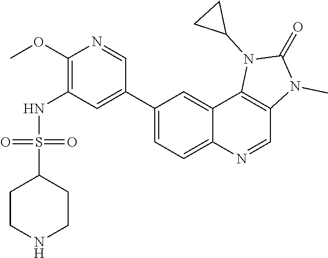 [SMILES: COc1ncc(-c2ccc3ncc4c(c3c2)n(C2CC2)c(=O)n4C)cc1NS(=O)(=O)C1CCNCC1] | 509.0 [M + H]⁺ |
| Compound 15 | Intermediate-8; Intermediate-20; | 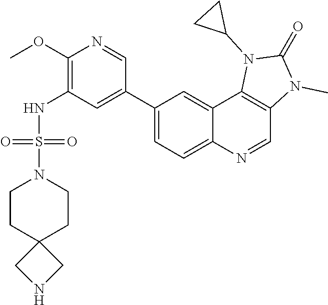 [SMILES: COc1ncc(-c2ccc3ncc4c(c3c2)n(C2CC2)c(=O)n4C)cc1NS(=O)(=O)N1CCC2(CC1)CNC2] | 549.8 [M + H]⁺ |
| Compound 16 | Intermediate-13; Intermediate-1; | 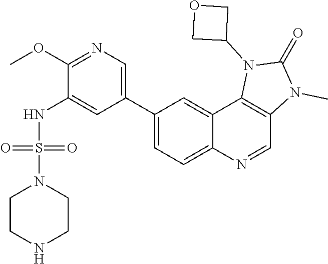 [SMILES: COc1ncc(-c2ccc3ncc4c(c3c2)n(C2COC2)c(=O)n4C)cc1NS(=O)(=O)N1CCNCC1] | 526.4 [M + H]⁺ |
| Compound 17 | Intermediate-15; Intermediate-1; | 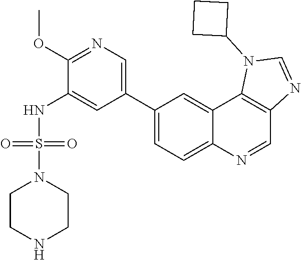 [SMILES: COc1ncc(-c2ccc3ncc4ncn(C5CCC5)c4c3c2)cc1NS(=O)(=O)N1CCNCC1] | 494.2 [M + H]⁺ |
| Compound 18 | Intermediate-6; Intermediate-1; | 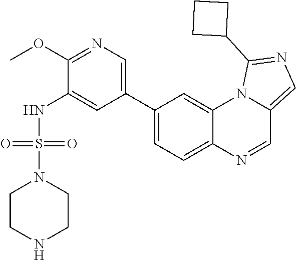 [SMILES: COc1ncc(-c2ccc3ncc4cnc(C5CCC5)n4c3c2)cc1NS(=O)(=O)N1CCNCC1] | 493.8 [M + H]⁺ |
| Compound 19 | Intermediate-14; Intermediate-1; | 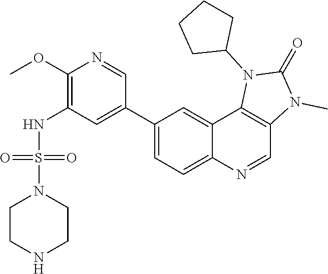 [SMILES: COc1ncc(-c2ccc3ncc4c(c3c2)n(C2CCCC2)c(=O)n4C)cc1NS(=O)(=O)N1CCNCC1] | 538.4 [M + H]⁺ |
| Compound 20 | Intermediate-7; Intermediate-1; | 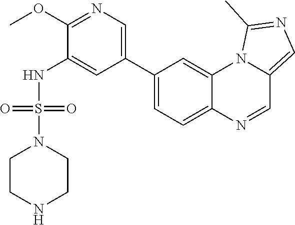 [SMILES: COc1ncc(-c2ccc3ncc4cnc(C)n4c3c2)cc1NS(=O)(=O)N1CCNCC1] | 453.8 [M + H]⁺ |
| Compound 21 | Intermediate-22; Intermediate-1; | 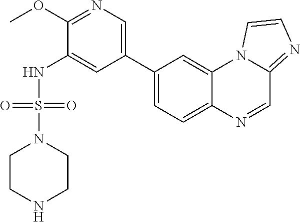 [SMILES: COc1ncc(-c2ccc3ncc4nccn4c3c2)cc1NS(=O)(=O)N1CCNCC1] | 440.2 [M + H]⁺ |
| Compound 24 | Intermediate-24; Intermediate-1; | 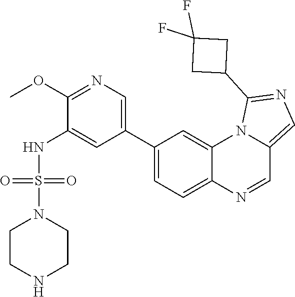 [SMILES: COc1ncc(-c2ccc3ncc4cnc(C5CC(F)(F)C5)n4c3c2)cc1NS(=O)(=O)N1CCNCC1] | 529.8 [M + H]⁺ |
| Compound 25 | Intermediate-25; Intermediate-1; | 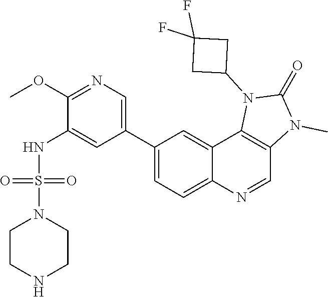 [SMILES: COc1ncc(-c2ccc3ncc4c(c3c2)n(C2CC(F)(F)C2)c(=O)n4C)cc1NS(=O)(=O)N1CCNCC1] | 560.2 [M + H]⁺ |
| Compound 27 | Intermediate-27; Intermediate-1; | 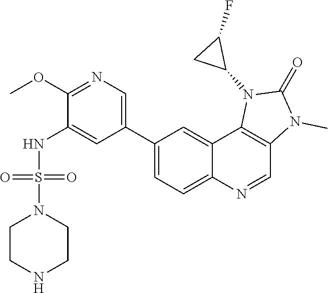 [SMILES: COc1ncc(-c2ccc3ncc4c(c3c2)n([C@@H]2C[C@@H]2F)c(=O)n4C)cc1NS(=O)(=O)N1CCNCC1] | 528.2 [M + H]⁺ |
| Compound 28 | Intermediate-28; Intermediate-1; | 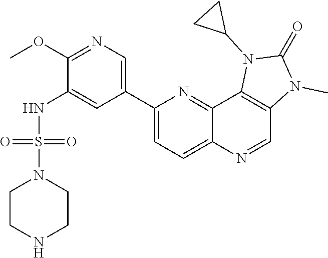 [SMILES: COc1ncc(-c2ccc3ncc4c(c3n2)n(C2CC2)c(=O)n4C)cc1NS(=O)(=O)N1CCNCC1] | 511.4 [M + H]⁺ |
| Compound 29 | Intermediate-29; Intermediate-1; | 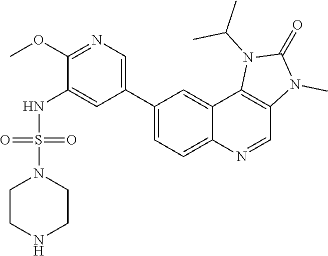 [SMILES: COc1ncc(-c2ccc3ncc4c(c3c2)n(C(C)C)c(=O)n4C)cc1NS(=O)(=O)N1CCNCC1] | 512.2 [M + H]⁺ |
| Compound 30 | Intermediate-30; Intermediate-1; | 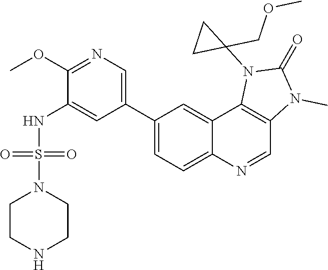 [SMILES: COCC1(n2c(=O)n(C)c3cnc4ccc(-c5cnc(OC)c(NS(=O)(=O)N6CCNCC6)c5)cc4c32)CC1] | 554.2 [M + H]⁺ |
| Compound 31 | Intermediate-31; Intermediate-1; | 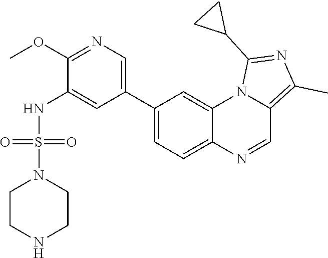 [SMILES: COc1ncc(-c2ccc3ncc4c(C)nc(C5CC5)n4c3c2)cc1NS(=O)(=O)N1CCNCC1] | 494.2 [M + H]⁺ |
| Compound 32 | Intermediate-32; Intermediate-1; | 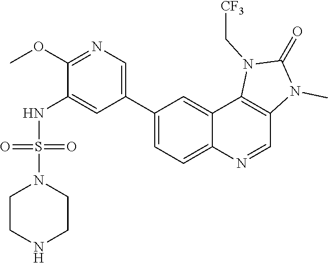 [SMILES: COc1ncc(-c2ccc3ncc4c(c3c2)n(CC(F)(F)F)c(=O)n4C)cc1NS(=O)(=O)N1CCNCC1] | 552.2 [M + H]⁺ |
| Compound 33 | Intermediate-33; Intermediate-1; | 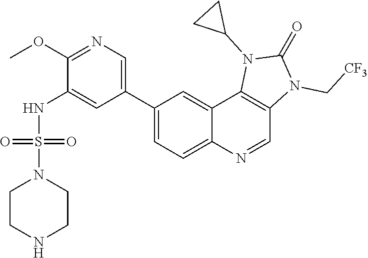 [SMILES: COc1ncc(-c2ccc3ncc4c(c3c2)n(C2CC2)c(=O)n4CC(F)(F)F)cc1NS(=O)(=O)N1CCNCC1] | 578.1 [M + H]⁺ |
| Compound 34 | Intermediate-34; Intermediate-26; | 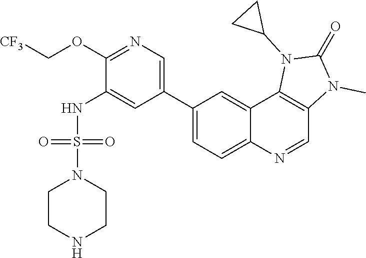 [SMILES: Cn1c(=O)n(C2CC2)c2c3cc(-c4cnc(OCC(F)(F)F)c(NS(=O)(=O)N5CCNCC5)c4)ccc3ncc21] | 578.2 [M + H]⁺ |
| Compound 35 | Intermediate-35; Intermediate-1; | 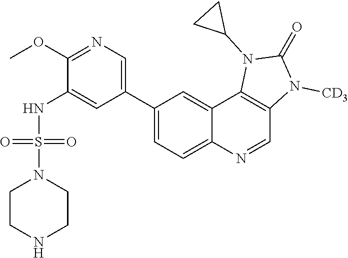 [SMILES: [2H]C([2H])([2H])n1c(=O)n(C2CC2)c2c3cc(-c4cnc(OC)c(NS(=O)(=O)N5CCNCC5)c4)ccc3ncc21] | 513.2 [M + H]⁺ |

### Preparation of Compound 22:

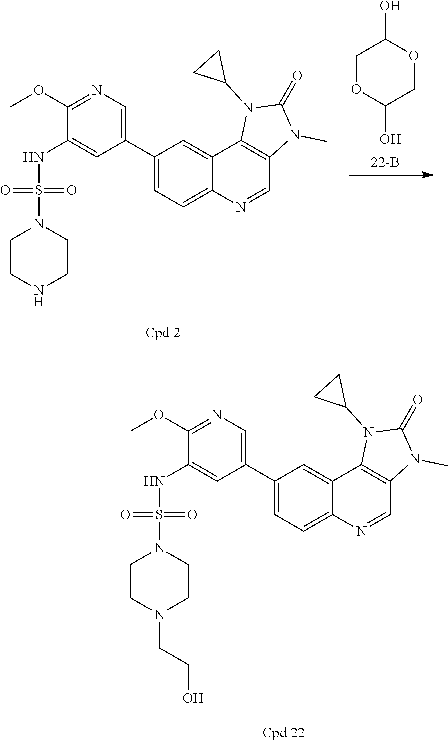 [SMILES: COc1ncc(-c2ccc3ncc4c(c3c2)n(C2CC2)c(=O)n4C)cc1NS(=O)(=O)N1CCNCC1 ; OC1COC(O)CO1 ; COc1ncc(-c2ccc3ncc4c(c3c2)n(C2CC2)c(=O)n4C)cc1NS(=O)(=O)N1CCN(CCO)CC1]

Under nitrogen protection, compound 2 (100 mg, 0.196 mmol), compound 22-B (CAS: 23147-58-2) (58 mg, 0.392 mmol), and sodium cyanoborohydride (62 mg, 0.98 mmol) were dissolved in methanol (8 mL). The reaction solution was stirred at room temperature for 18 hours. After the reaction was ended, the reaction solution was concentrated under reduced pressure, and the residue was purified by ISCO (mobile phase: water (0.1% trifluoroacetic acid):acetonitrile=100:0-0:100) to afford compound 22 as a yellow solid (32 mg, 29% yield). MS (m/z)=554.0 [M+1]⁺

### Preparation of Compound 23:

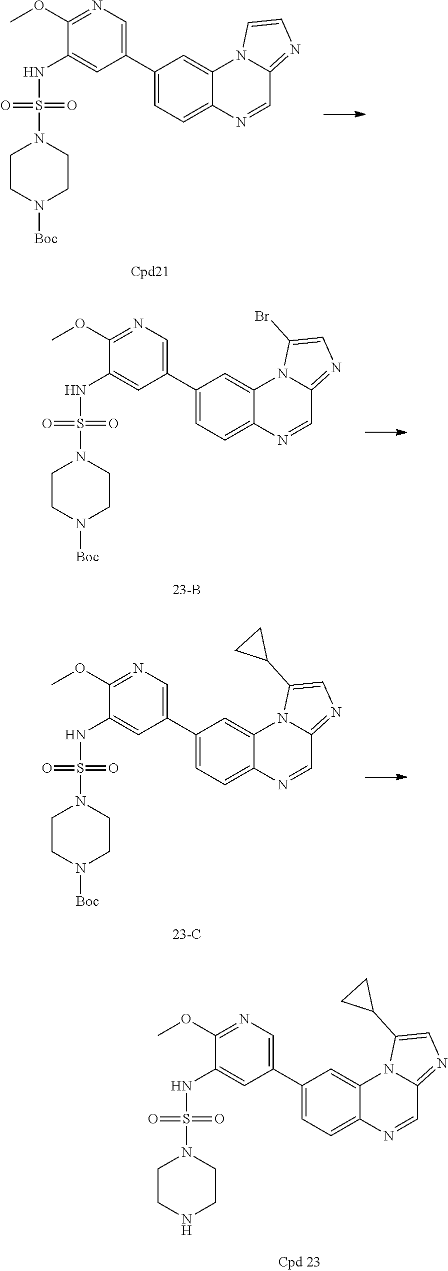 [SMILES: COc1ncc(-c2ccc3ncc4nccn4c3c2)cc1NS(=O)(=O)N1CCN(C(=O)OC(C)(C)C)CC1 ; COc1ncc(-c2ccc3ncc4ncc(Br)n4c3c2)cc1NS(=O)(=O)N1CCN(C(=O)OC(C)(C)C)CC1 ; COc1ncc(-c2ccc3ncc4ncc(C5CC5)n4c3c2)cc1NS(=O)(=O)N1CCN(C(=O)OC(C)(C)C)CC1 ; COc1ncc(-c2ccc3ncc4ncc(C5CC5)n4c3c2)cc1NS(=O)(=O)N1CCNCC1]

### 1) Synthesis of Compound 23-B

Under nitrogen protection, compound 21 (100 mg, 0.185 mmol) and N-bromosuccinimide (36 mg, 0.2 mmol) were added to chloroform. A drop of TFA was added, and the reaction was stirred at room temperature for 12 h. After the reaction was completed, the reaction solution was concentrated, and the residue was purified by ISCO (mobile phase: CH₃CN:H₂O (TFA)=0:100-100:0) to afford compound 23-B as a yellow solid (60 mg, 52% yield). MS (m/z)=617.6 [M+H]⁺

### 2) Synthesis of Compound 23-C

Under nitrogen protection, compound 23-B (50 mg, 0.08 mmol), cyclopropylboronic acid (14 mg, 0.16 mmol), palladium acetate (4 mg, 0.016 mmol), potassium phosphate (51 mg, 0.24 mmol), and tricyclohexylphosphine (5 mg, 0.016 mmol) were added to a mixed solution of toluene and water (5:1) at room temperature sequentially, and the reaction was stirred at 90° C. for 3 h. After the reaction was completed, the reaction solution was concentrated, and the residue was purified by ISCO (mobile phase: CH₃CN:H₂O (TFA)=0:100-100:0) to afford compound 23-C as a yellow solid (20 mg, 43% yield). MS (m/z)=579.8 [M+H]⁺

### 3) Synthesis of Compound 23

Under nitrogen protection, compound 23-C (20 mg, 0.035 mmol) was dissolved into a mixed solution of TFA:DCM (1:1, 5 mL), and the mixture was stirred for 20 minutes. After the reaction was completed, the reaction solution was concentrated, and the residue was purified by ISCO (water (0.1% TFA):acetonitrile=100:0-0:100 gradient elution) to afford compound 23 as a white solid (10 mg, 60.4% yield). MS (m/z)=479.8 [M+H]⁺

### Preparation of Compound 26:

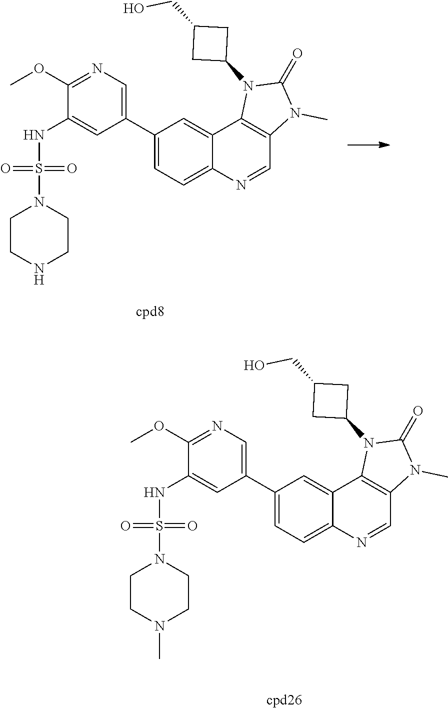 [SMILES: COc1ncc(-c2ccc3ncc4c(c3c2)n([C@H]2C[C@H](CO)C2)c(=O)n4C)cc1NS(=O)(=O)N1CCNCC1 ; COc1ncc(-c2ccc3ncc4c(c3c2)n([C@H]2C[C@H](CO)C2)c(=O)n4C)cc1NS(=O)(=O)N1CCN(C)CC1]

Compound 8 (30 mg, 0.054 mmol), NaBH₃CN (20 mg, 0.325 mmol), and 37% aqueous formaldehyde solution (0.5 mL) were dissolved in methanol (4 mL). The reaction was carried out at room temperature for 3 h. After the reaction was completed, the reaction solution was concentrated under reduced pressure and the residue was purified by ISCO (mobile phase: (MeOH:H₂O (TFA)=0:100-100:0) to afford compound 26 as a light-yellow solid (12 mg, 39% yield). MS (m/z)=568.2 [M+H]⁺

The ¹H NMR data of the compounds prepared in Example 2 are shown in the following table:

| Compound No. | ¹H NMR |
|---|---|
| Compound 1 | ¹H NMR (400 MHz, DMSO) δ 8.93 (d, J = 1.9 Hz, 1H), 8.90 (s, |
| | 1H), 8.09 (s, 1H), 8.00-7.93 (m, 2H), 7.81 (dd, J = 8.5, 1.9 Hz, |
| | 1H), 7.74 (s, 1H), 3.88 (s, 3H), 2.94-2.89 (m, 4H), 2.70-2.60 (m, |
| | 5H), 1.35-1.30 (m, 2H), 1.16-1.12 (m, 2H). |
| Compound 2 | ¹H NMR (400 MHz, DMSO) δ 8.89 (s, 1H), 8.83 (s, 1H), 8.39-8.32 |
| | (m, 1H), 8.11-8.05 (m, 2H), 7.93-7.86 (m, 1H), 3.95 (s, 3H), 3.50- |
| | 3.49 (m, 1H), 3.45 (s, 3H), 3.02-2.98 (s, 4H), 2.67-2.63 (s, 4H), |
| | 1.36-1.31 (m, 2H), 1.22-1.18 (m, 2H). |
| Compound 3 | ¹H NMR (400 MHz, DMSO) δ 8.84 (s, 1H), 8.35 (d, J = 2.2 Hz, |
| | 1H), 8.34 (d, J = 1.7 Hz, 1H), 8.08 (d, J = 8.9 Hz, 1H), 8.05 (d, J = |
| | 2.3 Hz, 1H), 7.85 (dd, J = 8.9, 1.8 Hz, 1H), 5.44-5.33 (m, 1H), |
| | 3.95 (s, 3H), 3.46 (s, 3H), 3.10-3.04 (m, 2H), 3.03-2.99 (m, 4H), |
| | 2.73-2.61 (m, 4H), 2.45-2.38 (m, 2H), 1.87 (dt, J = 19.3, 8.6 Hz, |
| | 2H). |
| Compound 4 | ¹H NMR (400 MHz, DMSO) δ 9.29 (s, 1H), 8.39 (d, J = 2.3 Hz, |
| | 1H), 8.19-8.10 (m, 2H), 8.07 (d, J = 2.2 Hz, 1H), 7.96 (d, J = 8.4 |
| | Hz, 1H), 4.51 (t, J = 7.8 Hz, 1H), 3.98 (s, 3H), 3.06 (s, 4H), 2.74 (s, |
| | 4H), 2.68-2.61 (m, 4H), 2.22-2.14 (m, 1H), 2.05-1.96 (m, 1H). |
| Compound 5 | ¹H NMR (400 MHz, DMSO) δ 8.84 (s, 1H), 8.40-8.32 (m, 2H), |
| | 8.12-8.04 (m, 2H), 7.88-7.82 (m, 1H), 5.46-5.35 (m, 1H), 3.97 (s, |
| | 3H), 3.47 (s, 3H), 3.12-3.03 (m, 4H), 3.02-2.97 (m, 4H), 2.68-2.61 |
| | (m, 4H), 1.93-1.82 (m, 2H). |
| Compound 6 | ¹H NMR (400 MHz, DMSO) δ 8.85 (s, 1H), 8.41 (d, J = 1.8 Hz, |
| | 1H), 8.35 (d, J = 2.2 Hz, 1H), 8.09 (d, J = 8.9 Hz, 1H), 8.01 (d, J = |
| | 2.3 Hz, 1H), 7.83 (dd, J = 8.9, 1.9 Hz, 1H), 3.94 (s, 3H), 3.89 (s, |
| | 3H), 3.51 (s, 3H), 3.03-2.98 (m, 4H), 2.68-2.64 (m, 4H). |
| Compound 7 | ¹H NMR (400 MHz, DMSO) δ 8.85 (s, 1H), 8.37 (d, J = 2.2 Hz, |
| | 1H), 8.24 (d, J = 1.6 Hz, 1H), 8.09 (d, J = 8.8 Hz, 1H), 8.00 (d, J = |
| | 2.2 Hz, 1H), 7.81 (dd, J = 8.8, 1.6 Hz, 1H), 5.54-5.46 (m, 1H), |
| | 4.51-4.44 (m, 1H), 3.97 (s, 3H), 3.47 (s, 3H), 3.23-3.19 (m, 2H), |
| | 3.09-3.04 (m, 4H), 2.75-2.71 (m, 4H), 2.44-2.38 (m, 2H). |
| Compound 8 | ¹H NMR (400 MHz, DMSO) δ 8.85 (s, 1H), 8.35-8.29 (m, 2H), |
| | 8.10 (d, J = 8.8 Hz, 1H), 7.99 (d, J = 1.8 Hz, 1H), 7.81 (dd, J = 8.8, |
| | 1.8 Hz, 1H), 5.60-5.56 (m, 1H), 3.95 (s, 3H), 3.58 (m, 2H), 3.48 (s, |
| | 3H), 3.14-3.07 (m, 3H), 3.02-2.98 (m, 4H), 2.67-2.64 (m, 4H), |
| | 2.35-2.27 (m, 2H). |
| Compound 9 | ¹H NMR (400 MHz, DMSO) δ 9.28 (s, 1H), 8.90 (s, 1H), 8.39 (s, |
| | 1H), 8.18 (d, J = 8.4 Hz, 1H), 8.08 (d, J = 1.8 Hz, 1H), 8.01 (d, J = |
| | 8.4 Hz, 1H), 3.96 (s, 3H), 3.02 (s, 4H), 2.81 (s, 1H), 2.71 (s, 4H), |
| | 1.35 (d, J = 7.9 Hz, 1H), 1.22 (d, J = 6.0 Hz, 4H). |
| Compound 10 | ¹H NMR (400 MHz, CD₃OD) δ 8.89-8.83 (m, 1H), 8.70 (s, 1H), |
| | 8.31-8.29 (m, 1H), 8.18-8.13 (m, 1H), 8.07-8.01 (m, 1H), 7.89-7.83 |
| | (m, 1H), 4.08 (s, 3H), 3.53 (s, 3H), 3.41-3.34 (m, 1H), 3.32-3.30 |
| | (m, 4H), 2.69-2.61 (m, 4H), 2.40 (s, 3H), 1.48-1.42 (m, 2H), 1.19- |
| | 1.14 (m, 2H). |
| Compound 11 | ¹H NMR (400 MHz, DMSO) δ 8.89 (s, 1H), 8.63 (s, 1H), 8.45-8.39 |
| | (m, 1H), 8.14-8.09 (m, 1H), 8.07-8.04 (m, 1H), 7.87-7.83 (m, 1H), |
| | 5.90-5.79 (m, 1H), 4.38-4.31 (m, 2H), 4.10-4.04 (m, 2H), 3.94 (s, |
| | 3H), 3.50 (s, 3H), 3.08-3.04 (m, 4H), 2.28-2.23 (m, 4H), 2.10 (s, |
| | 3H). |
| Compound 12 | ¹H NMR (400 MHz, DMSO) δ 8.90-8.84 (m, 1H), 8.80 (s, 1H), |
| | 8.08-8.02 (m, 2H), 7.96-7.92 (m, 1H), 7.88-7.83 (m, 1H), 3.89 (s, |
| | 3H), 3.45 (s, 3H), 3.43-3.39 (m, 1H), 3.34-3.26 (m, 4H), 3.05- |
| | 3.00 (m, 2H), 2.97-2.91 (m, 2H), 2.87-2.80 (m, 2H), 1.43-1.35 (m, |
| | 2H), 1.15-1.07 (m, 2H). |
| Compound 13 | ¹H NMR (400 MHz, DMSO) δ 9.15 (s, 1H), 8.96 (d, J = 2.1 Hz, |
| | 1H), 8.43 (s, 1H), 8.23-8.16 (m, 2H), 8.07 (d, J = 2.3 Hz, 1H), |
| | 8.00-7.95 (m, 1H), 4.10-4.04 (m, 1H), 3.89 (s, 3H), 2.93 (t, J = |
| | 4.8 Hz, 4H), 2.62 (t, J = 4.8 Hz, 4H), 1.47-1.41 (m, 2H), 1.33- |
| | 1.28 (m, 2H). |
| Compound 14 | ¹H NMR (400 MHz, DMSO) δ 8.89 (d, J = 1.9 Hz, 1H), 8.82 (s, |
| | 1H), 8.27 (d, J = 2.0 Hz, 1H), 8.07 (d, J = 8.9 Hz, 1H), 8.05 (d, J = |
| | 2.3 Hz, 1H), 7.91 (dd, J = 8.9, 2.0 Hz, 1H), 3.91 (s, 3H), 3.52- |
| | 3.49 (m, 1H), 3.45 (s, 3H), 3.07 (dd, J = 14.9, 11.9 Hz, 3H), 2.51 |
| | (d, J = 12.4 Hz, 2H), 1.97 (d, J = 11.0 Hz, 2H), 1.56 (dd, J = 12.4, |
| | 3.8 Hz, 2H), 1.35 (d, J = 5.4 Hz, 2H), 1.13 (dd, J = 6.4, 4.1 Hz, |
| | 2H). |
| Compound 15 | ¹H NMR (400 MHz, DMSO) δ 8.87 (d, J = 1.8 Hz, 1H), 8.81 (s, |
| | 1H), 8.38 (s, 1H), 8.05 (d, J = 8.9 Hz, 2H), 7.98 (d, J = 2.2 Hz, |
| | 1H), 7.86 (dd, J = 8.9, 1.8 Hz, 1H), 3.86 (s, 3H), 3.54 (s, 4H), 3.45 |
| | (s, 3H), 2.97-2.92 (m, 4H), 1.74-1.63 (m, 4H), 1.38 (q, J = 6.9 |
| | Hz, 2H), 1.13 (m, 2H). |
| Compound 16 | ¹H NMR (400 MHz, DMSO) δ 8.78 (s, 1H), 8.39 (d, J = 2.3 Hz, |
| | 1H), 8.05 (d, J = 2.0 Hz, 1H), 8.01-7.96 (m, 2H), 7.82-7.78 (m, |
| | 1H), 4.81-4.74 (m, 1H), 4.42-4.38 (m, 1H), 4.00 (s, 4H), 3.63- |
| | 3.60 (m, 1H), 3.39-3.27 (m, 5H), 3.20 (s, 3H), 3.17-3.11 (m, |
| | 4H). |
| Compound 17 | ¹H NMR (400 MHz, DMSO) δ 9.45 (s, 1H), 8.82 (s, 1H), 8.51 (dd, |
| | J = 8.3, 2.1 Hz, 2H), 8.31 (d, J = 8.8 Hz, 1H), 8.19-8.09 (m, 2H), |
| | 5.62 (s, 1H), 4.00 (s, 3H), 3.33-3.28 (m, 4H), 3.14-3.04 (m, 4H), |
| | 2.74-2.59 (m, 4H), 2.07-1.90 (m, 2H). |
| Compound 18 | ¹H NMR (400 MHz, DMSO) δ 8.95 (s, 1H), 8.42 (d, J = 2.2 Hz, |
| | 1H), 8.12 (d, J = 1.9 Hz, 1H), 8.06 (d, J = 2.3 Hz, 1H), 7.96 (d, J = |
| | 8.3 Hz, 1H), 7.89-7.81 (m, 2H), 4.47-4.37 (m, 1H), 3.99 (s, 3H), |
| | 3.19-3.15 (m, 4H), 2.95-2.90 (m, 4H), 2.63-2.53 (m, 4H), 2.19- |
| | 2.08 (m, 1H), 2.01-1.90 (m, 1H). |
| Compound 19 | ¹H NMR (400 MHz, DMSO) δ 8.84 (s, 1H), 8.32 (s, 1H), 8.23 (s, |
| | 1H), 8.09 (d, J = 8.8 Hz, 1H), 7.98 (d, J = 2.1 Hz, 1H), 7.83 (d, J = |
| | 8.7 Hz, 1H), 5.44-5.31 (m, 1H), 3.92 (s, 3H), 3.47 (s, 3H), 2.97 |
| | (m, 4H), 2.64 (m, 4H), 2.30 (m, 2H), 2.12-2.02 (m, 2H), 1.93 (m, |
| | 2H), 1.77-1.66 (m, 2H). |
| Compound 20 | ¹H NMR (400 MHz, DMSO) δ 8.92 (s, 1H), 8.39 (d, J = 2.3 Hz, |
| | 1H), 8.35 (d, J = 1.9 Hz, 1H), 8.01 (d, J = 2.3 Hz, 1H), 7.95 (d, J = |
| | 8.3 Hz, 1H), 7.80-7.75 (m, 2H), 3.95 (s, 3H), 3.10 (s, 3H), 3.06 (t, |
| | J = 4.9 Hz, 4H), 2.75 (t, J = 4.9 Hz, 4H). |
| Compound 21 | ¹H NMR (400 MHz, DMSO) δ 9.09 (s, 1H), 8.96 (s, 1H), 8.68 (d, |
| | J = 1.9 Hz, 1H), 8.46 (d, J = 2.3 Hz, 1H), 8.14-8.07 (m, 2H), 7.88- |
| | 7.83 (m, 2H), 3.97 (s, 3H), 2.99 (t, J = 4.9 Hz, 4H), 2.66 (t, J = 4.9 |
| | Hz, 4H). |
| Compound 22 | ¹H NMR (400 MHz, DMSO) δ 9.94 (s, 1H), 9.17 (s, 1H), 9.11-9.01 |
| | (m, 1H), 8.61-8.51 (m, 1H), 8.31-8.19 (m, 2H), 8.17-8.09 (m, 1H), |
| | 4.01 (s, 3H), 3.80-3.60 (m, 5H), 3.58-3.41 (m, 5H), 3.28-2.91 (m, |
| | 6H), 1.42-1.32 (m, 2H), 1.21-1.17 (m, 2H). |
| Compound 23 | ¹H NMR (400 MHz, DMSO) δ 9.09 (d, J = 1.9 Hz, 1H), 8.99 (d, J = |
| | 0.8 Hz, 1H), 8.36 (d, J = 2.3 Hz, 1H), 8.12 (d, J = 8.5 Hz, 1H), |
| | 8.05 (d, J = 2.3 Hz, 1H), 7.93 (dd, J = 8.6, 1.9 Hz, 1H), 7.61 (s, |
| | 1H), 3.94 (s, 3H), 3.03-2.98 (m, 4H), 2.69-2.57 (m, 5H), 1.30- |
| | 1.24 (m, 2H), 1.01-0.94 (m, 2H). |
| Compound 24 | ¹H NMR (400 MHz, CD₃OD) δ 8.94 (d, J = 1.2 Hz, 1H), 8.40 (dd, J = |
| | 2.4, 1.1 Hz, 1H), 8.22-8.15 (m, 2H), 8.06-8.00 (m, 1H), 7.92 |
| | (d, J = 1.0 Hz, 1H), 7.85 (dd, J = 8.3, 1.7 Hz, 1H), 4.42-4.31 (m, |
| | 1H), 4.12 (s, 3H), 3.53-3.47 (m, 4H), 3.37-3.31 (m, 4H), 3.27- |
| | 3.23 (m, 4H). |
| Compound 25 | ¹H NMR (400 MHz, CD₃OD) δ 8.81 (s, 1H), 8.43-8.37 (m, 1H), |
| | 8.35-8.30 (m, 1H), 8.21-8.18 (m, 1H), 8.17-8.13 (m, 1H), 7.90 (m, |
| | 1H), 5.51-5.39 (m, 1H), 4.12 (s, 3H), 3.98-3.86 (m, 2H), 3.58 (s, |
| | 3H), 3.52-3.48 (m, 4H), 3.26-3.23 (m, 4H), 3.21-3.12 (m, 2H). |
| Compound 26 | ¹H NMR (400 MHz, CD₃OD) δ 8.62 (s, 1H), 8.22-8.18 (m, 1H), |
| | 8.13-8.10 (m, 1H), 8.04-8.01 (m, 1H), 7.97-7.91 (m, 1H), 7.71-7.66 |
| | (m, 1H), 5.47-5.36 (m, 1H), 4.09 (s, 3H), 3.79-3.73 (m, 2H), 3.51 |
| | (s, 3H), 3.28-3.17 (m, 6H), 2.66-2.56 (m, 1H), 2.46-2.37 (m, |
| | 6H), 2.23 (s, 3H). |
| Compound 27 | ¹H NMR (400 MHz, DMSO) δ 8.87 (s, 1H), 8.78 (s, 1H), 8.37 (d, J = |
| | 2.2 Hz, 1H), 8.11 (d, J = 8.9 Hz, 1H), 8.07 (d, J = 2.3 Hz, 1H), |
| | 7.90 (dd, J = 8.9, 2.0 Hz, 1H), 5.42 (s, 1H), 5.24 (s, 1H), 3.96 (s, |
| | 3H), 3.67-3.62 (m, 1H), 3.51 (s, 3H), 3.04-2.98 (m, 4H), 2.69- |
| | 2.63 (m, 4H), 1.93-1.73 (m, 2H). |
| Compound 28 | ¹H NMR (400 MHz, DMSO) δ 8.89 (s, 1H), 8.80 (d, J = 2.1 Hz, |
| | 1H), 8.64 (d, J = 2.2 Hz, 1H), 8.42 (d, J = 9.0 Hz, 1H), 8.24 (d, J = |
| | 9.0 Hz, 1H), 3.98 (s, 3H), 3.66-3.62 (m, 1H), 3.47 (s, 3H), 3.00- |
| | 2.95 (m, 4H), 2.66-2.60 (m, 4H), 1.30-1.25 (m, 2H), 1.14-1.09 |
| | (m, 2H). |
| Compound 29 | ¹H NMR (400 MHz, DMSO) δ 8.86 (s, 1H), 8.36 (dd, J = 5.4, 2.0 |
| | Hz, 2H), 8.12 (d, J = 8.8 Hz, 1H), 8.04 (d, J = 2.3 Hz, 1H), 7.86 |
| | (dd, J = 9.0, 1.8 Hz, 1H), 5.28 (dt, J = 13.8, 6.8 Hz, 1H), 3.96 (s, |
| | 3H), 3.48 (s, 3H), 3.03-3.00 (m, 4H), 2.67-2.64 (m, 4H), 1.65 |
| | (d, J = 6.8 Hz, 6H). |
| Compound 30 | ¹H NMR (400 MHz, DMSO) δ 10.18 (s, 1H), 8.99 (d, J = 2.0 Hz, |
| | 1H), 8.85 (s, 1H), 8.41 (d, J = 2.2 Hz, 1H), 8.16 (d, J = 2.4 Hz, 1H), |
| | 8.08 (d, J = 8.8 Hz, 1H), 7.93 (dd, J = 8.9, 2.0 Hz, 1H), 3.99 (s, |
| | 3H), 3.87 (d, J = 10.7 Hz, 1H), 3.53 (s, 1H), 3.49 (s, 3H), 3.18 (s, |
| | 3H), 3.04 (t, J = 5.1 Hz, 4H), 2.71 (d, J = 5.6 Hz, 4H), 1.49 (s, 1H), |
| | 1.35 (q, J = 7.1 Hz, 2H), 1.22 (s, 1H). |
| Compound 31 | ¹H NMR (400 MHz, DMSO) δ 8.89 (s, 1H), 8.86-8.82 (m, 1H), |
| | 8.36-8.32 (m, 1H), 8.06-8.01 (m, 1H), 7.93-7.90 (m, 1H), 7.81-7.76 |
| | (m, 1H), 3.96 (s, 3H), 3.02-2.98 (m, 4H), 2.69-2.65 (m, 5H), |
| | 2.47 (s, 3H), 1.28-1.23 (m, 2H), 1.15-1.10 (m, 2H). |
| Compound 32 | ¹H NMR (400 MHz, DMSO) δ 8.96 (s, 1H), 8.39-8.35 (m, 2H), |
| | 8.15 (d, J = 8.9 Hz, 1H), 8.05 (d, J = 2.3 Hz, 1H), 7.87 (dd, J = 8.9, |
| | 1.9 Hz, 1H), 5.39 (q, J = 9.0 Hz, 2H), 3.98 (s, 3H), 3.57 (s, 3H), |
| | 3.04-2.98 (m, 4H), 2.70-2.63 (m, 4H). |
| Compound 33 | ¹H NMR (400 MHz, DMSO) δ 8.97-8.92 (m, 2H), 8.39 (d, J = 2.3 |
| | Hz, 1H), 8.14-8.08 (m, 2H), 7.99-7.93 (m, 1H), 5.04-4.93 (m, |
| | 2H), 3.97 (s, 3H), 3.63-3.56 (m, 1H), 3.06-3.00 (m, 4H), 2.71- |
| | 2.67 (m, 4H), 1.41-1.31 (m, 2H), 1.19-1.15 (m, 2H). |
| Compound 34 | ¹H NMR (400 MHz, DMSO) δ 8.91 (d, J = 2.0 Hz, 1H), 8.84 (d, J = |
| | 0.8 Hz, 1H), 8.22 (s, 1H), 8.14 (d, J = 2.3 Hz, 1H), 8.11 (d, J = |
| | 2.3 Hz, 1H), 8.09 (d, J = 8.9 Hz, 1H), 7.90 (dd, J = 8.8, 2.0 Hz, |
| | 1H), 5.04-4.94 (m, 2H), 3.46 (s, 3H), 3.0-3.01 (m, 4H), 2.80-2.72 |
| | (m, 4H), 2.01-1.90 (m, 1H), 1.41-1.33 (m, 2H), 1.15-1.10 (m, 2H). |
| Compound 35 | ¹H NMR (400 MHz, DMSO) δ 8.89 (s, 1H), 8.83 (s, 1H), 8.36 (s, |
| | (m, 1H), 3.04-2.98 (m, 4H), 2.69-2.63 (m, 4H), 1.37-1.30 (m, 2H), |
| | 1H), 8.11-8.05 (m, 2H), 7.93-7.87 (m, 1H), 3.96 (s, 3H), 3.51-3.47 |
| | 1.16-1.09 (m, 2H). |

The following reference compounds were also synthesized in the present invention according to the method in WO 2012034526 A1:

| No. | Structure |
|---|---|
| Reference compound-1 | 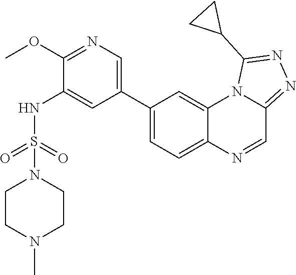 [SMILES: COc1ncc(-c2ccc3ncc4nnc(C5CC5)n4c3c2)cc1NS(=O)(=O)N1CCN(C)CC1] |

### Example 3: Preparation of Linker-Payloads (LPs)

### Preparation of LP-1:

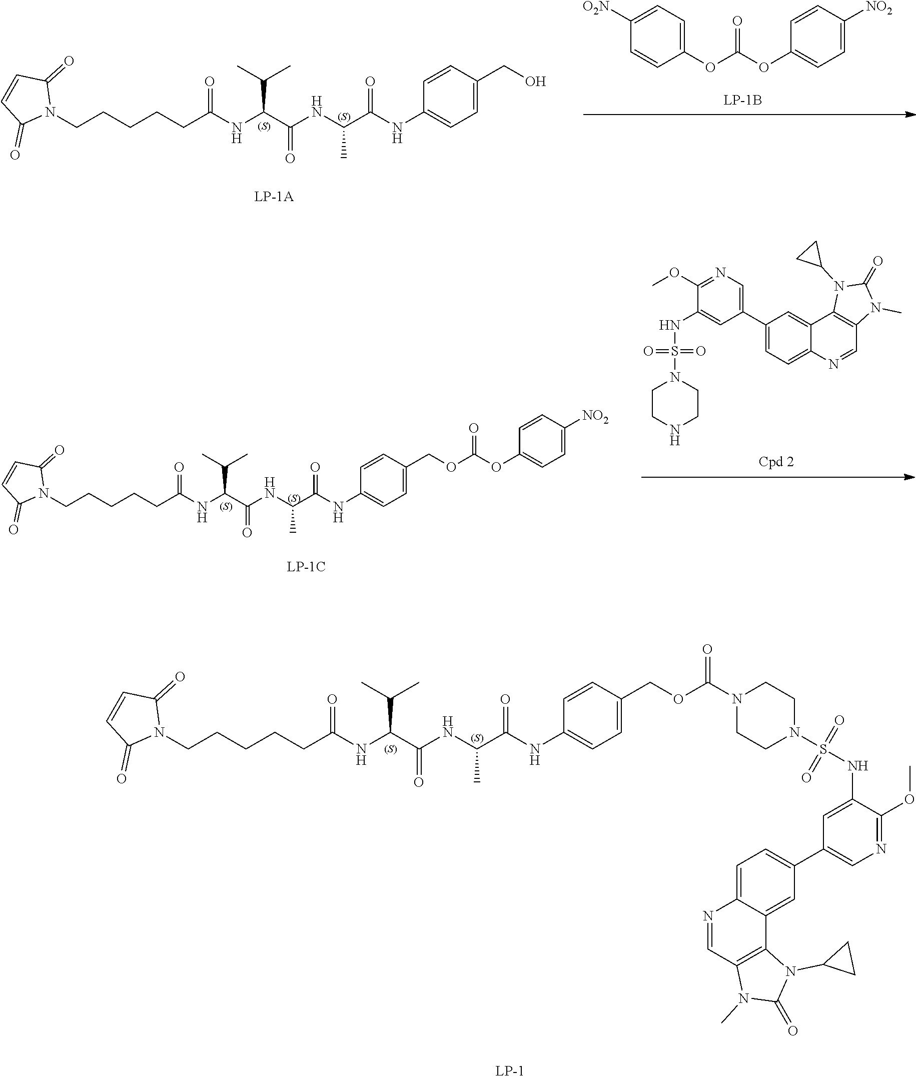

### 1) Synthesis of Compound LP-1C

In an ice-water bath and under nitrogen protection, DIEA (108 μL, 0.62 mmol) was added to a solution of compound LP-1A (CAS: 1870916-87-2) (150 mg, 0.31 mmol) and compound LP-1B (CAS: 5070-13-3) (188 mg, 0.62 mmol) in DMA (3 mL). After the addition was completed, the reaction was stirred at room temperature overnight. After the reaction was completed, the reaction solution was concentrated to remove the solvent, and the residue was purified by ISCO (mobile phase: MeOH:DCM=0:100-50:50) to afford compound LP-1C as a yellow solid (100 mg, 50% yield). MS (m/z)=673.8 [M+23]⁺

### 2) Synthesis of LP-1

In an ice-water bath and under nitrogen protection, to a solution of compound LP-1C (70 mg, 0.11 mmol) and compound 2 (60 mg, 0.12 mmol) in DMF (2 mL) was successively added pyridine (0.5 mL) followed by HOBt (7.4 mg, 0.055 mmol) and DIEA (38 μL, 0.22 mmol). After the addition was completed, the reaction solution was stirred at room temperature overnight. After the reaction was completed, the reaction solution was directly purified by ISCO (mobile phase: MeOH:H₂O (0.1% TFA)=0:100-100:0) to afford compound LP-1 as a white solid (12 mg, 11% yield). MS (m/z)=1021.8 [M+H]⁺

### Preparation of LP-2:

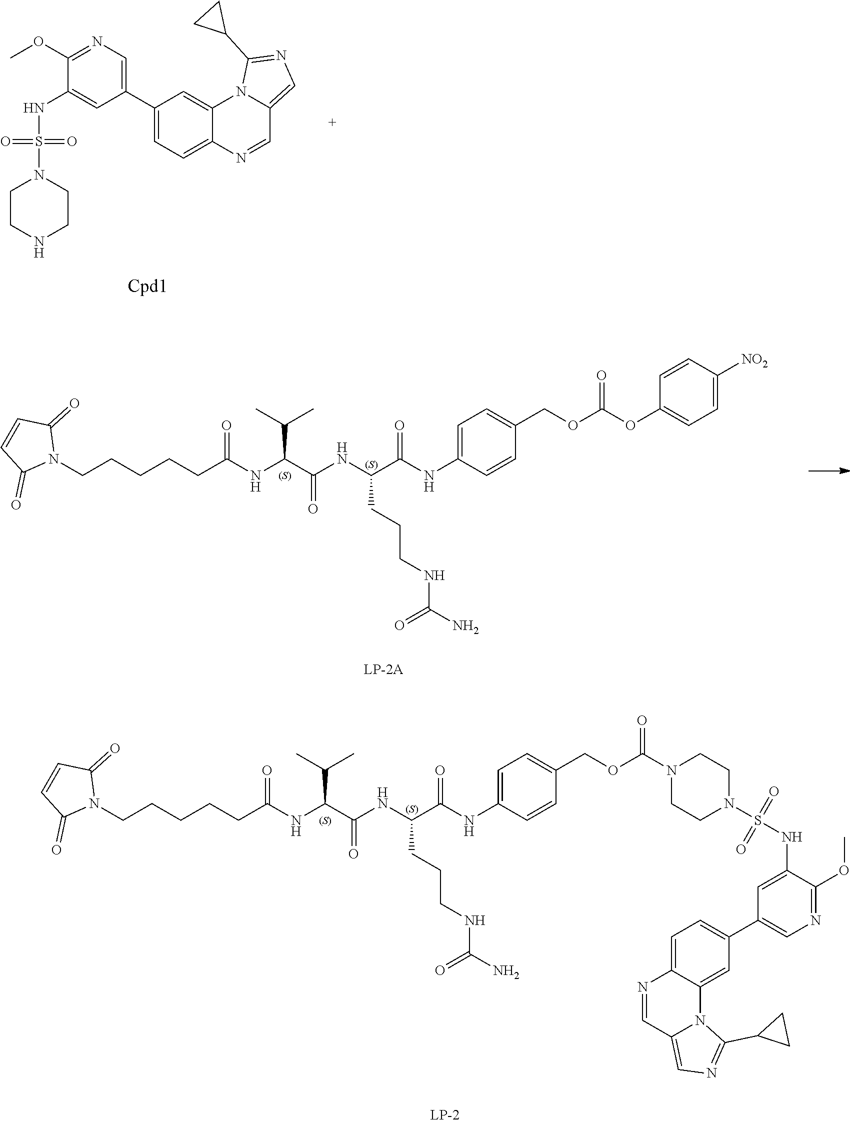

Compound 1 (41 mg, 0.085 mmol), compound LP-2A (52 mg, 0.07 mmol) (CAS: 159857-81-5), HOBt (5.7 mg, 0.04 mmol), pyridine (6 mg, 0.07 mmol), and DIEA (27 mg, 0.21 mmol) were added to a DMF solution under nitrogen protection, and the mixture was stirred at room temperature overnight. After the reaction was completed, the reaction solution was concentrated, and the residue was purified by ISCO (water (0.1% TFA):acetonitrile=100:0-0:100 gradient elution) to afford the target compound as a white solid (66 mg, 86.9% yield). MS (m/z)=539.4 [½M+H]⁺

The following compounds were prepared according to the preparation procedure of compound LP-2 using corresponding intermediates and reagents under appropriate conditions recognized by a person skilled in the art.

| LP | Payloads | | |
|---|---|---|---|
| No. | used | Structural formula | MS |
| LP-3 | Compound 2 | 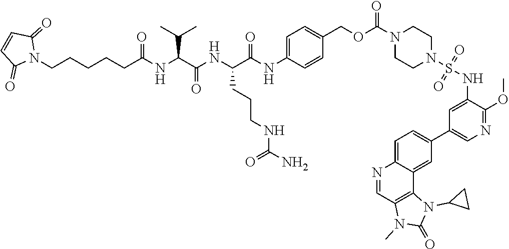 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1108.0 [M + H]⁺ |
| LP-4 | Compound 3 | 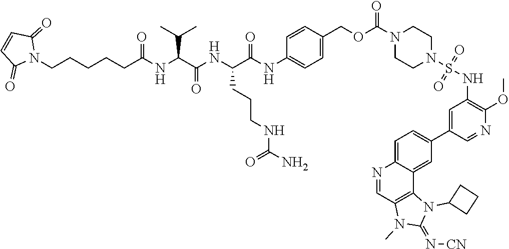 [SMILES: COc1ncc(-c2ccc3ncc4c(c3c2)n(C2CCC2)/c(=N\C#N)n4C)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1122.6 [M − 24]⁺ |
| LP-5 | Compound 4 | 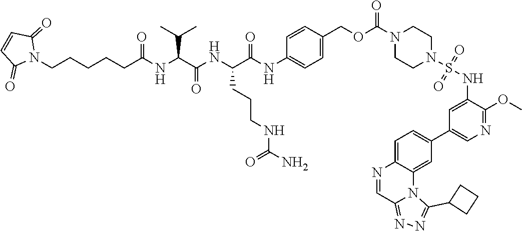 [SMILES: COc1ncc(-c2ccc3ncc4nnc(C5CCC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1092.8 [M + H]⁺ |
| LP-6 | Compound 5 | 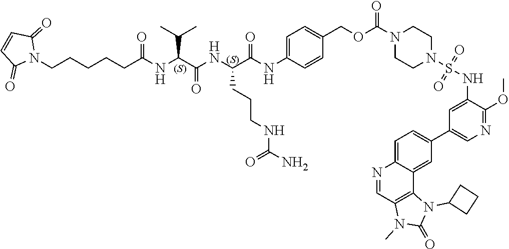 | 561.4 [1/2M + H]⁺ |
| LP-7 | Compound 6 | 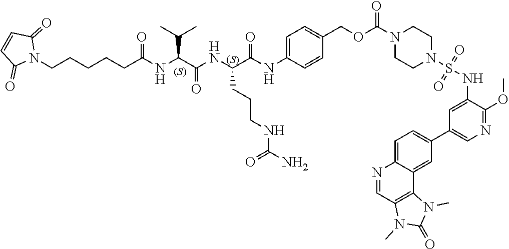 | 541.4 [1/2M + H]⁺ |
| LP-8 | Compound 7 | 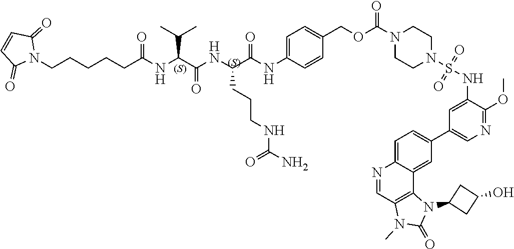 | 569.4 [1/2M + H]⁺ |
| LP-9 | Compound 8 | 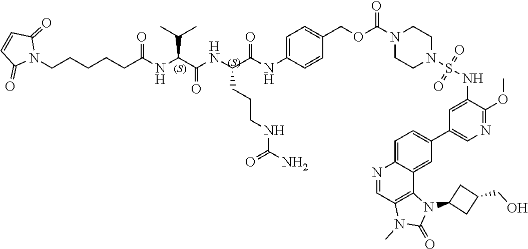 | 576.4 [1/2M + H]⁺ |
| LP-11 | Compound 23 | 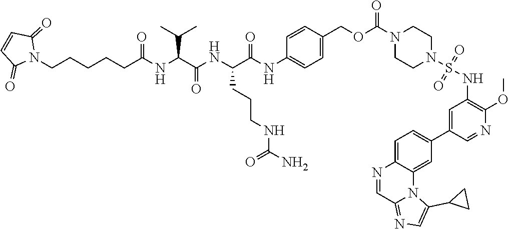 [SMILES: COc1ncc(-c2ccc3ncc4ncc(C5CC5)n4c3c2)cc1NS(=O)(=O)N1CCN(C(=O)OCc2ccc(NC(=O)[C@H](CCCNC(N)=O)NC(=O)[C@@H](NC(=O)CCCCCN3C(=O)C=CC3=O)C(C)C)cc2)CC1] | 1078.6 [M + H]⁺ |
| LP-17 | Compound 18 | 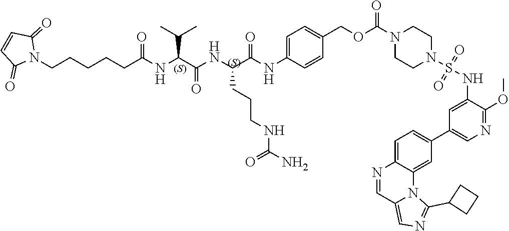 | 546.4 [1/2M + H]⁺ |
| LP-18 | Compound 20 | 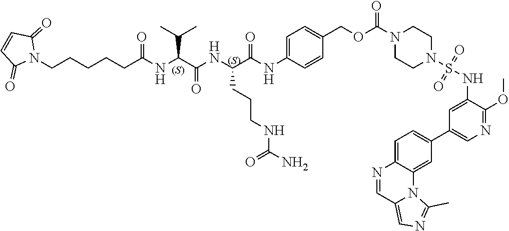 | 526.4 [1/2M + H]⁺ |
| LP-19 | Compound 14 | 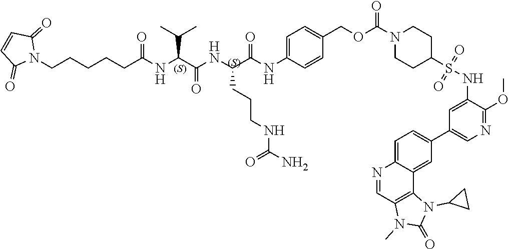 | 553.8 [1/2M + H]⁺ |
| LP-22 | Compound 28 | 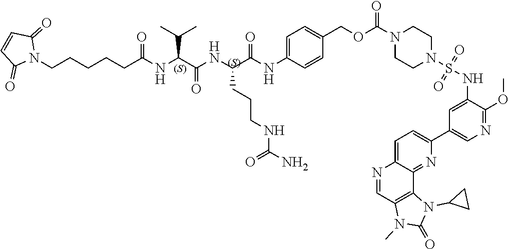 | 1109.8 [M + H]⁺ |
| LP-23 | Compound 30 | 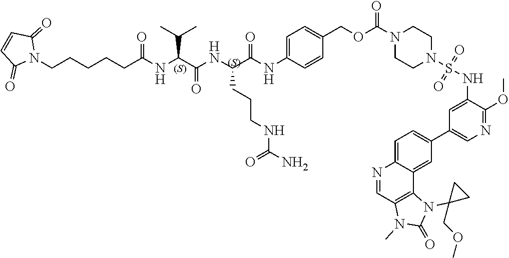 | 576.9 [1/2M + H]⁺ |
| LP-24 | Compound 29 | 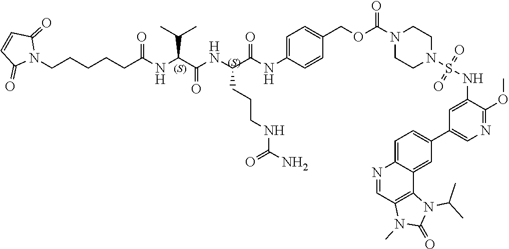 | 1110.4 [M + H]⁺ |
| LP-25 | Compound 27 | 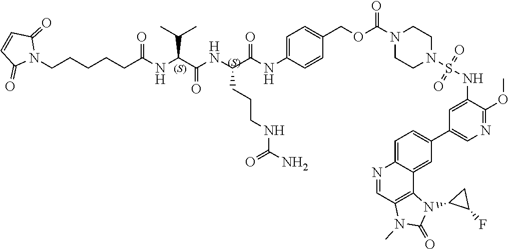 | 1126.4 [M + H]⁺ |
| LP-26 | Compound 32 | 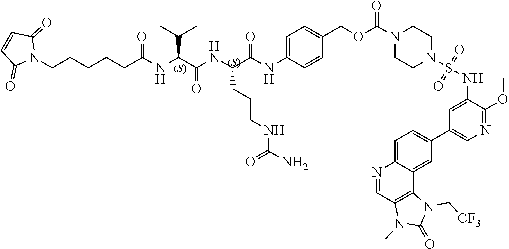 | 1150.4 [M + H]⁺ |
| LP-27 | Compound 33 | 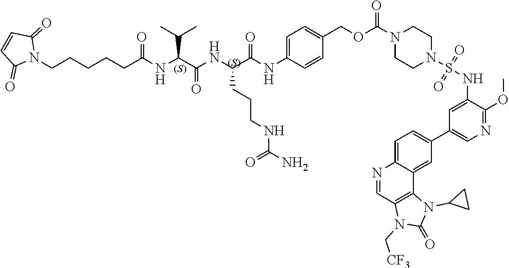 | 1176.6 [M + H]⁺ |
| LP-28 | Compound 31 | 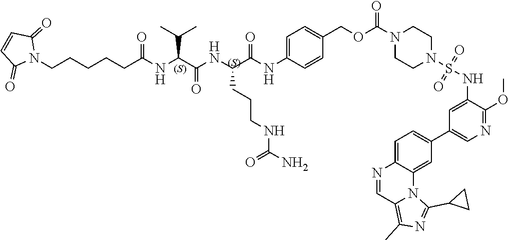 | 1092.6 [M + H]⁺ |

### Preparation of LP-10:

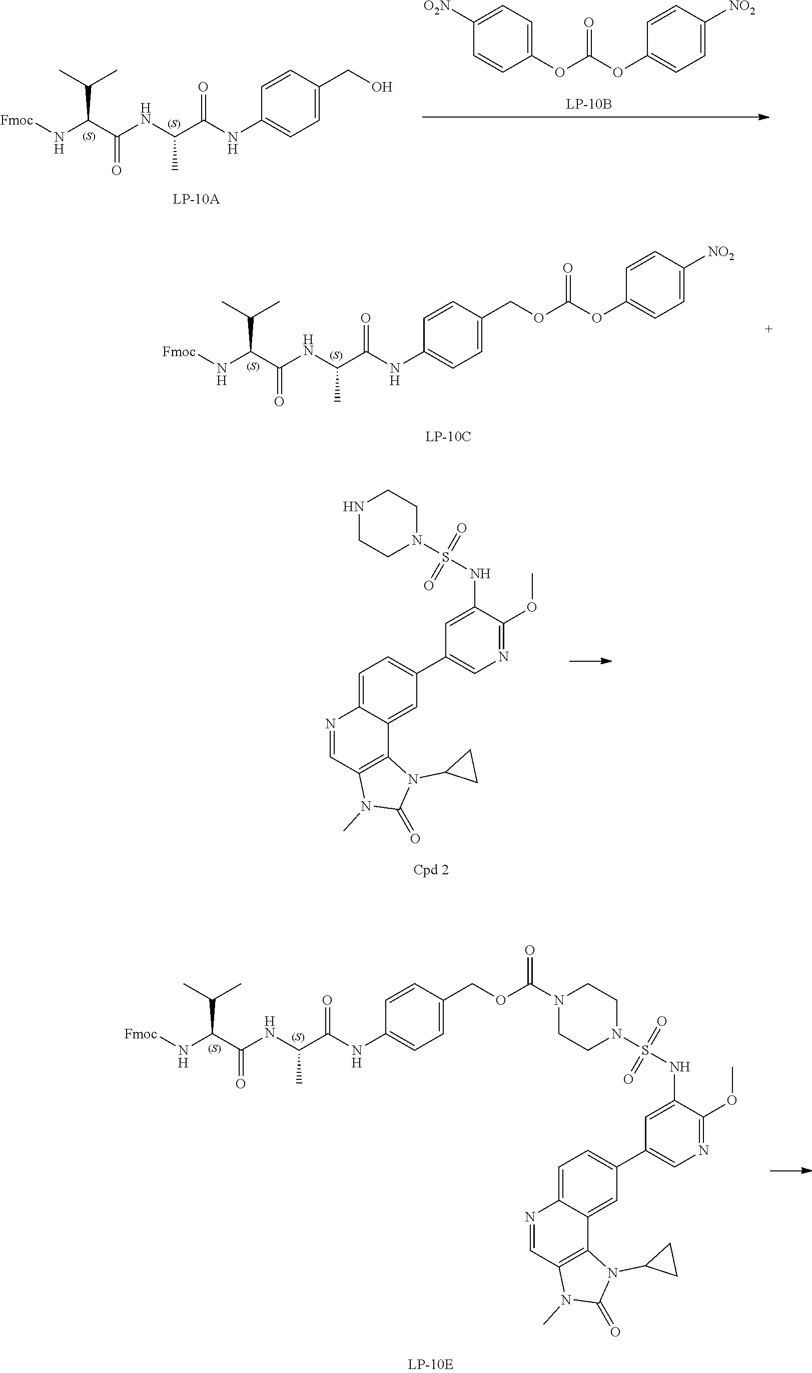

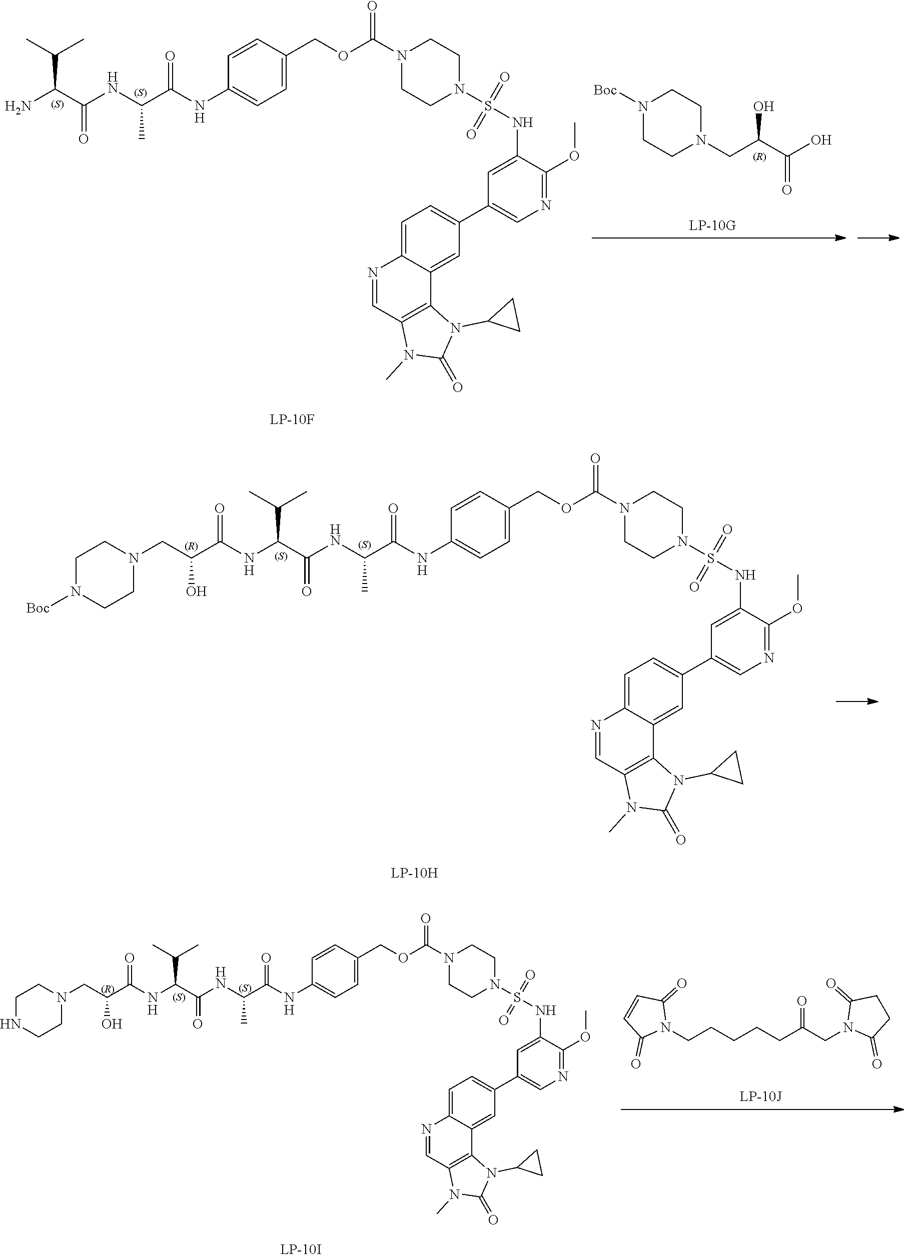

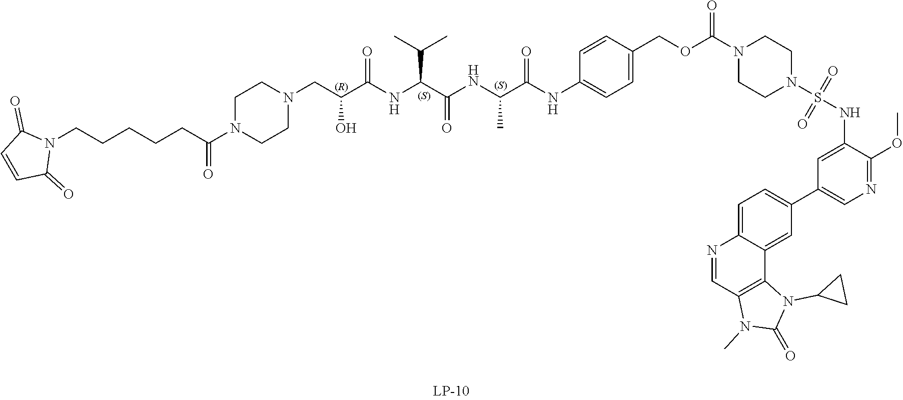

### 1) Synthesis of Compound LP-10C

In an ice-water bath and under nitrogen protection, DIEA (151 μL, 0.87 mmol) was added to a solution of compound LP-10A (CAS: 1394238-91-5) (300 mg, 0.58 mmol) and compound LP-10B (CAS: 5070-13-3) (353 mg, 1.16 mmol) in DMF (3 mL). After the addition was completed, the reaction was stirred at room temperature overnight. After the reaction was completed, the reaction solution was concentrated to remove the solvent, and the residue was purified by ISCO (mobile phase: MeOH:DCM=0:100-50:50) to afford compound LP-10C as a yellow solid (203 mg, 51% yield). MS (m/z)=703.3 [M+23]⁺

### 2) Synthesis of Compound LP-10E

In an ice-water bath and under nitrogen protection, to a solution of compound LP-10C (150 mg, 0.22 mmol) and compound 2 (135 mg, 0.26 mmol) in DMF (3 mL) was successively added pyridine (0.5 mL) followed by HOBt (15 mg, 0.11 mmol) and DIEA (77 μL, 0.44 mmol). After the addition was completed, the reaction solution was stirred at room temperature overnight. After the reaction was completed, the reaction solution was directly purified by ISCO (mobile phase: MeOH:H₂O (0.1% TFA)=0:100-100:0) to afford compound LP-10E as a white solid (110 mg, 48% yield). MS (m/z)=1050.8 [M+H]⁺

### 3) Synthesis of Compound LP-10F

In an ice-water bath and under nitrogen protection, to a solution of compound LP-10E (110 mg, 0.11 mmol) in DMA (3 mL) was added DEA (0.5 mL). After the addition was completed, the reaction solution was stirred at room temperature for 1 h. After the reaction was completed, the reaction solution was directly purified by ISCO (mobile phase: CH₃CN:H₂O (0.1% TFA)=0:100-100:0) to afford compound LP-10F as a yellow solid 98 mg. MS (m/z)=829.0 [M+H]⁺

### 4) Synthesis of Compound LP-10H

In an ice-water bath and under nitrogen protection, to a solution of compound LP-10F (98 mg, 0.12 mmol) and compound LP-10G (synthesized according to the preparation of intermediate-17) (42 mg, 0.16 mmol) in DMA (3 mL) was added HOBt (24 mg, 0.18 mmol) and EDCI (68 mg, 0.36 mmol), the mixture was stirred for 2 minutes, and then DIEA (82 μL, 0.47 mmol) was added. After the addition was completed, the mixture was stirred at room temperature for 5 h. After the reaction was completed, the reaction solution was directly purified by ISCO (mobile phase: CH₃CN:H₂O (0.1% TFA)=0:100-100:0) to afford compound LP-10H as a white solid (105 mg, 82% yield). MS (m/z)=1085.8 [M+H]⁺

### 5) Synthesis of Compound LP-10I

In an ice-water bath and under nitrogen protection, to a solution of compound LP-10H (105 mg, 0.10 mmol) in DCM (2 mL) was added TFA (1 mL), and the mixture was stirred for 15 minutes. After the reaction was completed, the reaction solution was concentrated, and the residue was purified by ISCO (mobile phase: CH₃CN:H₂O (0.1% TFA)=0:100-100:0) to afford compound LP-10I as a yellow solid (87 mg, 91% yield). MS (m/z)=984.8 [M+H]⁺

### 6) Synthesis of LP-10

Compound LP-10I (87 mg, 0.088 mmol), compound LP-10J (CAS: 55750-63-5) (35 mg, 0.115 mmol), and DIEA (31 μl, 0.176 mmol) were dissolved in DMA (2 mL), and the mixture was stirred at room temperature for 4 hours. After the reaction was completed, the reaction solution was directly isolated by ISCO (mobile phase: CH₃CN:H₂O (0.1% TFA)=0:100-100:0) to afford LP-10 as a yellow solid (14 mg, 13% yield). MS (m/z)=1177.8 [M+H]⁺

The following compounds were prepared according to the preparation procedure of compound LP-10 using corresponding intermediates and reagents under appropriate conditions recognized by a person skilled in the art.

| | Payloads | | |
|---|---|---|---|
| LP No. | used | Structural formula | MS |
| LP-12 | Compound 1 | 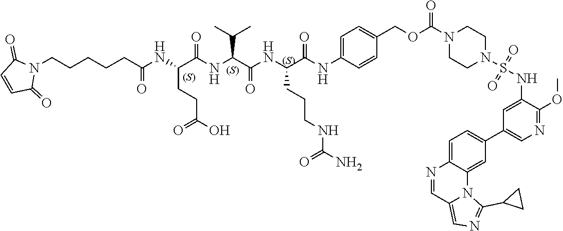 | 604.4 [1/2M + H]⁺ |
| LP-13 | Compound 2 | 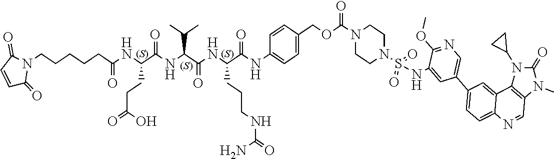 | 1237.8 [M + H]⁺ |

### Preparation of LP-14:

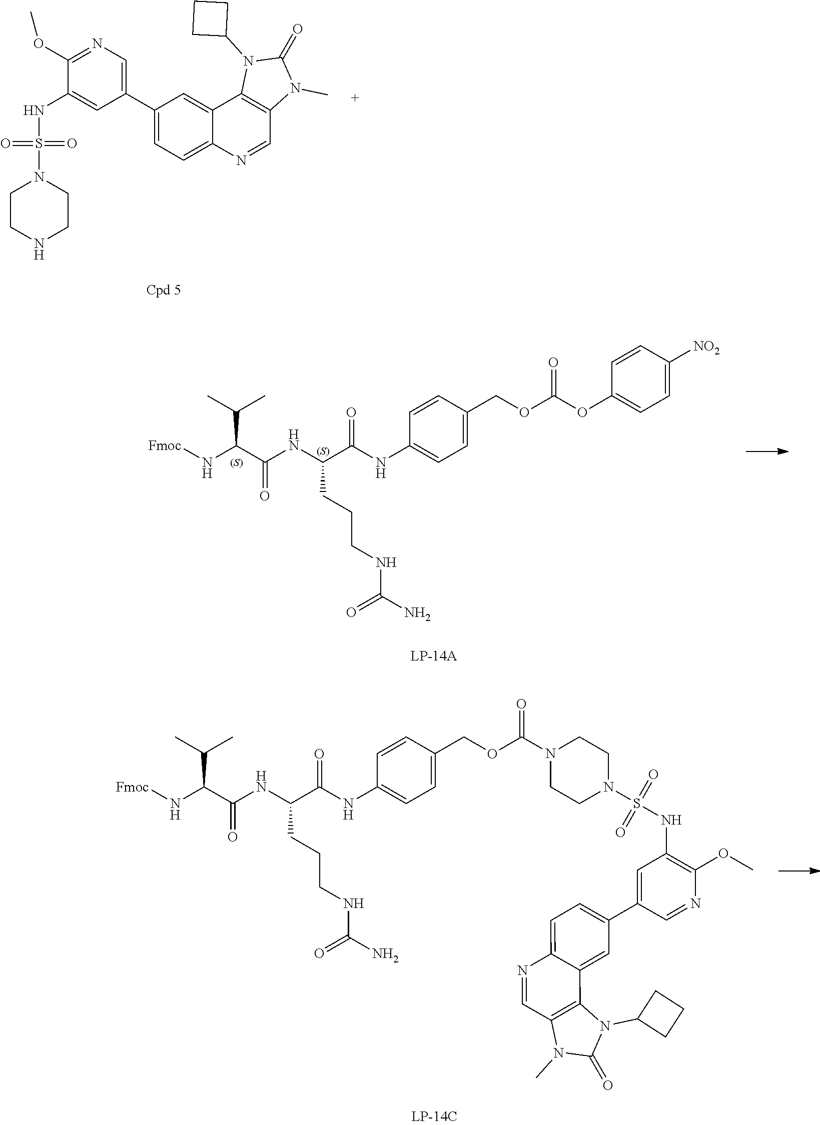

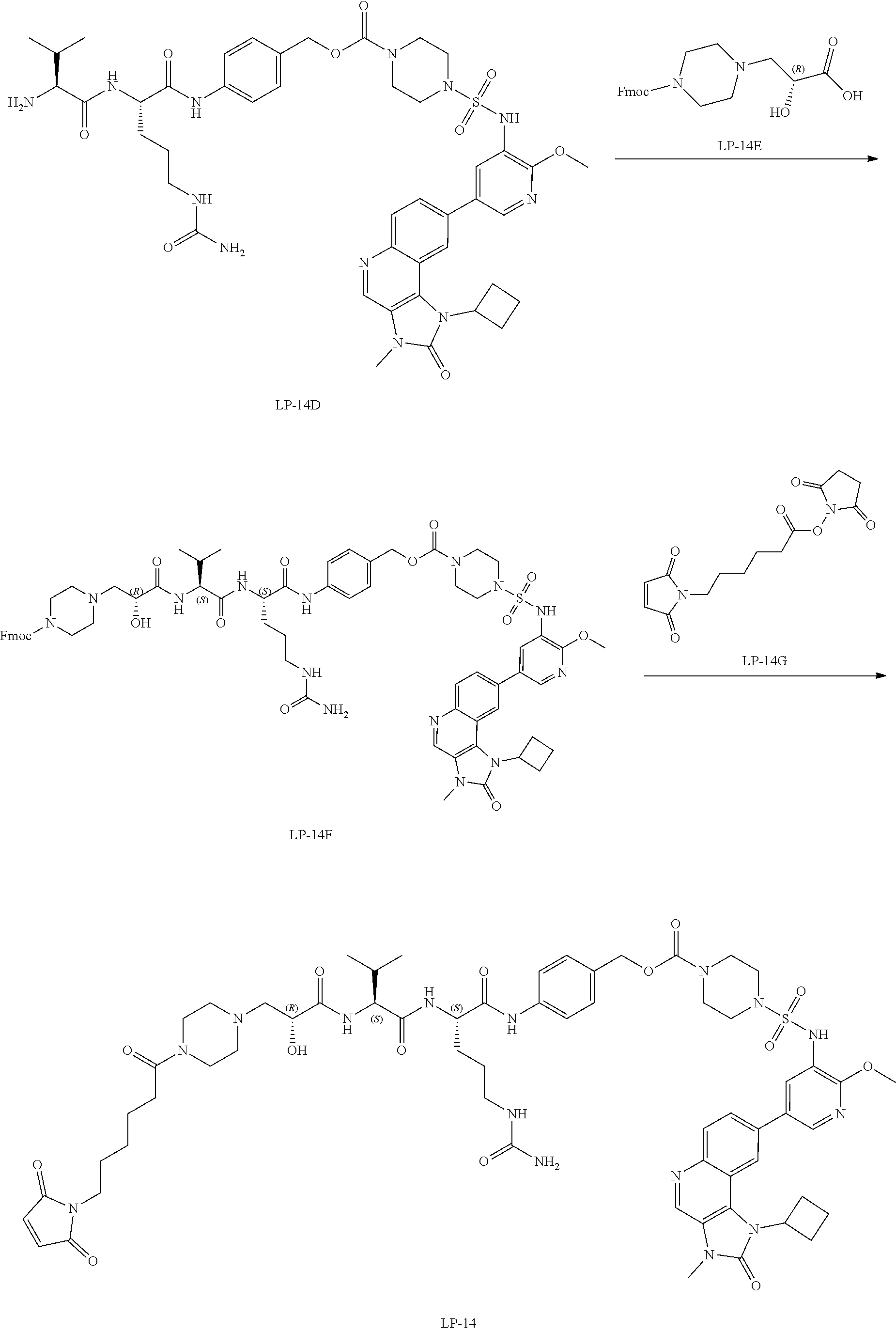

### 1) Synthesis of Compound LP-14C

At room temperature and under nitrogen protection, pyridine (25 mg, 0.32 mmol) and HOBt (15 mg, 0.11 mmol) were added to a solution of compound 5 (55 mg, 0.11 mmol) and compound LP-14A (CAS: 863971-53-5) (81 mg, 0.11 mmol) in anhydrous DMF, respectively. After the addition was completed, the reaction solution was stirred at room temperature overnight. After the reaction was completed, the reaction solution was concentrated under reduced pressure, and the residue was purified by ISCO (mobile phase: MeOH:H₂O=0:100-100:0) to afford compound LP-14C as a white solid (58 mg, 48% yield). MS (m/z)=1151.8 [M+H]⁺

### 2) Synthesis of Compound LP-14D

Diethylamine (1 mL) was added to a solution of compound LP-14C (58 mg, 0.050 mmol) in DCM/MeOH (2 mL, 3:1) at room temperature. The reaction was stirred at room temperature for 3 h. After the reaction was completed, the reaction solution was concentrated under reduced pressure. The residue was stirred with THF/PE (20 mL, 1:5) for 30 minutes, and filtered. The filter cake was collected and dried to afford compound LP-14D as a white solid (45 mg, 96% yield). MS (m/z)=929.4 [M+H]⁺

### 3) Synthesis of Compound LP-14F

In an ice-water bath and under nitrogen protection, DIEA (19 mg, 0.15 mmol), and HATU (22 mg, 0.058 mmol) were added to a solution of compound LP-14D (45 mg, 0.048 mmol) and compound LP-14E (intermediate-17) (20 mg, 0.048 mmol) in DCM (10 mL), respectively. The reaction solution was stirred at room temperature overnight. After the reaction was completed, the reaction solution was poured into water, and extracted with DCM (20 mL×3). The organic phases were combined and dried over anhydrous sodium sulfate, concentrated under reduced pressure, and purified by ISCO (mobile phase: MeOH:H₂O=0:100-100:0) to afford compound LP-14F as a white solid (32 mg, 51% yield). MS (m/z)=1307.8 [M+H]⁺

### 4) Synthesis of LP-14

Compound LP-14F (32 mg, 0.024 mmol), compound LP-14G (CAS: 55750-63-5) (15 mg, 0.049 mmol), and triethylamine (75 mg, 0.734 mmol) were dissolved in DMF (2 mL), and the mixture was stirred at room temperature overnight. After the reaction was completed, the reaction solution was concentrated under reduced pressure and isolated by ISCO (mobile phase: H₂O (0.1% HCOOH):CH₃CN=100:0-0:100) to afford compound LP-14 as a white solid (3 mg, 9% yield). MS (m/z)=1278.6 [M+H]⁺

The following compounds were prepared according to the preparation procedure of compound LP-14 using corresponding intermediates and reagents under appropriate conditions recognized by a person skilled in the art.

| | Payloads | | |
|---|---|---|---|
| LP No. | used | Structural formula | MS |
| LP-15 | Compound 1 | 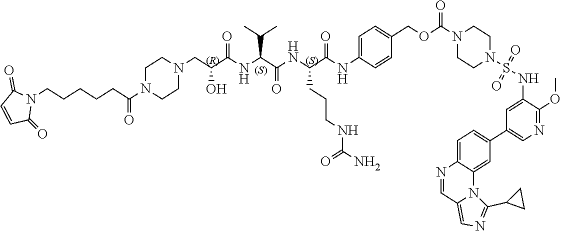 | 617.8 [1/2M + H]⁺ |
| LP-16 | Compound 2 | 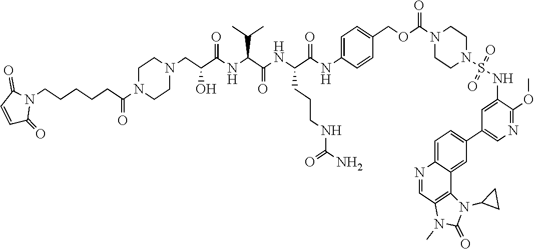 [SMILES: COc1ncc(-c2ccc3ncc4c(c3c2)n(C2CC2)c(=O)n4C)cc1NS(=O)(=O)N1CCN(C(=O)OCc2ccc(NC(=O)[C@H](CCCNC(N)=O)NC(=O)[C@@H](NC(=O)[C@H](O)CN3CCN(C(=O)CCCCCN4C(=O)C=CC4=O)CC3)C(C)C)cc2)CC1] | 1264.0 [M + H]⁺ |

### Preparation of LP-21:

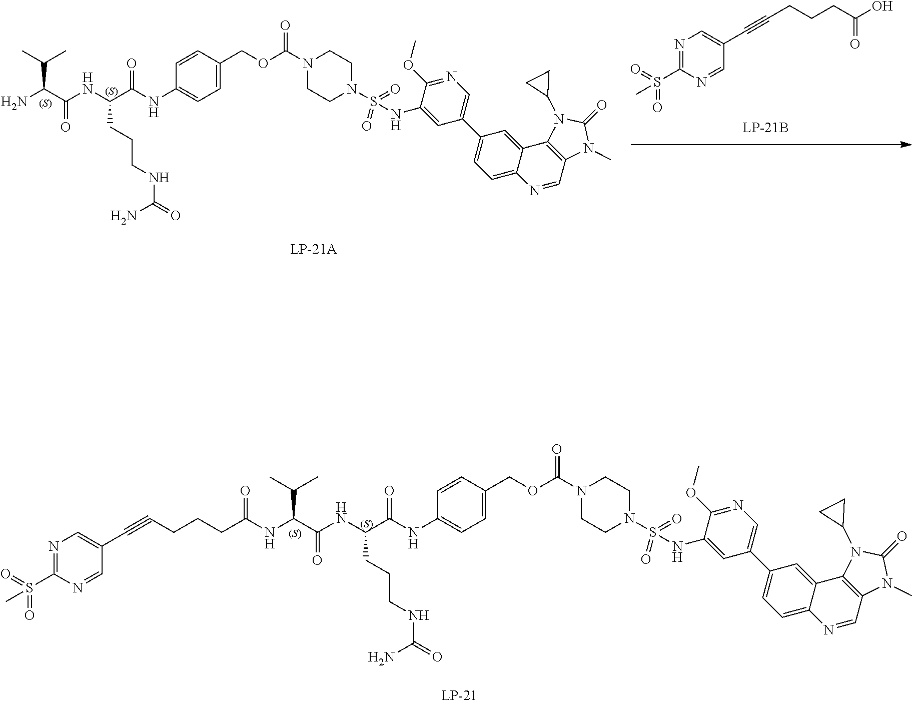

DIEA (8 mg, 0.054 mmol) and HATU (16 mg, 0.041 mmol) were added to a solution of compound LP-21A (25 mg, 0.027 mmol) (synthesized according to the synthesis of compound LP-10F in the preparation of LP-10) and compound LP-21B (CAS: 2356229-58-6) (10 mg, 0.037 mmol) in DCM, and the mixture was stirred overnight at room temperature under nitrogen protection. After the reaction was completed, the reaction solution was poured into water, and extracted with DCM (20 mL×3). The organic phases were combined, dried over anhydrous sodium sulfate, concentrated under reduced pressure, and isolated by ISCO (mobile phase: water (0.1% formic acid):acetonitrile=100:0-0:100) to afford compound LP-21 as a white solid (8 mg, 24% yield). MS (m/z)=1165.6 [M+H]⁺

The following compounds were prepared according to the preparation procedure of compound LP-21 using corresponding intermediates and reagents under appropriate conditions recognized by a person skilled in the art.

| | Payloads | | |
|---|---|---|---|
| LP No. | used | Structural formula | MS |
| LP-20 | Compound 1 | 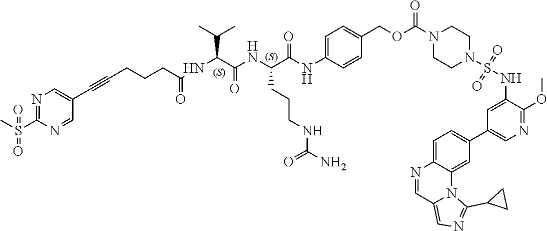 | 1136.9 [M + H]⁺ |

### Example 4: Preparation of ADCs

### Preparation of ADC-6:

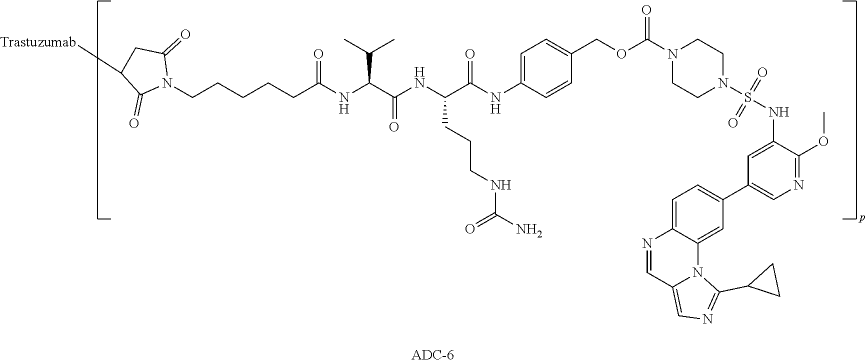

His buffer (363.5 μL), TCEP solution (5 μL, 10 mmol/L in His buffer, 2.5 eq), and DTPA solution (7.5 μL, 10 mmol/L in His buffer) were successively added to 8 mg/mL trastuzumab solution (375 μL, 3 mg) formulated with His buffer (10 mmol/L, pH 7.0). The resulting solution was reacted at 22° C. for 60 minutes, and then a solution of compound LP-2 in DMSO:H₂O (volume ratio: 3:1) (40 μL, 5 mmol/L, 10 eq) was added. The mixture was further reacted at 22° C. for 60 minutes. The reaction solution was transferred to an ultrafiltration centrifuge tube (Millipore Amicon® Ultra), and a His/His·HCl buffer (10 mmol/L, pH 6.2) was added for solution exchange, so that a target product was obtained. As detected by HIC-HPLC, the drug/antibody ratio (DAR value) was calculated as 4.0.

The following antibody-drug conjugates were prepared according to the preparation procedure of ADC-6 using the corresponding antibodies (e.g., the sequences of the heavy chain and light chain of mAb1 can be found in SEQID: 39 and SEQID: 40; the heavy chain and light chain sequences of mAb2 can be found in SEQID: 59 and SEQID: 60; the heavy and light chain sequences of mAb3 can be found in SEQID: 49 and SEQID: 50) and linker-payloads under appropriate conditions recognized by those skilled in the art (e.g., the commonly used conjugation methods described in the specification).

| | | Measured |
|---|---|---|
| ADC No. | Structure | DAR |
| ADC-1 | 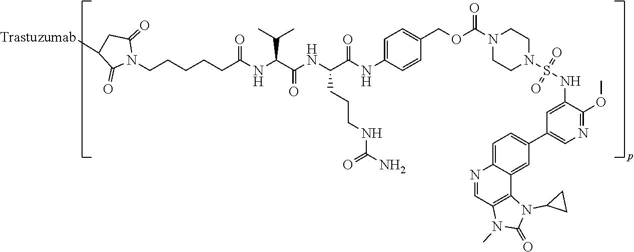 | 3.8 |
| ADC-2 | 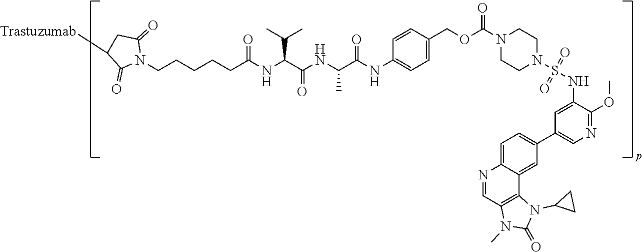 | 3.9 |
| ADC-3 | 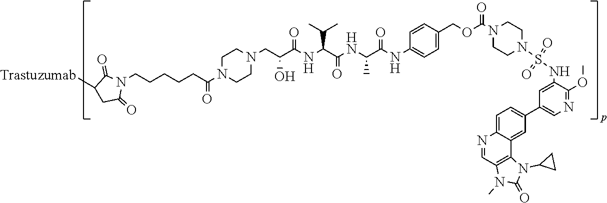 | 4.0 |
| ADC-4 | 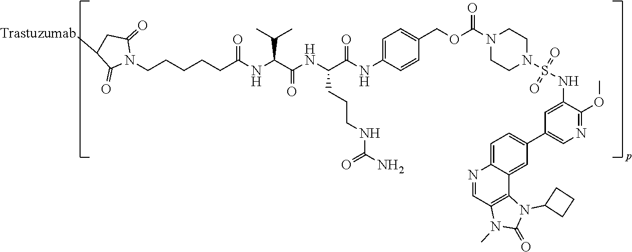 | 4.0 |
| ADC-5 | 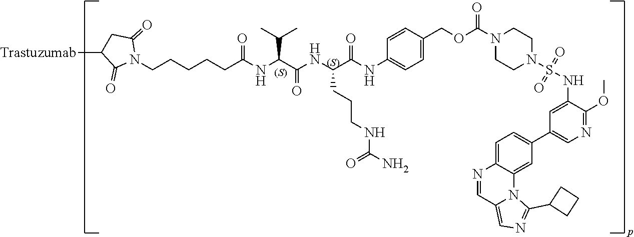 | 4.0 |
| ADC-7 | 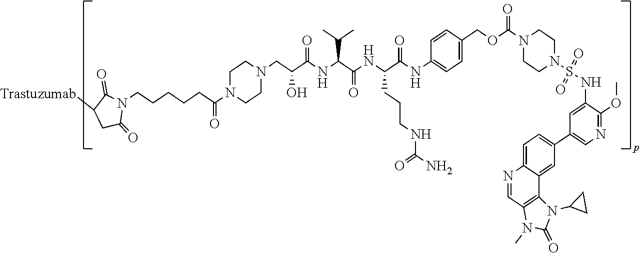 | 4.1 |
| ADC-8 | 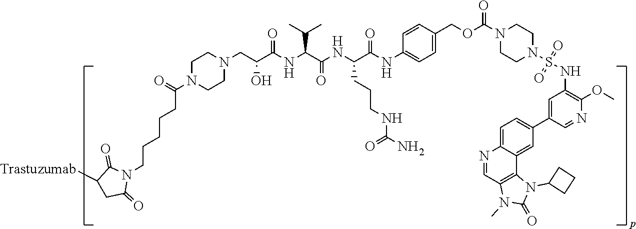 | 4.0 |
| ADC-9 | 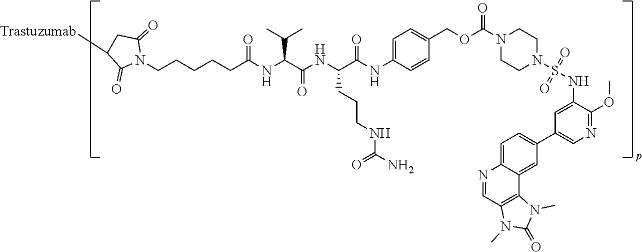 | 4.0 |
| ADC-10 | 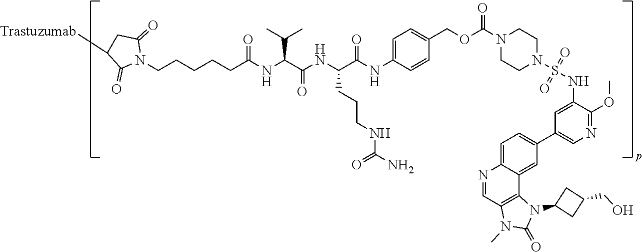 | 4.0 |
| ADC-11 | 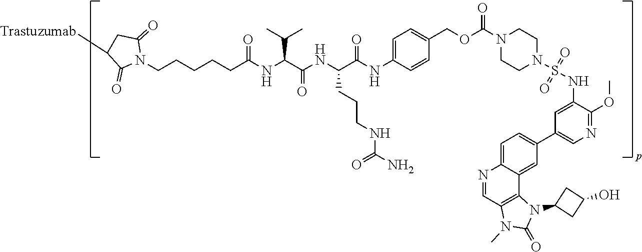 | 4.0 |
| ADC-12 | 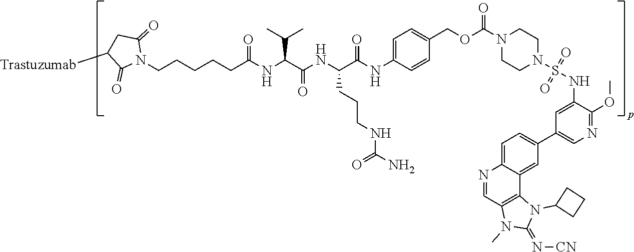 | 4.17 |
| ADC-13 | 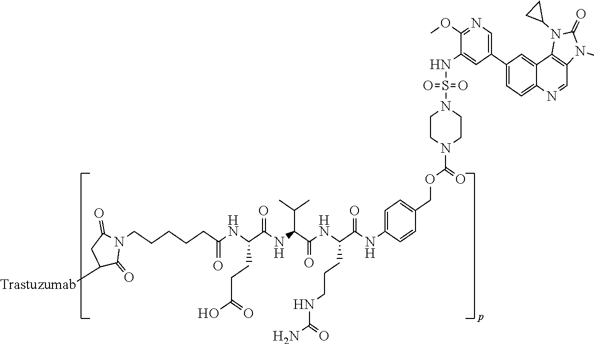 | 4.14 |
| ADC-14 | 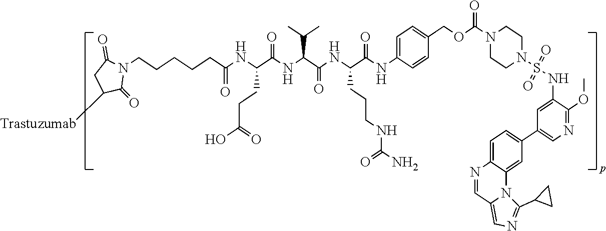 | 4.18 |
| ADC-15 | 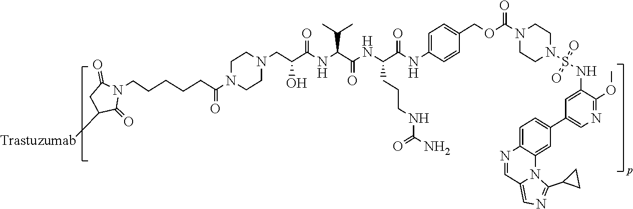 | 4.12 |
| ADC-16 | 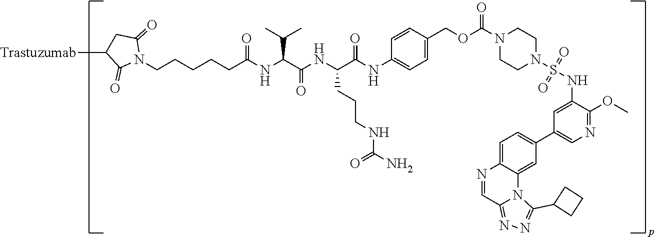 | 3.9 |
| ADC-17 | 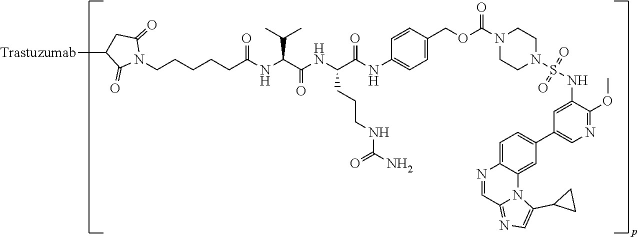 | 4.0 |
| ADC-19 | 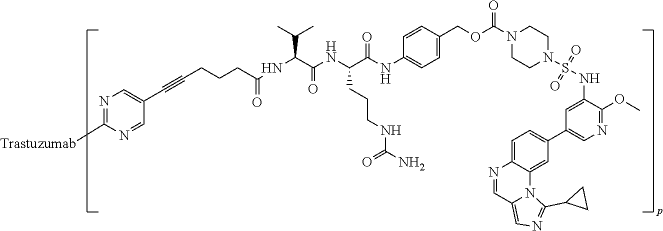 | 4.36 |
| ADC-20 | 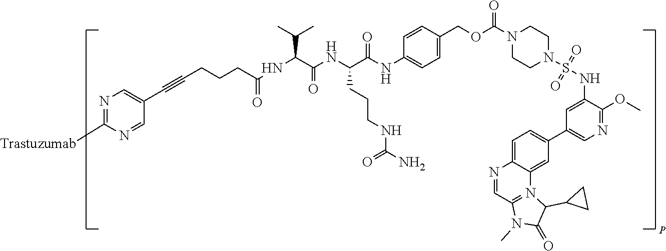 | 4.21 |
| ADC-21 | 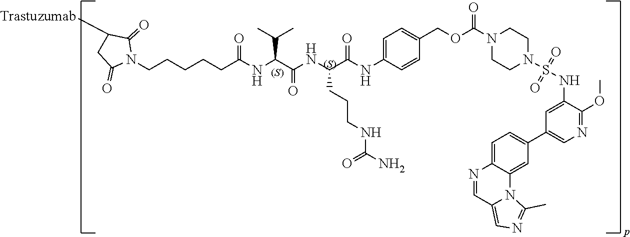 | 4.0 |
| ADC-22 | 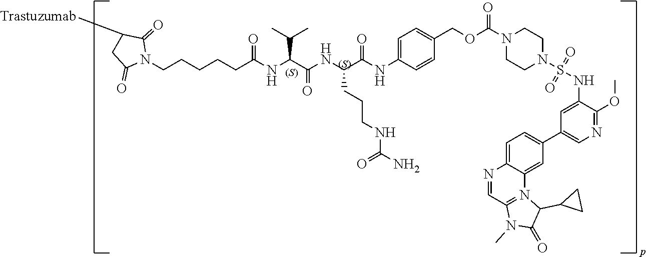 | 4.0 |
| ADC-23 | 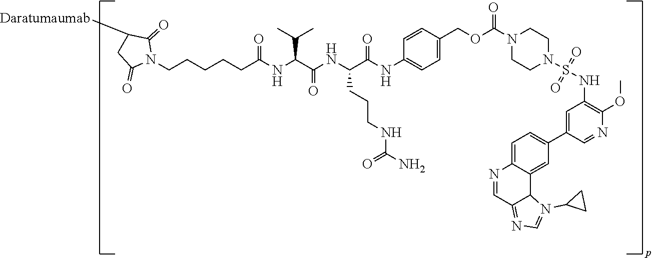 | 4.2 |
| ADC-24 | 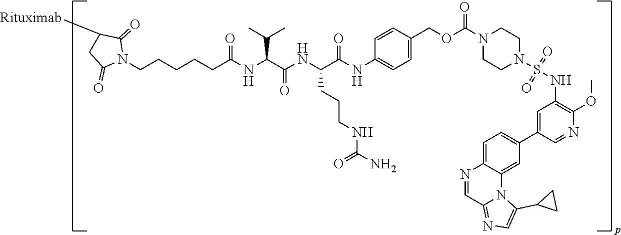 | 4.2 |
| ADC-25 | 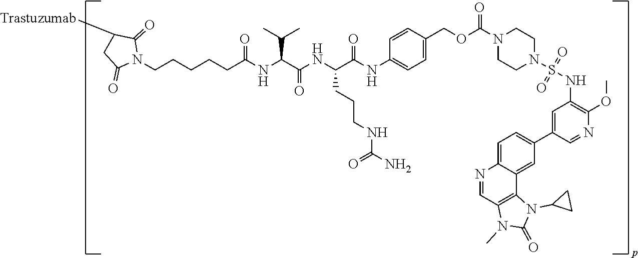 | 3.9 |
| ADC-26 | 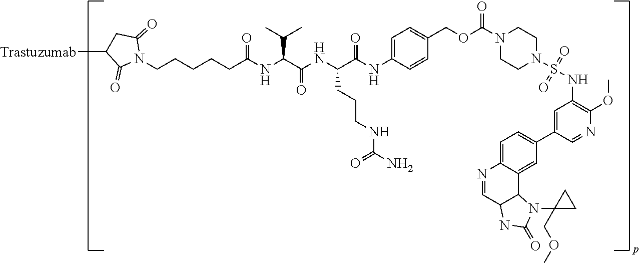 | 4.0 |
| ADC-27 | 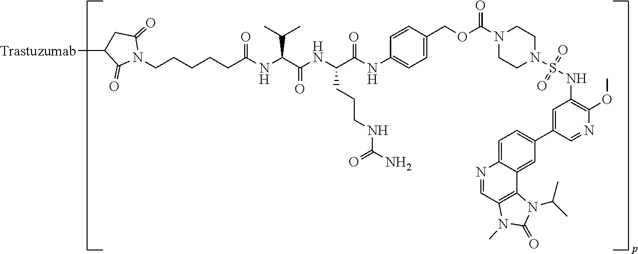 | 3.9 |
| ADC-28 | 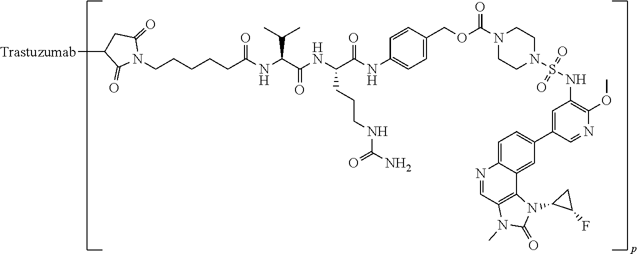 | 3.8 |
| ADC-29 | 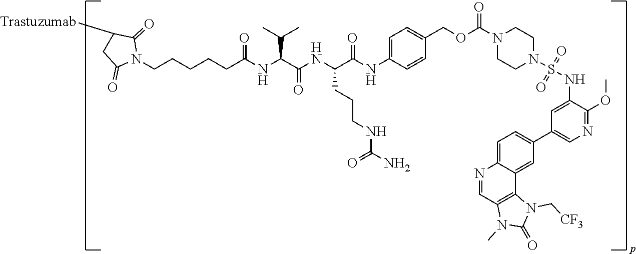 | 4.0 |
| ADC-30 | 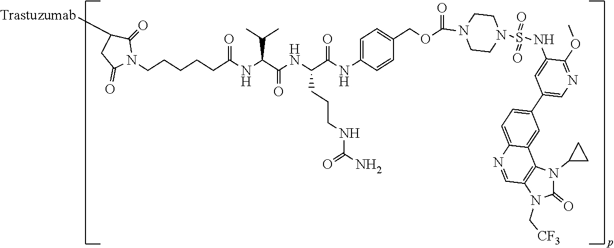 | 4.1 |
| ADC-31 | 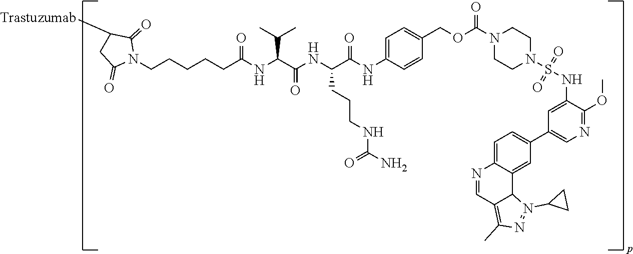 | 4.0 |
| ADC-32 | 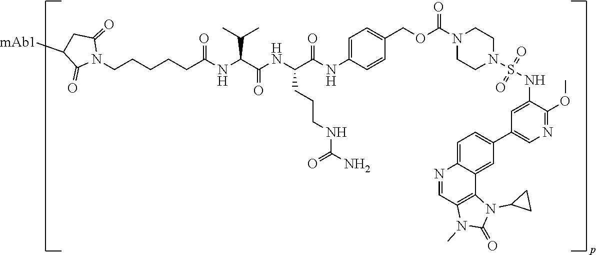 | 3.8 |
| ADC-33 | 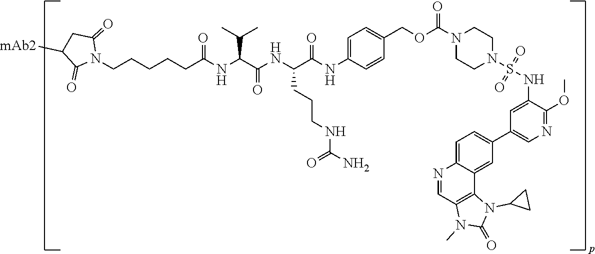 | 3.8 |
| ADC-34 | 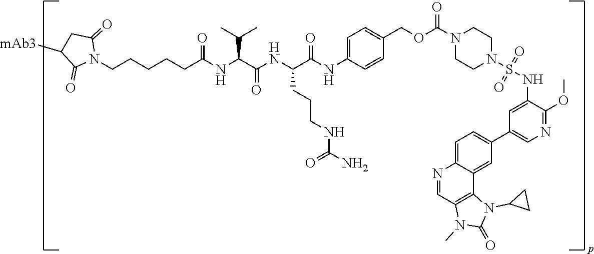 | 4.0 |

### Preparation of ADC-18

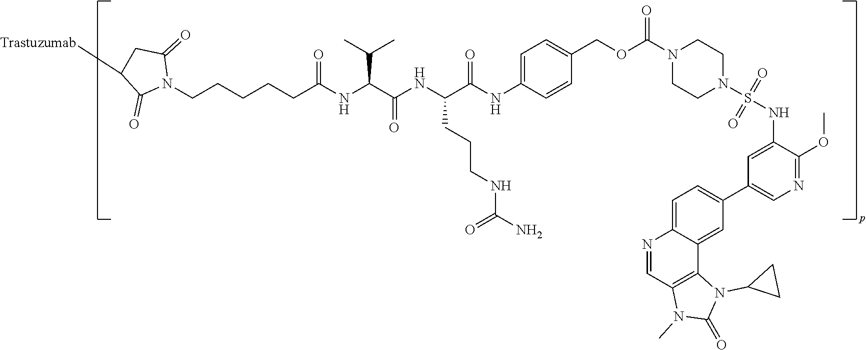

His buffer (595.85 μL), TCEP solution (16.65 μL, 10 mmol/L in His buffer, 5 eq), and DTPA solution (12.5 μL, 10 mmol/L in His buffer) were successively added to 8 mg/mL trastuzumab solution (625 μL, 5 mg) formulated with His buffer (10 mmol/L, pH 7.0). The resulting solution was reacted at 37° C. for 120 minutes, and then a solution of compound LP-3 in DMSO:H₂O (volume ratio: 3:1) (100 μL, 5 mmol/L, 15 eq) was added. The mixture was further reacted at 37° C. for 60 minutes. The reaction solution was transferred to an ultrafiltration centrifuge tube (Millipore Amicon® Ultra), and a His/His·HCl buffer (10 mmol/L, pH 6.2) was added for solution exchange, so that a target product was obtained. As detected by HIC-HPLC, the drug/antibody ratio (DAR value) was calculated as 7.80.

### Example 5: Testing the Binding Activity of the ADCs of the Present Invention to Corresponding Positive Cells

### Experimental Methods

First of all, HCC1954 cells (HER2 positive, Cat. No. CRL-2338) (purchased from the cell bank of American Type Culture Collection, ATCC) were normally cultured in an RPMI-1640 medium (Gibco, Cat. No. A1049101) with 10% fetal bovine serum (FBS) (Gibco, Cat. No. A5669701) in a cell culture incubator at 37° C. and 5% CO₂. The adhered cells were digested with 0.25% Trypsin-EDTA (Gibco, Cat. No. 25200-056). The dissociated cells were collected, and centrifuged. The cells were resuspended in a staining buffer (PBS (Gibco, Cat. No. C20012500BT)+2% FBS) at 4° C., and were added to a V-bottom 96-well plate (BD Falcon) at 1.0×10⁵ cells/well and 50 μL/well. The antibody or the antibody-drug conjugate was diluted with a staining buffer in 4-fold gradient to the corresponding concentrations, and then 50 μL/well of the diluted antibody or antibody-drug conjugate at different concentrations (final concentrations: 150 nM, 37.5 nM, 9.38 nM, 2.34 nM, 0.586 nM, 0.146 nM, 0.037 nM, and 0.0092 nM) or 50 μL/well of the human IgG1 isotype reference antibody (Biolegend, 403502) were added to the corresponding cell wells, respectively. The plate was incubated at 4° C. for 1.0 hour. After the centrifugation at 300 g for 5 minutes, the supernatant was aspirated, and the pellet was resuspended in 200 μL/well of staining buffer and washed twice. The PE-labeled anti-human IgG Fc antibody (Biolegend, 410708) diluted in a staining buffer was added at 100 μL/well and incubated in the dark at 4° C. for 45 minutes. After the centrifugation at 300 g for 5 minutes, the supernatant was aspirated, and the pellet was resuspended in 200 μL/well of staining buffer and washed twice. Then, the cells were resuspended in 200 μL/well of FACS fixation buffer (PBS+2% FBS+1% tissue cell fixation buffer (Beijing Dingguo Changsheng Biotechnology Co., Ltd., AR-0211)) and fixed at room temperature for 15 minutes or at 4° C. overnight.

### Detection and Data Analysis

Fluorescence signals were detected on a BD FACSCanto™ II flow cytometer (BD Biosciences) to obtain the mean fluorescence intensity (MFI) of the binding of the antibody or antibody-drug conjugate to cells, and the half maximal effective concentration (EC₅₀) values were then obtained using the Log (agonist) vs. response equation in the GraphPad Prism software (GraphPad Software).

The source and catalog number of cells selected for the binding experiments of other antibodies or antibody-drug conjugates to the corresponding tumor cell surface antigens were: Ramos cells (ATCC, Cat. No. CRL-1596), H2122 (ATCC, Cat. No. CRL-5985), T47D (ATCC, Cat. No. HTB-133). The experimental methods were similar to those described above and were within the scope known to those skilled in the art, and the detection was carried out using suitable reagents and test methods.

The resulting data was showed in the following table:

TABLE 1-1

| Antibody or ADC | EC₅₀, nM HCC1954 (HER2⁺) |
|---|---|
| Trastuzumab | 2.468 |
| ADC-1 | 3.890 |
| ADC-2 | 4.688 |
| ADC-3 | 3.956 |
| ADC-4 | 3.914 |
| ADC-6 | 2.085 |
| ADC-7 | 3.585 |
| ADC-8 | 3.907 |
| ADC-9 | 3.781 |
| ADC-10 | 3.407 |
| ADC-11 | 4.594 |
| ADC-12 | 1.772 |
| ADC-13 | 2.950 |
| ADC-14 | 1.988 |
| ADC-15 | 3.145 |
| ADC-18 | 4.400 |
| ADC-21 | 3.137 |
| ADC-22 | 2.411 |
| ADC-25 | 2.107 |
| ADC-26 | 3.759 |
| ADC-27 | 2.559 |
| ADC-28 | 3.123 |
| ADC-29 | 2.868 |
| ADC-30 | 2.689 |
| ADC-31 | 3.430 |

TABLE 1-2

| Antibody or ADC | EC₅₀, nM Ramos (CD38⁺) |
|---|---|
| Daratumumab | 3.510 |
| ADC-23 | 2.242 |

TABLE 1-3

| Antibody or ADC | EC₅₀, nM Ramos (CD20⁺) |
|---|---|
| Rituximab | 0.6905 |
| ADC-24 | 0.4567 |

TABLE 1-4

| | EC₅₀, nM H2122 |
|---|---|
| Antibody or ADC | (CEACAM5/6⁺) |
| mAb1 | 7.942 |
| ADC-32 | 4.261 |

TABLE 1-5

| | EC₅₀, nM T47D |
|---|---|
| Antibody or ADC | (FRα⁺) |
| mAb2 | 0.2013 |
| ADC-33 | 0.1855 |

TABLE 1-6

| | EC₅₀, nM T47D |
|---|---|
| Antibody or ADC | (Nectin-4⁺) |
| mAb3 | 0.0433 |
| ADC-34 | 0.0496 |

Conclusions: From the data in Table 1-1, it could be seen that the ADC products of the present invention exhibit a cell binding activity similar to trastuzumab in the HCC1954 (HER2⁺) cell line. From the data in Table 1-2 to Table 1-6, it could be seen that the ADC products of the present invention targeting other tumor cell surface antigens such as CD38, CD20, CEACAM5/6, FR a and Nectin-4 exhibit a similar cell binding activity to the corresponding naked antibodies in the cell lines expressing the relevant antigens.

### Example 6: Inhibition of Cell Proliferation by Payloads Prepared According to the Present Invention In Vitro

### Experimental Methods

MDA-MB-453 (Cat. No. HTB-131, purchased from the cell bank of ATCC) and MDA-MB-468 (purchased from Cell Resource Center, Shanghai Academy of Biological Sciences, Chinese Academy of Sciences) were normally cultured in a DMEM medium (Gibco, Cat. No. 11965-092) with 10% fetal bovine serum (FBS) in a cell culture incubator at 37° C. and 5% CO₂. The cells were added to a 96-well plate at 1.0×10³ cells/well and 100 μL/well and incubated in a cell culture incubator at 37° C. and 5% CO₂. The test compounds were diluted with a DMEM medium containing 2% DMSO (Sigma-Aldrich, Cat. No. 34869) in a 4-fold gradient to the corresponding concentrations. Then 10 μL/well of the diluted test compounds at different concentrations (final concentrations: 250 nM, 62.5 nM, 15.6 nM, 3.91 nM, 0.98 nM, 0.24 nM, 0.061 nM, and 0.015 nM) or 10 μL/well of the control solution (DMEM containing 2% DMSO) were added to a 100 μL/well of cell culture system. The final concentration of DMSO was 0.2%. The cells were incubated in a cell culture incubator at 37° C. and 5% CO₂ for 6 days.

After 6 days of culture, 10 μL/well of Cell Counting Kit-8 reagent (CCK-8, Dojindo, Cat. No. CK04) was added to the wells and mixed well with shaking. The cells were incubated in a cell culture incubator at 37° C. and 5% CO₂ for 0.50-4.0 hours.

### Detection and Data Analysis

The OD450 optical density value was read on an EnVision™ multi-function microplate reader (PerkinElmer), and the inhibition rates (%) of the compounds were calculated. Then, the half-maximal inhibitory concentration (IC₅₀) values were obtained using the XLfit5 software (ID Business Solutions Limited). All samples were tested in duplicate wells.

$$Inhibition rate \% = ( 1 - ( OD 45 {0}_{test compound} - OD 45 {0}_{background} ) / ( OD 45 {0}_{cell} - OD 45 {0}_{background} ) ) \times 100 \%$$

wherein
OD450_{test compound} represents the optical density value in the wells of the compounds to be tested;
OD450_{cell} represents the optical density value in the wells of cell controls;
OD450_{background} represents the optical density value in the wells of cell-free controls.

The resulting data were showed in the following table:

TABLE 2

IC₅₀, nM

| Compound No. | MDA-MB-453 | MDA-MB-468 |
|---|---|---|
| Reference compound 1 | 7.9 | 6.0 |
| Compound 1 | 0.23 | 0.39 |
| Compound 2 | 0.31 | 0.41 |
| Compound 3 | 0.074 | 0.076 |
| Compound 4 | 0.89 | 0.71 |
| Compound 5 | 0.064 | 0.090 |
| Compound 6 | 0.25 | 0.32 |
| Compound 7 | 0.30 | 0.22 |
| Compound 8 | 0.32 | 0.21 |
| Compound 9 | 0.79 | 1.0 |
| Compound 10 | 2.3 | 1.5 |
| Compound 11 | 25.2 | 28.1 |
| Compound 12 | 3.3 | 7.9 |
| Compound 13 | 1.3 | 1.8 |
| Compound 14 | 0.39 | 0.41 |
| Compound 15 | 14.9 | 18.4 |
| Compound 16 | 61.6 | 91.4 |
| Compound 17 | 1.8 | 3.4 |
| Compound 18 | 0.15 | 0.091 |
| Compound 19 | 0.12 | 0.055 |
| Compound 20 | 0.43 | 1.0 |
| Compound 21 | 6.6 | 4.9 |
| Compound 22 | 6.5 | 9.2 |
| Compound 23 | 0.55 | 1.4 |
| Compound 24 | 0.24 | 0.20 |
| Compound 25 | 0.34 | 0.20 |
| Compound 26 | 1.8 | 1.9 |
| Compound 27 | 0.24 | 0.29 |
| Compound 28 | 0.16 | 0.24 |
| Compound 29 | 0.056 | 0.078 |
| Compound 30 | 0.41 | 0.64 |
| Compound 31 | 0.24 | 0.47 |
| Compound 32 | 0.22 | 0.24 |
| Compound 33 | 2.3 | 3.0 |
| Compound 34 | 2.8 | 3.4 |
| Compound 35 | 0.22 | 0.20 |

### Example 7: Inhibition of Cell Proliferation by the ADC Products of the Present Invention In Vitro

### Experimental Methods

HCC1954 cells (HER2 positive, purchased from the cell bank of ATCC, Cat. No. CRL-2338) were normally cultured in a RPMI-1640 medium (Gibco, Cat. No. A1049101) with 1000 fetal bovine serum (FBS) (Gibco, Cat. No. A5669701) in a cell culture incubator at 37° C. and 500 CO₂; and MDA-MB-468 cells (HER2 negative, purchased from Cell Resource Center, Shanghai Academy of Biological Sciences, Chinese Academy of Sciences) were both normally cultured in a DMEM medium with 10% fetal bovine serum (FBS) in a cell culture incubator at 37° C. and 5% CO₂. The cells were added to a 96-well plate at 1.0×10³ cells/well and 100 μL/well and incubated in a cell culture incubator at 37° C. and 500 CO₂. The test antibody-conjugated drugs were diluted with PBS (Gibco, Cat. No. C20012500BT) in a 4-fold gradient to the corresponding concentrations, and then 10 μL/well of the diluted test antibody-conjugated drugs at different concentrations (final concentrations: 500 nM, 125 nM, 31.3 nM, 7.8 nM, 2.0 nM, 0.49 nM, 0.12 nM, and 0.031 nM) or 10 μL/well of the control solution (PBS) were added to a 100 μL/well of cell culture system. The cells were incubated in a cell culture incubator at 37° C. and 5% CO₂ for 6 days.

After 6 days of culture, 10 μL/well of Cell Counting Kit-8 reagent (CCK-8, Dojindo, Cat. No. CK04) was added to the wells and mixed well with shaking. The cells were incubated in a cell culture incubator at 37° C. and 5% CO₂ for 0.50-4.0 hours.

### Detection and Data Analysis

The OD450 optical density values were read on an EnVision™ multi-function microplate reader (PerkinElmer), and the inhibition rates (%) of the test antibody-conjugated drugs were calculated. Then, the half-maximal inhibitory concentration (IC₅₀) values were obtained using the XLfit5 software (ID Business Solutions Limited). All samples were tested in duplicate wells.

$$Inhibition rate \% = ( 1 - ( OD 45 {0}_{test antibody - conjucated drug} - OD 45 {0}_{background} ) / ( OD 45 {0}_{cell} - OD 45 {0}_{background} ) ) \times 100 \%$$

wherein
OD450_{test antibody-conjugated drug} represents the optical density value in the wells of the antibody-conjugated drugs to be tested;
OD450_{cell} represents the optical density value in the wells of cell controls;
OD450_{background} represents the optical density value in the wells of cell-free controls.

For some cell proliferation experiments, the CTG method was used for testing, and the method was as follows:

After 6 days of treatment with the antibody-conjugated drugs to be tested, 50 μL/well of CellTiter-Glo® reagent (Promega, Cat. No. G9243) was added to the wells, mixed thoroughly with shaking and incubated for 10 minutes. Chemiluminescence values were read on an EnVision™ multi-function microplate reader (PerkinElmer). Others process were similar to the tests described above. The source and catalog number of cells selected for the inhibitory effect of other antibody-drug conjugates on cell proliferation were: Ramos cells (ATCC, Cat. No. CRL-1596), SU-DHL-6 cells (ATCC, Cat. No. CRL-2959), SU-DHL-2 cells (ATCC, Cat. No. CRL-2956), OPM-2 cells (Deutsche Sammlung von Mikroorganismen und Zelikulturen (DSMZ), Cat. No. ACC 50/G), H2122 cells (ATCC, Cat. No. CRL-5985), H1650 cells (ATCC, Cat. No. CRL-5883), T47D cells (ATCC, Cat. No. HTB-133), AMA-MB-436 cells (ATCC, Cat. No. HTB-130). The experimental methods were similar to those described above and were within the scope known to those skilled in the art, and the detection was carried out using suitable reagents and test methods. The resulting data was showed in the following table:

TABLE 3-1

IC₅₀, nM

| | HCC1954 | MDA-MB- | MDA-MB- |
|---|---|---|---|
| ADC No. | (HER2⁺) | 453 (HER2⁺) | 468 (HER2⁻) |
| ADC-1 | 0.20 | 0.73 | 29.5 |
| ADC-2 | | 0.65 | 28.1 |
| ADC-3 | | 0.28 | 22.0 |
| ADC-4 | 0.18 | 0.47 | 22.3 |
| ADC-5 | 0.10 | 0.048 | 56.8 |
| ADC-6 | 0.066 | 0.15 | 55.8 |
| ADC-7 | 0.46 | 0.54 | 32.6 |
| ADC-8 | 1.0 | 1.1 | 47.0 |
| ADC-9 | 30.1 | 5.3 | 75.0 |
| ADC-10 | 50.5 | 1.1 | 29.2 |
| ADC-11 | 30.3 | 0.38 | 35.3 |
| ADC-12 | 0.33 | 0.43 | 28.0 |
| ADC-13 | 0.36 | 0.47 | 30.7 |
| ADC-14 | 0.16 | 0.14 | 44.5 |
| ADC-15 | 0.15 | 0.15 | 47.0 |
| ADC-16 | 0.75 | 0.70 | 69.5 |
| ADC-17 | 0.61 | 0.55 | 84.4 |
| ADC-18 | | 0.50 | 32.8 |
| ADC-19 | 0.083 | 0.064 | 79.5 |
| ADC-20 | 0.20 | 0.11 | 75.3 |
| ADC-21 | 0.77 | 0.50 | 44.3 |
| ADC-22 | 8.0 | 0.51 | 36.1 |
| ADC-25 | 0.17 | 0.062 | 36.8 |
| ADC-26 | 0.81 | 0.52 | 66.8 |
| ADC-27 | 0.15 | 0.15 | 39.4 |
| ADC-28 | 0.36 | 0.27 | 39.8 |
| ADC-29 | 0.58 | 0.49 | 76.6 |
| ADC-30 | 139.4 | 2.4 | 97.6 |
| ADC-31 | 0.041 | <0.018 | 45.3 |

TABLE 3-2

IC₅₀, nM

| | Ramos cell | SU-DHL-6 cell | SU-DHL-2 cell |
|---|---|---|---|
| ADC No. | line (CD38⁺) | line (CD38⁺) | line (CD38⁻) |
| ADC-23 | 9.8 | 1.1 | 117.7 |

TABLE 3-3

IC₅₀, nM

| | Ramos cell | SU-DHL-6 cell | OPM2 cell |
|---|---|---|---|
| ADC No. | line (CD20⁺) | line (CD20⁺) | line (CD20⁻) |
| ADC-24 | 27.7 | 1.0 | 111.8 |

TABLE 3-4

IC₅₀, nM

| | H2122 cell line | H1650 cell line |
|---|---|---|
| ADC No. | (CEACAM 5/6⁺) | (CEACAM 5/6⁻) |
| ADC-32 | 0.29 | 57.5 |

TABLE 3-5

IC₅₀, nM

| | T47D cell line | MDA-MB-436 cell |
|---|---|---|
| ADC No. | (FRα⁺) | line (FRα⁻) |
| ADC-33 | 5.5 | 16.4 |

TABLE 3-6

IC₅₀, nM

| | T47D cell line | MDA-MB-436 cell |
|---|---|---|
| ADC No. | (Nectin-4⁺) | line (Nectin-4⁻) |
| ADC-34 | 1.1 | 15.3 |

Conclusions: According to the data in Table 3-1, the ADC products of the present invention exhibit proliferation inhibitory activity in vitro dependent on cellular HER2 expression, with strong proliferation inhibitory activity against HER2 positive cells and weak proliferation inhibitory activity against HER2 negative cells. From the data in Table 3-2 to Table 3-6, it could be seen that the ADC products targeting other tumor surface antigens also exhibit in vitro proliferation inhibitory activity dependent on the expression of tumor cell surface antigens (such as CD38, CD20, CEACAM5/6, FRα and Nectin-4), with strong proliferation inhibitory activity against antigen-positive cells and weak proliferation inhibitory activity against antigen-negative cells.

### Example 8: Plasma Stability Study of the Antibody-Drug Conjugates of the Present Invention

Objective: The stability of the antibody-drug conjugates of the present invention in plasma was evaluated by measuring the percent of payloads release from the antibody-drug conjugates in human plasma.

### Experimental Methods:

First, the ADC product solution was added to human plasma to achieve a concentration of 100 nM;
A 50 μL aliquot was added to a 96-well deep-well plate, and was incubated in a 5% CO₂ incubator at 37° C.;
The samples were transferred to ice at preset time points (0, 24, and 96 hours) to terminate the reaction, and then the samples were stored in a refrigerator at −80° C., in which 0-hour sample was collected within one minute of adding the ADC sample to the plasma
The concentration of the free payloads was determined by LC-MS/MS and the percent of payload release was calculated using the following formula:

$$Percent of payload release = free payload concentration / nominal conjucated payload concentration * 100 \%$$

The resulting data are shown in Table 4.

TABLE 4

The percent of payload release in human
plasma at different incubation times (%)

| ADC No. | 0 hour | 24 hours | 96 hours |
|---|---|---|---|
| ADC-1 | <0.0326% | 0.0619% | 0.0898% |
| ADC-2 | <0.0644% | <0.0644% | 0.112% |
| ADC-4 | 0.111% | <0.0633% | <0.0633% |
| ADC-6 | <0.0620% | <0.0620% | <0.0620% |
| ADC-13 | <0.0604% | <0.0604% | 0.0710% |
| ADC-14 | <0.0598% | <0.0598% | <0.0598% |
| ADC-15 | <0.0607% | <0.0607% | <0.0607% |
| ADC-25 | <0.0641% | <0.0641% | 0.118% |
| ADC-27 | <0.0635% | <0.0635% | <0.0635% |

Conclusions: From the data in Table 4, it can be seen that the percent of payload release from the ADCs described above in human plasma does not exceed 0.118%, and there is no significant difference in the payload release between different ADCs. The above results indicate that the antibody-drug conjugates of the present invention are stable in human plasma.

### Example 9: In Vivo Anti-Tumor Activity of the Antibody-Drug Conjugates of the Present Invention

Objective: The anti-tumor activity of the antibody-drug conjugates of the present invention was evaluated in HCC1954 subcutaneous tumor model in BALB/c nude mice.

Methods: Human breast ductal carcinoma cell line HCC1954 (purchased from ATCC) was cultured and passaged in RPMI1640 medium containing 10% fetal bovine serum. Tumor cells in the logarithmic growth phase were inoculated subcutaneously into the right flank of each female BALB/c nude mice (Shanghai Lingchang Biotechnology Co., Ltd.) at 7.5×10⁶ cells per mouse.

Mice were randomized into treatment and control groups according to the tumor volume. The antibody-drug conjugates ADC-1 and ADC-6 were diluted with normal saline, and administered according to the study design shown in Table 5. The day of grouping was defined as Day 0, and the next day after grouping was defined as Day 1. The ADC-1 and ADC-6 were prepared according to the method of Example 4.

Two orthogonal diameters of subcutaneous tumors and mouse body weight were measured 2-3 times per week. Tumor volume was calculated by the following formula:

$$TV = 1 / 2 {ab}^{2}$$

wherein a and b represent the length and width diameters of the tumor, respectively.

The tumor growth inhibition rate (TGI) was calculated by the following formula:

$$TGI = [ 1 - {( {TV}_{t} - {TV}_{0} )}_{treatment} / {( {TV}_{t} - {TV}_{0} )}_{control} ] \times 100 \%$$

wherein TV₀ represents the average tumor volume of the group prior to dosing (at day 0 of grouping), and TVₜ represents average tumor volume of the group after dosing.

Statistical analysis between treatment and control group was performed with Student's t test, p<0.05 were considered statistically significant.

### Results:

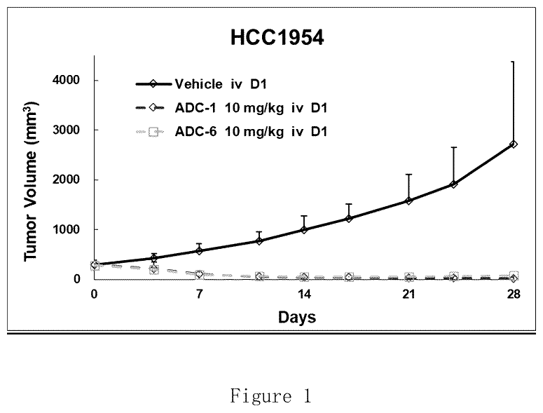

In the HCC1954 subcutaneous model, the test articles ADC-1 and ADC-6 were administered once at 10 mg/kg IV (injection via tail vein) on Day 1. On day 28, ADC-1 and ADC-6 treatment exhibited potent anti-tumor activity (FIG. 1), with tumor growth inhibition rate (TGI) of 111.3% and 108.2%, respectively. Both treatment groups were able to induce tumor regression, with reduced average tumor volume prior to treatment. No decrease in average body weight was observed in each group of mice treated with the test articles, indicating well tolerance of animals.

In conclusion, both of the ADC-1 and ADC-6 exhibited potent anti-tumor effects in the HCC1954 subcutaneous xenograft model (p<0.01).

TABLE 5
Dosage regimen of ADC-1 and ADC-6 in the
HCC1954 subcutaneous xenograft model

| | Dosage of | | |
|---|---|---|---|
| | administration | Dosage | Number of |
| Group | (mg/kg) | regimen | animals |
| Vehicle | 0 | IV, Day 1 | 9 |
| ADC-1 | 10 | IV, Day 1 | 9 |
| ADC-6 | 10 | IV, Day 1 | 9 |

Notes:
Vehicle: vehicle control; IV: intravenous injection; Day 1, the first day.

### Example 10: Kinase Activity Assay for Payloads Prepared According to the Present Invention

Determination of FRAP1 (mTOR) Kinase Activity

### 1. Reagents and Materials

Human FRAP1 (mTOR) recombinant protein (GST tag): Invitrogen, Cat. No. PV4753;
GFP-4E-BP1: Thermo Scientific, Cat. No. PV4759;
LanthaScreen™ Tb-p4E-BP1 (pThr46) antibody: Thermo Scientific, Cat. No. PV4755;
TR-FRET dilution buffer: Thermo Scientific, Cat. No. PV3574;
ATP solution (10 mM): Thermo Scientific, Cat. No. PV3227;
HEPES (1 M): Gibco, Cat. No. 15630-106;
EDTA (0.5 M), pH 8.0, RNase-free: Invitrogen, Cat. No. AM9260G;
Manganese chloride solution: Sigma-Aldrich, Cat. No. M1787;
EGTA: Sigma-Aldrich, Cat. No. E4378;
TWEEN®20: Sigma-Aldrich, Cat. No. P2287;
DTT: Sigma-Aldrich, Cat. No. DTT-RO;
384-well plate: Corning, Cat. No. 3676;
384-well plate: Corning, Cat. No. 4514;
Envision multi-mode plate reader: PerkinElmer;

### 2. Methods

### (1) Principle:

4EBP1 is the substrate of mTOR, and when mTOR is activated, it will phosphorylate Thr at position 46 of the 4EBP1 protein. LanthaScreen™ kinase assay is used to detect the amount of phosphorylated substrate GFP-4E-BP1 produced in kinase activity experiments, thereby judging the mTOR kinase activity, and through the strength of the kinase activity, distinguishing the inhibitory effects of different compounds on the activity of FRAP1 (mTOR) kinase. LanthaScreen™ kinase assay is typically divided into two steps: the kinase first converts ATP into ADP and phosphorylates the substrate GFP-4E-BP1; then a mixture of EDTA and Tb-p4E-BP1 (pThr46) antibody is added to terminate the kinase reaction. Because the added antibody is able to bind to the phosphorylation site of the substrate GFP-4E-BP1, it thus results in FRET from Tb to GFP. A strong signal of FRET indicates that more phosphorylated substrates are produced, and the inhibitory effect of the compound on the kinase is weak. On the contrary, it indicates that the compound has a strong inhibitory effect on the kinase.

### (2) Reagent Preparation:

1.33× kinase buffer: 5× kinase buffer (250 mM HEPES, 0.05% Tween 20, 5 mM EGTA, and 50 mM manganese chloride) was diluted with water to 1.33× kinase buffer, and 2.67 mM DTT was added;
FRAP1 (mTOR) enzyme solution: the kinase was dissolved in the 1.33× kinase buffer to achieve a final working concentration of 0.15 ng/μl;
ATP/GFP-4EBP1 mixture: The ATP stock solution (10 mM aqueous solution) and the kinase substrate GFP-4EBP1 were dissolved in the 1.33× kinase buffer, to achieve a final working concentration of 10 μM for ATP and a working concentration of 0.4 μM for the kinase substrate GFP-4EBP1;
EDTA and Tb-p4E-BP1 (pThr46) antibody mixture: EDTA solution and Tb-p4E-BP1 (pThr46) antibody were dissolved in TR-FRET dilution buffer, to achieve a final working concentration of 10 mM for EDTA and a working concentration of 2 nM for the Tb-p4E-BP1 (pThr46) antibody;
4× compound formulation: the compound was diluted in 3-fold gradient concentrations, and finally 4% DMSO aqueous solution containing different concentrations of the compound was obtained. The final concentrations of the test compound were 3 μM, 1 μM, 0.33 μM, 0.11 μM, 0.037 μM, 0.012 μM, 0.0041 μM, 0.0014 μM, 0.00046 μM, 0.00015 μM, and 0.000051 μM.

### (3) Specific Steps of the Experiment:

In the experiment, there were two control groups, one was 100% inhibition group (no kinase), another was 0% inhibition group (no inhibitor), each control group contained 16 replicate wells. The serially diluted compound was added to a 384-well plate at 2.5 μl/well with 2 replicate wells, and 4% aqueous DMSO solution was added to control wells. After that 2.5 μl FRAP1 (mTOR) enzyme solution was added to each well, except for the 100% inhibition group to which equal volume of buffer was added; then, 5 μl ATP/GFP-4EBP1 mixture was added to each well, and centrifuged at 1000 rpm for 60 seconds; Then, the 384-well plate was placed in a constant temperature incubator protected from light and incubated at room temperature for 1 hour. After the enzymatic reaction was completed, 10 μl of a mixture of EDTA and Tb-p4E-BP1 (pThr46) antibody was added to each well, the mixture was centrifuged at 1000 rpm for 60 seconds, then the 384-well plate was placed in a constant temperature incubator protected from light, and incubated at room temperature for 1 hour.

### 3. Detection

The 384-well plate was taken out and the plate was read using an Envision multi-mode plate reader (the excitation light wavelength was 320 nm and the emission light wavelengths were 495 nm and 520 nm).

### 4. Calculation

The inhibition rate was calculated using the formula as below:

$$Inhibition rate ( \% ) = 100 \% \times ( mean signal value of 0 \% inhibition group - signal value of testing well ) / ( mean signal value of 0 \% inhibition group - mean signal value of 100 \% inhibition group )$$

The mean of the signal values of the 100% inhibition group and the 0% inhibition group was used as the reference value, the inhibition rate (%) of each concentration of every compound was calculated according to the signal value of each well, and the IC₅₀ value was obtained by model 205 in XL-Fit 5.5 software (ID Business Solutions Limited).

Determination of PIK3CA/PIK3R1 (p110α/p85α) Kinase Activity

### 1. Reagents and Materials

Human PIK3CA/PIK3R1 (p110α/p85α) recombinant protein (His tag): Invitrogen, Cat. No. PV4788
Transcreener® ADP2 FP kit: BellBrook Labs, Cat. No. 3010-10K;
CHAPS hydrate: Sigma-Aldrich, Cat. No. C5070;
EGTA: Sigma-Aldrich, Cat. No. E4378;
DTT: Sigma-Aldrich, Cat. No. DTT-RO;
HEPES (1 M): Gibco, Cat. No. 15630-106;
EDTA (0.5 M), pH 8.0, RNase-free: Invitrogen, Cat. No. AM9260G;
ATP solution (10 mM): Thermo Scientific, Cat. No. PV3227;
PIP2: PS lipid kinase substrate: Thermo Scientific, Cat. No. PV5100;
MgCl₂ (1 M): Invitrogen, Cat. No. AM9530G;
NaCl (5 M), no RNase: Invitrogen, Cat. No. AM9759;
96-well plate: Greiner, Cat. No. 675076;
Envision multi-mode plate reader: PerkinElmer;

### 2. Methods

### (1) Principle:

Transcreener® ADP2 FP kit can be used to detect the amount of ADP produced in a kinase activity experiment, thereby judging the PIK3CA/PIK3R1 (p110 α/p85 α) kinase activity, and through the strength of the kinase activity, distinguishing the inhibitory effects of different compounds on the activity of PIK3CA/PIK3R1 (p110 α/p85 α) kinase. Transcreener® ADP2 FP assay is typically divided into two steps: the kinase first converts ATP into ADP and phosphorylates the substrate PIP2: PS; then, the ADP detection mixture (the mixture contains stop and detection buffers, as well as the ADP Alexa633 Tracer and ADP² antibody) is added, and it could terminate the kinase reaction. The complex formed by the binding of the ADP Alexa633 Tracer to the ADP2 antibody therein could produce fluorescence polarization. The kinase-generated ADP replaces the tracer-labeled ADP, and binds to the ADP² antibody, causing the substituted tracer to be free, which results in a decrease in the fluorescence polarization signal. A strong fluorescence polarization signal indicates that little ADP is produced, and the compound has a strong inhibitory effect on the kinase; on the contrary, it indicates that the compound has a weak inhibitory effect on the kinase. Assay is carried out according to the instruction of the kit.

### (2) Reagent Preparation:

1.25× kinase buffer: comprising 62.5 mM HEPES, 125 mM sodium chloride, 1.25 mM EGTA, 0.0375% CHAPS, 3.75 mM magnesium chloride, and 1.25 mM DTT;
PIK3CA/PIK3R1 (p110α/p85α) enzyme solution: the kinase was dissolved in the 1.25× kinase buffer to achieve a final working concentration of 0.096 ng/ul;
A mixed solution of ATP and PIP2:PS lipid kinase substrate: the ATP stock solution (10 mM aqueous solution) and PIP2:PS lipid kinase substrate were dissolved in the 1.25× kinase buffer, to achieve a final working concentration of 10 μM for ATP and a working concentration of 30 μM for the PIP2:PS lipid kinase substrate;
5× compound formulation: the compound was diluted in 3-fold gradient concentrations, and finally 10% DMSO aqueous solution containing different concentrations of the compound was obtained. The final concentrations of the test compound were 100 nM, 33.3 nM, 11.1 nM, 3.70 nM, 1.23 nM, 0.41 nM, 0.14 nM, and 0.046 nM.

### (3) Specific Steps of the Experiment:

In the experiment, there were two control groups, one was 100% inhibition group (with 90 mM EDTA), another was 0% inhibition group (no inhibitor), each control group contained 4 replicate wells. 5 μl serially diluted compound was added to each well of a 96-well plate with 2 replicate wells, and 5 μl 10% aqueous DMSO solution was added for 0% inhibition group and equal volume of a mixture of 0.45 M EDTA and 10% DMSO was added for 100% inhibition group. After that, 10 μl PIK3CA/PIK3R1 (p110α/p85α) enzyme solution was added to each well; then, 10 μl of a mixed solution of ATP and PIP2:PS lipid kinase substrate was added to each well, and the mixture was centrifuged at 1000 rpm for 60 seconds; Then, the 96-well plate was placed in a constant temperature incubator protected from light and incubated at room temperature for 1 hour. After the enzymatic reaction was completed, 25 μl of ADP detection mixture was added to each well, the mixture was centrifuged at 1000 rpm for 60 seconds, then the 96-well plate was placed in a constant temperature incubator protected from light, and incubated at room temperature for 1.5 hour.

10 μM ATP/ADP standard curve: 5 μl of 10% DMSO solution and 10 μl of 1.25× kinase buffer were added to the wells of the standard curve plate, respectively. After that, ATP and ADP were formulated into a mixture according to different conversion rates, and 10 μl of this mixture (single well) was added to the well of the corresponding plate. After the enzymatic reaction was completed, 25 μl of ADP detection mixture was added to each well, the mixture was centrifuged at 1000 rpm for 60 seconds, then the 96-well plate was placed in a constant temperature incubator protected from light, and incubated at room temperature for 1.5 hour. In the standard curve, the ATP conversion rates were 100%, 50%, 20%, 10%, 5%, 2.5%, 1%, 0.8%, 0.6%, 0.4%, 0.2%, and 0.1%, respectively.

Fluorescence polarization signal was calculated in the control groups, including negative control group, positive control group, and blank control group. 5 μl of 10% DMSO solution and 20 μl of 1.25× kinase buffer were added to the 96-well plate, respectively. After the enzymatic reaction was completed, 25 μl of ADP detection mixture was added to the positive control group, 25 μl of ADP detection mixture without ADP² antibody was added to the negative control group, and 25 μl of ADP detection mixture without tracer-label ADP and ADP² antibody was added to the blank control group. Then centrifugation was performed at 1000 rpm for 60 seconds. The 96-well plate was placed in a constant temperature incubator protected from light, and incubated at room temperature for 1.5 hours.

### 3. Detection

The 96-well plates were taken out and the plates were read using an Envision multi-mode plate reader to detect the vertical polarization S value and the horizontal polarization P value (the excitation light wavelength was 620 nm and the emission light wavelength was 688 nm).

### 4. Calculation

$$Calculation of G factor \text{:} G = ( ( 1000 - tracer mP ) \times negative control group corrected mean S ) / ( ( 1000 + tracer mP ) \times negative control group corrected mean P )$$

Corrected S is the S value of the detection well−the mean S of the blank control group

Corrected P is the P value of the test well−the mean P of the blank control group

$$Fluorescence polarization value mP \text{:} mP = [ \text{} ( corrected S - corrected P \times G ) / ( corrected S + corrected P \times G ) ] \times 1000$$

The inhibition rate was calculated using the formula as below:

$$\% inhibition rate ( IR ) = [ \text{} ( mP sample to be tested - mP 0 \% inhibition group ) / ( mP 100 \% inhibition group - mP 0 \% inhibition group ) ] \times 100 \%$$

The mean of the signal values of the 1006 inhibition group and the 00 inhibition group was used as the reference value, the inhibition rate (0%) of each concentration of every compound was calculated according to the signal value of each well, and the C₅₀ value was obtained by model 205 in XL-Fit 5.5 software (ID Business Solutions Limited).

### Test Results

TABLE 6

| | PIK3CA/PIK3R1 | FRAP1 (mTOR) |
|---|---|---|
| | (p110α/p85α) | (Enzyme, |
| Compound No. | (Enzyme, IC₅₀, μM) | IC₅₀, μM) |
| Reference compound 1 | 0.0011 | 0.0053 |
| Compound 1 | 0.0005 | 0.0036 |
| Compound 2 | 0.0007 | 0.0063 |
| Compound 5 | 0.0016 | 0.0075 |
| Compound 7 | 0.0005 | 0.0027 |
| Compound 14 | 0.0006 | 0.0112 |
| Compound 18 | 0.0021 | 0.0062 |
| Compound 19 | 0.0014 | 0.0086 |
| Compound 23 | 0.0007 | 0.0034 |
| Compound 24 | 0.0018 | 0.0053 |
| Compound 25 | 0.0026 | 0.0108 |
| Compound 27 | 0.0015 | 0.0064 |
| Compound 28 | 0.0006 | 0.0028 |
| Compound 29 | 0.0014 | 0.0014 |
| Compound 30 | 0.0023 | 0.0046 |
| Compound 32 | 0.0028 | 0.0022 |

### Determination of ATM(h)/ATR/ATRIP(h)/DNA-PK(h) Kinase Activity

### 1. Reagents and Materials

The kinase sources are shown in the table below.

| | Cat. | | Kinase | |
|---|---|---|---|---|
| Kinase name | No. | Species | type | Registration No. |
| ATM(h) | 14-933 | Human | Atypical | GenBank NM_000051.3 |
| | | derived | | |
| ATR/ATRIP(h) | 14-953 | Human | Atypical | GenBank NM_001184.3 |
| | | derived | | (ATR)/GenBank |
| | | | | AF451323.1 (ATRIP) |
| DNA-PK(h) | 14-950 | Human | Atypical | GenBank NM_006904.6 |
| | | derived | | |

### 2. Methods

The compounds synthesized according to the present invention (compound 27, compound 28, compound 29, compound 30, compound 31, and compound 32) were commissioned to an external institution Eurofins Cerep (https://www.eurofins.com/) to perform corresponding protein kinase experiments. The lipid kinases, ATM(h), ATR/ATRIP(h), and DNA-PK (h) were detected by HTRFR.

### 3. Data Analysis

The calculation formula was as follows:

### HTRFR Method:

$$Remained kinase activity = \frac{\begin{matrix}Average readout of sample wells - \\ Average readout of wells without enzymes\end{matrix}}{\begin{matrix}Average readout of control wells - \\ Average readout of wells without enzymes\end{matrix}}$$

For IC₅₀ determination, the data were analyzed using XLFit version 5.3 (ID Business Solutions).

### 4. Test Results

TABLE 7

| | ATM(h) | ATR/ATRIP(h) | DNA-PK(h) |
|---|---|---|---|
| | (enzyme, | (enzyme, | (enzyme, |
| Compound | IC₅₀, μM) | IC₅₀, μM) | IC₅₀, μM) |
| Compound 1 | 0.0005 | 0.009 | 0.0001 |
| Compound 2 | 0.001 | 0.013 | 0.0004 |
| Compound 27 | 0.0007 | 0.017 | 0.0002 |
| Compound 28 | 0.0006 | 0.013 | 0.0003 |
| Compound 29 | 0.0007 | 0.020 | 0.0003 |
| Compound 30 | 0.001 | 0.036 | 0.0006 |
| Compound 31 | 0.0004 | 0.015 | 0.0002 |
| Compound 32 | 0.0006 | 0.017 | 0.0003 |

### Sequence Information

| SEQ ID | | |
|---|---|---|
| NO | | |
| | Trastuzumab | |
| 1 | HCDR1 | DTYIH |
| 2 | HCDR2 | RIYPTNGYTRYADSVKG |
| 3 | HCDR3 | WGGDGFYAMDY |
| 4 | LCDR1 | RASQDVNTAVA |
| 5 | LCDR2 | SASFLYS |
| 6 | LCDR3 | QQHYTTPPT |
| 7 | heavy chain | EVQLVESGGGLVQPGGSLRLSCAASGFNIKDTYIHWVRQAPGKGLEWV |
| | variable | ARIYPTNGYTRYADSVKGRFTISADTSKNTAYLQMNSLRAEDTAVYYC |
| | region | SRWGGDGFYAMDYWGQGTLVTVSS |
| 8 | light chain | DIQMTQSPSSLSASVGDRVTITCRASQDVNTAVAWYQQKPGKAPKLLI |
| | variable | YSASFLYSGVPSRFSGSRSGTDFTLTISSLQPEDFATYYCQQHYTTPPTF |
| | region | GQGTKVEIK |
| 9 | heavy | EVQLVESGGGLVQPGGSLRLSCAASGFNIKDTYIHWVRQAPGKGLEW |
| | chain | VARIYPTNGYTRYADSVKGRFTISADTSKNTAYLQMNSLRAEDTAVY |
| | | YCSRWGGDGFYAMDYWGQGTLVTVSSASTKGPSVFPLAPSSKSTSGG |
| | | TAALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALP |
| | | APIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSV |
| | | MHEALHNHYTQKSLSLSPGK |
| 10 | light | DIQMTQSPSSLSASVGDRVTITCRASQDVNTAVAWYQQKPGKAPKLLIYSASF |
| | chain | LYSGVPSRFSGSRSGTDFTLTISSLQPEDFATYYCQQHYTTPPTFGQGTKVEIKR |
| | | TVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | Daratumumab | |
| 11 | HCDR1 | SFAMS |
| 12 | HCDR2 | AISGSGGGTYYADSVKG |
| 13 | HCDR3 | DKILWFGEPVFDY |
| 14 | LCDR1 | RASQSVSSYLA |
| 15 | LCDR2 | DASNRAT |
| 16 | LCDR3 | QQRSNWPPT |
| 17 | heavy chain | EVQLLESGGGLVQPGGSLRLSCAVSGFTFNSFAMSWVRQAPGKGLEW |
| | variable | VSAISGSGGGTYYADSVKGRFTISRDNSKNTLYLQMNSLRAEDTAVYF |
| | region | CAKDKILWFGEPVFDYWGQGTLVTVSS |
| 18 | light chain | EIVLTQSPATLSLSPGERATLSCRASQSVSSYLAWYQQKPGQAPRLLIYD |
| | variable | ASNRATGIPARFSGSGSGTDFTLTISSLEPEDFAVYYCQQRSNWPPTFGQ |
| | region | GTKVEIK |
| 19 | heavy | EVQLLESGGGLVQPGGSLRLSCAVSGFTFNSFAMSWVRQAPGKGLEW |
| | chain | VSAISGSGGGTYYADSVKGRFTISRDNSKNTLYLQMNSLRAEDTAVYF |
| | | CAKDKILWFGEPVFDYWGQGTLVTVSSASTKGPSVFPLAPSSKSTSGG |
| | | TAALGCLVKDYFPEPVTVSWNSGALTSGVHTFPAVLQSSGLYSLSSVV |
| | | TVPSSSLGTQTYICNVNHKPSNTKVDKRVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALP |
| | | APIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSV |
| | | MHEALHNHYTQKSLSLSPGK |
| 20 | light | EIVLTQSPATLSLSPGERATLSCRASQSVSSYLAWYQQKPGQAPRLLIY |
| | chain | DASNRATGIPARFSGSGSGTDFTLTISSLEPEDFAVYYCQQRSNWPPTF |
| | | GQGTKVEIKRTVAAPSVFIFPPSDEQLKSGTASVVCLLNNFYPREAKVQ |
| | | WKVDNALQSGNSQESVTEQDSKDSTYSLSSTLTLSKADYEKHKVYACE |
| | | VTHQGLSSPVTKSFNRGEC |
| | Rituximab | |
| 21 | HCDR1 | SYNMH |
| 22 | HCDR2 | AIYPGNGDTSYNQKFKG |
| 23 | HCDR3 | STYYGGDWYFNV |
| 24 | LCDR1 | RASSSVSYIH |
| 25 | LCDR2 | ATSNLAS |
| 26 | LCDR3 | QQWTSNPPT |
| 27 | heavy chain | QVQLQQPGAELVKPGASVKMSCKASGYTFTSYNMHWVKQTPGRGLE |
| | variable | WIGAIYPGNGDTSYNQKFKGKATLTADKSSSTAYMQLSSLTSEDSAVY |
| | region | YCARSTYYGGDWYFNVWGAGTTVTVSA |
| 28 | light chain | QIVLSQSPAILSASPGEKVTMTCRASSSVSYIHWFQQKPGSSPKPWIYAT |
| | variable | SNLASGVPVRFSGSGSGTSYSLTISRVEAEDAATYYCQQWTSNPPTFGG |
| | region | GTKLEIK |
| 29 | heavy | QVQLQQPGAELVKPGASVKMSCKASGYTFTSYNMHWVKQTPGRGLEWIGAI |
| | chain | YPGNGDTSYNQKFKGKATLTADKSSSTAYMQLSSLTSEDSAVYYCARSTYYG |
| | | GDWYFNVWGAGTTVTVSAASTKGPSVFPLAPSSKSTSGGTAALGCLVKDYFP |
| | | EPVTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 30 | light | QIVLSQSPAILSASPGEKVTMTCRASSSVSYIHWFQQKPGSSPKPWIYATSNLAS |
| | chain | GVPVRFSGSGSGTSYSLTISRVEAEDAATYYCQQWTSNPPTFGGGTKLEIKRTV |
| | | AAPSVFIFPPSDEQLKSGTASVVCLLNNFYPREAKVQWKVDNALQSGNSQESV |
| | | TEQDSKDSTYSLSSTLTLSKADYEKHKVYACEVTHQGLSSPVTKSFNRGEC |
| | NEO-201 | |
| | (mAb1) | |
| 31 | HCDR1 | DYAMH |
| 32 | HCDR2 | GDYSGSRYWFAYNQNFQG |
| 33 | HCDR3 | GDYSGSRYWFAY |
| 34 | LCDRI | QASENIYGALN |
| 35 | LCDR2 | GASNLAT |
| 36 | LCDR3 | QQVLSSPYT |
| 37 | heavy chain | QVQLVQSGAEVKKPGASVKVSCKASGYTFTDYAMHWVRQAPGQRLEWMGL |
| | variable | ISTYSGDTKYNQNFQGRVTMTVDKSASTAYMELSSLRSEDTAVYYCARGDYS |
| | region | GSRYWFAYWGQGTLVTVSS |
| 38 | light chain | DIQMTQSPSSLSASVGDRVTITCQASENIYGALNWYQRKPGKSPKLLIYGASNL |
| | variable | ATGMPSRFSGSGSGTDYTFTISSLQPEDIATYYCQQVLSSPYTFGGGTKLEIK |
| | region | |
| 39 | heavy | QVQLVQSGAEVKKPGASVKVSCKASGYTFTDYAMHWVRQAPGQRLEWMGL |
| | chain | ISTYSGDTKYNQNFQGRVTMTVDKSASTAYMELSSLRSEDTAVYYCARGDYS |
| | | GSRYWFAYWGQGTLVTVSSASTKGPSVFPLAPSSKSTSGGTAALGCLVKDYF |
| | | PEPVTVSWNSGALTSGVHTFPAVLQSSGLYSLSSVVTVPSSSLGTQTYICNVNH |
| | | KPSNTKVDKKVEPKSCDKTHTCPPCPAPELLGGPSVFLFPPKPKDTLMISRTPE |
| | | VTCVVVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSRDELTKNQ |
| | | VSLTCLVKGFYPSDIAVEWESNGQPENNYKTTPPVLDSDGSFFLYSKLTVDKS |
| | | RWQQGNVFSCSVMHEALHNHYTQKSLSLSPGK |
| 40 | light | DIQMTQSPSSLSASVGDRVTITCQASENIYGALNWYQRKPGKSPKLLIYGASNL |
| | chain | ATGMPSRFSGSGSGTDYTFTISSLQPEDIATYYCQQVLSSPYTFGGGTKLEIKRT |
| | | VAAPSVFIFPPSDEQLKSGTASVVCLLNNFYPREAKVQWKVDNALQSGNSQES |
| | | VTEQDSKDSTYSLSSTLTLSKADYEKHKVYACEVTHQGLSSPVTKSFNRGEC |
| | Enfortumab | |
| | (mAb3) | |
| 41 | HCDR1 | SYNMN |
| 42 | HCDR2 | YISSSSSTIYYADSVKG |
| 43 | HCDR3 | AYYYGMDV |
| 44 | LCDR1 | RASQGISGWLA |
| 45 | LCDR2 | AASTLQS |
| 46 | LCDR3 | QQANSFPPT |
| 47 | heavy chain | EVQLVESGGGLVQPGGSLRLSCAASGFTFSSYNMNWVRQAPGKGLEWVSYIS |
| | variable | SSSSTIYYADSVKGRFTISRDNAKNSLSLQMNSLRDEDTAVYYCARAYYYGM |
| | region | DVWGQGTTVTVSS |
| 48 | light chain | DIQMTQSPSSVSASVGDRVTITCRASQGISGWLAWYQQKPGKAPKFLIYAAST |
| | variable | LQSGVPSRFSGSGSGTDFTLTISSLQPEDFATYYCQQANSFPPTFGGGTKVEIK |
| | region | |
| 49 | heavy | EVQLVESGGGLVQPGGSLRLSCAASGFTFSSYNMNWVRQAPGKGLEWVSYIS |
| | chain | SSSSTIYYADSVKGRFTISRDNAKNSLSLQMNSLRDEDTAVYYCARAYYYGM |
| | | DVWGQGTTVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKRVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQ |
| | | GNVFSCSVMHEALHNHYTQKSLSLSPGK |
| 50 | light | DIQMTQSPSSVSASVGDRVTITCRASQGISGWLAWYQQKPGKAPKFLIYAAST |
| | chain | LQSGVPSRFSGSGSGTDFTLTISSLQPEDFATYYCQQANSFPPTFGGGTKVEIKR |
| | | TVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| | mirvetuximab | |
| | (mAb2) | |
| 51 | HCDR1 | GYFMN |
| 52 | HCDR2 | RIHPYDGDTFYNQKFQG |
| 53 | HCDR3 | YDGSRAMDY |
| 54 | LCDR1 | KASQSVSFAGTSLMH |
| 55 | LCDR2 | RASNLEA |
| 56 | LCDR3 | QQSREYPYT |
| 57 | heavy chain | QVQLVQSGAEVVKPGASVKISCKASGYTFTGYFMNWVKQSPGQSLEWIGRIH |
| | variable | PYDGDTFYNQKFQGKATLTVDKSSNTAHMELLSLTSEDFAVYYCTRYDGSRA |
| | region | MDYWGQGTTVTVSS |
| 58 | light chain | DIVLTQSPLSLAVSLGQPAIISCKASQSVSFAGTSLMHWYHQKPGQQPRLLIYR |
| | variable | ASNLEA |
| | region | GVPDRFSGSGSKTDFTLTISPVEAEDAATYYCQQSREYPYTFGGGTKLEIK |
| 59 | heavy | QVQLVQSGAEVVKPGASVKISCKASGYTFTGYFMNWVKQSPGQSLEWIGRIH |
| | chain | PYDGDTFYNQKFQGKATLTVDKSSNTAHMELLSLTSEDFAVYYCTRYDGSRA |
| | | MDYWGQGTTVTVSSASTKGPSVFPLAPSSKSTSGGTAALGCLVKDYFPEPVTV |
| | | SWNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQDWL |
| | | NGKEYKCKVSNKALPAPIEKTISKAKGQPREPQVYTLPPSRDELTKNQVSLTCL |
| | | VKGFYPSDIAVEWESNGQPENNYKTTPPVLDSDGSFFLYSKLTVDKSRWQQG |
| | | NVFSCSVMHEALHNHYTQKSLSLSPGK |
| 60 | light | DIVLTQSPLSLAVSLGQPAIISCKASQSVSFAGTSLMHWYHQKPGQQPRLLIYR |
| | chain | ASNLEAGVPDRFSGSGSKTDFTLTISPVEAEDAATYYCQQSREYPYTFGGGTK |
| | | LEIKRTVAAPSVFIFPPSDEQLKSGTASVVCLLNNFYPREAKVQWKVDNALQS |
| | | GNSQESVTEQDSKDSTYSLSSTLTLSKADYEKHKVYACEVTHQGLSSPVTKSF |
| | | NRGEC |

1.-81. (canceled)

82. A drug conjugate having a structure represented by formula (I): 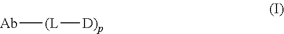
or a pharmaceutically acceptable salt thereof, or a solvate, a racemic mixture, an enantiomer, a diastereomer or a tautomer thereof, wherein
Ab is an anti-HER2 antibody or the antigen-binding fragment thereof comprises: 1, 2, 3, 4, 5, or 6 CDRs of trastuzumab; 1, 2, and 3 CDRs of the heavy chain variable region, i.e., HCDR1, HCDR2, and HCDR3, of trastuzumab; 1, 2, and 3 CDRs of the light chain variable region, i.e., LCDR1, LCDR2, and LCDR3, of trastuzumab; 3 CDRs of the heavy chain variable region and 3 CDRs of the light chain variable region of trastuzumab; a heavy chain variable region of trastuzumab; a light chain variable region of trastuzumab; a heavy chain variable region and a light chain variable region of trastuzumab; a heavy chain of trastuzumab; a light chain of trastuzumab; a heavy chain and a light chain of trastuzumab; alternatively, the anti-HER2 antibody or the antigen-binding fragment thereof comprises or consists of the two said heavy chains and the two said light chains of trastuzumab;
the structure of the D is: 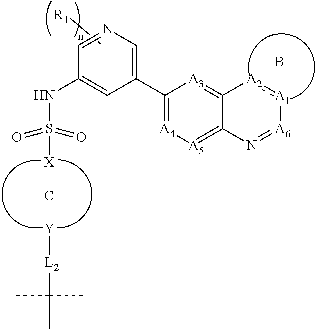
wherein:
the structure of 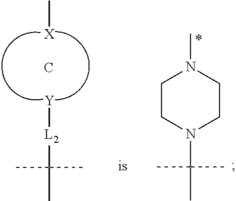
wherein * represents the site to which the parent core structure is linked; [symbol]
represents the site to which the linker L is linked;
R₁ is selected from the group consisting of: —O(C₁₋₆ alkyl), —OC₁₋₆ haloalkyl, or C₃₋₉ cycloalkyl, wherein the C₁₋₆ alkyl is optionally substituted with one or more deuterium;
u, at each occurrence, is independently 0, 1, 2, or 3;
A₃, A₄, A₅, and A₆ are all CH, or A₃ is N, and A₄, A₅, and A₆ are all CH; 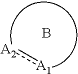
is selected from the following structures: 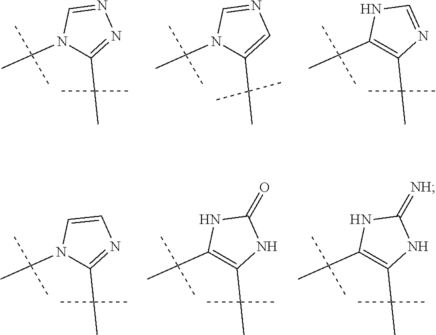
each of which is optionally substituted with 1, 2, or 3 R₃, each R₃ is independently selected from the group consisting of: H, CN, C₁₋₆ alkyl, C₃₋₉ cycloalkyl, and 3- to 12-membered heterocyclyl, wherein the C₁₋₆ alkyl and C₃₋₉ cycloalkyl can each optionally be substituted with one or more groups independently selected from the group consisting of: deuterium, —OH, C₁₋₆ alkylene-OH, halogen, and —C₁₋₆ alkylene-OC₁₋₆ alkyl; and
the structure of -L-D is: 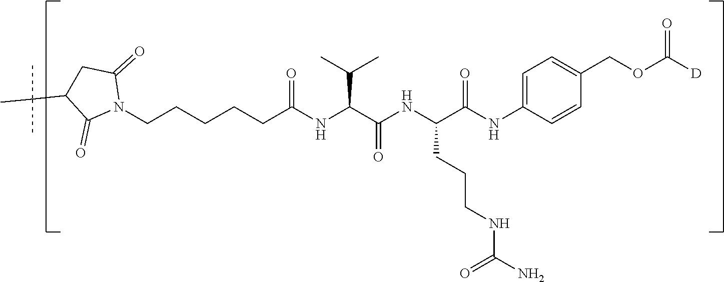
wherein the dashed lines represent the site to which Ab is linked; and
p is an integer from 1 to 20.

83. The drug conjugate or the pharmaceutically acceptable salt thereof, or the solvate, the racemic mixture, the enantiomer, the diastereomer or the tautomer thereof according to claim 82, wherein 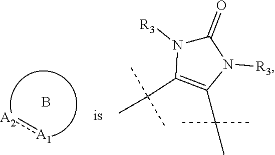
and each R₃ is independently as defined in claim 82.

84. The drug conjugate or the pharmaceutically acceptable salt thereof, or the solvate, the racemic mixture, the enantiomer, the diastereomer or the tautomer thereof according to claim 82, wherein 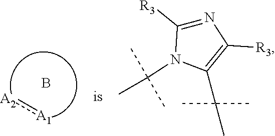
and each R₃ is independently H, —CH₃, or C₃₋₉ cycloalkyl.

85. The drug conjugate or the pharmaceutically acceptable salt thereof, or the solvate, the racemic mixture, the enantiomer, the diastereomer or the tautomer thereof according to claim 82, wherein R₁ is —O(C₁₋₆ alkyl).

86. The drug conjugate or the pharmaceutically acceptable salt thereof, or the solvate, the racemic mixture, the enantiomer, the diastereomer or the tautomer thereof according to claim 82, wherein each R₃ is independently selected from the group consisting of: hydrogen, —CH₃, —CD₃, —CH(CH₃)₂, —CH₂CF₃, or the following groups:

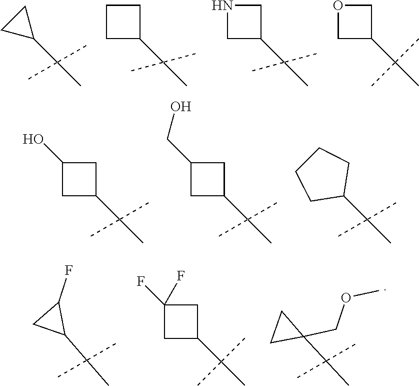

87. The drug conjugate or the pharmaceutically acceptable salt thereof, or the solvate, the racemic mixture, the enantiomer, the diastereomer or the tautomer thereof according to claim 82, wherein D is selected from the group consisting of: 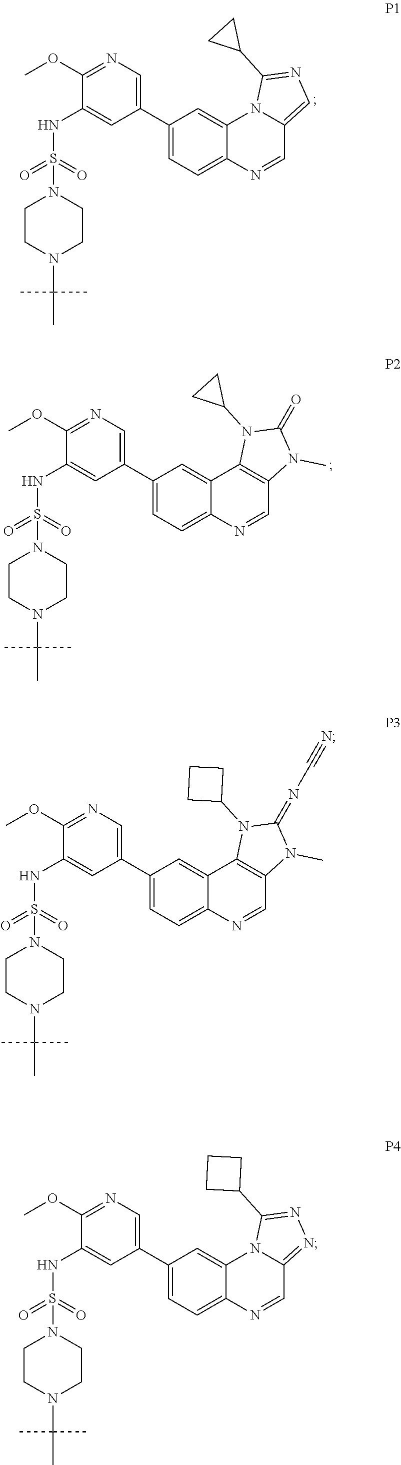 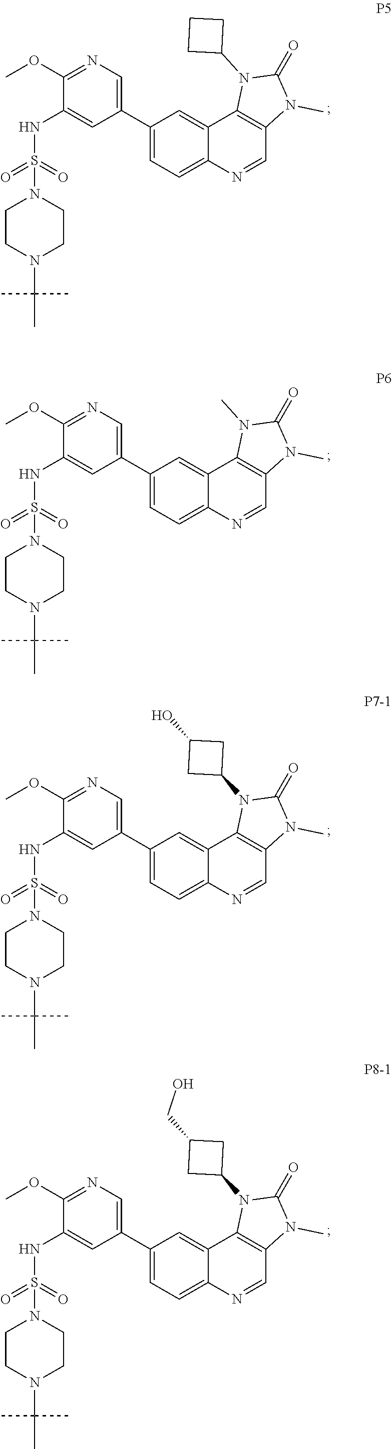 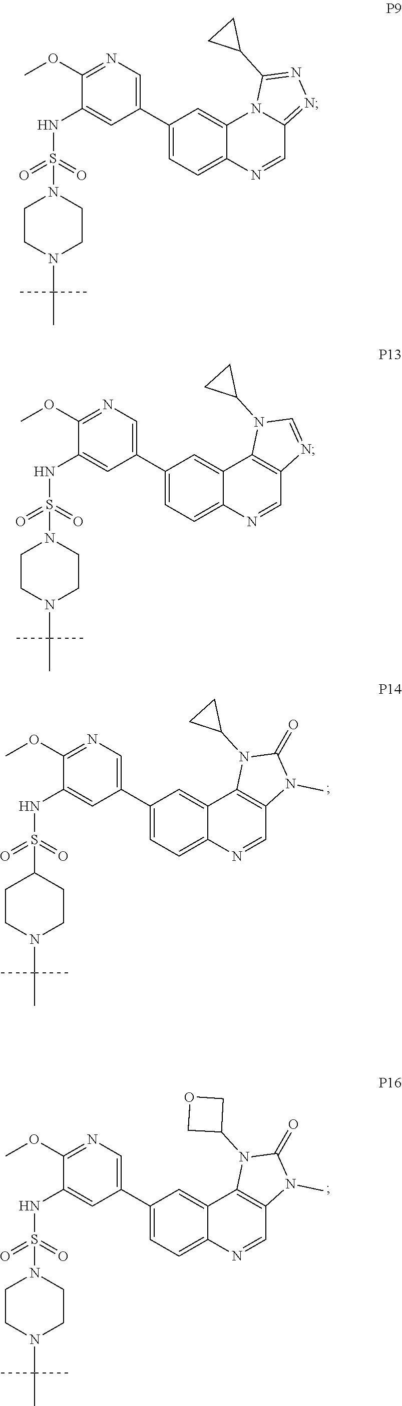 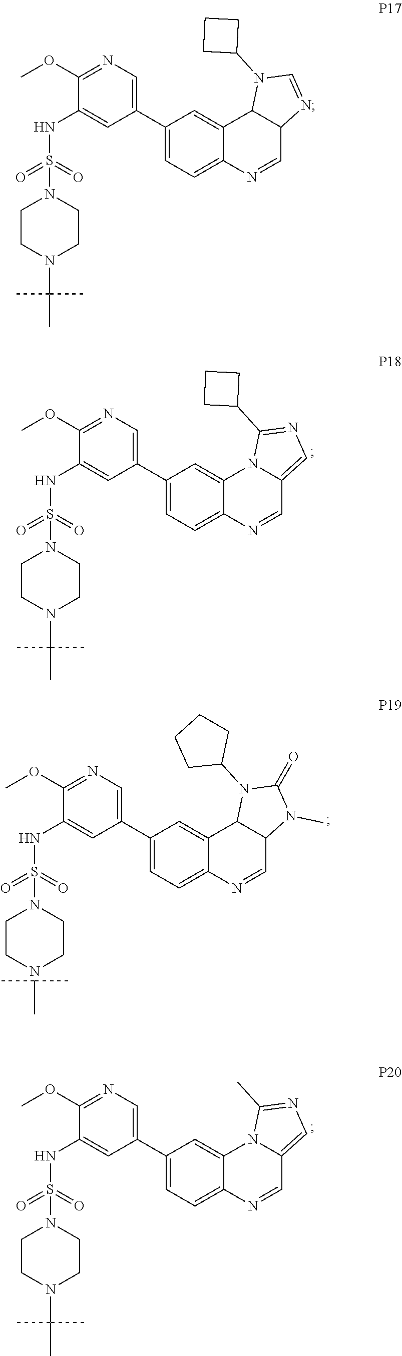 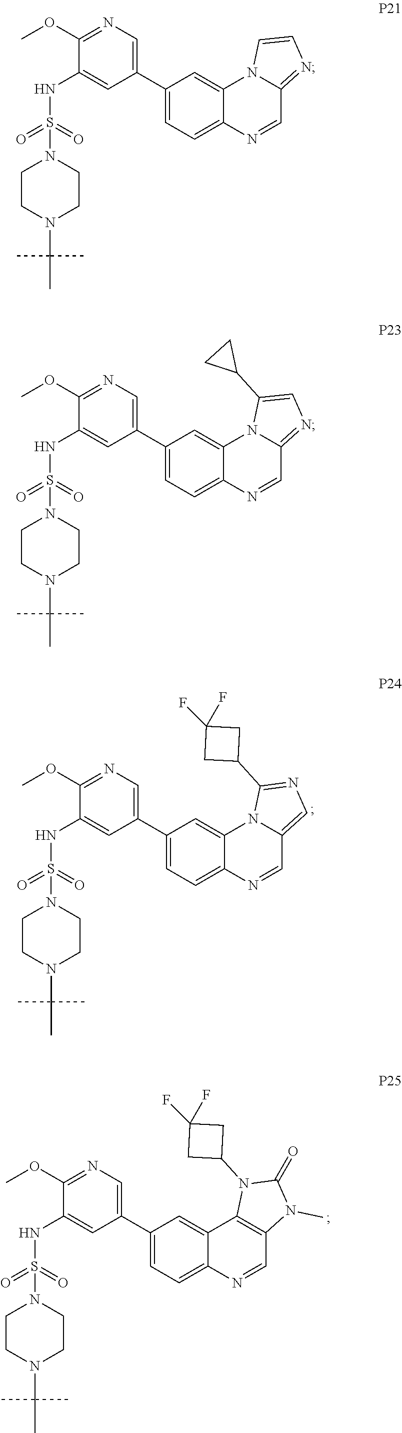 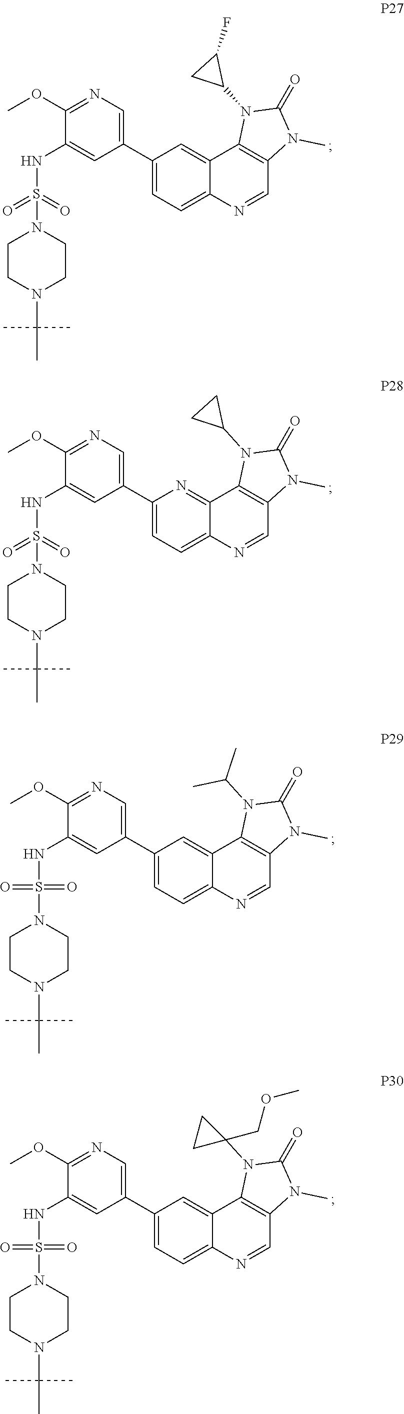 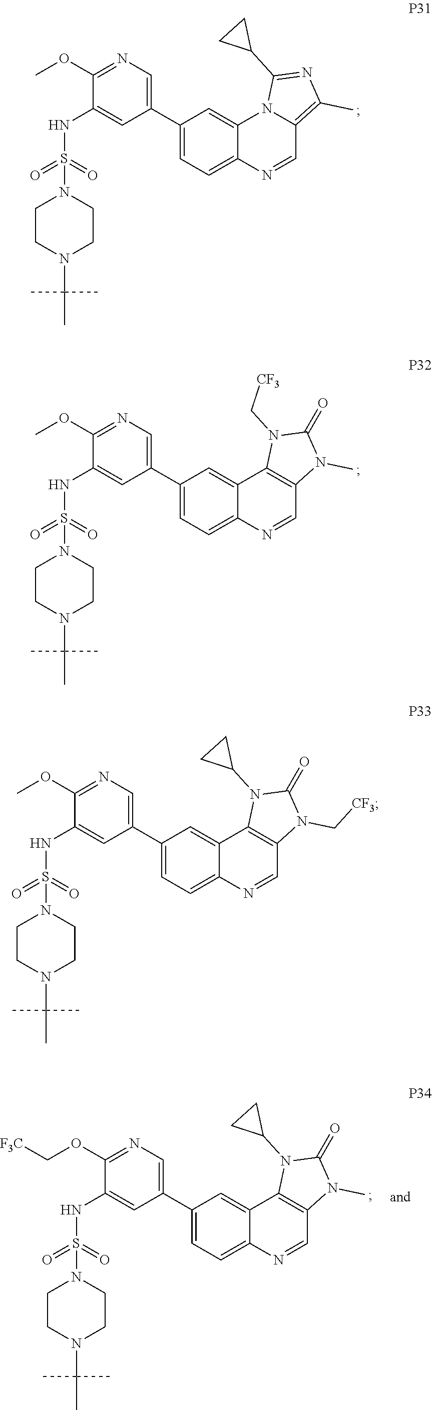 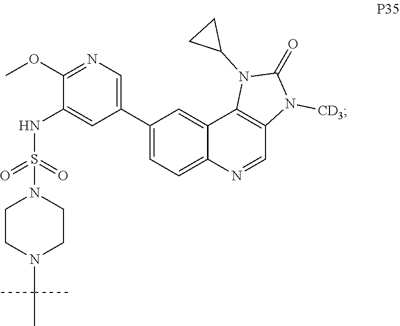
wherein the dashed lines represent the site where D is linked to the linker L.

88. The drug conjugate or the pharmaceutically acceptable salt thereof, or the solvate, the racemic mixture, the enantiomer, the diastereomer or the tautomer thereof according to claim 82, wherein -L-D is selected from the group consisting of:
Structure 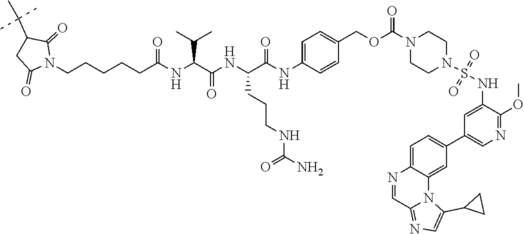 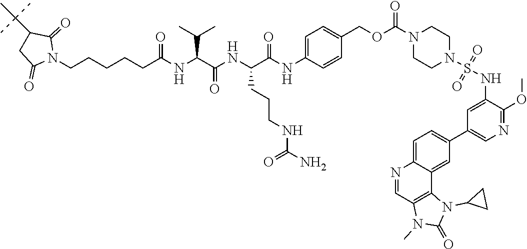 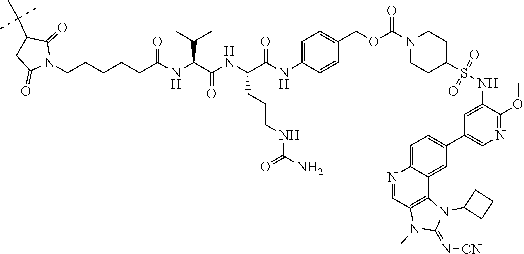 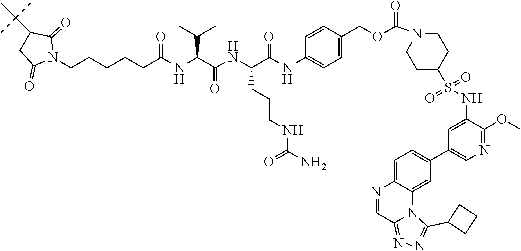 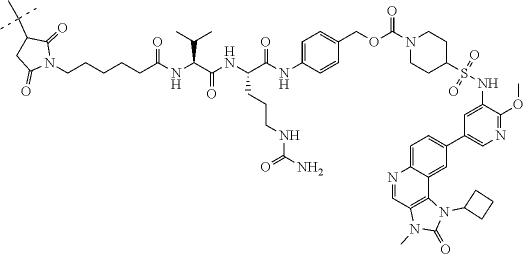 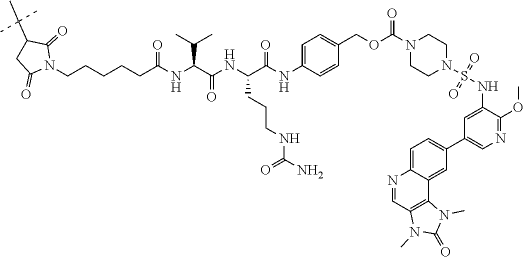 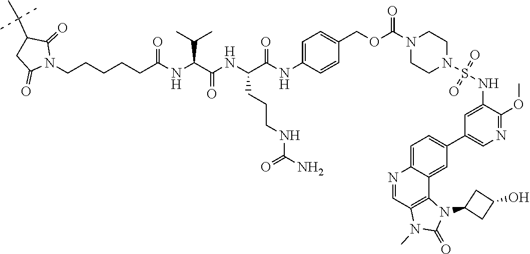 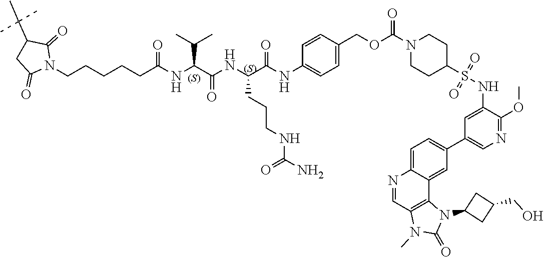 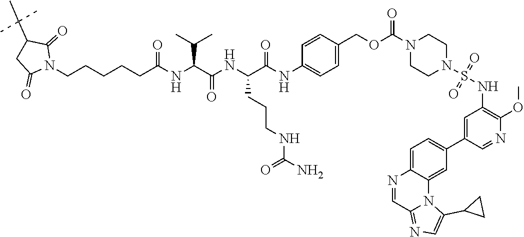 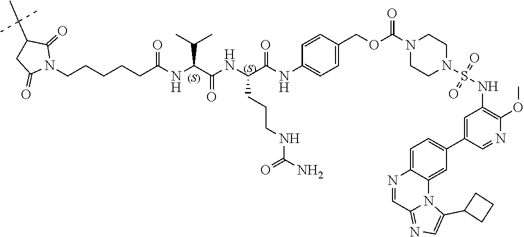 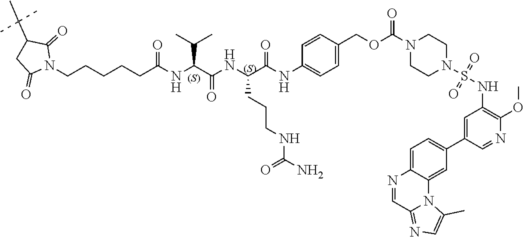 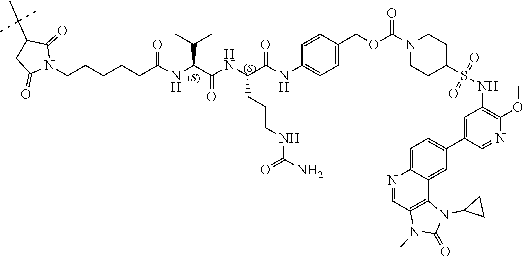 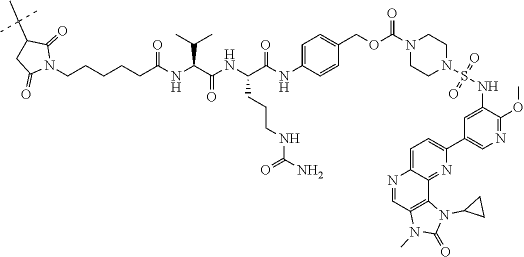 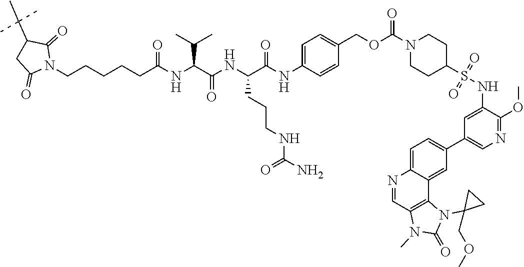 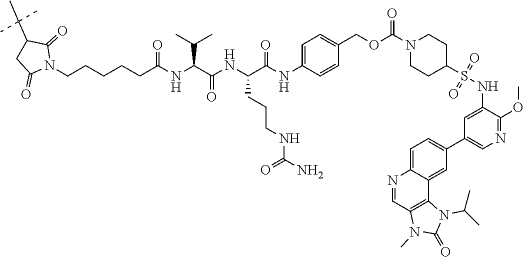 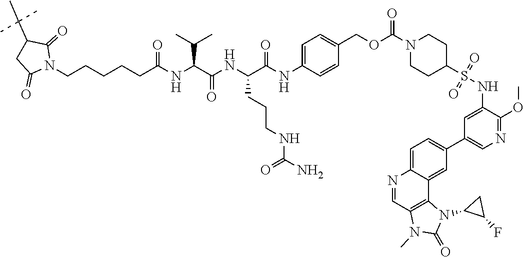 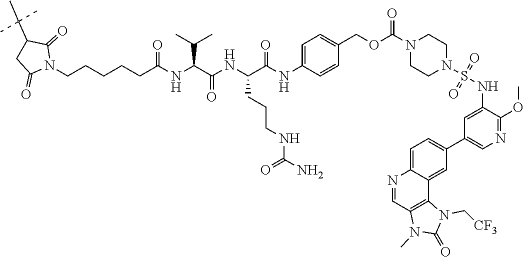 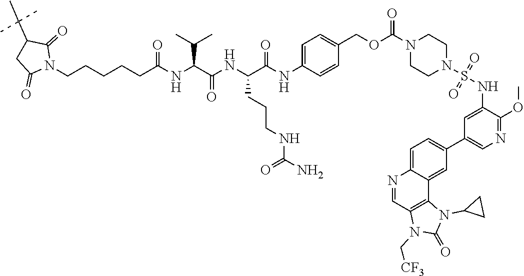 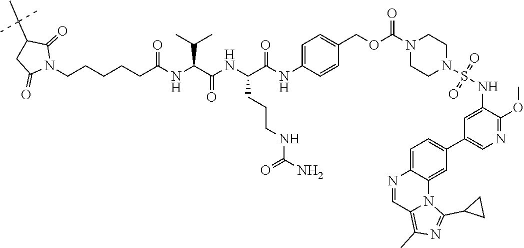
wherein the dashed lines represent the site to which Ab is linked.

89. The drug conjugate or the pharmaceutically acceptable salt thereof, or the solvate, the racemic mixture, the enantiomer, the diastereomer or the tautomer thereof according to claim 88, wherein the drug conjugate is selected from the group consisting of:
Structure

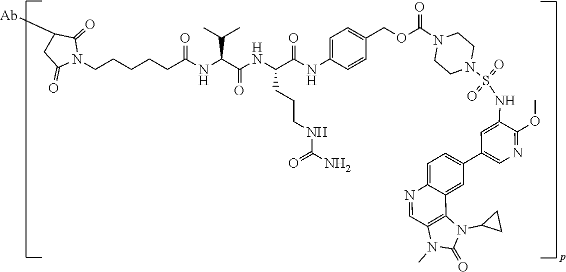

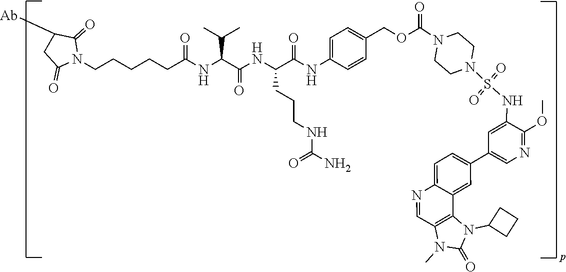

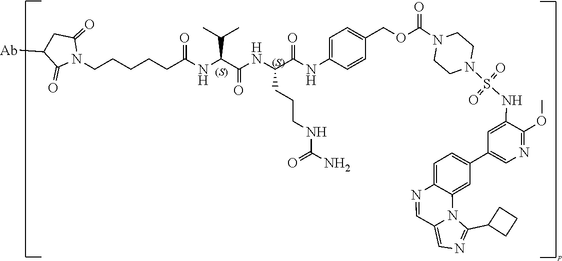

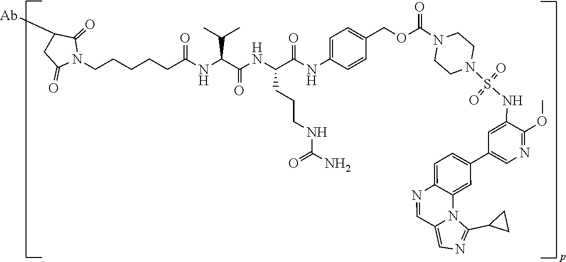

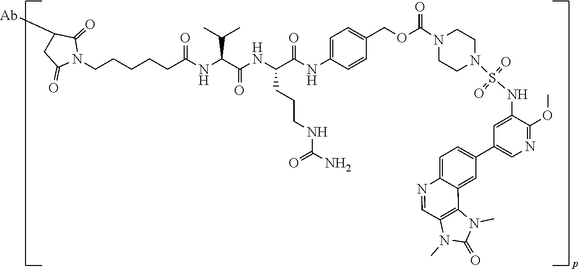

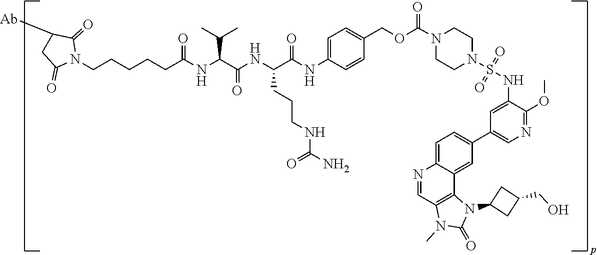

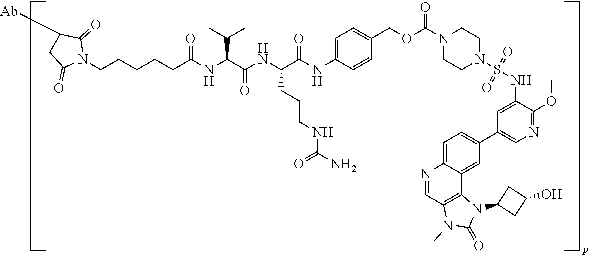

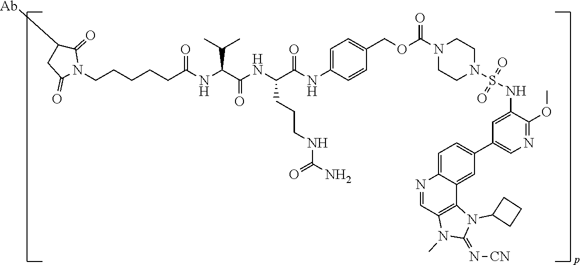

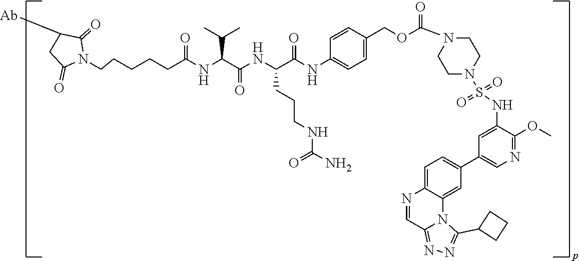

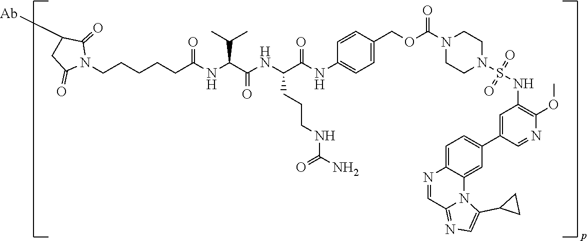

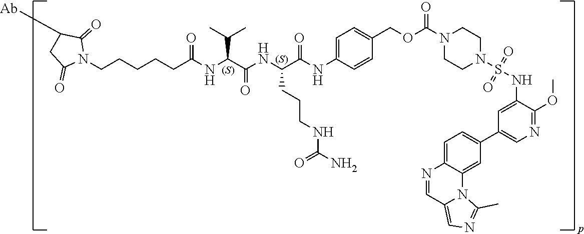

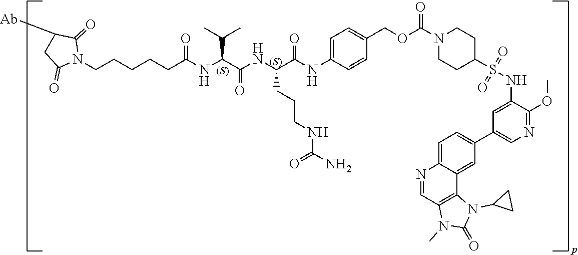

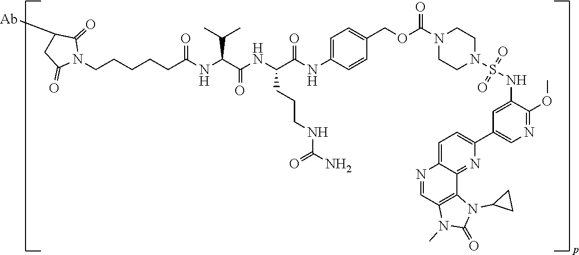

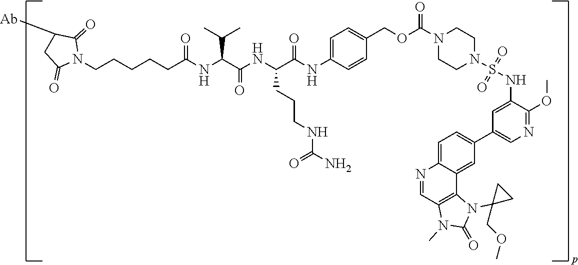

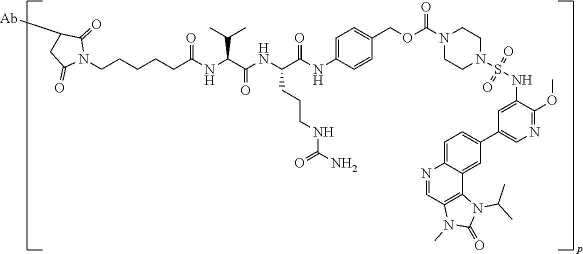

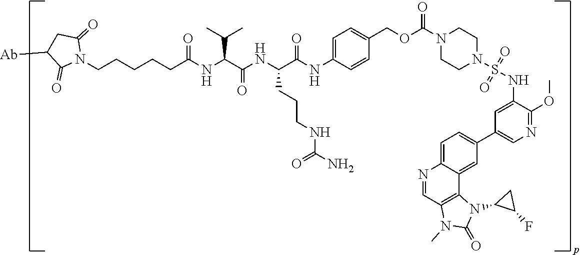

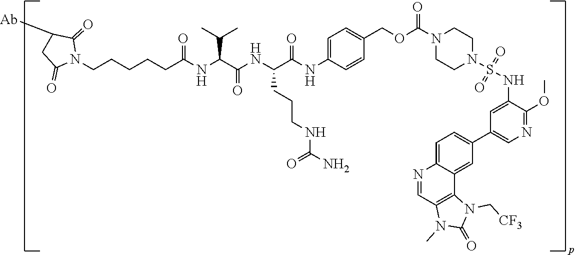

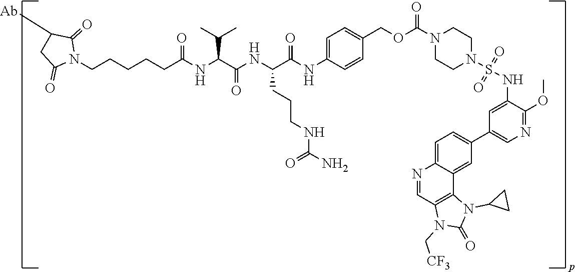

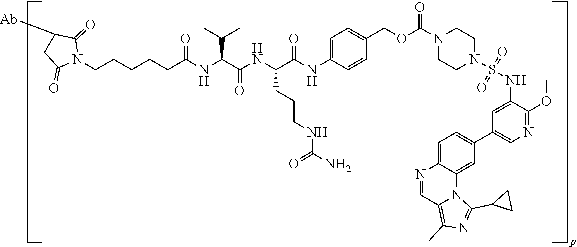

90. The drug conjugate or the pharmaceutically acceptable salt thereof, or the solvate, the racemic mixture, the enantiomer, the diastereomer or the tautomer thereof according to claim 89, wherein p is selected from 2, 3, 4, 5, 6, 7, and 8.

91. The drug conjugate or the pharmaceutically acceptable salt thereof, or the solvate, the racemic mixture, the enantiomer, the diastereomer or the tautomer thereof according to claim 90, wherein Ab is trastuzumab.

92. The drug conjugate or the pharmaceutically acceptable salt thereof, or the solvate, the racemic mixture, the enantiomer, the diastereomer or the tautomer thereof according to claim 82, having an average DAR of 2-8.

93. The drug conjugate or the pharmaceutically acceptable salt thereof, or the solvate, the racemic mixture, the enantiomer, the diastereomer or the tautomer thereof according to claim 92, having an average DAR of 3-4, 3.8-4.2, 3-5, 4-5.5, 5-7, 6.5-8, or 6-8.

94. The drug conjugate or the pharmaceutically acceptable salt thereof, or the solvate, the racemic mixture, the enantiomer, the diastereomer or the tautomer thereof according to claim 82, wherein the drug conjugate is selected from the group consisting of:

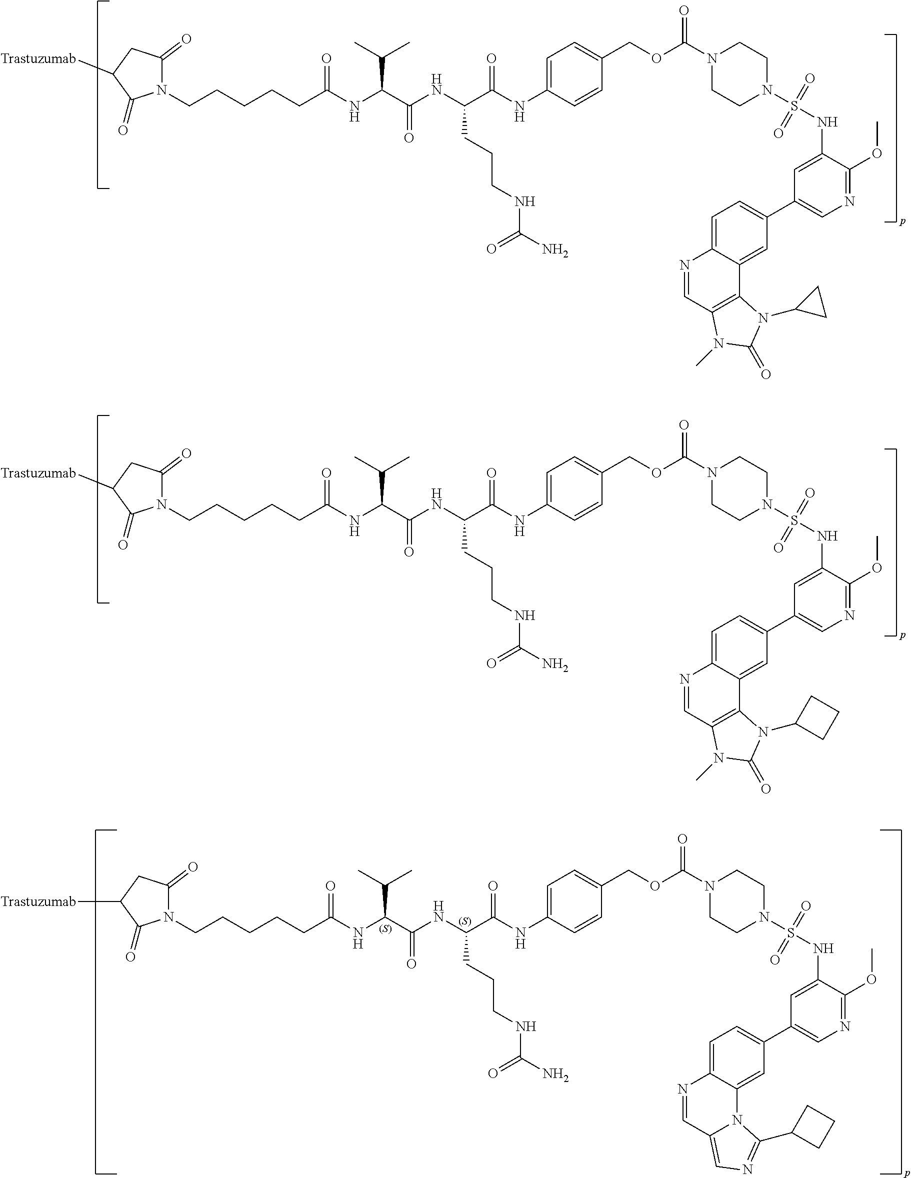

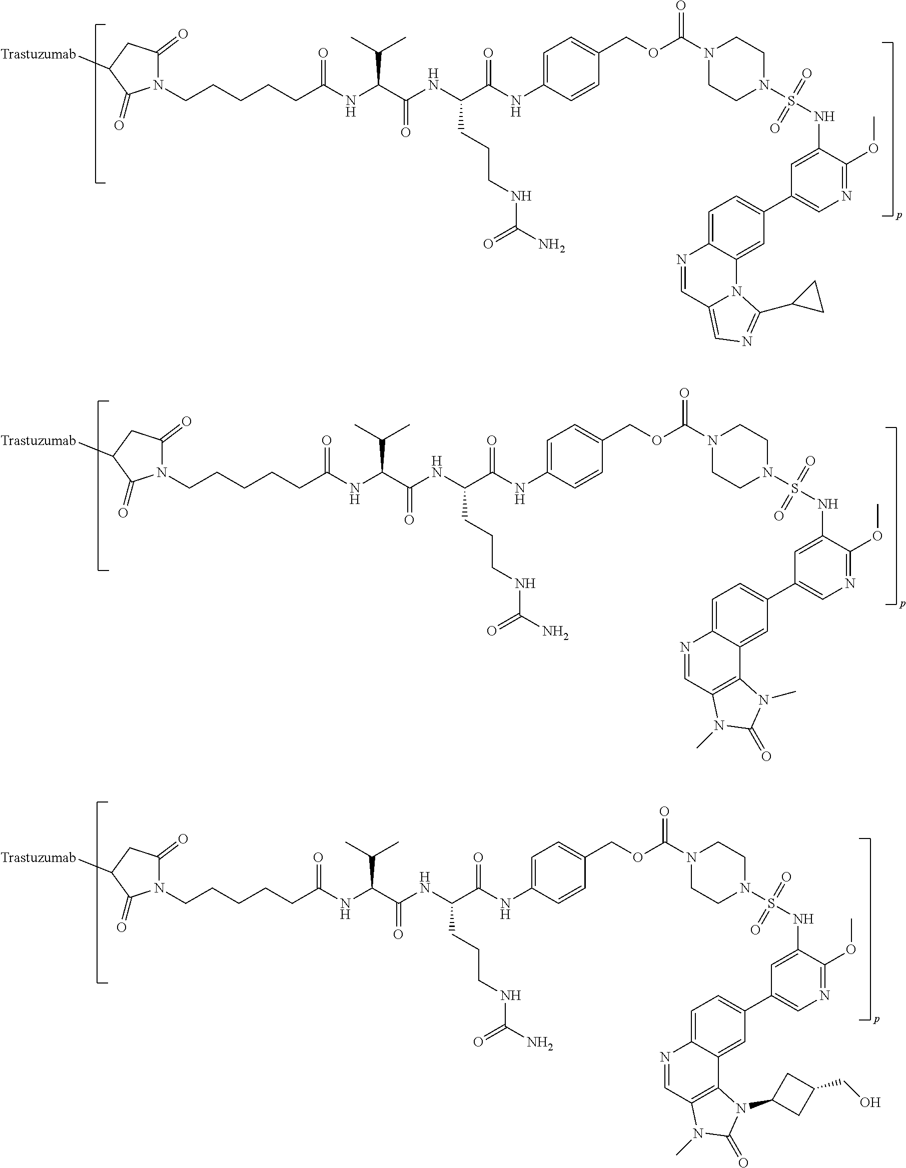

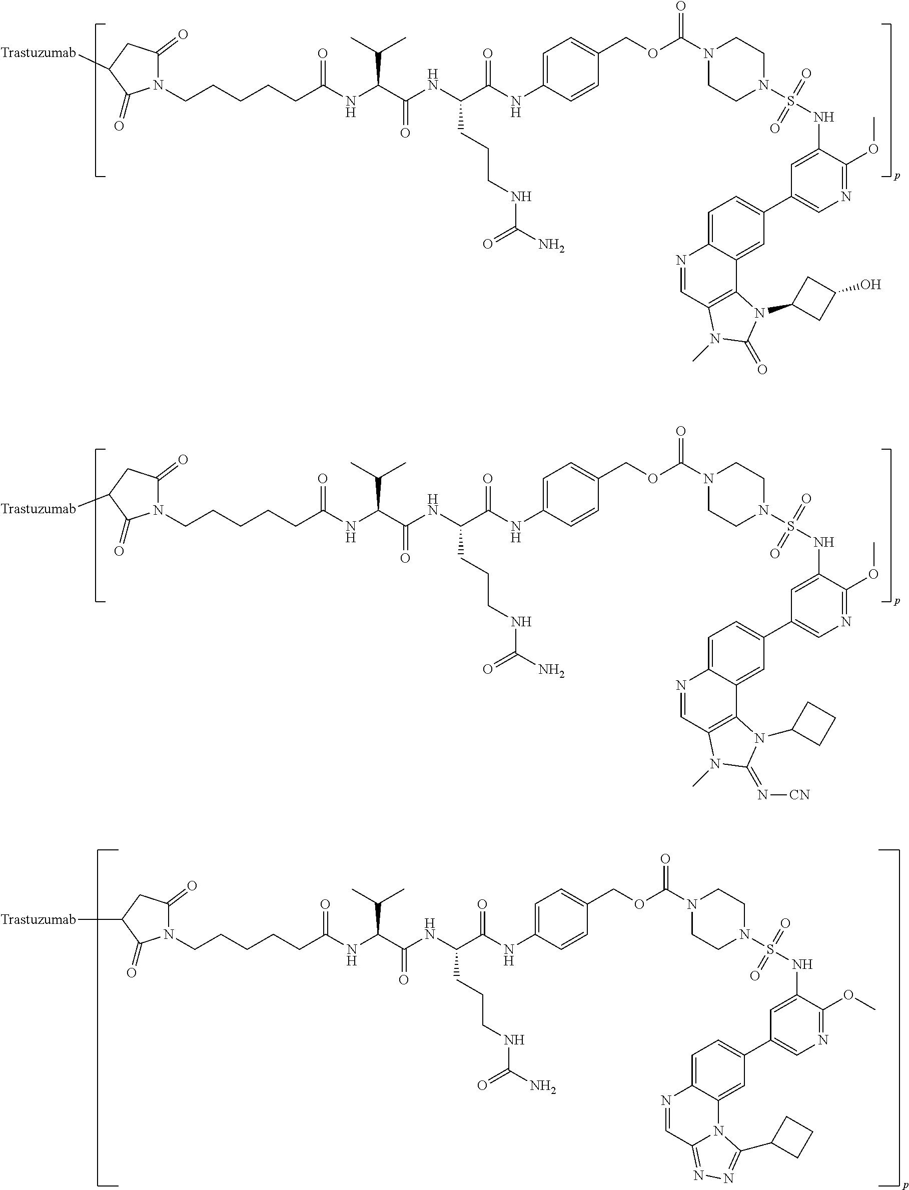

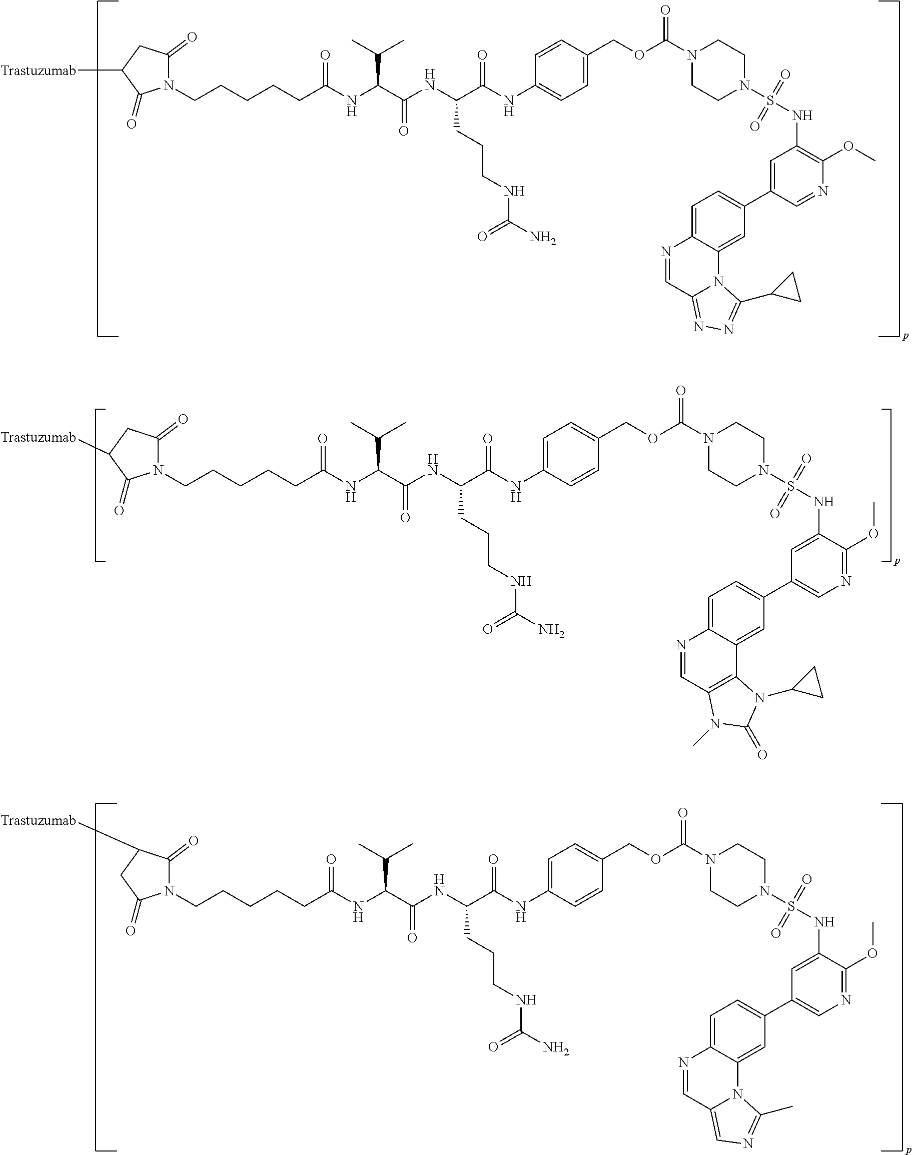

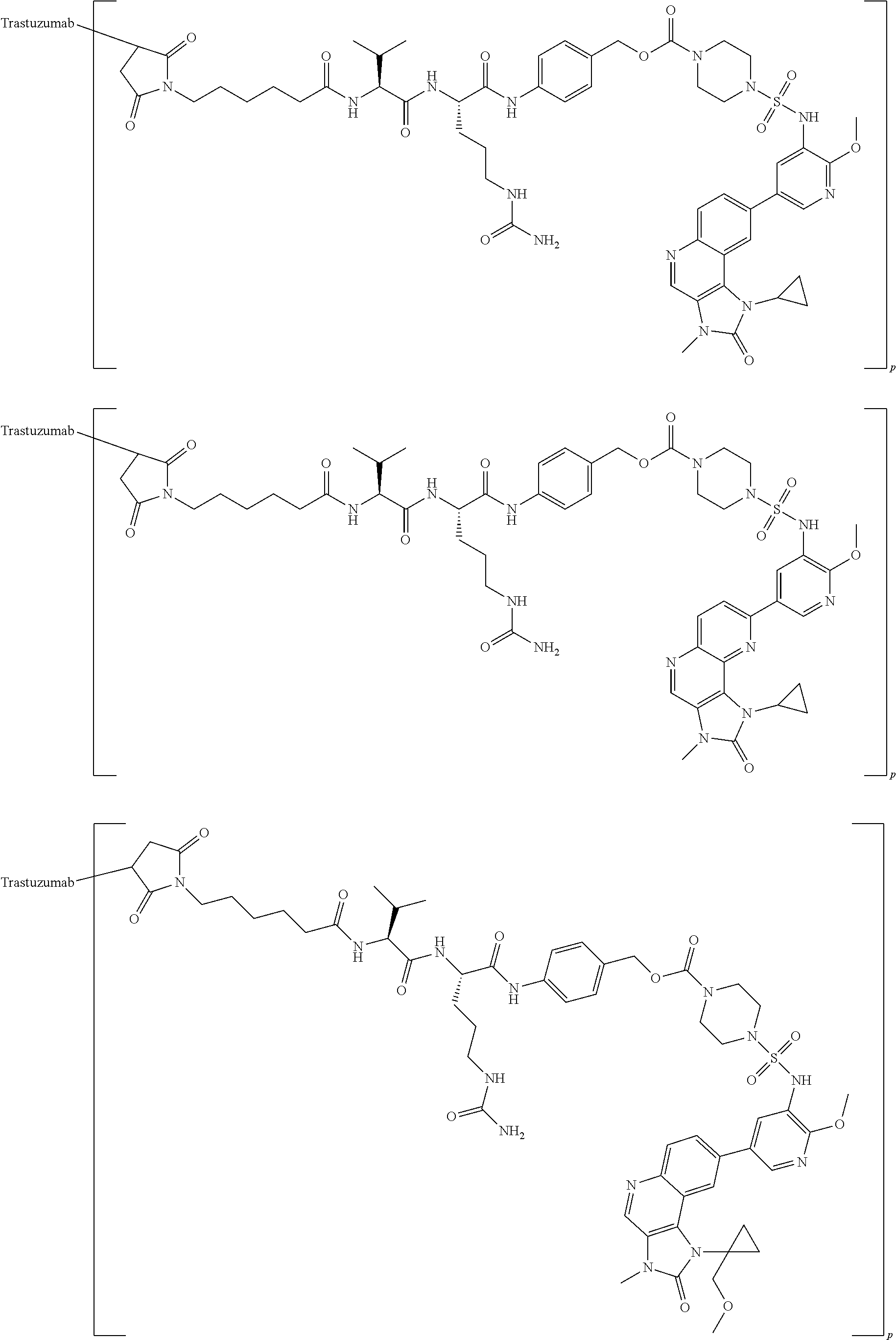

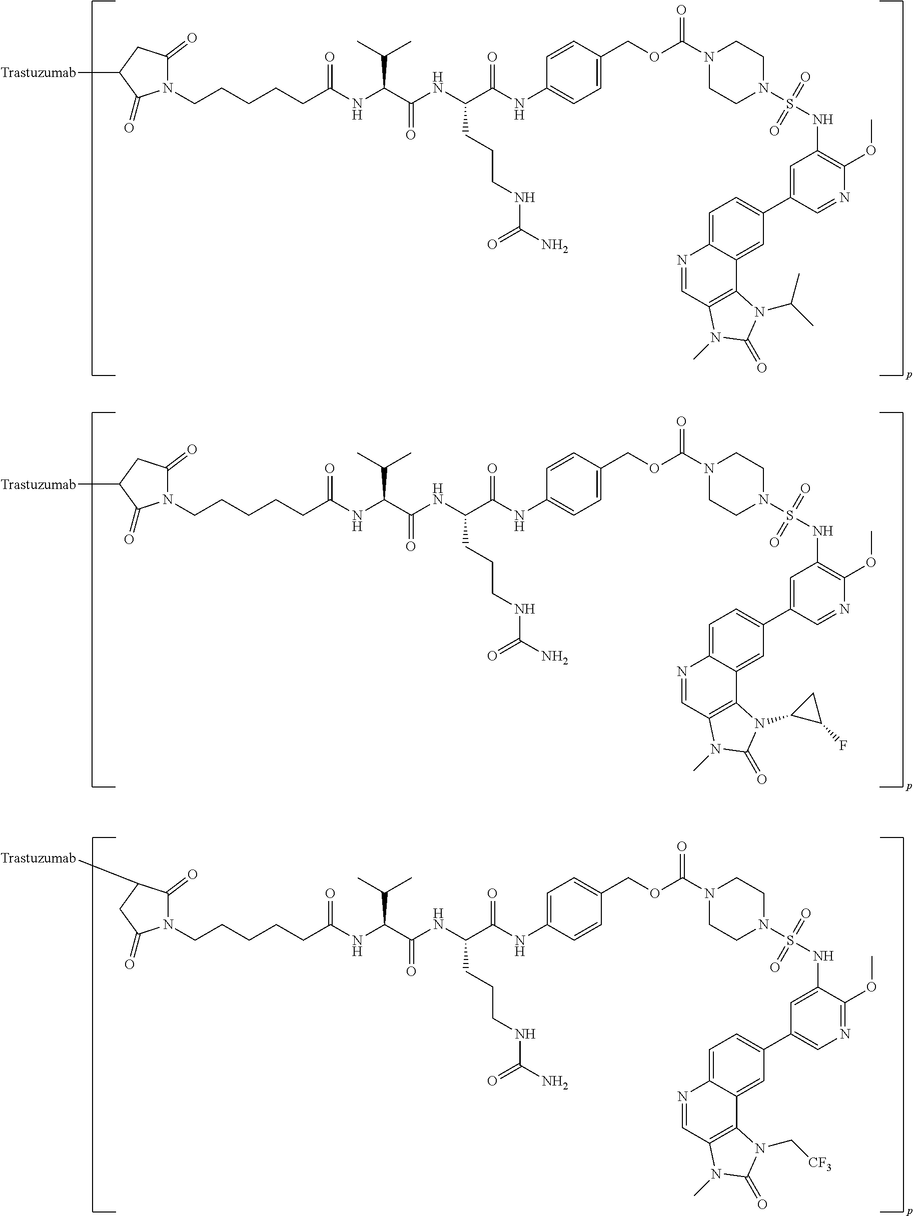

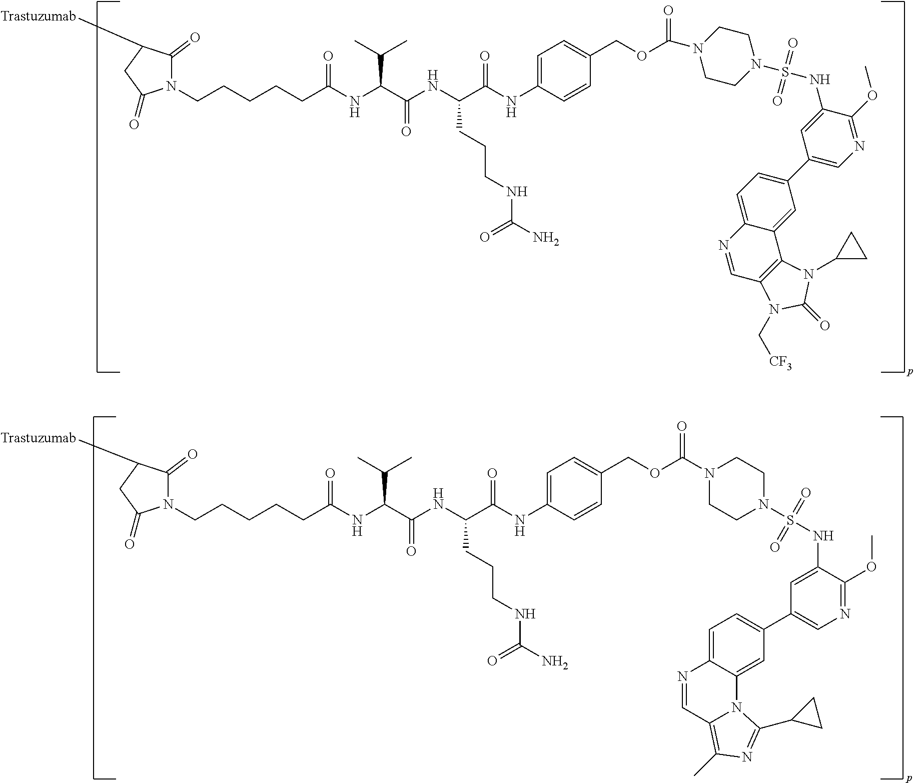

95. A pharmaceutical composition comprising the drug conjugate or the pharmaceutically acceptable salt thereof, or the solvate, the racemic mixture, the enantiomer, the diastereomer or the tautomer thereof according to claim 82, and a pharmaceutically acceptable carrier.

96. A method of treating a disease or condition responsive to the inhibition of PI3K and/or PIKK, comprising administering an effective amount of the drug conjugate or the pharmaceutically acceptable salt thereof according to claim 82 to a subject in need thereof.

97. The method according to claim 96, wherein the disease or condition is selected from: a cancer or an autoimmune disease.

98. The method according to claim 97, wherein the autoimmune disease is selected from: rheumatoid arthritis, chronic obstructive pulmonary disease, allergic rhinitis, asthma, acquired hemophilia A (AHA), idiopathic thrombocytopenia (ITP), and activated phosphoinositide 3-kinase-delta syndrome (APDS).

99. The method according to claim 97, wherein the cancer is a solid tumor or a hematological malignancy, preferably breast cancer, multiple myeloma, Burkitt lymphoma, diffuse large B-cell lymphoma, or non-small cell lung cancer.


SEQUENCE LISTING (60 sequences)

SEQ ID NO: 1 - protein, 5 aa, synthetic construct
DTYIH

SEQ ID NO: 2 - protein, 17 aa, synthetic construct
RIYPTNGYTRYADSVKG

SEQ ID NO: 3 - protein, 11 aa, synthetic construct
WGGDGFYAMDY

SEQ ID NO: 4 - protein, 11 aa, synthetic construct
RASQDVNTAVA

SEQ ID NO: 5 - protein, 7 aa, synthetic construct
SASFLYS

SEQ ID NO: 6 - protein, 9 aa, synthetic construct
QQHYTTPPT

SEQ ID NO: 7 - protein, 120 aa, synthetic construct [sequence omitted]

SEQ ID NO: 8 - protein, 107 aa, synthetic construct [sequence omitted]

SEQ ID NO: 9 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
SFAMS

SEQ ID NO: 12 - protein, 17 aa, synthetic construct
AISGSGGGTYYADSVKG

SEQ ID NO: 13 - protein, 13 aa, synthetic construct
DKILWFGEPVFDY

SEQ ID NO: 14 - protein, 11 aa, synthetic construct
RASQSVSSYLA

SEQ ID NO: 15 - protein, 7 aa, synthetic construct
DASNRAT

SEQ ID NO: 16 - protein, 9 aa, synthetic construct
QQRSNWPPT

SEQ ID NO: 17 - protein, 122 aa, synthetic construct [sequence omitted]

SEQ ID NO: 18 - protein, 107 aa, synthetic construct [sequence omitted]

SEQ ID NO: 19 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
SYNMH

SEQ ID NO: 22 - protein, 17 aa, synthetic construct
AIYPGNGDTSYNQKFKG

SEQ ID NO: 23 - protein, 12 aa, synthetic construct
STYYGGDWYFNV

SEQ ID NO: 24 - protein, 10 aa, synthetic construct
RASSSVSYIH

SEQ ID NO: 25 - protein, 7 aa, synthetic construct
ATSNLAS

SEQ ID NO: 26 - protein, 9 aa, synthetic construct
QQWTSNPPT

SEQ ID NO: 27 - protein, 121 aa, synthetic construct [sequence omitted]

SEQ ID NO: 28 - protein, 106 aa, synthetic construct [sequence omitted]

SEQ ID NO: 29 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 5 aa, synthetic construct
DYAMH

SEQ ID NO: 32 - protein, 18 aa, synthetic construct
GDYSGSRYWFAYNQNFQG

SEQ ID NO: 33 - protein, 12 aa, synthetic construct
GDYSGSRYWFAY

SEQ ID NO: 34 - protein, 11 aa, synthetic construct
QASENIYGALN

SEQ ID NO: 35 - protein, 7 aa, synthetic construct
GASNLAT

SEQ ID NO: 36 - protein, 9 aa, synthetic construct
QQVLSSPYT

SEQ ID NO: 37 - protein, 121 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSS

SEQ ID NO: 38 - protein, 107 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIK

SEQ ID NO: 39 - protein, 451 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 214 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, synthetic construct
SYNMN

SEQ ID NO: 42 - protein, 17 aa, synthetic construct
YISSSSSTIYYADSVKG

SEQ ID NO: 43 - protein, 8 aa, synthetic construct
AYYYGMDV

SEQ ID NO: 44 - protein, 11 aa, synthetic construct
RASQGISGWLA

SEQ ID NO: 45 - protein, 7 aa, synthetic construct
AASTLQS

SEQ ID NO: 46 - protein, 9 aa, synthetic construct
QQANSFPPT

SEQ ID NO: 47 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 48 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 49 - protein, 447 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 214 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 5 aa, synthetic construct
GYFMN

SEQ ID NO: 52 - protein, 17 aa, synthetic construct
RIHPYDGDTFYNQKFQG

SEQ ID NO: 53 - protein, 9 aa, synthetic construct
YDGSRAMDY

SEQ ID NO: 54 - protein, 15 aa, synthetic construct
KASQSVSFAGTSLMH

SEQ ID NO: 55 - protein, 7 aa, synthetic construct
RASNLEA

SEQ ID NO: 56 - protein, 9 aa, synthetic construct
QQSREYPYT

SEQ ID NO: 57 - protein, 118 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 58 - protein, 111 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 59 - protein, 448 aa, synthetic construct
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 218 aa, synthetic construct
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC